(19) United States
(12) Patent Application Publication
(10) Pub. No.: US 2024/0103007 A1
(43) Pub. Date: Mar. 28, 2024

(54) MASS SPECTROMETRY SAMPLE PROCESSING METHODS, CHROMATOGRAPHY DEVICES, AND DATA ANALYSIS TECHNIQUES FOR BIOMARKER ANALYSIS

(71) Applicant: Proteas Bioanalytics, Inc., Glendale, CA (US)

(72) Inventors: Spiros D. GARBIS, Glendale, CA (US); Dimitrios ILIOPOULOS, Glendale, CA (US)

(73) Assignee: Proteas Bioanalytics, Inc., Glendale, CA (US)

(21) Appl. No.: 18/267,411

(22) PCT Filed: Dec. 14, 2021
(86) PCT No.: PCT/US2021/063407

§ 371 Date: Jun. 14, 2023

Related U.S. Application Data
(60) Provisional application No. 63/125,955, filed on Dec. 15, 2020.

(51) Int. Cl. G01N 33/68; B01L 3/00

(52) U.S. Cl. CPC G01N 33/6848; B01L 3/502715; B01L 3/502746; B01L 2400/086; G01N 2800/324

(57) ABSTRACT

In certain aspects, the present disclosure is directed to platforms, including methods, devices, and components thereof, for processing samples for mass spectrometry. In other aspects, provided herein are analysis platforms for analyzing mass spectrometry data, including that obtained from mass spectrometry analysis of the samples obtained from the methods and devices described herein. In other aspects, provided are identified proteomic signatures of a condition in an individual, such as a coronary artery disease (CAD) proteomic signature.

### CROSS-REFERENCE TO RELATED APPLICATIONS

This application claims priority to and the benefit of U.S. Provisional Patent Application No. 63/125,955, filed on Dec. 15, 2020, which is incorporated herein by reference in its entirety.

### TECHNICAL FIELD

In certain aspects, the present disclosure is directed to platforms, including methods, devices, and components thereof, for processing samples for mass spectrometry. In other aspects, provided herein are analysis platforms for analyzing mass spectrometry data, including that obtained from mass spectrometry analysis of the samples obtained from the methods and devices described herein.

### BACKGROUND

Mass spectrometry is a useful tool for analyzing samples containing an array of different types of components ranging from small molecules to nucleic acids to polypeptides. Samples, such as those from biological or environmental origin, can be highly complex and contain components at extremely different concentrations having different physical and chemical properties. For example, common samples are known to contain components exceeding 10 orders of magnitude in dynamic range, and be composed of hydrophilic and hydrophobic peptides and proteins, primary and secondary metabolites, native peptides, small molecule metabolites, and nucleic acids, such as RNA and DNA, including microRNA, circular and long non-coding RNA, and mitochondrial RNA. Existing methods are not entirely satisfactory in the unbiased capture of a wide spectrum of proteins and other biomolecules in fluid samples. Improved methods are needed for the discovery of biomolecules from biological samples as biomarkers associated with biological phenomenon, such as disease. The provided embodiments address these needs.

### BRIEF SUMMARY

In some aspects, provided herein is a method for processing a test sample, the method comprising: (a) subjecting the test sample to a size-exclusion chromatography (SEC) technique using a SEC microfluidic device, wherein the test sample comprises one or more biomolecules and a chaotropic agent, and wherein the SEC microfluidic device comprises a plurality of interconnected channels; (b) collecting one or more fractions eluted from the SEC microfluidic device; (c) subjecting one or more of the fractions from the SEC microfluidic device to a proteolytic technique; and (d) subjecting one or more of the fractions to a reversed-phase liquid chromatography (RPLC) technique to prepare a fraction for introduction to a mass spectrometer, wherein the one or more RPLC-fractions comprise (i) zero or more fractions obtained from the SEC microfluidic device; and (ii) zero or more fractions subjected to the proteolytic technique.

In some aspects, provided herein is a method for processing a test sample for a mass spectrometry analysis, the method comprising: (a) subjecting the test sample to a size-exclusion chromatography (SEC) technique using a SEC microfluidic device, wherein the test sample comprises one or more biomolecules and a chaotropic agent, and wherein the SEC microfluidic device comprises a plurality of interconnected channels; (b) collecting a plurality of fractions eluted from the SEC microfluidic device; (c) subjecting one or more of the plurality of fractions from the SEC microfluidic device to a proteolytic technique; and (d) individually subjecting one or more fractions from one or both of steps (b) and (c) to a reversed-phase liquid chromatography (RPLC) technique using a RPLC microfluidic device under conditions to prepare a component of each of the one or more fractions for introduction to a mass spectrometer, wherein the RPLC microfluidic device comprises a plurality of interconnected channels comprising a reversed-phase medium, and wherein the RPLC microfluidic device is coupled to an electrospray ionization source.

In some embodiments, the test sample a biological sample. In some embodiments, the test sample is from an individual. In some embodiments, the test sample has a concentration of the chaotropic agent of about 5 M to about 8 M. In some embodiments, the chaotropic agent comprises guanidine or a salt thereof, guanidinium or a salt thereof, potassium or a salt thereof, lithium or a salt thereof, magnesium or a salt thereof, or sodium or a salt thereof. In some embodiments, the chaotropic agent is guanidine hydrochloride or guanidinium chloride. In some embodiments, the chaotropic agent in the test sample is from a liquid fixative.

In some embodiments, the test sample has a concentration of a viscosity modifying agent of about 5% to about 40%. In some embodiments, the viscosity modifying agent is glycerol. In some embodiments, the test sample comprises at least about 6 M guanidine and about 10% to about 30% glycerol.

In some embodiments, the test sample subjected to the SEC technique using the SEC microfluidic device has a volume of about 1 μL to about 200 μL. In some embodiments, the range of the concentration of the mobile phase chaotropic agent of the SEC technique is within about +/−40% of the pre-determined concentration of the chaotropic agent of the test sample. In some embodiments, the SEC technique comprises use of a SEC mobile phase having a concentration of a mobile phase chaotropic agent within a range of the chaotropic agent in the test sample.

In some embodiments, the mobile phase chaotropic agent of the SEC technique is the same as the chaotropic agent of the test sample. In some embodiments, the mobile phase chaotropic agent of the SEC technique is different than the chaotropic agent of the test sample.

In some embodiments, the SEC mobile phase comprises a mobile phase chaotropic agent at a concentration of about 4 M to about 8 M. In some embodiments, the mobile phase chaotropic agent of the SEC technique comprises guanidine or a salt thereof, guanidinium or a salt thereof, lithium or a salt thereof, magnesium or a salt thereof, or sodium or a salt thereof. In some embodiments, the mobile phase chaotropic agent of the SEC technique is selected from the group consisting of guanidine hydrochloride, guanidinium chloride, guanidinium thiocynante, lithium perchlorate, lithium acetate, magnesium chloride, potassium acetate, and sodium iodide.

In some embodiments, the SEC mobile phase comprises a mobile phase viscosity modifying agent. In some embodiments, the mobile phase viscosity modifying agent of the SEC technique has a concentration of about 5% to about 40%. In some embodiments, the viscosity modifying agent is glycerol. In some embodiments, the mobile phase viscosity modifying agent of the SEC technique is the same as the viscosity modifying agent of the liquid fixative. In some embodiments, the mobile phase viscosity modifying agent of the SEC technique is different than the viscosity modifying agent of the liquid fixative. In some embodiments, the test sample comprises at least about 6 M guanidine and about 10% to about 30% glycerol.

In some embodiments, the SEC technique is an isocratic SEC technique. In some embodiments, the SEC technique comprises use of a mobile phase flow rate of about 1 μL/minute to about 5 μL/minute.

In some embodiments, the SEC technique is performed at an elevated temperature. In some embodiments, the SEC technique is performed at a temperature of about 45° C. to about 60° C. In some embodiments, the SEC technique is performed at a substantially consistent temperature.

In some embodiments, the SEC microfluidic device comprises a SEC medium. In some embodiments, the SEC medium is a material having an average pore size of about 10 nm to about 500 nm. In some embodiments, the SEC medium is an inner surface of each of the plurality of interconnected channels. In some embodiments, the inner surface material of the plurality of interconnected channels of the SEC microfluidic device has a thickness of about 0.5 μm to about 2 μm.

In some embodiments, the plurality of interconnected channels of the SEC microfluidic device are configured in an open tubular format. In some embodiments, the plurality of interconnected channels of the SEC microfluidic device comprises 8 or more channels. In some embodiments, the plurality of interconnected channels of the SEC microfluidic device comprises 32 channels. In some embodiments, the plurality of interconnected channels of the SEC microfluidic device comprises 64 channels.

In some embodiments, each of the plurality of interconnected channels of the SEC microfluidic device are in fluidic communication with an input port of the SEC microfluidic device via an upstream network of connection channels. In some embodiments, the upstream network of connection channels, or portions thereof, is connected to a proximal region of each of the plurality of interconnected channels. In some embodiments, the upstream network of connection channels comprises a series of diverging channels configured to split fluid flow from the input port of the SEC microfluidic device to each of the plurality of interconnected channels.

In some embodiments, each of the plurality of interconnected channels of the SEC microfluidic device is in fluidic communication with an output port of the SEC microfluidic device via a downstream network of connection channels. In some embodiments, the downstream network of connection channels, or portions thereof, is connected to a distal region of each of the plurality of interconnected channels. In some embodiments, the downstream network of connection channels comprises a series of converging channels configured to combine fluid flow from the plurality of interconnected channels of the SEC microfluidic device to the output port. In some embodiments, the plurality of interconnected channels of the SEC microfluidic device are only connected via the upstream network of connection channels or the downstream network of connection channels.

In some embodiments, each of the plurality of interconnected channels of the SEC microfluidic device has a length of about 2 cm to about 50 cm. In some embodiments, each of the plurality of interconnected channels of the SEC microfluidic device has a width of about 1 μm to about 15 μm.

In some embodiments, each of the plurality of interconnected channels of the SEC microfluidic device has a depth of about 1 μm to about 15 μm.

In some embodiments, the plurality of interconnected channels of the SEC microfluidic device are formed via a pillar array. In some embodiments, the pillar array is an amorphous pillar array. In some embodiments, the pillar array is a non-amorphous pillar array. In some embodiments, the pillar array forms an inner surface of each of the plurality of interconnected channels of the SEC microfluidic device.

In some embodiments, the SEC microfluidic device comprises a quartz substrate. In some embodiments, the SEC microfluidic device comprises a monolithic substrate forming the plurality of interconnected channels. In some embodiments, the SEC microfluidic device comprises a quartz monolithic substrate.

In some embodiments, the SEC microfluidic device comprises a three-dimensional (3D) printed substrate.

In some embodiments, collecting the plurality of fractions eluted from the SEC microfluidic device is performed using a fraction collector. In some embodiments, each of the plurality of fractions is collected from the SEC microfluidic device based on time. In some embodiments, each of the plurality of fractions is collected from the SEC microfluidic device for a period of about 30 seconds to about 5 minutes. In some embodiments, each of the plurality of fractions is collected from the SEC microfluidic device for a uniform amount of time. In some embodiments, a fraction of the plurality of fractions is collected from the SEC microfluidic device for a different amount of time than another fraction of the plurality of fractions.

In some embodiments, each of the plurality of fractions is collected from the SEC microfluidic device based on volume of eluate from the SEC microfluidic device. In some embodiments, each of the plurality of fractions collected from the SEC microfluidic device has a volume of about 1 μL to about 20 μL. In some embodiments, the plurality of fractions collected from the SEC microfluidic device has a uniform volume. In some embodiments, a fraction of the plurality of fractions collected from the SEC microfluidic device has different volume than another fraction of the plurality of fractions.

In some embodiments, the plurality of fraction is about 5 to about 50 fractions. In some embodiments, the plurality of fraction is about 12 to about 24 fractions.

In some embodiments, the proteolytic technique comprises an enzyme-based digestion technique. In some embodiments, the enzyme-based digestion technique comprises the use of an enzyme selected from the group consisting of trypsin, chymotrypsin, pepsin, LysC, LysN, AspN, GluC and ArgC, or a combination thereof.

In some embodiments, the enzyme-based digestion technique comprises a step of diluting the fraction eluted from the SEC microfluidic device. In some embodiments, the diluting comprises admixing the fraction eluted from the SEC microfluidic device with water to reach a concentration of the chaotropic agent. In some embodiments, the final concentration of the concentration of the chaotropic agent for the enzymatic digestion is about 0.5 M.

In some embodiments, the enzyme-based digestion technique does not comprise a buffer exchange step. In some embodiments, the enzyme-based digestion technique does not comprise an alkylation step. In some embodiments, the enzyme-based digestion technique does not comprise a reduction step.

In some embodiments, the proteolytic technique comprises a non-enzyme-based approach.

In some embodiments, the method further comprises subjecting one or more of the plurality of fractions from the SEC microfluidic device and/or one or more of the plurality of fractions subjected to the proteolytic technique to a quantitative labeling technique, wherein the quantitative labeling technique is performed prior to the reversed-phase liquid chromatography (RPLC) technique using the RPLC microfluidic device.

In some embodiments, the quantitative labeling technique comprises use of an isobaric mass tag. In some embodiments, the quantitative labeling technique comprises use of a Tandem Mass Tag (TMT).

In some embodiments, the quantitative labeling technique comprises a desalting step.

In some embodiments, the method further comprises admixing an internal standard with one or more of the plurality of fractions from the SEC microfluidic device and/or one or more of the plurality of fractions subjected to the proteolytic technique, wherein the admixing of the internal standard is performed prior to the reversed-phase liquid chromatography (RPLC) technique using the RPLC microfluidic device. In some embodiments, the internal standard is an isotopically-labeled peptide.

In some embodiments, the one or more fractions subjected to the RPLC technique comprises one or more fractions, or portions thereof, obtained from: (i) zero or more fractions obtained from the SEC microfluidic device; and (ii) one or more of the plurality of fractions subjected to the proteolytic technique. In some embodiments, each of the one or more fractions subjected to the RPLC technique comprises the respective fraction of origin admixed with an aqueous solution.

In some embodiments, the fraction subjected to the RPLC technique has a volume of about 1 μL to about 50 μL.

In some embodiments, the RPLC technique comprise use of a RPLC mobile phase. In some embodiments, the RPLC technique comprises a mobile phase flow rate of the RPLC mobile phase of about 0.05 μL/minute to about 2 μL/minute. In some embodiments, the RPLC technique is a gradient RPLC technique.

In some embodiments, the RPLC technique is performed at an elevated temperature. In some embodiments, the RPLC technique is performed at a temperature of about 30° C. to about 100° C. In some embodiments, the RPLC technique is performed at a substantially consistent temperature.

In some embodiments, the reversed-phased medium comprises a RPLC moiety mixture comprising two or more of the following alkyl moieties: C₂, C₄, C₈, and C₁₈. In some embodiments, the RPLC moiety mixture comprises three or more of the following alkyl moieties: C₂, C₄, C₈, and C₁₈. In some embodiments, the RPLC moiety mixture comprises the following alkyl moieties: C₂, C₄, C₈, and C₁₈. In some embodiments, the alkyl moieties of the RPLC moiety mixture are present in equimolar amounts.

In some embodiments, the alkyl moieties of the RPLC moiety mixture are covalently coupled to surfaces of each of the interconnected plurality of channels of the RPLC microfluidic device. In some embodiments, surfaces of each of the interconnected plurality of channels comprise silica (SiO₂).

In some embodiments, the plurality of interconnected channels of the RPLC microfluidic device comprises 8 or more channels. In some embodiments, the plurality of interconnected channels of the RPLC microfluidic device comprises 32 channels. In some embodiments, the plurality of interconnected channels of the RPLC microfluidic device comprises 64 channels.

In some embodiments, each of the plurality of interconnected channels of the RPLC microfluidic device are in fluidic communication with an input port of the RPLC microfluidic device via an upstream network of connection channels. In some embodiments, the upstream network of connection channels, or portions thereof, is connected to a proximal region of each of the plurality of interconnected channels. In some embodiments, the upstream network of connection channels comprises a series of diverging channels configured to split fluid flow from the input port of the RPLC microfluidic device to each of the plurality of interconnected channels.

In some embodiments, each of the plurality of interconnected channels of the RPLC microfluidic device is in fluidic communication with an output port of the RPLC microfluidic device via a downstream network of connection channels. In some embodiments, the downstream network of connection channels, or portions thereof, is connected to a distal region of each of the plurality of interconnected channels. In some embodiments, the downstream network of connection channels comprises a series of converging channels configured to combine fluid flow from the plurality of interconnected channels of the RPLC microfluidic device to the output port. In some embodiments, the plurality of interconnected channels of the RPLC microfluidic device are only connected via the upstream network of connection channels or the downstream network of connection channels.

In some embodiments, each of the plurality of interconnected channels of the RPLC microfluidic device has a length of about 2 cm to about 50 cm. In some embodiments, each of the plurality of interconnected channels of the RPLC microfluidic device has a width of about 1 μm to about 15 μm. In some embodiments, each of the plurality of interconnected channels of the RPLC microfluidic device has a depth of about 1 μm to about 15 μm.

In some embodiments, the plurality of interconnected channels of the RPLC microfluidic device are formed via a pillar array. In some embodiments, the pillar array is an amorphous pillar array. In some embodiments, the pillar array is a non-amorphous pillar array. In some embodiments, the pillar array forms an inner surface of each of the plurality of interconnected channels of the RPLC microfluidic device comprises.

In some embodiments, the RPLC microfluidic device comprises an online divert feature. In some embodiments, the online divert feature is a valve and/or a channel. In some embodiments, the online divert feature is positioned between the plurality of interconnected channels of the RPLC microfluidic device and the electrospray ionization device.

In some embodiments, the RPLC microfluidic device comprises a quartz substrate. In some embodiments, the RPLC microfluidic device comprises a monolithic substrate forming the plurality of interconnected channels. In some embodiments, the RPLC microfluidic device comprises a quartz monolithic substrate.

In some embodiments, the RPLC microfluidic device comprises a three-dimensional (3D) printed substrate.

In some embodiments, the RPLC microfluidic device is configured in an open tubular format.

In some embodiments, the RPLC microfluidic device is configured for online desalting.

In some embodiments, the electrospray ionization source is a nano-electrospray ionization source. In some embodiments, the electrospray ionization source is a heated electrospray ionization source.

In some embodiments, the sample is selected from the group consisting of a blood sample, cerebrospinal fluid (CSF) sample, ascitic fluid sample, seminal fluid sample, and nipple aspirate fluid sample. In some embodiments, the sample has a volume of about 10 μL to about 200 μL. In some embodiments, the sample is a blood sample.

In some embodiments, when the sample from the individual is a blood sample, the method further comprises preparing a plasma sample. In some embodiments, preparing the plasma sample comprises subjecting the blood sample to a plasma generation technique. In some embodiments, the plasma generation technique comprises subjecting the sample to a polysulphone medium. In some embodiments, the polysulphone medium is an asymmetric polysulphone material.

In some embodiments, the plasma generation technique is a capillary action filtration technique. In some embodiments, the volume of the blood sample subjected to the plasma generation technique is about 10 μL to about 200 μL.

In some embodiments, the method further comprises admixing the generated plasma sample with the liquid fixative to generate the test sample. In some embodiments, the test sample is not further depleted prior to subjecting the test sample to the SEC technique.

In some embodiments, the plasma generation technique is performed at an ambient temperature.

In some embodiments, the sample has not been subjected to a depletion step prior to the plasma generation technique.

In some embodiments, the method further comprises subjecting the components, or products thereof, eluted from the RPLC microfluidic device to the mass spectrometer. In some embodiments, the method further comprises performing a mass spectrometry analysis of the components, or products thereof, of the sample using the mass spectrometer. In some embodiments, the mass spectrometry analysis comprises an analysis of each fraction subjected to the RPLC technique using the RPLC microfluidic device. In some embodiments, the mass spectrometry analysis comprises obtaining one or more data sets comprising information obtained from the mass spectrometer for each fraction subjected to the RPLC technique using the RPLC microfluidic device.

In some embodiments, a single data set comprises information obtained from the mass spectrometer from a single fraction subjected to the RPLC technique using the RPLC microfluidic device. In some embodiments, each of the one or more data set comprises mass-to-charge (rn/z) and abundance information for ions of the components, or products thereof, introduced to the mass spectrometer.

In some embodiments, each composition of a collection of compositions obtained from any of the methods described herein, is a RPLC microfluidic device eluate.

In some aspects, provided herein is a method of analyzing a composition, the method comprising: (a) subjecting the compositions to a mass spectrometer; and (b) performing a mass spectrometry analysis of the composition, wherein the composition is obtained from a processing technique comprising fractionation of a sample using a SEC technique comprising use of a SEC microfluidic device followed by application of each fraction, or a product thereof, to a RPLC technique.

In some aspects, provided herein is a method of analyzing a collection of compositions using mass spectrometry, the method comprising: (a) subjecting each composition of the collection of compositions to a mass spectrometer; and (b) performing a mass spectrometry analysis of each composition of the collection of compositions, wherein the collection of compositions is obtained from a processing technique comprising fractionation of a test sample using a SEC technique comprising use of a SEC microfluidic device followed by application of each fraction, or a product thereof, to a RPLC technique comprising use of a RPLC microfluidic device.

In some embodiments, the SEC fraction is further processed via a proteolysis technique.

In some embodiments, the method further comprises, based on at least one of the one or more data sets, determining the identities of each of a plurality of the one or more biomolecules in the test sample. In some embodiments, the method further comprises, based on at least one of the one or more data sets, measuring the quantities of each of a plurality of the one or more biomolecules in the test sample.

In some embodiments, the method further comprises identifying a signature comprising one or more identified biomolecules from the determined identities. In some embodiments, the identifying further comprises selecting a subset of the one or more identified biomolecules based on the measured quantities of the one or more identified biomolecules. In some embodiments, the subset of the one or more identified biomolecules is selected based on differential measured quantities of the one or more identified biomolecules compared to a reference sample.

In some embodiments, the method further comprises identifying a signature comprising one or more identified biomolecules, the identifying comprising: based on at least one of the one or more data sets, measuring the quantities of each of a plurality of the one or more biomolecules in the test sample; selecting a subset of the plurality of the one or more biomolecules in the sample based on the measured quantities; and determining the identities of each of the subset of the plurality of the one or more biomolecules in the test sample.

In some embodiments, the subset of the plurality of the one or more biomolecules in the test sample is selected based on differential measured quantities of the plurality of the one or more biomolecules in the test sample compared to a reference sample. In some embodiments, the test sample is a sample from a diseased subject and the reference sample is a sample from a healthy subject or a control subject. In some embodiments, the test sample is a sample from a subject having a pre-condition related to a disease and the reference sample is a sample from a healthy subject or a control subject. In some embodiments, the test sample is a sample from a subject with a disease in an active state and the reference sample is a sample from a subject with the disease in an inactive state, optionally wherein the inactive state is remission. In some embodiments, the test sample is a sample from a subject with a disease at an advanced stage and the reference sample is a sample from a subject with the disease at an early stage.

In some embodiments, a signature comprising a plurality of the identified biomolecules or a subset thereof is identified by a method described herein. In some embodiments, a signature comprising the subset of identified biomolecules is identified by a method described herein.

In some embodiments, the method further comprises providing all or a subset of the identified biomolecules of the signature as input to one or more processes configured to perform gene enrichment analysis, one or more processes configured to perform pathway analysis, and/or one or more processes configured to perform network analysis.

In some embodiments, a method of analyzing biomolecules of a sample comprises providing the identified biomolecules of the signature as input to one or more processes configured to perform gene enrichment analysis, one or more processes configured to perform pathway analysis, and/or one or more processes configured to perform network analysis. In some embodiments, identified biomolecules of one or more molecular types of the signature are provided as the input. In some embodiments, the one or more molecular types comprise proteins. In some embodiments, the one or more molecular types consist only of proteins.

In some aspects, provided herein is a method of analyzing a signature of identified components, comprising performing gene enrichment analysis, pathway analysis, and network analysis in any order, wherein: the signature of identified components comprises a protein set, a transcriptomic set, a peptide set, and/or a metabolite set; and the performing comprises: a process configured to perform gene enrichment analysis; a process configured to perform pathway analysis; a process configured to perform gene enrichment analysis; and a process configured to perform network analysis to identify drug targets.

In some embodiments, the one or more processes configured to perform gene enrichment analysis comprise a process configured to identify one or more gene ontologies each associated with at least one of the identified biomolecules of the signature provided as input, or at least one of the products thereof. In some embodiments, the one or more processes configured to perform gene enrichment analysis comprise: a process configured to identify one or more cellular component gene ontologies each associated with at least one of the identified biomolecules of the signature provided as input, or at least one of the products thereof; a process configured to identify one or more molecular pathway gene ontologies each associated with at least one of the identified biomolecules of the signature provided as input, or at least one of the products thereof; and/or a process configured to identify one or more biological process gene ontologies each associated with at least one of the identified biomolecules of the signature provided as input, or at least one of the products thereof. In some embodiments, the one or more processes configured to perform gene enrichment analysis comprise a process configured to identify one or more regulators of at least one of the identified biomolecules of the signature provided as input, or at least one of the products thereof.

In some embodiments, the one or more processes configured to perform gene enrichment analysis comprise: a process configured to identify one or more transcription factors regulating at least one of the identified biomolecules of the signature provided as input, or at least one of the products thereof; and/or a process configured to identify one or more kinases regulating at least one of the identified biomolecules of the signature provided as input, or at least one of the products thereof.

In some embodiments, the one or more processes configured to perform pathway analysis comprise a process configured to identify one or more pathways each associated with at least one of the identified biomolecules of the signature provided as input, or at least one of the products thereof. In some embodiments, the one or more processes configured to perform pathway analysis comprise: a process configured to identify one or more molecular pathways each associated with at least one of the identified biomolecules of the signature provided as input, or at least one of the products thereof; a process configured to identify one or more signaling pathways each associated with at least one of the identified biomolecules of the signature provided as input, or at least one of the products thereof; and/or a process configured to identify one or more metabolic pathways each associated with at least one of the identified biomolecules of the signature provided as input, or at least one of the products thereof.

In some embodiments, the one or more processes configured to perform network analysis comprise a process configured to identify one or more networks each associated with at least one of the identified biomolecules of the signature provided as input, or at least one of the products thereof. In some embodiments, the one or more processes configured to perform network analysis comprise: a process configured to identify one or more molecular pathways each associated with at least one of the identified biomolecules of the signature provided as input, or at least one of the products thereof; and/or a process configured to identify one or more signaling pathways each associated with at least one of the identified biomolecules of the signature provided as input, or at least one of the products thereof.

In some embodiments, the one or more processes configured to perform network analysis comprise a process configured to identify one or more hubs of one or more networks each associated with at least one of the identified biomolecules of the signature provided as input, or at least one of the products thereof. In some embodiments, the one or more processes configured to perform network analysis comprise a process configured to identify one or more drugs each targeting at least one of the identified biomolecules of the signature provided as input, or at least one of the products thereof, optionally wherein the process is configured to identify one or more drugs each targeting at least one hub of a network comprising a plurality of the identified biomolecules of the signature provided as input. In some embodiments, the one or more processes configured to perform network analysis comprises two processes configured to identify one or more drugs each targeting at least one of the identified biomolecules of the signature provided as input, or at least one of the products thereof, optionally wherein the two processes are configured to identify one or more drugs each targeting at least one hub of a network comprising a plurality of the identified biomolecules of the signature provided as input.

In some aspects, provided herein is a method of analyzing a signature of identified biomolecules, comprising providing a plurality of identified biomolecules to each of a plurality of processes each configured to perform gene enrichment analysis, pathway analysis, or network analysis, wherein: the providing is performed in any order; the plurality of identified biomolecules comprises a protein set, a transcriptomic set, a peptide set, and/or a metabolite set; and the plurality of processes comprise: a process configured to perform gene enrichment analysis to identify one or more gene ontologies each associated with at least one of the plurality of identified biomolecules provided as input, or at least one of the products thereof; a process configured to perform pathway analysis to identify one or more signaling pathways each associated with at least one of the plurality of identified biomolecules provided as input, or at least one of the products thereof; a process configured to perform gene enrichment analysis to identify one or more transcription factors regulating at least one of the plurality of identified biomolecules provided as input, or at least one of the products thereof; a process configured to perform gene enrich analysis to identify one or more kinases regulating a gene product of at least one of the plurality of identified biomolecules provided as input, or at least one of the products thereof; a process configured to perform network analysis to identify one or more networks each associated with at least one of the plurality of identified biomolecules provided as input, or at least one of the products thereof; and each of two processes configured to perform network analysis to identify one or more drugs each targeting at least one of the plurality of identified biomolecules provided as input, or at least one of the products thereof.

In some aspects, provided herein is a method of analyzing a protein signature, comprising providing a plurality of proteins to each of a plurality of processes each configured to perform gene enrichment analysis, pathway analysis, or network analysis, wherein the providing is performed in any order, and the plurality of processes comprise: a process configured to perform gene enrichment analysis to identify one or more gene ontologies each associated with at least one of the plurality of proteins provided as input, or at least one of the products thereof; a process configured to perform pathway analysis to identify one or more signaling pathways each associated with at least one of the plurality of proteins provided as input, or at least one of the products thereof; a process configured to perform gene enrichment analysis to identify one or more transcription factors regulating at least one of the plurality of proteins provided as input, or at least one of the products thereof; a process configured to perform gene enrich analysis to identify one or more kinases regulating a gene product of at least one of the plurality of proteins provided as input, or at least one of the products thereof; a process configured to perform network analysis to identify one or more networks each associated with at least one of the plurality of proteins provided as input, or at least one of the products thereof; and each of two processes configured to perform network analysis to identify one or more drugs each targeting at least one of the plurality of proteins provided as input, or at least one of the products thereof.

In some aspects, provided herein is a size-exclusion chromatography (SEC) microfluidic device comprising: an input port; an upstream network of connection channels; and a plurality of interconnected channels, wherein each channel of the plurality of interconnected channels is in an open tubular format, wherein each channel of the plurality of interconnected channels comprises an inner surface comprising a SEC medium, and wherein each channel of the plurality of interconnected channels is in fluidic communication with the input port via the upstream network of connection channels.

In some embodiments, the inner surface comprising the SEC medium of the SEC microfluidic device has a thickness of about 0.5 μm to about 2 μm. In some embodiments, the SEC medium of the SEC microfluidic device is a material having an average pore size of about 10 nm to about 500 nm.

In some embodiments, the plurality of interconnected channels of the SEC microfluidic device comprises between 8 and 100 interconnected channels. In some embodiments, the plurality of interconnected channels of the SEC microfluidic device comprises 8 or more interconnected channels. In some embodiments, the plurality of interconnected channels of the SEC microfluidic device comprises 32 interconnected channels. In some embodiments, the plurality of interconnected channels of the SEC microfluidic device comprises 64 interconnected channels.

In some embodiments, the upstream network of connection channels, or portions thereof, of the SEC microfluidic device is connected to a proximal region of each of the plurality of interconnected channels. In some embodiments, the upstream network of connection channels of the SEC microfluidic device comprises a series of diverging channels configured to split fluid flow from the input port of the SEC microfluidic device to each of the plurality of interconnected channels. In some embodiments, each of the plurality of interconnected channels of the SEC microfluidic device is in fluidic communication with an output port of the SEC microfluidic device via a downstream network of connection channels. In some embodiments, the downstream network of connection channels of the SEC microfluidic device comprises a series of converging channels configured to combine fluid flow from the plurality of interconnected channels of the SEC microfluidic device to the output port.

In some embodiments, each of the plurality of interconnected channels of the SEC microfluidic device has a length of about 2 cm to about 50 cm. In some embodiments, each of the plurality of interconnected channels of the SEC microfluidic device has a width of about 1 μm to about 15 μm. In some embodiments, each of the plurality of interconnected channels of the SEC microfluidic device has a depth of about 1 μm to about 15 μm.

In some embodiments, the plurality of interconnected channels of the SEC microfluidic device are formed via a pillar array. In some embodiments, the pillar array of the SEC microfluidic device is an amorphous pillar array. In some embodiments, the pillar array of the SEC microfluidic device is a non-amorphous pillar array. In some embodiments, the pillar array of the SEC microfluidic device forms an inner surface of each of the plurality of interconnected channels of the SEC microfluidic device.

In some embodiments, the SEC microfluidic device comprises a quartz substrate. In some embodiments, the SEC microfluidic device comprises a monolithic substrate forming the plurality of interconnected channels. In some embodiments, the SEC microfluidic device comprises a quartz monolithic substrate.

In some embodiments, the SEC microfluidic device comprises a three-dimensional (3D) printed substrate.

In some aspects, provided herein is a reversed-phase liquid chromatography (RPLC) microfluidic device comprising: an input port; an upstream network of connection channels; and a plurality of interconnected channels, wherein each channel of the plurality of interconnected channels is in an open tubular format, wherein each channel of the plurality of interconnected channels comprises an inner surface comprising a RPLC medium, and wherein each channel the plurality of interconnected channels is in fluidic communication with the input port via the upstream network of connection channels.

In some embodiments, the RPLC medium of the RPLC microfluidic device comprises an alkyl moiety having about 2 to about 20 carbons. In some embodiments, the RPLC medium of the RPLC microfluidic device comprises one or more of C₂, C₄, C₈, and C₁₈. In some embodiments, the RPLC medium of the RPLC microfluidic device comprises a RPLC moiety mixture comprising two or more of the following alkyl moieties: C₂, C₄, C₈, and C₁₈. In some embodiments, the RPLC moiety mixture of the RPLC microfluidic device comprises three or more of the following alkyl moieties: C₂, C₄, C₈, and C₁₈. In some embodiments, the RPLC moiety mixture of the RPLC microfluidic device comprises the following alkyl moieties: C₂, C₄, C₈, and C₁₈. In some embodiments, the alkyl moieties of the RPLC moiety mixture of the RPLC microfluidic device are present in equimolar amounts.

In some embodiments, the RPLC medium of the RPLC microfluidic device is conjugated to the inner surface of each channel of the plurality of interconnected channels via silica (SiO₂). In some embodiments, the plurality of interconnected channels of the RPLC microfluidic device comprises between 8 and 100 interconnected channels. In some embodiments, the plurality of interconnected channels of the RPLC microfluidic device comprises 8 or more interconnected channels. In some embodiments, the plurality of interconnected channels of the RPLC microfluidic device comprises 32 interconnected channels. In some embodiments, the plurality of interconnected channels of the RPLC microfluidic device comprises 64 interconnected channels.

In some embodiments, the upstream network of connection channels, or portions thereof, of the RPLC microfluidic device is connected to a proximal region of each of the plurality of interconnected channels. In some embodiments, the upstream network of connection channels of the RPLC microfluidic device comprises a series of diverging channels configured to split fluid flow from the input port of the RPLC microfluidic device to each of the plurality of interconnected channels.

In some embodiments, each of the plurality of interconnected channels of the RPLC microfluidic device is in fluidic communication with an output port of the RPLC microfluidic device via a downstream network of connection channels. In some embodiments, the downstream network of connection channels of the RPLC microfluidic device comprises a series of converging channels configured to combine fluid flow from the plurality of interconnected channels of the RPLC microfluidic device to the output port.

In some embodiments, each of the plurality of interconnected channels of the RPLC microfluidic device has a length of about 2 cm to about 50 cm. In some embodiments, each of the plurality of interconnected channels of the RPLC microfluidic device has a width of about 1 μm to about 15 μm. in some embodiments, each of the plurality of interconnected channels of the RPLC microfluidic device has a depth of about 1 μm to about 15 μm.

In some embodiments, the plurality of interconnected channels of the RPLC microfluidic device are formed via a pillar array. In some embodiments, the pillar array of the RPLC microfluidic device is an amorphous pillar array. In some embodiments, the pillar array of the RPLC microfluidic device is a non-amorphous pillar array. In some embodiments, the pillar array of the RPLC microfluidic device forms an inner surface of each of the plurality of interconnected channels of the RPLC microfluidic device.

In some embodiments, the RPLC microfluidic device comprises a quartz substrate. In some embodiments, the RPLC microfluidic device comprises a monolithic substrate forming the plurality of interconnected channels. In some embodiments, the RPLC microfluidic device comprises a quartz monolithic substrate.

In some embodiments, the RPLC microfluidic device comprises a three-dimensional (3D) printed substrate.

In other aspects, provided herein is a method of analyzing a signature of identified components, comprising performing gene enrichment analysis, pathway analysis, and network analysis in any order, wherein: the signature of identified components comprises a protein set, a transcriptomic set, a peptide set, and/or a metabolite set; and the performing comprises: a process configured to perform gene enrichment analysis; a process configured to perform pathway analysis; a process configured to perform gene enrichment analysis; and a process configured to perform network analysis to identify drug targets.

In other aspects, provided herein is a method of subjecting an individual to a coronary artery disease (CAD) diagnosis determination, the method comprising: (a) obtaining mass spectrometry (MS) data from a sample, or a derivative thereof, obtained from the individual; and (b) analyzing the MS data according to a CAD proteomic signature, wherein the CAD proteomic signature comprises one or more biomarkers of Table 1; and (c) determining whether the individual has the CAD proteomic signature. In some embodiments, if the individual has the CAD proteomic signature, the individual is diagnosed has having CAD.

In other aspects, provided herein is a method of diagnosing an individual as having coronary artery disease (CAD), the method comprising: (a) obtaining mass spectrometry (MS) data from a sample, or a derivative thereof, obtained from the individual; and (b) analyzing the MS data according to a CAD proteomic signature, wherein the CAD proteomic signature comprises one or more biomarkers of Table 1; and (c) diagnosing the individual as having CAD based on the presence of the CAD proteomic signature.

In other aspects, provided herein is a method of treating an individual having coronary artery disease (CAD), the method comprising: (a) diagnosing an individual as having CAD according to the presence of a CAD proteomic signature in a sample, or a derivative thereof, obtained from the individual, wherein the CAD proteomic signature comprises one or more biomarkers of Table 1; and (b) administering to the individual a CAD treatment.

In some embodiments, the presence of the CAD proteomic signature is determined by analyzing MS data according to the CAD proteomic signature.

In some embodiments, the method further comprises obtaining the MS data from the sample, or the derivative thereof, obtained from the individual.

In some embodiments, the CAD treatment comprises a life style adjustment. In some embodiments, the CAD treatment comprises a pharmaceutical intervention. In some embodiments, the pharmaceutical intervention comprises administration of a drug selected from the group consisting of a calcium channel blocker, histone deacetylase (HDAC) inhibitor (such as HDAC6), Ca2+/calmodulin (CaM)-dependent protein kinase II (CaMK II) inhibitor, guanylyl cyclase (sGC) activator, MMP inhibitor, statin, and anti-hypertesnive. In some embodiments, the pharmaceutical intervention comprises a drug is selected from the group consisting of amlodipine, tubastatin-a, forskolin, trichostatin A, KN-93, CFM-1571, ilomastat, CAY-10603, and rosuvastatin, or a pharmaceutical salt thereof. In some embodiments, the drug is selected from the group consisting of BRD-K52306726, BRD-K71361154, acetazolamide, rolipram, ruxolitinib, BRD-A59808129-001-01-7, BRD-K76876037, ZM336372, trehalose, SCHEMBL3092652, BMS-387032, BRD-K01425431, 4-hydroxy-retinoic acid, CHEMBL585951, CHEMBL1673039, HY-11007, primidone, BRD-K81417919, SPECTRUM_000826, tamoxifen, BRD-K00544996, CID 67066889, CX-5461, BRD-K63944563, SCHEMBL6851809, BRD-A86146706, FR-180204, CHEMBL552425, hexachlorophene, Aggc, SUGA1_008424, BRD-K96640811, anastrozole, wortmannin, vandetanib, AC1NWALF, OTSSP167, WZ3105, dihydroergotamine, BRD-K99839793, SR 33805 oxalate, AT-7519, sulfadoxine, SPECTRUM_001319, MLS003329219, trichostatin A, and rotenone, or a pharmaceutical salt thereof.

In other aspects, provided is a method for detecting a coronary artery disease (CAD) proteomic signature of an individual, (a) obtaining mass spectrometry (MS) data from a sample, or a derivative thereof, obtained from the individual; and (b) analyzing the MS data according to a CAD proteomic signature to detect the CAD proteomic signature, wherein the CAD proteomic signature comprises one or more biomarkers of Table 1. In some embodiments, the individual is suspected of having CAD.

In some embodiments, the CAD proteomic signature comprises increased expression, as compared to a reference, of the one or more biomarkers according to Table 1. In some embodiments, the CAD proteomic signature comprises decreased expression, as compared to a reference, of the one or more biomarkers according to Table 1.

In some embodiments, the CAD proteomic signature comprises one or more biomarkers associated with a calcium signaling pathway, histone regulation, HIF-1 signaling pathway, cAMP signaling pathway, beta-adrenergic signaling pathway, PI3K-Akt signaling pathway, complement and/or coagulation cascade, sphingolipid signaling pathway, natural killer cell mediated cytotoxicity, adipocytoknie signaling pathway, DNA damage, calcium energy, metaboloimcs, cellular adhesion, inflammation, hypoxia, and histone methylation. In some embodiments, the one or more biomarkers comprise a subset thereof comprising one or more biomarkers associated with a transcription factor. In some embodiments, the one or more biomarkers comprise a subset thereof comprising one or more biomarkers associated with a kinase.

In some embodiments, the one or more biomarkers comprise at least 10 biomarkers of Table 1. In some embodiments, the one or more biomarkers comprise at least 25 biomarkers of Table 1. In some embodiments, the one or more biomarkers comprise at least 50 biomarkers of Table 1. In some embodiments, the one or more biomarkers comprise all biomarkers of Table 1.

In some embodiments, the method further comprises obtaining the sample from the individual. In some embodiments, the sample, or the derivative thereof, is a blood sample or a derivative thereof. In some embodiments, the sample, or the derivative thereof, is a plasma sample. In some embodiments, the sample, or the derivative thereof, comprises a liquid fixative.

In some embodiments, the obtaining MS data from the sample, or the derivative thereof, comprises performing a mass spectrometry analysis of the sample, or the derivative thereof, using a mass spectrometer. In some embodiments, the mass spectrometry analysis is performed according to any of methods provided herein for performing a mass spectrometry analysis. In some embodiments, the mass spectrometry analysis is performed according to the method of embodiments 140-143. In some embodiments, the analyzing the MS data according to the CAD proteomic signature comprises subjecting the MS data to a method of any one of embodiments 161-177. In some embodiments, the analyzing the MS data according to the CAD proteomic signature comprises assessing the presence or absence or level of each of the one or more biomarkers of the CAD proteomic signature in the MS data.

In some embodiments, the method further comprises performing one or more of the following factor assessments of the individual: sex, age, body mass index (BMI), systolic blood pressure, diastolic blood pressure, total cholesterol, HDL, LDL, triglycerides, hyperlipidemia, hypertension, diabetes mellitus, insulin resistance, kidney disease, smoking status, level of physical activity, level of sleep, or quality of nutrition.

In some embodiments, the method further comprises performing a medical procedure on the individual to assess the presence of CAD.

### BRIEF DESCRIPTION OF THE DRAWINGS

FIG. 1 shows an exemplary workflow 100 for obtaining a sample and analyzing components therein using mass spectrometry. As shown in FIG. 1, the exemplary workflow 100 includes sample acquisition 105, preliminary sample processing 110, liquid chromatography and, optionally, proteolysis 115, ionization for mass spectrometry 120, mass spectrometry data acquisition 125, and mass spectrometry data analysis 130.

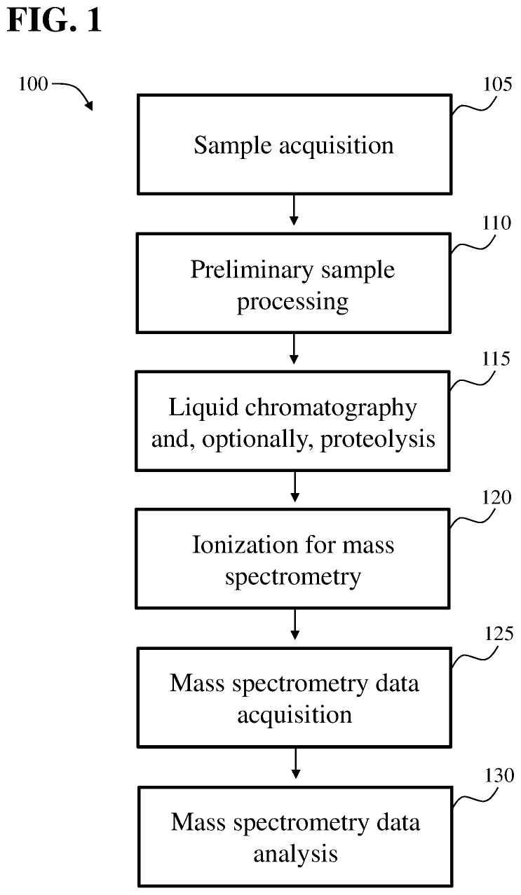

FIG. 2 shows an exemplary workflow 200 for obtaining a sample and analyzing components therein using mass spectrometry. As shown in FIG. 2, the exemplary workflow 200 includes blood sample acquisition 205, plasma generation 210, size-exclusion chromatography 215, proteolysis using enzymatic digestion 220, reversed-phase liquid chromatography (RPLC) coupled with online ionization for mass spectrometry 225, mass spectrometry data acquisition 230, and mass spectrometry data analysis 235.

FIG. 3 shows a schematic of an exemplary microfluidic device 300 configured for separation of components of a sample.

FIG. 4 shows a representative size-exclusion track of non-depleted human plasma. Fraction size is exemplified using dashed lines.

FIG. 5 shows a schematic of an exemplary size-exclusion chromatography microfluidic device.

FIG. 6 shows an exemplary cellular component analysis of the 292-protein CAD signature using ToppGene software.

FIG. 7 shows an exemplary molecular pathway analysis of the 292-protein CAD signature using ToppGene software.

FIG. 8 shows an exemplary Transcription Factor Enrichment Analysis (TFEA) algorithm of the 292-protein CAD signature.

FIG. 9 shows an exemplary Kinase Enrichment Analysis (KEA) of the 292-protein CAD signature.

FIG. 10 shows an exemplary 292-protein CAD signature interaction network produced using the GeneMANIA algorithm from the 292-protein CAD signature.

FIG. 11 shows an exemplary CAD complement pathway protein interaction subnetwork produced using the GeneMANIA algorithm from the 292-protein CAD signature.

FIG. 12 shows an exemplary CAD histone regulation protein interaction subnetwork produced using the GeneMANIA algorithm from the 292-protein CAD signature.

FIG. 13 shows an exemplary CAD DNA damage protein interaction subnetwork produced using the GeneMANIA algorithm from the 292-protein CAD signature.

FIG. 14 shows an exemplary CAD calcium energy protein interaction subnetwork produced using the GeneMANIA algorithm from the 292-protein CAD signature.

FIG. 15 shows an exemplary CAD metabolomics protein interaction subnetwork produced using the GeneMANIA algorithm from the 292-protein CAD signature.

FIG. 16 shows an exemplary CAD cellular adhesion protein interaction subnetwork produced using the GeneMANIA algorithm from the 292-protein CAD signature.

FIG. 17 shows an exemplary CAD inflammation protein interaction subnetwork produced using the GeneMANIA algorithm from the 292-protein CAD signature.

FIG. 18 shows an exemplary CAD hypoxia protein interaction subnetwork produced using the GeneMANIA algorithm from the 292-protein CAD signature.

FIG. 19 shows an exemplary CAD histone methylation protein interaction subnetwork produced using the GeneMANIA algorithm from the 292-protein CAD signature.

FIGS. 20A-20B shows an exemplary L1000 FWD algorithm analysis identifying FDA-approved drugs that target the hubs of protein networks (FIG. 20B) represented in the 292-protein CAD signature (FIG. 20A).

FIG. 21 shows an exemplary ILINCs chemical perturbation algorithm analysis identifying novel drugs that target the hubs of protein networks represented in the 292-protein CAD signature.

### DETAILED DESCRIPTION

In some aspects, provided herein is a method of processing a test sample for mass spectrometry analysis. In other aspects, provided herein are microfluidic devices useful for separation of components, such as a size-exclusion chromatography microfluidic device or a reversed-phase liquid chromatography microfluidic device. In other aspects, provided herein is a collection of obtained compositions using the methods and/or devices described herein. In other aspects, provided herein is a method of analyzing a collection of compositions using a mass spectrometry technique. In other aspects, provided herein is a method of identifying a signature comprising one or more identified biomolecules. In other aspects, provided herein is a signature identified using the methods and/or devices disclosed herein. In other aspects, provided herein is a method of analyzing the components of the signature for a function, activity, and/or attribute.

The provided embodiments relate to a non-priori, agnostic methods using mass spectrometry to achieve high proteome coverage that includes the capture of a diverse set of proteins, such as secreted, endogenous cleavage products, soluble proteins, and exosome or lipid microvesicle-enriched proteins, as well as other non-protein components of a sample. These biomolecules can span a large linear dynamic range (e.g., typically 12-orders of magnitude or more). Such an analytical strategy as achieved by the provided methods and/or devices allows the unbiased capture and analysis of a wide spectrum of proteins with diverse physico-chemical and biological properties as well as other non-protein components of a sample. The provided methods also minimize pre-analytical variables so as to reproducibly analyze the majority of the observable components of a sample, such as the proteome including those proteins naturally occurring at low abundance level.

In some embodiments, the provided methods and/or devices can be used for the unbiased discovery and follow-up targeted analysis of specific molecular signatures, including protein biosignatures (e.g., disease specific protein biosignatures), from a small biological sample, including from just a prick-test procured blood specimen. The plasma extraction from a single blood drop may be achieved with capillary action filtration through a commercially available material and directly mixed with a chaotropic liquid fixative. In some embodiments, the liquid fixative solubilizes and preserves the protein and other biological analytes from the blood sample, including primary and secondary metabolites, native peptides, and microRNAs. Due to its strong chaotropic activity, this liquid fixative eliminates protease activity, achieves maximum preservation of chemical integrity of metabolites, eliminates protein-protein binding, and affords a maximum hydrodynamic radius and liquid viscosity for their efficient size-exclusion chromatographic (SEC) separation. Further, the specimen procurement and preservation device thoroughly neutralizes all human pathogens (e.g., viruses, bacteria, fungi, etc.) with minimum chemical or toxicological hazards. This configuration is amenable to point-of-care devices for the procurement and chemical fixation of blood plasma or serum, and its protein, native peptide, metabolite content, and nucleic acid, e.g., RNA, content.

In some embodiments, the methods and/or devices provide microfluidic size-exclusion chromatography that achieves efficient flow dynamics (minimum turbulence), low operation back-pressure, optimum surface-to-volume ratios, and affords excellent sampling of a wide range of hydrodynamic radii or molecular weights observed in the diverse set of biomolecular species found in samples, such as whole, non-depleted blood plasma/serum including proteins, endogenous peptides, metabolites, and nucleic acids, e.g., RNA. Importantly, such microfluidic based partitioning utilizes the liquid fixative from sample procurement in order to create a highly integrated and orthogonal pipeline.

In some embodiments, the biomarker discovery methods provided herein additionally comprises a relative quantitative analysis of a fractionated sample, through stoichiometrically normalized isobaric stable isotope tagging. In some embodiments, the method is also amendable to label-free approaches. In contrast with standard protein digestion with proteases, no reduction step and/or alkylation step are required due to the liquid fixative properties present in samples, or fractions thereof, to be subjected to proteolysis. The fractions generated from the original sample may be further separated using a modified, reversed-phased liquid chromatography device with an open-tubular configuration as provided herein. The devices described herein may be useful for the separation of, e.g., proteolytic peptides derived from proteins, native peptides (e.g., MHC Class I and II, insulin, glucagon, troponins, etc.), and primary (e.g., enzyme co-factors, sugars, amino acids, nucleic acids, lipids, etc.) or secondary metabolites (e.g., derived from drugs or other xenobiotic agents, etc.) and nucleic acids, e.g., RNA species. The ability to co-analyze native peptides, metabolites and RNA species, as they occur for example to exosomes or other lipid microvesicles naturally occurring in biological fluids such as blood plasma or serum, may constitute enzyme or kinase co-factors and thus help decipher and validate their functional state and serve as surrogate markers thereof. In some embodiments, the open-tubular reversed-phased liquid chromatography may be configured and is performed on a lab chip device.

As described in the present disclosure, in some embodiments, the open-tubular reversed-phased liquid chromatography microfluidic device include a long combined column length, can be constructed from quartz material, and a chemically modified surface with any one or more of C₂, C₄, C₈, and C₁₈ alkyl groups. The open-tubular reversed-phased liquid chromatography microfluidic devices described herein provide an increase in the number of theoretical plates and therefore separation efficiency at higher binding capacity, as well as the ability to separate for a wide range of hydrophobic, amphipathic and hydrophobic peptides, thus facilitating their downstream analysis (e.g., electrospray ionization and mass spectrometric analysis).

In some embodiments, provided herein are methods for robust and comprehensive discovery of biomarkers associated with a particular biological phenomenon, such as disease, disease stage or severity, responsiveness to a particular drug, are important for enabling assessment, monitoring or prediction of the biological phenomenon. In particular, biomarkers can serve as diagnostic markers, prognostic markers or stratification markers. For instance, biomarkers are important for the assessment of disease risk and progression, and for monitoring, or even, predicting patients' responses to treatments. The ability to co-analyze native peptides, metabolites and RNA species, as they occur for example to exosomes or other lipid microvesicles naturally occurring in biological fluids such as blood plasma or serum, may constitute enzyme or kinase co-factors and thus help decipher and validate their functional state and serve as surrogate markers thereof. Proteins regulate biochemical reactions in the human body and can integrate the effects of genes with epigenetic factors associated with the environment, age, comorbidities, behaviors, and drugs. As such, proteins exhibit great endophenotypic biomarker potential.

Nevertheless, proteins used in the clinic as biomarkers represent only a very small fraction of the circulating proteome. Further, other biomolecules such as certain metabolites in fluid sample, such as blood, may also be a relevant biomarker of biological phenomenona, such as disease. Thus, existing methods generally fail to capture the extent of coverage of relevant biomarkers. In addition, despite advances in the development of multidimensional data integration algorithms and other computer based machine-learning tools, the flexibility, effectiveness and robustness of data integration to extract mechanistic insights into biomarkers remains restricted. As such, many existing methods fail to capture proteins present in a biological sample that are of pathophysiologic relevance to a particular biological phenomenon, such as a particular disease. Thus, available approaches for biomarker discovery and mechanistic analysis are not entirely satisfactory.

Additionally, the utility of existing mass spectrometry methods is limited by a number of aspects, including the ability to introduce a component species of a sample (such as low-abundant population of a single type of peptide from the sample) to the mass spectrometer in such a concentrated form that the component species reaches the detector of the mass spectrometer and is analyzed. This challenge is confounded in the presence of very highly abundant component species, such as is the case with human blood samples and the relatively high concentration of, e.g., albumin, IgG, antitrypsin, IgA, transferrin, haptoglobin, and fibrinogen. In addition to the challenges of efficiently separating and concentrating components of a sample, many components may be lost during sample preparation prior to mass spectrometry analysis.

The provided embodiments address one or more of these problems. For example, in some aspects, described herein is a comprehensive plasma discovery and validation pipeline that is completely independent of affinity-depletion and affinity enrichment steps, and represents a quantitative application to a diverse range of biomedical applications in non-depleted blood serum and/or plasma. Additionally, the identified components of can be analyzed according to the methods described herein to identify and/or use disease-specific biosignatures as a novel and highly accurate tool having, e.g., diagnostic and/or prognostic value.

Furthermore, the methods and devices provided herein comprise a technological platform that is amenable to automation and scale-up. Such a premise becomes essential to achieve statistical power through the comprehensive analysis of hundreds or even thousands of samples. The high-volume and reproducible analysis of samples, such as plasma proteomes, accomplished by the provided embodiments allow maximum exploitation of a diversity of artificial intelligence, machine learning algorithms that can decipher, e.g., functional and clinically relevant endophenotypic evidence at the protein and derivative metabolite level (e.g., an integrated proteometabolomic profile described herein) despite the large heterogeneity of clinical presentation of high-risk patients at the early, initiation stage and their subsequent safe and effective treatment.

In some aspects, an additional advantage to the platform embodied by the provided method is that its technological components constitute a unitary, vertically integrated, pipeline given their high-degree of complimentary principles of operation. Furthermore, as the pipeline is highly amenable to automation it can be scaled-up to increase analysis capacity with minimum human intervention. Such features collectively facilitate the effective and comprehensive analysis of protein biosignatures in blood plasma derived from any disease. Importantly, the platform may operate in both discovery mode for the unbiased or agnostic quantification of a broad spectrum of components, such as proteins, as they are differentially expressed/exist in a disease specific manner, or alternatively in a targeted absolute quantitative analysis mode for the high-throughput parallel interrogation of components identified from a discovery analysis. Both discovery and derivative targeted mode of analysis of the platform makes no use of expensive and unreliable antibody and/or aptamer-based depletion or enrichment of proteins prior to measurement.

The result of the disclosed methods and/or devices is a platform that provides sensitive, robust, and reproducible results capable of identifying and/or quantifying components from a sample, such as proteins including those that are difficult such as from the exosome. Furthermore, the methods and/or devices are suitable for miniaturization and integration, including as necessary for a unitary lab chip device.

Thus, in some aspects, provided herein is a method for processing a test sample for a mass spectrometry analysis, the method comprising: (a) subjecting the test sample to a size-exclusion chromatography (SEC) technique using a SEC microfluidic device, wherein the test sample comprises one or more biomolecules and a chaotropic agent, and wherein the SEC microfluidic device comprises a plurality of interconnected channels; (b) collecting a plurality of fractions eluted from the SEC microfluidic device; (c) subjecting one or more of the plurality of fractions from the SEC microfluidic device to a proteolytic technique; and (d) individually subjecting one or more fractions from one or both of steps (b) and (c) to a reversed-phase liquid chromatography (RPLC) technique using a RPLC microfluidic device under conditions to prepare a component of each of the one or more fractions for introduction to a mass spectrometer, wherein the RPLC microfluidic device comprises a plurality of interconnected channels comprising a reversed-phase medium, and wherein the RPLC microfluidic device is coupled to an electrospray ionization source.

In some aspects, provided herein is a method for processing components, or products thereof, of a sample, such as a biological sample, for mass spectrometry analysis. In some embodiments, the method comprises (a) subjecting a test sample to a size-exclusion chromatography (SEC) technique using a SEC microfluidic device; (b) collecting a plurality of fractions eluted from the SEC microfluidic device; (c) subjecting one or more of the plurality of fractions from the SEC microfluidic device to a proteolytic technique; and (d) subjecting each of a set of RPLC-compatible fractions to a reversed-phase liquid chromatography (RPLC) technique using a RPLC microfluidic device to prepare the components, or products thereof, of the sample for introduction to a mass spectrometer.

In other aspects, provided herein is a method for processing components, or products thereof, of a biological sample for a mass spectrometry analysis, the method comprising: (a) subjecting a test sample to a size-exclusion chromatography (SEC) technique using a SEC microfluidic device, wherein the test sample comprises the sample admixed with a liquid fixative, wherein the test sample has a pre-determined concentration of a chaotropic agent originating from the liquid fixative, wherein the SEC technique comprises use of a SEC mobile phase having a concentration of a mobile phase chaotropic agent within a range of the pre-determined concentration of the chaotropic agent in the test sample, and wherein the SEC microfluidic device comprises a plurality of interconnected channels, each channel being configured in an open tubular format having an inner surface material having an average pore size of about 10 nm to about 500 nm; (b) collecting a plurality of fractions eluted from the SEC microfluidic device; (c) subjecting one or more of the plurality of fractions from the SEC microfluidic device to a proteolytic technique; and (d) subjecting each of a set of RPLC-compatible fractions to a reversed-phase liquid chromatography (RPLC) technique using a RPLC microfluidic device to prepare the components, or products thereof, of the sample for introduction to a mass spectrometer, wherein the set of RPLC-compatible fractions comprises fractions obtained from: (i) zero or more of the plurality of fractions from the SEC microfluidic device; and (ii) one or more of the plurality of fractions subjected to the proteolytic technique, wherein the RPLC technique and RPLC microfluidic device are configured for online desalting, wherein the RPLC microfluidic device comprises a plurality of interconnected channels, each channel being configured in an open tubular format having an inner surface material comprising a reversed-phase medium, wherein the RPLC microfluidic device is coupled to an electrospray ionization source.

In other aspects, provided herein is a collection of compositions obtained from any one of the methods described herein. In some embodiments, each composition of the collection of compositions is a RPLC microfluidic device eluate.

In other aspects, provided herein is a method of analyzing a collection of compositions using mass spectrometry, the method comprising: (a) subjecting each composition of the collection of compositions to a mass spectrometer; and (b) performing a mass spectrometry analysis of each composition of the collection of compositions, wherein the collection of compositions is obtained from a processing technique comprising fractionation of a test sample using a SEC technique comprising use of a SEC microfluidic device followed by application of each fraction, or a product thereof, to a RPLC technique comprising use of a RPLC microfluidic device.

In other aspects, provided herein is a size-exclusion chromatography (SEC) microfluidic device comprising: an input port; an upstream network of connection channels; and a plurality of interconnected channels, wherein each channel of the plurality of interconnected channels is in an open tubular format, wherein each channel of the plurality of interconnected channels comprises an inner surface comprising a SEC medium, and wherein each channel of the plurality of interconnected channels is in fluidic communication with the input port via the upstream network of connection channels.

In other aspects, provided herein is a reversed-phase liquid chromatography (RPLC) microfluidic device comprising: an input port; an upstream network of connection channels; and a plurality of interconnected channels, wherein each channel of the plurality of interconnected channels is in an open tubular format, wherein each channel of the plurality of interconnected channels comprises an inner surface comprising a RPLC medium, and wherein each channel the plurality of interconnected channels is in fluidic communication with the input port via the upstream network of connection channels.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

The term “about” as used herein refers to the usual error range for the respective value readily known in this technical field. Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human.

As used herein, the term “treating” and “treatment” includes administering to a subject an effective amount of an agent or prescribing a life style adjustment, such as cessation of smoking, described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease.

Those skilled in the art will recognize that several embodiments are possible within the scope and spirit of the present disclosure. The following description illustrates the disclosure and, of course, should not be construed in any way as limiting the scope of the inventions described herein.

### II. Methods for Processing Components, or Products Thereof, of a Sample

In some aspects, provided herein are methods for processing components, or products thereof, of a sample to separate, at least to a degree, the components, or products thereof, from one another for a downstream application. In some embodiments, the processing methods described herein are useful for efficiently and efficaciously separating and concentrating components, or products thereof, for a mass spectrometry analysis.

In some embodiments, the methods for processing components, or products thereof, of a sample for a mass spectrometry analysis comprehensively include all steps from sample acquisition to introduction of the components, or products thereof, to a mass spectrometer. In some embodiments, the methods described herein comprise certain aspects involved in the overall processing of components, or products thereof, for a mass spectrometry analysis, such as one or more liquid chromatography steps and/or a preliminary processing step. In some embodiments, the methods for processing described herein are configured to interface, such as immediately precede, a downstream application including a mass spectrometry analysis. Aspects of the methods disclosed herein are described in more detail below in a modular fashion. Such presentation is not to be construed as limiting the scope of combinations of the various aspects encompassed by the disclosure of the present application to form a method for processing components, or products thereof, of a sample.

### A. Samples, Components Thereof, Sample Acquisition, and Preliminary Sample Processing

The methods disclosed herein are useful for processing components, or products thereof, of various samples from a diverse array of sources containing a multitude of different combinations of components.

In some embodiments, the sample is a biological sample, such as a sample comprising an organism or a portion or product thereof. In some embodiments, the biological sample is from an individual, such as a human. In some embodiments, the individual is a mammal, such as a human, bovine, horse, feline, canine, rodent, or primate. In some embodiments, the sample is a human sample. In some embodiments, the biological sample comprises material from an organism classified in the Eubacteria kingdom, Archaebacterial kingdom, Protista kingdom, Plantae kingdom, Fungi kingdom, or Animalia kingdom. In some embodiments, the sample is an environmental sample.

In some embodiments, the sample comprises a fluid and/or solid (e.g., a cell) of an individual. In some embodiments, the sample is a liquid biopsy. In some embodiments, the sample comprises a bodily fluid, such as a sample comprising a blood sample, serum sample, convalescent plasma sample, oropharyngeal sample, including that obtained from an oropharyngeal swab, nasopharyngeal sample, including that obtained from a nasopharyngeal swab, buccal sample, bronchoalveolar lavage sample, including that obtained from an endotracheal aspirator, sweat sample, sputum sample, salivary sample, tear sample, bodily excretion sample, or cerebrospinal fluid sample. In some embodiments, the sample comprise a solid, such as a sample comprising a fecal sample. In some embodiments, the sample is selected from the group consisting of a blood sample, cerebrospinal fluid (CSF) sample, ascetic fluid sample (proximal fluid adjacent an organ), seminal fluid sample, and nipple aspirate fluid sample.

In some embodiments, the sample is a complex sample, such as a complex biological sample. In some embodiments, the sample comprises components having concentrations spanning at least about 2 orders of magnitude, such as at least about any of 3 orders of magnitude, 4 orders of magnitude, 5 orders of magnitude, 6 orders of magnitude, 7 orders of magnitude, 8 orders of magnitude, 9 orders of magnitude, or 10 orders of magnitude.

In some embodiments, the sample comprises a component, such as a biomolecule or a derivative thereof. In some embodiments, features of a sample and/or any fraction described herein (such as a portion of a fluid obtained from a method step and/or device described herein), such as a protein, peptide, nucleic acid, metabolite, or derivatives thereof (such as a processed and/or labeled form thereof), may be described as components.

In some embodiments, the component is a polypeptide (such as a protein, a naturally occurring peptide, or endogenous protein cleavage product), a polynucleotide (such as a DNA or RNA), or a metabolite. In some embodiments, the sample comprises proteins, naturally occurring peptides, and metabolites. In some embodiments, the component comprises a post-translational modification.

In some embodiments, the product of a component of a sample is any derivative of the component generated at or after sample acquisition. For example, in some embodiments, the product of a protein component of a sample includes any modification to the protein component, or resulting parts, that occurs during and/or as a result of a sample processing, including a protein component having an altered physical structure or composition (e.g., having a post-translational modification), a polypeptide or peptide resulting from proteolysis of the protein component, and a polypeptide or peptide having an altered physical structure of composition (e.g., having a post-translational modification and/or quantitative label).

In some embodiments, the sample is a non-depleted sample, e.g., a sample that has not been processed to remove certain components thereof such as high abundant proteins.

In some embodiments, the sample is a blood sample or a sample derived therefrom, e.g., a plasma sample. In some embodiments, the sample comprises a blood sample. In some embodiments, the blood sample is a whole blood sample. In some embodiments, the blood sample is a non-depleted blood sample, e.g., a blood sample that has not been processed to remove certain components thereof such as high abundant proteins. In some embodiments, the blood sample comprises a plasma sample. In some embodiments, the plasma sample is a non-depleted plasma sample, e.g., a plasma sample that has not been processed to remove certain components thereof such as high abundant proteins, but has been processed to remove other generally removed when generating a plasma sample from a whole blood ample. In some embodiments, the blood sample comprises a serum sample. In some embodiments, the serum sample is a non-depleted serum sample, e.g., a serum sample that has not been processed to remove certain components thereof such as high abundant proteins. In some embodiments, the blood sample, including a plasma sample or serum sample obtained therefrom, has not been processed to remove any one or more of seven common highly abundant blood proteins (albumin, IgG, antitrypsin, IgA, transferrin, haptoglobin, fibrinogen). In some embodiments, the blood sample, including a plasma sample or serum sample obtained therefrom, has not been process to remove any one or more of fourteen common highly abundant blood proteins (albumin, IgG, antitrypsin, IgA, transferrin, haptoglobin, fibrinogen, alpha2-macroglobulin, alpha1-acid glycoprotein, IgM, apolipoprotein AI, apolipoprotein AII, complement C3, transthyretin).

In some embodiments, the sample has a volume (such as the volume of the sample obtained from an individual) of about 10 μL to about 200 μL, such as about any of about 10 μL to about 100 μL, about 10 μL to about 75 μL, about 25 μL to about 75 μL, or about 30 μL to about 60 μL. In some embodiments, the sample has a volume of at least about 10 μL, such as at least about any of 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 105 μL, 110 μL, 115 μL, 120 μL, 125 μL, 130 μL, 135 μL, 140 μL, 145 μL, 150 μL, 155 μL, 160 μL, 165 μL, 170 μL, 175 μL, 180 μL, 185 μL, 190 μL, 195 μL, or 200 μL. In some embodiments, the sample has a volume of less than about 200 μL, such as less than about any of 195 μL, 190 μL, 185 μL, 180 μL, 175 μL, 170 μL, 165 μL, 160 μL, 155 μL, 150 μL, 145 μL, 140 μL, 135 μL, 130 μL, 125 μL, 120 μL, 115 μL, 110 μL, 105 μL, 100 μL, 95 μL, 90 μL, 85 μL, 80 μL, 75 μL, 70 μL, 65 μL, 60 μL, 55 μL, 50 μL, 45 μL, 40 μL, 35 μL, 30 μL, 25 μL, 20 μL, 15 μL, or 10 μL. In some embodiments, the sample has a volume of about any of 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 105 μL, 110 μL, 115 μL, 120 μL, 125 μL, 130 μL, 135 μL, 140 μL, 145 μL, 150 μL, 155 μL, 160 μL, 165 μL, 170 μL, 175 μL, 180 μL, 185 μL, 190 μL, 195 μL, or 200 μL.

In some embodiments, the method comprises obtaining a sample from an individual. In some embodiments, the method comprises one or more preliminary sample processing steps. In some embodiments, the preliminary sample processing step comprises admixing a sample with an agent that preserves the sample in a state for later analysis. In some embodiments, the preliminary sample processing step comprises admixing a sample (such as a blood sample) with an anti-coagulation agent. In some embodiments, the preliminary sample processing step comprises admixing a sample (such as a blood sample) with an enzyme inhibitor, e.g., a protease inhibitor. In some embodiments, the preliminary sample processing step comprises subjecting a sample to a condition to preserve the sample in a state for later analysis. In some embodiments, the preliminary sample processing step comprises subjecting a sample to a reduced temperature, such as a temperature of about any of 10° C. or less, 4° C. or less, 0° C. or less, −20° C. or less, or −80° C. or less.

In some embodiments, the sample is obtained at a point-of-care.

In some embodiments, the preliminary sample processing step comprises admixing a sample with a liquid fixative to generate a test sample. In some embodiments, the liquid fixative components and/or concentrations thereof and/or ratio of sample volume to liquid fixative volume can be adjusted to meet the needs of the methods described herein, such as to achieve a pre-determined concentration of one or more components of the liquid fixative in a test sample.

In some embodiments, the liquid fixative comprises a chaotropic agent. In some embodiments, the chaotropic agent comprises guanidine or a salt thereof, guanidinium or a salt thereof, potassium or a salt thereof, lithium or a salt thereof, magnesium or a salt thereof, or sodium or a salt thereof. In some embodiments, the chaotropic agent is selected from the group consisting of guanidine hydrochloride, guanidinium chloride, guanidinium thiocynante, lithium perchlorate, lithium acetate, magnesium chloride, potassium acetate, and sodium iodide. In some embodiments, the chaotropic agent is a guanidine salt. In some embodiments, the chaotropic agent is guanidine hydrochloride.

In some embodiments, the test sample comprises a concentration of a chaotropic agent originating from a liquid fixative of about 5 M to about 8 M, such as any of about 5.5 M to about 8 M, about 5.5 M to about 7 M, or about 5.5 M to about 6.5 M. In some embodiments, the test sample comprises a concentration of a chaotropic agent originating from a liquid fixative of at least about 5.5 M, such as at least about any of 6 M, 6.5 M, 7 M, 7.5 M, or 8 M. In some embodiments, the test sample comprises a concentration of a chaotropic agent originating from a liquid fixative of about 8 M or less, such as about any of 7.5 M or less, 6.5 M or less, or 6 M or less. In some embodiments, the test sample comprises a concentration of a chaotropic agent originating from a liquid fixative of about any of 5.5 M, 6 M, 6.5 M, 7 M, 7.5 M, or 8 M.

In some embodiments, the liquid fixative comprises a viscosity modulating agent. In some embodiments, the viscosity modulating agent is selected from the group consisting of glycerol, propylene glycol, sorbitol, and polyethylene glycol (PEG). In some embodiments, the viscosity modulating agent is glycerol.

In some embodiments, the test sample comprises a concentration of a viscosity modulating agent originating from a liquid fixative of about 5% to about 40%, such as any of about 5% to about 20%, about 10% to about 30%, about 20% to about 30%, or about 20% to about 40%. In some embodiments, the test sample comprises a concentration of a viscosity modulating agent originating from a liquid fixative of at least about 5%, such as at least about any of 10%, 15%, 20%, 25%, 30%, 35%, or 40%. In some embodiments, the test sample comprises a concentration of a viscosity modulating agent originating from a liquid fixative of about 40% or less, such as about any of 35% or less, 30% or less, 25% or less, 20% or less, 15% or less, 10% or less, or 5% or less. In some embodiments, the test sample comprises a concentration of a viscosity modulating agent originating from a liquid fixative of about any of 5%, 10%, 15%, 20%, 25%, 30%, 35%, 40%, 45%, or 50%. In some embodiments, the amount of a viscosity modulating agent in a test sample is based on the desired viscosity of the test sample (such as for processing via aspects of the methods described herein, including a SEC microfluidic device).

In some embodiments, the test sample comprises a concentration of a chaotropic agent (such a guanidine hydrochloride) originating from a liquid fixative of about 5 M to about 8 M, such as any of about 5.5 M to about 7.5 M, about 5.5 M to about 7 M, or about 5.5 M to about 6.5 M, and a concentration of a viscosity modulating agent (such as glycerol) originating from a liquid fixative of about 5% to about 40%, such as any of about 5% to about 20%, about 10% to about 30%, about 20% to about 30%, or about 20% to about 40%. In some embodiments, the test sample comprises a concentration of a chaotropic agent (such a guanidine hydrochloride) originating from a liquid fixative of at least about 5 M, such as at least about any of 5.5 M, 6 M, 6.5 M, 7 M, 7.5 M, or 8 M, and a concentration of a viscosity modulating agent (such as glycerol) originating from a liquid fixative of at least about 5%, such as at least about any of 10%, 15%, 20%, 25%, 30%, 35%, or 40%. In some embodiments, the test sample comprises a concentration of a chaotropic agent (such a guanidine hydrochloride) originating from a liquid fixative of about 8 M or less, such as about any of 7.5 M or less, 6.5 M or less, or 6 M or less, and a concentration of a viscosity modulating agent (such as glycerol) originating from a liquid fixative of about 40% or less, such as about 35% or less, 30% or less, 25% or less, 20% or less, 15% or less, 10% or less, or 5% or less. In some embodiments, the test sample comprises a concentration of a chaotropic agent (such a guanidine hydrochloride) originating from a liquid fixative of about any of 5 M, 5.5 M, 6 M, 6.5 M, 7 M, 7.5 M, or 8 M, and a concentration of a viscosity modulating agent (such as glycerol) originating from a liquid fixative of about any of 5%, 10%, 15%, 20%, 25%, 30%, 35%, 40%, 45%, or 50%.

In some embodiments, the test sample comprises a concentration of a chaotropic agent (e.g., guanidine hydrochloride) originating from a liquid fixative of about 5.5 M to about 8 M, such as about 6 M or more, and a concentration of a viscosity modifying agent (e.g., glycerol) originating from a liquid fixative of about 5% to about 40%, such about 10% to about 30%.

In some embodiments, the test sample is a non-depleted sample, e.g., a test sample that has not been processed to remove certain components thereof such as high abundant proteins. In some embodiments, the test sample, including test sample obtained from a blood sample, a plasma sample, or serum sample, has not been process to remove any one or more of seven common highly abundant blood proteins (albumin, IgG, antitrypsin, IgA, transferrin, haptoglobin, fibrinogen). In some embodiments, the test sample, including test sample obtained from a blood sample, a plasma sample, or serum sample, has not been process to remove any one or more of fourteen common highly abundant blood proteins (albumin, IgG, antitrypsin, IgA, transferrin, haptoglobin, fibrinogen, alpha2-macroglobulin, alpha1-acid glycoprotein, IgM, apolipoprotein AI, apolipoprotein AII, complement C3, transthyretin).

In some embodiments, the liquid fixative may be diluted with a solution, such as water, to reach the desired concentration, e.g., such as when prepared from a stock formulation (wet or dry). In some embodiments, the viscosity modifying agent of a liquid fixative is admixed with water to achieve the desired concentration of a liquid fixative. For example, in some embodiments, the liquid fixative comprises 7 M of a chaotropic agent admixing in a 10% viscosity modifying agent/90% water solution.

As discussed herein, concentrations of one or more components of a liquid fixative may be based on the desired component concentration from the liquid fixative in the test sample and/or the ratio of sample volume to liquid fixative volume. For example, in some embodiments, the liquid fixative comprises a concentration of a chaotropic agent and/or a concentration of a viscosity modifying agent such that when admixed with a sample to generate a test sample, the chaotropic agent and/or the viscosity modifying agent originating from the liquid fixative are at concentrations as described herein.

In some aspects, provided herein is a method for preparing a test sample of plasma from a blood sample of an individual. In some embodiments, the method for preparing a test sample of plasma is integrated with other methods described herein. In some embodiments, when the sample from an individual is a blood sample, the method further comprises preparing a plasma sample. In some embodiments, preparing the plasms sample comprises subjecting the blood sample to a plasma generation technique. In some embodiments, the plasma generation technique comprises subjecting the sample to a polysulphone medium. In some embodiments, the polysulphone medium is an asymmetric polysulphone material. In some embodiments, the plasma generation technique is a capillary action filtration technique. In some embodiments, the plasma generation technique is a polysulphone (such as an asymmetric polysulphone) capillary action filtration technique.

Additional techniques are known in the art for generating a plasma sample, and such techniques are may be using in the methods described herein. For example, in some embodiments, the plasma generation technique comprises subjecting a blood sample from an individual to centrifugation, wherein the centrifugation of the blood sample is performed in the presence of an anticoagulant (e.g., any one or more of ethylenediaminetetraacetic acid (EDTA), heparin, and citrate) to allow for separation of plasma from whole blood. In some embodiments, the plasma generation technique comprises subjecting a blood sample from an individual to agglutination. In some embodiments, the plasma generation technique comprises subjecting a blood sample from an individual to passive or active microfluidic-based separation. In some embodiments, the plasma generation technique comprises subjecting a blood sample from an individual to a medium comprising any one or more of polysulphone, polyethersulphone, and cellulose acetate.

In some embodiments, the volume of a blood sample subjected to the plasma generation technique is about 10 μL to about 200 μL, such as any of 10 μL to about 100 μL, such as about 25 μL to about 75 μL. In some embodiments, the volume of a blood sample subjected to the plasma generation technique is at least about 10 μL, such as at least about any of 20 μL, 30 μL, 40 μL, 50 μL, 60 μL, 70 μL, 80 μL, 90 μL, 100 μL, 110 μL, 120 μL, 130 μL, 140 μL, 150 μL, 160 μL, 170 μL, 180 μL, 190 μL, or 200 μL. In some embodiments, the volume of a blood sample subjected to the plasma generation technique is at least about 10 μL, such as at least about any of 20 μL, 30 μL, 40 μL, 50 μL, 60 μL, 70 μL, 80 μL, 90 μL, 100 μL, 110 μL, 120 μL, 130 μL, 140 μL, 150 μL, 160 μL, 170 μL, 180 μL, 190 μL, or 200 μL, and less than about 500 μL. In some embodiments, the volume of a blood sample subjected to the plasma generation technique is at less than about 200 μL, such as less than any of 190 μL, 180 μL, 170 μL, 160 μL, 150 μL, 140 μL, 130 μL, 120 μL, 110 μL, 100 μL, 90 μL, 80 μL, 70 μL, 60 μL, 50 μL, 40 μL, 30 μL, 20 μL, or 10 μL. In some embodiments, the volume of a blood sample subjected to the plasma generation technique is about any of 10 μL, 20 μL, 30 μL, 40 μL, 50 μL, 60 μL, 70 μL, 80 μL, 90 μL, 100 μL, 110 μL, 120 μL, 130 μL, 140 μL, 150 μL, 160 μL, 170 μL, 180 μL, 190 μL, or 200 μL.

In some embodiments, the volume of generated plasma is about 1 μL to about 100 μL. In some embodiments, the volume of generated plasma is at least about 1 μL, such as at least about any of 2 μL, 3 μL, 4 μL, 5 μL, 6 μL, 7 μL, 8 μL, 9 μL, 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, or 100 μL. In some embodiments, the volume of generated plasma is about any of 1 μL, 2 μL, 3 μL, 4 μL, 5 μL, 6 μL, 7 μL, 8 μL, 9 μL, 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, or 100 μL.

In some embodiments, the plasma sample, such as a generated plasma sample, is admixed with a liquid fixative to generate the test sample (test plasma sample). In some embodiments, the plasma sample is admixed with a liquid fixative directly (such as immediately) after preparation of the plasma sample. In some embodiments, the method comprises admixing a plasma sample (such as a generated plasma sample) with a liquid fixative to generate a test sample (test plasma sample). As discussed herein, the concentration of components of a liquid fixative may be adjusted based on, at least in part, a desired concentration of components (such as a chaotropic agent and/or a viscosity modifying agent) in the test sample originating from the liquid fixative. In some embodiments, the volume of a sample (such as a plasma sample) to a liquid fixative admixed in the methods described herein may be based on, at least in part, a desired concentration of components (such as a chaotropic agent and/or a viscosity modifying agent) in the test sample originating from the liquid fixative, a desired final volume of the test sample, and/or limitations of the concentrations of certain components in the liquid fixative.

In some embodiments, the test sample, such as the test plasma sample generated using the methods described herein, is not further depleted prior to subjecting the test sample to the separation technique described herein, such as a SEC technique using a SEC microfluidic device. In some embodiments, such depletion methods comprise use of any one or more of an antibody, aptamer, other affinity reagent, and molecular membrane ultrafiltration. In some embodiments, the test sample, such as the test plasma sample generated using the methods described herein, is not further depleted to remove any one or more of seven common highly abundant blood proteins (albumin, IgG, antitrypsin, IgA, transferrin, haptoglobin, fibrinogen) prior to subjecting the test sample to the separation technique described herein, such as a SEC technique using a SEC microfluidic device. In some embodiments, the test sample, such as the test plasma sample generated using the methods described herein, is not further depleted to remove any one or more of fourteen common highly abundant blood proteins (albumin, IgG, antitrypsin, IgA, transferrin, haptoglobin, fibrinogen, alpha2-macroglobulin, alpha1-acid glycoprotein, IgM, apolipoprotein AI, apolipoprotein AII, complement C3, transthyretin) prior to subjecting the test sample to the separation technique described herein, such as a SEC technique using a SEC microfluidic device.

In some embodiments, the plasma generation technique is performed at an ambient temperature, such as at or around room temperature. In some embodiments, the plasma generation technique is performed at a temperature of about 20° C. to about 40° C. In some embodiments, the plasma generation technique is performed at a temperature of about any of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

### B. Liquid Chromatography

In certain aspects, the methods described herein comprise a liquid chromatography method (such as a liquid chromatography step) designed to separate and/or concentrate a component, or a product thereof, of a sample. In some embodiments, the methods for processing components, or products thereof, of a biological sample, such as a sample from an individual, for a mass spectrometry analysis comprise one or more dimensions of chromatography, including two, three, and four dimensions of chromatography. In some embodiments, for a method comprising more than one dimension of chromatography, the chromatography dimensions are performed offline, and may optionally include one more processing steps before, after, or between. In some embodiments, for a method comprising more than dimension of chromatography, the chromatography dimensions are performed online. In some embodiments, the dimensions of chromatography of the methods described herein are orthogonal. In some embodiments, the liquid chromatography methods described herein are completed using a microfluidic device having a plurality of interconnected channels as described herein.

In some embodiments, two or more chromatography steps (such as a size-exclusion chromatography step followed by a reversed-phase chromatography step) is completed sequentially, e.g., on the same chip, for applications not requiring an intermediary proteolysis step (e.g., for the analysis of native peptides or metabolites that can serve as surrogate markers of protein pathways and networks). In some embodiments, such an integrated two-dimensional μSEC-RP lab-chip can be directly interfaced to an atmospheric pressure ionization source for a mass spectrometer.

i. Size-Exclusion Chromatography (SEC)

Provided herein, in certain aspects, are methods comprising a size-exclusion chromatography (SEC) technique, such as a SEC technique completed using a SEC microfluidic device described herein. In some embodiments, the SEC technique comprises introducing a fluid input to a SEC microfluidic device. In some embodiments, the fluid input is a test sample or a derivative thereof, such as a product of some further processing step.

In some embodiments, the fluid input, such as a test sample, subjected to the SEC technique using the SEC microfluidic device has a volume of about 1 μL to about 200 μL. In some embodiments, the fluid input, such as a test sample, subjected to the SEC technique using the SEC microfluidic device has a volume of at least about 1 μL, such as at least about any of 5 μL, 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, or 190 μL. In some embodiments, the fluid input, such as a test sample, subjected to the SEC technique using the SEC microfluidic device has a volume of less than about 200 μL, such less than about any of 190 μL, 180 μL, 170 μL, 160 μL, 150 μL, 140 μL, 130 μL, 120 μL, 110 μL, 100 μL, 95 μL, 90 μL, 85 μL, 80 μL, 75 μL, 70 μL, 65 μL, 60 μL, 55 μL, 50 μL, 45 μL, 40 μL, 35 μL, 30 μL, 20 μL, 10 μL, or 5 μL. In some embodiments, the fluid input, such as a test sample, subjected to the SEC technique using the SEC microfluidic device has a volume of about any of 1 μL, 5 μL, 10 μL, 15 μL, 20 μL, 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, or 200 μL.

In some embodiments, the SEC technique comprises use of a SEC mobile phase having a concentration of a mobile phase chaotropic agent within a range of a pre-determined concentration of a chaotropic agent in a test sample. In some embodiments, the range of the concentration of a mobile phase chaotropic agent of a SEC technique is within about +/−40%, such as about any of +/−35%, +/−30%, +/−25%, +/−20%, +/−15%, +/−10%, +/−8%, +/−6%, +/−5%, +/−4%, +/−3%, +/−2%, +/−1%, of a pre-determined concentration of a chaotropic agent of a test sample. For example, in some embodiments, for a test sample comprising 6 M guanidine hydrochloride, the SEC mobile phase comprises guanidine at +/−10% of 6 M, including 6 M.

In some embodiments, the mobile phase chaotropic agent of a SEC technique is the same as a chaotropic agent of a liquid fixative. In some embodiments, the mobile phase chaotropic agent of a SEC technique is different than a chaotropic agent of a liquid fixative. In some embodiments, the mobile phase chaotropic agent comprises guanidine or a salt thereof, guanidinium or a salt thereof, potassium or a salt thereof, lithium or a salt thereof, magnesium or a salt thereof, or sodium or a salt thereof. In some embodiments, the mobile phase chaotropic agent is selected from the group consisting of guanidine hydrochloride, guanidinium chloride, guanidinium thiocynante, lithium perchlorate, lithium acetate, magnesium chloride, potassium acetate, and sodium iodide. In some embodiments, the mobile phase chaotropic agent is a guanidine salt. In some embodiments, the mobile phase chaotropic agent is guanidine hydrochloride.

In some embodiments, the SEC mobile phase comprises a concentration of a mobile phase chaotropic agent of about 5 M to about 8 M, such as any of about 5.5 M to about 8 M, about 5.5 M to about 7 M, or about 5.5 M to about 6.5 M. In some embodiments, the SEC mobile phase comprises a concentration of a mobile phase chaotropic agent of at least about 5.5 M, such as at least about any of 6 M, 6.5 M, 7 M, 7.5 M, or 8 M. In some embodiments, the SEC mobile phase comprises a concentration of a mobile phase chaotropic agent of about 8 M or less, such as about any of 7.5 M or less, 6.5 M or less, or 6 M or less. In some embodiments, the SEC mobile phase comprises a concentration of a mobile phase chaotropic agent of 5.5 M, 6 M, 6.5 M, 7 M, 7.5 M, or 8 M.

In some embodiments, the SEC mobile phase comprises a mobile phase viscosity modulating agent. In some embodiments, the mobile phase viscosity modulating agent is selected from the group consisting of glycerol, propylene glycol, sorbitol, and polyethylene glycol (PEG). In some embodiments, the mobile phase viscosity modulating agent is glycerol.

In some embodiments, the SEC mobile phase comprises a concentration of a mobile phase viscosity modulating agent of about 5% to about 40%, such as any of about 5% to about 20%, about 10% to about 30%, about 20% to about 30%, or about 20% to about 40%. In some embodiments, the SEC mobile phase comprises a concentration of a mobile phase viscosity modulating agent of at least about 5%, such as at least about any of 10%, 15%, 20%, 25%, 30%, 35%, or 40%. In some embodiments, the SEC mobile phase comprises a concentration of a mobile phase viscosity modulating agent of about 40% or less, such as about any of 35% or less, 30% or less, 25% or less, 20% or less, 15% or less, 10% or less, or 5% or less. In some embodiments, the SEC mobile phase comprises a concentration of a mobile phase viscosity modulating agent of 5%, 10%, 15%, 20%, 25%, 30%, 35%, 40%, 45%, or 50%. In some embodiments, the amount of a viscosity modulating agent in a mobile phase is based on the desired viscosity of the mobile phase (such as for processing via aspects of the methods described herein, including a SEC microfluidic device).

In some embodiments, the SEC mobile phase comprises a concentration of a mobile phase chaotropic agent (such a guanidine hydrochloride) of about 5 M to about 8 M, such as any of about 5.5 M to about 7.5 M, about 5.5 M to about 7 M, or about 5.5 M to about 6.5 M, and a concentration of a mobile phase viscosity modulating agent (such as glycerol) of about 5% to about 40%, such as any of about 5% to about 20%, about 10% to about 30%, about 20% to about 30%, or about 20% to about 40%. In some embodiments, the SEC mobile phase comprises a concentration of a mobile phase chaotropic agent (such a guanidine hydrochloride) of at least about 5 M, such as at least about any of 5.5 M, 6 M, 6.5 M, 7 M, 7.5 M, or 8 M, and a concentration of a mobile phase viscosity modulating agent (such as glycerol) of at least about 5%, such as at least about any of 10%, 15%, 20%, 25%, 30%, 35%, or 40%. In some embodiments, the SEC mobile phase comprises a concentration of a mobile phase chaotropic agent (such a guanidine hydrochloride) of about 8 M or less, such as about any of 7.5 M or less, 6.5 M or less, or 6 M or less, and a concentration of a mobile phase viscosity modulating agent (such as glycerol) of about 40% or less, such as about 35% or less, 30% or less, 25% or less, 20% or less, 15% or less, 10% or less, or 5% or less. In some embodiments, the SEC mobile phase comprises a concentration of a mobile phase chaotropic agent (such a guanidine hydrochloride) of about any of 5 M, 5.5 M, 6 M, 6.5 M, 7 M, 7.5 M, or 8 M, and a concentration of a mobile phase viscosity modulating agent (such as glycerol) of about any of 5%, 10%, 15%, 20%, 25%, 30%, 35%, 40%, 45%, or 50%.

In some embodiments, the SEC mobile phase comprises a concentration of a mobile phase chaotropic agent (e.g., guanidine hydrochloride) of about 5.5 M to about 8 M, such as about 6 M or more, and a concentration of a mobile phase viscosity modifying agent (e.g., glycerol) of about 5% to about 40%, such about 10% to about 30%.

In some embodiments, the mobile phase viscosity modifying agent of a SEC technique is the same as a viscosity modifying agent of a liquid fixative. In some embodiments, the mobile phase viscosity modifying agent of a SEC technique is different than a viscosity modifying agent of a liquid fixative.

In some embodiments, the SEC technique is an isocratic SEC technique (i.e., a single SEC mobile phase is used and a gradient of component concentrations is not performed).

In some embodiments, the SEC technique comprises use of a mobile phase flow rate of about 1 μL/minute to about 5 μL/minute, such as about any of 1 μL/minute, 1.5 μL/minute, 2 μL/minute, 2.5 μL/minute, 3 μL/minute, 3.5 μL/minute, 4 μL/minute, 4.5 μL/minute, or 5 μL/minute. In some embodiments, the mobile phase may be introduced and the flow rate controlled by systems known in the art, such as a syringe pump or an ultra-high performance liquid chromatography pump.

In some embodiments, the SEC technique described herein is performed at an ambient temperature (such as based on a column temperature), such as at or around room temperature. In some embodiments, the SEC technique is performed at an elevated temperature. In some embodiments, the SEC technique is performed at a temperature of about 15° C. to about 60° C., such as any of about 15° C. to about 45° C., about 23° C. to about 45° C., about 30° C. to about 50° C., or about 45° C. to about 60° C. In some embodiments, the SEC technique is performed at a temperature of at least about 15° C., such as at least about any of 20° C., 25° C., 30° C., 35° C., 40° C., 45° C., 50° C., 55° C., or 60° C. In some embodiments, the SEC technique is performed at a temperature of less than about 60° C., such as less than about any of 55° C., 50° C., 45° C., 40° C., 35° C., 30° C., 25° C., 20° C., or 15° C. In some embodiments, the SEC technique is performed at about any of 15° C., 20° C., 25° C., 30° C., 35° C., 40° C., 45° C., 50° C., 55° C., or 60° C.

In some embodiments, the SEC technique is performed at a substantially consistent temperature. For example, in some embodiments, the SEC technique is performed with a range of a desired temperature. In some embodiments, the range is about any of +/−8° C., +/−6° C., +/−5° C., +/−4° C., +/−3° C., +/−2° C., or +/−1° C., of a desired temperature. For example, in some embodiments, the SEC technique is performed with a range of +/−5° C. of 21° C.

In some embodiments, the SEC technique comprises use of a SEC medium selected based on a desired separation. In some embodiments, the SEC technique comprises selecting a SEC medium based on a characteristic thereof, such as compatibility with components of a SEC microfluidic device and/or pore size.

ii. Reversed-Phase Liquid Chromatography

Provided herein are methods comprising a reversed-phase liquid chromatography (RPLC) technique, such as a RPLC technique completed using a RPLC microfluidic device described herein. In some embodiments, the RPLC technique comprises introducing a fluid input to a RPLC microfluidic device. In some embodiments, the fluid input is a RPLC-compatible fluid, such as a RPLC-compatible fraction, include those obtained from a method described herein, e.g., from a SEC technique completed using a SEC microfluidic device described herein, and optionally subjected to proteolytic technique.

In some embodiments, the fraction subjected to a RPLC technique is modulated from its source. For example, in some embodiments, the fraction subjected to a RPLC technique comprises at least a portion of a SEC fraction, wherein the SEC fraction is further processed prior being subjected to the RPLC technique. In some embodiments, the fraction subjected to a RPLC technique comprises at least a portion of a fraction subjected to a proteolysis technique, wherein the fraction subjected to the proteolysis technique is further processed prior being subjected to the RPLC technique. In some embodiments, the fraction subjected to a RPLC technique comprises at least a portion of a fraction subjected to a quantitative labeling technique, wherein the fraction subjected to the quantitative labeling technique is further processed prior being subjected to the RPLC technique. In some embodiments, the fraction subjected to a RPLC technique has undergone a desalting step. In some embodiments, the fraction subjected to a RPLC technique has undergone a dilution step, such as dilution with a RPLC compatible solution.

In some embodiments, each of a set of fractions, or portions thereof, are subjected to a RPLC technique described herein, including a RPLC chromatography technique completed using a RPLC microfluidic device. In some embodiments, the set of fractions comprises a fraction obtained from a SEC microfluidic device following a SEC technique, or a processed derivative thereof. In some embodiments, the set of fractions comprises a fraction obtained from a proteolytic technique, or a processed derivative thereof. In some embodiments, the set of fractions comprises a portion of a fraction from a SEC microfluidic device, and another portion of the fraction from the SEC microfluidic device subjected to a proteolytic technique.

In some embodiments, the fluid input, such as a fraction, to a RPLC microfluidic device has a volume of about 1 μL to about 50 μL, such as about 1 μL to about 25 μL, or about 5 μL to about 20 μL. In some embodiments, the fluid input, such as a fraction, to a RPLC microfluidic device has a volume of at least about 1 μL, such as at least about any of 2 μL, 3 μL, 4 μL, 5 μL, 6 μL, 7 μL, 8 μL, 9 μL, 10 μL, 15 μL, 20 μL, 25 μL, 30 μL, 35 μL, 40 μL, 45 μL, or 50 μL. In some embodiments, the fluid input, such as a fraction, to a RPLC microfluidic device has a volume of at less than about 50 μL, such as less than about any of 45 μL, 40 μL, 35 μL, 30 μL, 25 μL, 20 μL, 15 μL, 10 μL, 9 μL, 8 μL, 7 μL, 6 μL, 5 μL, 4 μL, 3 μL, 2 μL, or 1 μL. In some embodiments, the fluid input, such as a fraction, to a RPLC microfluidic device has a volume of about any of 1 μL, 2 μL, 3 μL, 4 μL, 5 μL, 6 μL, 7 μL, 8 μL, 9 μL, 10 μL, 15 μL, 20 μL, 25 μL, 30 μL, 35 μL, 40 μL, 45 μL, or 50 μL.

In some embodiments, the RPLC technique comprise use of a RPLC mobile phase. RPLC mobile phases are well known in the art and are compatible with the methods and devices described herein. In some embodiments, the RPLC mobile phase is a dynamic mobile phase that is adjusted over the course of a RPLC technique, such as to facilitate elution of component, or a product thereof, of a sample. For example, in some embodiments, the RPLC mobile phase comprises a concentration of an aqueous solution and a concentration of an organic solution. In some embodiments, the aqueous solution comprises water, such as ultrapure water. In some embodiments, the organic solution comprises acetonitrile. In some embodiments, the RPLC mobile phase comprises an additional component useful for the RPLC technique and/or mass spectrometry. For example, in some embodiments, the RPLC mobile phase is adjusted with a weak acid to have an acidic pH. In some embodiments, the RPLC mobile phase comprises a weak acid, such as formic acid, trifluoroacetic acid, or acetic acid. In some embodiments, the concentration of the weak acid in a RPLC mobile phase is less than about 0.5%, such as about any 0.4%, 0.3%, 0.2%, or 0.1%.

In some embodiments, the RPLC technique is a gradient RPLC technique (i.e., a gradient of mobile phase components, such as increasing an amount of the organic phase of the mobile phase is used for elution).

In some embodiments, the RPLC technique comprises use of a mobile phase flow rate of about 0.05 μL/minute to about 2 μL/minute, such as about any of 0.1 μL/minute, 0.2 μL/minute, 0.3 μL/minute, 0.4 μL/minute, 0.5 μL/minute, 0.6 μL/minute, 0.7 μL/minute, 0.8 μL/minute, 0.9 μL/minute, 1 μL/minute, 1.1 μL/minute, 1.2 μL/minute, 1.3 μL/minute, 1.4 μL/minute, 1.5 μL/minute, 1.6 μL/minute, 1.7 μL/minute, 1.8 μL/minute, 1.9 μL/minute, or 2 μL/minute. In some embodiments, the mobile phase may be introduced and the flow rate controlled by systems known in the art, such as a syringe pump or an ultra-high performance liquid chromatography pump.

In some embodiments, the RPLC technique described herein is performed (such as evaluated by column temperature) at an ambient temperature, such as at or around room temperature. In some embodiments, the RPLC technique is performed at an elevated temperature. In some embodiments, the SEC technique is performed at a temperature of about 15° C. to about 100° C., such as any of about 15° C. to about 45° C., about 23° C. to about 45° C., about 30° C. to about 50° C., or about 45° C. to about 60° C. In some embodiments, the RPLC technique is performed at a temperature of at least about 15° C., such as at least about any of 20° C., 25° C., 30° C., 35° C., 40° C., 45° C., 50° C., 55° C., 60° C., 65° C., 70° C., 75° C., 80° C., 85° C., 90° C., 95° C., or 100° C. In some embodiments, the SEC technique is performed at a temperature of less than about 100° C., such as less than about any of 95° C., 90° C., 85° C., 80° C., 75° C., 70° C., 65° C., 60° C., 55° C., 50° C., 45° C., 40° C., 35° C., 30° C., 25° C., 20° C., or 15° C. In some embodiments, the RPLC technique is performed at about any of 15° C., 20° C., 25° C., 30° C., 35° C., 40° C., 45° C., 50° C., 55° C., 60° C., 65° C., 70° C., 75° C., 80° C., 85° C., 90° C., 95° C., or 100° C.

In some embodiments, the RPLC technique is performed at a substantially consistent temperature. For example, in some embodiments, the RPLC technique is performed with a range of a desired temperature. In some embodiments, the range is about any of +/−8° C., +/−6° C., +/−5° C., +/−4° C., +/−3° C., +/−2° C., or +/−1° C., of a desired temperature. For example, in some embodiments, the RPLC technique is performed with a range of +/−5° C. of 21° C.

### C. Fraction Collection

In some aspects, provided herein are fraction collection techniques and fraction collection devices useful for capturing fractions (e.g., individual segments) of a sample after some degree of separation using a chromatography technique described herein.

In some embodiments, a fraction characteristic (such as size or duration of collection) is based, at least in part, on a desired division of a separation performed by a liquid chromatography technique described herein. In some embodiments, the method comprises selecting a fraction based on a time of elution. For example, in some embodiments, the fractions is collected from a liquid chromatography technique, such as a SEC technique using a SEC microfluidic device described herein, for a period of about 30 seconds to about 5 minutes, such as any of about 30 seconds to about 3 min, about 1 minutes to about 2 minutes, about 1 minute to about 4 minutes, or about 2 minutes to about 5 minutes. In some embodiments, the fraction is collected from a liquid chromatography technique, such as a SEC technique using a SEC microfluidic device described herein, for a period of at least about 30 seconds, such as at least about any of 1 minute, 1.5 minutes, 2 minutes, 2.5 minutes, 3 minutes, 3.5 minutes, 4 minutes, 4.5 minutes, or 5 minutes. In some embodiments, the fraction is collected from a liquid chromatography technique, such as a SEC technique using a SEC microfluidic device described herein, for a period of about 5 minutes or less, such as a period of less than about any of 4.5 minutes or less, 4 minutes or less, 3.5 minutes or less, 3 minutes, 2.5 minutes, 2 minutes, 1.5 minutes, 1 minutes, or 30 seconds. In some embodiments, the fraction is collected from a liquid chromatography technique, such as a SEC technique using a SEC microfluidic device described herein, for a period of about any of 30 seconds, 1 minutes, 1.5 minutes, 2 minutes, 2.5 minutes, 3 minutes, 3.5 minutes, 4 minutes, 4.5 minutes, or 5 minutes. In some embodiments, each of the plurality of fraction is collected from a SEC microfluidic device for a period of about 1 minutes to about 2 minutes.

In some embodiments, each of a plurality of fractions is collected from a liquid chromatography technique, such as a SEC technique using a SEC microfluidic device described herein, for a uniform amount of time. In some embodiments, one fraction of a plurality of fractions is collected from a liquid chromatography technique, such as a SEC technique using a SEC microfluidic device described herein, for a different amount of time than another fraction of the plurality of fractions.

In some embodiments, the fraction is collected from a liquid chromatography technique, such as a SEC technique using a SEC microfluidic device described herein, based on volume of eluate therefrom. In some embodiments, the fraction has a volume of about 1 μL to about 20 μL, such as any of about 1 μL to about 8 μL, about 5 μL to about 15 μL, or about 10 μL to about 20 μL. In some embodiments, the fraction has a volume of least about 1 μL, such as at least about any of 2 μL, 3 μL, 4 μL, 5 μL, 6 μL, 7 μL, 8 μL, 9 μL, 10 μL, 11 μL, 12 μL, 13 μL, 14 μL, 15 μL, 16 μL, 17 μL, 18 μL, 19 μL, or 20 μL. In some embodiments, the fraction has a volume of about 20 μL or less, such as about any of 19 μL or less, 18 μL or less, 17 μL or less, 16 μL or less, 15 μL or less, 14 μL or less, 13 μL or less, 12 μL or less, 11 μL or less, 10 μL or less, 9 μL or less, 8 μL or less, 7 μL or les, 6 μL or less, 5 μL or less, 4 μL or less, 3 μL or less, 2 μL or less, or 1 μL or less. In some embodiments, the fraction has a volume of about any of 1 μL, 2 μL, 3 μL, 4 μL, 5 μL, 6 μL, 7 μL, 8 μL, 9 μL, 10 μL, 11 μL, 12 μL, 13 μL, 14 μL, 15 μL, 16 μL, 17 μL, 18 μL, 19 μL, or 20 μL. In some embodiments, each of a plurality of fractions collected from a liquid chromatography technique, such as a SEC technique using a SEC microfluidic device described herein, has a uniform volume. In some embodiments, one fraction of a plurality of fractions collected from a liquid chromatography technique, such as a SEC technique using a SEC microfluidic device described herein, has different volume than another fraction of the plurality of fractions.

In some embodiments, the method comprises collecting a plurality of fractions from a liquid chromatography technique, such as a SEC technique using a SEC microfluidic device described herein. In some embodiments, the plurality of fractions is about 5 fractions to about 50 fractions, such as about 5 fractions to about 30 fractions, about 12 fractions to about 24 fractions, or about 30 fractions to about 50 fractions. In some embodiments, the plurality of fractions is at least about 5 fractions, such as at least about any of 10 fractions, 11 fractions, 12 fractions, 13 fractions, 14 fractions, 15 fractions, 16 fractions, 17 fractions, 18 fractions, 19 fractions, 20 fractions, 21 fractions, 22 fractions, 23 fractions, 24 fractions, 25 fractions, 30 fractions, 35 fractions, 40 fractions, 45 fractions, or 50 fractions. In some embodiments, the plurality of fractions is about 50 or less fractions, such as about any of 45 or less fractions, 40 or less fractions, 35 or less fractions, 30 or less fractions, 25 or less fractions, 24 or less fractions, 23 or less fractions, 22 or less fractions, 21 or less fractions, 20 or less fractions, 19 or less fractions, 18 or less fractions, 17 or less fractions, 16 or less fractions, 15 or less fractions, 14 or less fractions, 13 or less fractions, 12 or less fractions, 11 or less fractions, 10 or less fractions, or 5 or less fractions. In some embodiments, the plurality of fractions is about any of 5 fractions, 10 fractions, 11 fractions, 12 fractions, 13 fractions, 14 fractions, 15 fractions, 16 fractions, 17 fractions, 18 fractions, 19 fractions, 20 fractions, 21 fractions, 22 fractions, 23 fractions, 24 fractions, 25 fractions, 30 fractions, 35 fractions, 40 fractions, 45 fractions, or 50 fractions. In some embodiments, a plurality of fractions is about 12 fractions to about 24 fractions, including about 12 fractions.

In some embodiments, the fractions are collected using fraction collector. In some embodiments, the fraction collector is connected to a liquid chromatography device described herein, such as a SEC microfluidic device. In some embodiments, the fractions are collected via a microfluidic or chip-based feature, such as a compartment of a microfluidic device (e.g., a lab-on-a-chip device). In some embodiments, the plurality of fractions eluted from a SEC microfluidic device described herein are collected using a chip-based fraction collector (e.g., lab-chip device).

### D. Lytic Techniques

In some embodiments, the method comprises a lytic technique, such as a proteolytic technique. In some embodiments, the lytic technique results in the separation of a parts of a component, or product thereof, of a sample. For example, in some embodiments, the lytic technique is a proteolytic technique that breaks down a polypeptide into two or more resulting products. In some embodiments, the lytic technique separates a metabolite (such as a post-translation modification) from a polypeptide. In some embodiments, the lytic technique separates a metabolite into two or more products.

Proteolytic techniques for producing polypeptide, such as peptide, products of a parent polypeptide of a sample for analysis via a mass spectrometry technique are known in the art. In some embodiments, the polypeptide, such as a peptide, products of a parent polypeptide are obtained via proteolysis (e.g., sample digestion) prior to subjecting the polypeptide products to a mass spectrometer. In some embodiments, the polypeptide, such as a peptide, products of a parent polypeptide are obtained within a mass spectrometer. In some embodiments, the proteolytic technique is performed on one or more, such as all, of a plurality of fractions obtained from a method described herein. In some embodiments, the proteolytic technique is performed on a sample or a portion of a fraction obtained from a method described herein.

In some embodiments, the proteolytic technique comprises an enzyme-based digestion technique. In some embodiments, the enzyme-based digestion technique comprises the use of a proteolytic enzyme, such as a protease. In some embodiments, the proteolytic enzyme is selected from the group consisting of trypsin, chymotrypsin, thermolysin, pepsin, elastase, Lys-C, Lys-N, Asp-N, Glu-C, Arg-C, TEV, IdeS, IdeZ, PNGase F, and Factor Xa, or a combination thereof.

In some embodiments, the proteolytic technique is a chemical-based proteolytic technique. In some embodiments, the chemical-based proteolytic technique comprises use of an acid, such as a strong acid.

In some embodiments, the proteolytic technique is a solution-phase proteolytic technique. In some embodiments, the proteolytic technique is a solid-phase or solid-state proteolytic technique. In some embodiments, the proteolytic technique is a gel-phase proteolytic technique.

Techniques for performing the lytic techniques, or portions thereof, encompassed in the disclosure of the present application are well known in the art. In some embodiments, considerations of such techniques include the environment of the reaction, such as a solution and components thereof, the temperature, the duration, the ratio of a digestive component, such as a protease, relative to the components of the sample. For example, in some embodiments, the solution-phase trypsin proteolytic technique comprises admixing trypsin with a diluted fraction from at about a 1:30 ratio, and incubating for about 8 hours at about 37° C.

In some embodiments, the lytic technique, such as a proteolytic technique, comprises a step of diluting the input to the technique, such as a fraction obtained from a method described herein. In some embodiments, the dilution is performed using water, an organic solvent, a weak buffer, a compatible buffer, or a combination thereof. In some embodiments, the dilution is performed to ensure compatibility of the resulting diluted material with a lytic technique. In some embodiments, the dilution step is based on an obtaining a final concentration of a chaotropic agent (such as guanidine hydrochloride) of about 0.1 to about 2 M, such as any of about 0.1 M to about 0.5 M, about 0.5 M to about 1.5 M, or about 1 M to about 2 M. In some embodiments, the dilution step is based on an obtaining a final concentration of a chaotropic agent of less than about 1 M, such as less than about any of 0.9 M, 0.8 M, 0.7 M, 0.6 M, 0.5 M, 0.4 M, 0.3 M, 0.2 M, 0.1 M, or 0.05 M.

In some embodiments, the enzyme-based digestion technique does not comprise a buffer exchange step. In some embodiments, the enzyme-based digestion technique does not comprise an alkylation step. In some embodiments, the enzyme-based digestion technique does not comprise a reduction step.

### E. Quantification Techniques

In some embodiments, the methods described herein comprise a quantification technique. In some embodiments, the quantification method provides a measure of the abundance of a component, or a product thereof, in a sample. In some embodiments, the quantification method is a relative quantification method. In some embodiments, the quantification method is a semi-relative quantification method. In some embodiments, the quantification method is an absolute quantification method. In some embodiments, the quantification method is a label-free quantification method. In some embodiments, the quantification method is a label-based quantification method, such as comprising use of isobaric tags, e.g., tandem mass tags. In some embodiments, the quantification method is a spike-in method, such as involving use of one or more standards, e.g., as isotopically labeled peptide. In some embodiments, the quantification method comprises any combinations of a quantification method.

In some embodiments, the quantification method comprises a clean-up step prior to starting a downstream step of the method For example, in some embodiments, the quantification method comprises a desalting step, such as to remove excess label not conjugated to a component, or a product thereof, of a sample.

Mass spectrometry quantification methods are well known in the art. See, e.g., Bantscheff et al., Anal Bioanal Chem, 389, 2007, which is hereby incorporated by reference herein in its entirety.

### F. Introduction of Components, or Products Thereof, of a Sample to a Mass Spectometer

Provided herein are techniques and devices (such as emitters) for introducing a component, or a product thereof, of a sample to a mass spectrometry. Introduction techniques, and devices thereof, are well known in the art and compatible with the methods described herein. In some embodiments, the introduction technique comprises an ionization technique. In some embodiments, the ionization technique is an electrospray ionization technique. In some embodiments, the electrospray ionization technique is based on the flow rate use with the technique. For example, in some embodiments, the electrospray ionization technique is a nano-electrospray ionization technique. In some embodiments, the electrospray ionization technique comprises use of an electrospray ionization source, such as a nano-electrospray ionization source. In some embodiments, the ionization technique is an atmospheric pressure chemical ionization technique. In some embodiments, the ionization technique is an atmospheric pressure photo ionization technique. In some embodiments, the ionization technique is an offline desorption electrospray ionization (DESI) technique. In some embodiment, the ionization technique is an offline matrix-assisted laser desorption ionization (MALDI) technique.

In some embodiments, the electrospray ionization source is a heated electrospray ionization source. In some embodiments, the electrospray ionization source is coupled with a gas drying features, such as a nitrogen stream or curtain.

In some embodiments, the ionization technique, such as the online ionization technique, is coupled with an atmospheric pressure high field asymmetric waveform ion mobility spectrometry (FAIMS) system retrofitted with a mass spectrometer.

### G. Mass Spectrometry and Data Acquisition Techniques

The present application contemplates a diverse array of mass spectrometry techniques suitable for use with methods and method steps disclosed herein, including determining a mass spectrometry profile. In some embodiments, the methods disclosed herein comprise analyzing a sample using one or more mass spectrometry techniques. As discussed herein, in some embodiments, mass spectrometry techniques are used to acquire data to provide and/or are useful to obtain a vast amount of information about components, or products thereof, a sample, including any combination of MS ion information (m/z and abundance), identification/sequence information, such as peptide and/or protein identification/sequence information, post-translation modification information, metabolite identity, and quantification information.

In some embodiments, the mass spectrometry technique comprises use of a mass spectrometry technique. Mass spectrometers contemplated by the present invention include high-resolution mass spectrometers and low-resolution mass spectrometers. In some embodiments, the mass spectrometer is a time-of-flight (TOF) mass spectrometer. In some embodiments, the mass spectrometer is a quadrupole time-of-flight (Q-TOF) mass spectrometer. In some embodiments, the mass spectrometer is a single quadrupole. In some embodiments, the mass spectrometer is a triple quadrupole (QQQ). In some embodiments, the mass spectrometer is a quadrupole ion trap time-of-flight (QIT-TOF) mass spectrometer. In some embodiments, the mass spectrometer is a quadrupole-linear ion trap (Q-LIT). In some embodiments, the mass spectrometer relies on the Fourier Transform-Orbitrap as one of its constituent ion optical components, such as the hybrid quadrupole-Orbitrap, linear ion trap-orbitrap, or the tribrid quadrupole-linear ion trap-Orbitrap variants. In some embodiments, the mass spectrometer is an FT-ion cyclotron resonance (FT) mass spectrometer. In some embodiments, the mass spectrometer is a quadrupole FT-ion cyclotron resonance (Q-FT) mass spectrometer. In some embodiments, the mass spectrometer magnetic sector mass spectrometer.

In some embodiments, the mass spectrometry technique comprises use of a positive ion mode. In some embodiments, the mass spectrometry technique comprises use of a negative ion mode. In some embodiments, the mass spectrometry technique comprises an ion mobility mass spectrometry technique.

In some embodiments, the mass spectrometry technique comprises a top-down mass spectrometry technique. In some embodiments, the mass spectrometry technique comprises a middle-down mass spectrometry technique. In some embodiments, the mass spectrometry technique comprises a bottom-up mass spectrometry technique. In some embodiments, the mass spectrometry technique is a tandem mass spectrometry technique. In some embodiments, the tandem mass spectrometry technique comprises a fragmentation technique. In some embodiments, the methods described herein encompass any combination thereof.

Various mass spectrometry data acquisition techniques are amenable with the methods described herein. For example, in some embodiments, the mass spectrometry data acquisition technique comprises data-dependent data acquisition, data-independent data acquisition, targeted data acquisition, or a combination thereof.

In some aspects, provided herein is a method of analyzing a collection of compositions using mass spectrometry, the method comprising: (a) subjecting each composition of the collection of compositions to a mass spectrometer; and (b) performing a mass spectrometry analysis of each composition of the collection of compositions, wherein the collection of compositions is obtained from a processing technique comprising fractionation of a test sample using a SEC technique comprising use of a SEC microfluidic device followed by application of each fraction, or a product thereof, to a RPLC technique comprising use of a RPLC microfluidic device. In some embodiments, the SEC fraction is further processed via a proteolysis technique.

### H. Discovery and Target-Based Modes of the Methods Described Herein

Encompassed in the methods described herein are discovery-mode methods, semi-targeted-mode methods, targeted-mode methods, and combinations thereof. Use, and selection thereof, of a type of mode may be based on the desired information to evaluate for in a sample. For example, in some embodiments, it is desirable to study a multitude of components of a sample (such as may be more amenable to a discovery-mode or semi-targeted mode), e.g., in a hypothesis-free evaluation of a sample. In some embodiments, it is desirable to study a small selection of components of a sample (such as may be more amenable to a targeted-mode). Based on the purpose and/or desired information, one of ordinary skill in the art will readily appreciate, based on the teachings provided herein, how to design and run a method described herein. For example, the purpose and/or desired information may be used to design how many fractions are produced and obtained from a SEC technique, how many SEC fractions are further analyzed and what, if any, further processing is performed (such as a proteolytic technique), and what mass spectrometer and mass spectrometry analysis technique are used.

### I. Exemplary Methods for the Analysis of Components of a Sample

In some embodiments, provided is a method for processing components, or products thereof, of a biological sample for a mass spectrometry analysis, the method comprising: (a) subjecting a test sample to a size-exclusion chromatography (SEC) technique using a SEC microfluidic device, wherein the test sample comprises the sample admixed with a liquid fixative, wherein the test sample has a pre-determined concentration of a chaotropic agent originating from the liquid fixative, wherein the SEC technique comprises use of a SEC mobile phase having a concentration of a mobile phase chaotropic agent within a range of the pre-determined concentration of the chaotropic agent in the test sample, and wherein the SEC microfluidic device comprises a plurality of interconnected channels, each channel being configured in an open tubular format having an inner surface material having an average pore size of about 10 nm to about 500 nm; (b) collecting a plurality of fractions eluted from the SEC microfluidic device; (c) subjecting one or more of the plurality of fractions from the SEC microfluidic device to a proteolytic technique; and (d) subjecting each of a set of RPLC-compatible fractions to a reversed-phase liquid chromatography (RPLC) technique using a RPLC microfluidic device to prepare the components, or products thereof, of the sample for introduction to a mass spectrometer, wherein the set of RPLC-compatible fractions comprises fractions obtained from: (i) zero or more of the plurality of fractions from the SEC microfluidic device; and (ii) one or more of the plurality of fractions subjected to the proteolytic technique, wherein the RPLC technique and RPLC microfluidic device are configured for online desalting, wherein the RPLC microfluidic device comprises a plurality of interconnected channels, each channel being configured in an open tubular format having an inner surface material comprising a reversed-phase medium, and wherein the RPLC microfluidic device is coupled to an electrospray ionization source. In some embodiments, the biological sample is a plasma sample from an individual, such as a human. In some embodiments, the chaotropic agent, such as found in the liquid fixative and the SEC mobile phase, is guanidine hydrochloride. In some embodiments, the method further comprises subjecting the eluate from the RPLC microfluidic device to the mass spectrometer. In some embodiments, the individual is a human.

In some embodiments, provided is a method for processing components, or products thereof, of a plasma sample from a human for a mass spectrometry analysis, the method comprising: (a) subjecting a test plasma sample to a size-exclusion chromatography (SEC) technique using a SEC microfluidic device, wherein the test sample comprises the sample admixed with a liquid fixative, wherein the test sample has at least about 5.5 M, such as at least about 6 M, guanidine (e.g., from guanidine hydrochloride) originating from the liquid fixative, wherein the SEC technique comprises use of a SEC mobile phase having at least about 5.5 M, such as at least about 6 M, guanidine (e.g., from guanidine hydrochloride), and wherein the SEC microfluidic device comprises a plurality of interconnected channels, each channel being configured in an open tubular format having an inner surface material having an average pore size of about 10 nm to about 500 nm; (b) collecting a plurality of fractions eluted from the SEC microfluidic device; (c) subjecting one or more of the plurality of fractions from the SEC microfluidic device to a proteolytic technique using a proteolytic enzyme, such as trypsin; and (d) subjecting each of a set of RPLC-compatible fractions to a reversed-phase liquid chromatography (RPLC) technique using a RPLC microfluidic device to prepare the components, or products thereof, of the sample for introduction to a mass spectrometer, wherein the set of RPLC-compatible fractions comprises fractions obtained from: (i) zero or more of the plurality of fractions from the SEC microfluidic device; and (ii) one or more of the plurality of fractions subjected to the proteolytic technique, wherein the RPLC technique and RPLC microfluidic device are configured for online desalting, wherein the RPLC microfluidic device comprises a plurality of interconnected channels, each channel being configured in an open tubular format having an inner surface material comprising a reversed-phase medium, wherein the reversed-phase medium comprises one or more of C₂, C₄, C₈, or C₁₈, and wherein the RPLC microfluidic device is coupled to an electrospray ionization source. In some embodiments, the method further comprises generating a plasma sample. In some embodiments, the method further comprises subjecting the eluate from the RPLC microfluidic device to the mass spectrometer.

In some embodiments, provided is a method for processing components, or products thereof, of a blood sample from a human for a mass spectrometry analysis, the method comprising: (a) generating a test plasma sample from the blood sample, wherein the test plasma sample comprises a plasma sample from the blood sample admixed with a liquid fixative, wherein the test plasma sample has at least about 5.5 M, such as at least about 6 M, guanidine (e.g., from guanidine hydrochloride) originating from the liquid fixative; (b) subjecting the test plasma sample to a size-exclusion chromatography (SEC) technique using a SEC microfluidic device, wherein the SEC technique comprises use of a SEC mobile phase having at least about 5.5 M, such as at least about 6 M, guanidine (e.g., from guanidine hydrochloride), and wherein the SEC microfluidic device comprises a plurality of interconnected channels, each channel being configured in an open tubular format having an inner surface material having an average pore size of about 10 nm to about 500 nm; (c) collecting a plurality of fractions eluted from the SEC microfluidic device; (d) subjecting one or more of the plurality of fractions from the SEC microfluidic device to a proteolytic technique using a proteolytic enzyme, such as trypsin; and (e) subjecting each of a set of RPLC-compatible fractions to a reversed-phase liquid chromatography (RPLC) technique using a RPLC microfluidic device to prepare the components, or products thereof, of the sample for introduction to a mass spectrometer, wherein the set of RPLC-compatible fractions comprises fractions obtained from: (i) zero or more of the plurality of fractions from the SEC microfluidic device; and (ii) one or more of the plurality of fractions subjected to the proteolytic technique, wherein the RPLC technique and RPLC microfluidic device are configured for online desalting, wherein the RPLC microfluidic device comprises a plurality of interconnected channels, each channel being configured in an open tubular format having an inner surface material comprising a reversed-phase medium, wherein the reversed-phase medium comprises one or more of C₂, C₄, C₈, or C₁₈, and wherein the RPLC microfluidic device is coupled to an electrospray ionization source. In some embodiments, the method further comprises subjecting the eluate from the RPLC microfluidic device to the mass spectrometer.

### III. Coronary Artery Disease (CAD) Signature and Methods of Use Thereof

In certain aspects, provided herein is a coronary artery disease (CAD) signature comprising a plurality of biomarkers identified using the methods and devices described herein based on evaluation of samples from individuals having CAD as compared to healthy individuals. A major disease sub-type of Cardiovascular Disease (CVD) is Coronary Artery Disease (CAD), which is characterized by the narrowing and stiffness of the cardiac arteries known as atherosclerosis. Atherosclerosis is caused by multiple pathologic mechanisms, including endothelial injury and subendothelial apoB-lipoprotein retention, insulin resistance, oxidative stress, DNA damage and aging, autophagy, lipid metabolism dysregulation, inflammation, and thrombosis, and identifying signatures thereof is challenging.

The discovery of a CAD signature enables the use of one or more biomarkers thereof in, e.g., analytical methods for detecting a CAD proteomic signature in an individual, methods of diagnosis, and methods of treatment. In some embodiments, the methods provided herein only utilize a subset of the biomarkers of the identified CAD signature, such as one or more biomarkers of the CAD signature. In some embodiments, provided is a CAD proteomic signature comprising one or more biomarkers of the CAD signature provided Table 1 (provided below). In some embodiments, the CAD proteomic signature is evaluated via polypeptides in a sample, such as using a mass spectrometry technique. In some embodiments, the CAD proteomic signature is evaluated via a non-mass spectrometry based technique, such as ELISA.

In some embodiments, the methods provided herein comprise analyzing mass spectrometry (MS) data according to a CAD proteomic signature, wherein the CAD proteomic signature comprises one or more biomarkers of Table 1. In some embodiments, each biomarker of the CAD proteomic signature includes the protein identity and the status of increased or decreased expression of the protein (as noted in the CAD Signature column of Table 1) as compared to a reference (the level of the protein in one or more healthy individual, e.g., an individual not having CAD). For example, in some embodiments, the methods provided herein for assessing a CAD proteomic signature evaluate a sample, or a derivative thereof, obtained from an individual for the presence of the one or more biomarkers of the CAD proteomic signature and whether the one or more biomarkers of the CAD proteomic signature substantially agree (such as at least about 70%, including at least about any of 75%, 80%, 85%, 90%, or 95%, of the one or more biomarkers) with the increased expression or decreased expression classification of Table 1. In some embodiments, the methods provided herein for assessing a CAD proteomic signature evaluate a sample, or a derivative thereof, obtained from an individual for the presence of the one or more biomarkers of the CAD proteomic signature and whether the one or more biomarkers of the CAD proteomic signature agree with the increased expression or decreased expression classification of Table 1.

In some embodiments, each biomarker of the CAD proteomic signature includes the protein identity and a level of increased or decreased expression of the protein (such as a level above a set threshold defined for increased or decreased expression) as compared to a reference (the level of the protein in one or more healthy individual, e.g., an individual not having CAD). In some embodiments, increased expression of a protein is a mean log 2 ratio, as measured in the individual as compared to a reference, of at least about 0.2, such as at least about any of 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0. In some embodiments, decreased expression of a protein is a mean log 2 ratio, as measured in the individual as compared to a reference, of less than or equal to about −0.2, such as less than or equal to about any of −0.3, −0.4, −0.5, −0.6, −0.7, −0.8, −0.9, −1.0, −1.1, −1.2, −1.3, −1.4, −1.5, −1.6, −1.7, −1.8, −1.9, or −2.0. In some embodiments, the increased or decreased expression of the one or more biomarkers of the CAD proteomic signature is within a standard deviation of about 0.1 or less of the mean log 2 ratio of Table 1. In some embodiments, the increased or decreased expression of the one or more biomarkers of the CAD proteomic signature is within a standard deviation of about 0.05 or less of the mean log 2 ratio of Table 1.

In some embodiments, status and/or degree of increased or decreased expression is based on comparison to a reference, e.g., a healthy individual, e.g., an individual not having CAD. In some embodiments, the reference is a literature value, such as published in a scientific reference. In some embodiments, the reference is based on a population of healthy individuals, e.g., an individual not having CAD. In some embodiments, the reference is an average expression level as measured from a population of healthy individuals, e.g., an individual not having CAD.

In some embodiments, the methods are based on one or more measurements from one or more samples, or derivative thereof, obtained from the individual. In some embodiments, when one or more measurements are performed to assess a biomarker, the method may be based on an average measurement of said biomarker.

In some embodiments, the CAD proteomic signature comprises one or more biomarkers associated with a calcium signaling pathway, histone regulation, HIF-1 signaling pathway, cAMP signaling pathway, beta-adrenergic signaling pathway, PI3K-Akt signaling pathway, complement and/or coagulation cascade, sphingolipid signaling pathway, natural killer cell mediated cytotoxicity, adipocytoknie signaling pathway, DNA damage, calcium energy, metaboloimcs, cellular adhesion, inflammation, hypoxia, and histone methylation.

In some embodiments, the one or more biomarkers of the CAD proteomic signature comprise a subset thereof comprising one or more biomarkers associated with a transcription factor. In some embodiments, the one or more biomarkers associated with a transcription factor are each selected from the group consisting of NF4A, FOXA2, LMO2, RUNX1, FLI1, EGR1, VDR, RCF21, GATA2, TP63, ELKS, FLI1, GATA1, CTNNB1, SIN3B, STATS, TAP1, AHR, MTF2, and SRY.

In some embodiments, the one or more biomarkers of the CAD proteomic signature comprise a subset thereof comprising one or more biomarkers associated with a kinase. In some embodiments, the one or more biomarkers associated with a kinase are each selected from the group consisting of HIPK2, MAPK1, MAPK3, GSK3B, MAPK8, TAF1, AKT1, CDK1, MAPK14, CDK9, CSNK2A1, CHUK, NLK, ABL1, CDK6, CDK2, CDK7, CDK4, TRIM24, and PRKCZ.

In some embodiments, the CAD proteomic signature comprises at least 5 biomarkers, such as at least any of 10 biomarkers, 15 biomarkers, 20 biomarkers, 25 biomarkers, 30 biomarkers, 35 biomarkers, 40 biomarkers, 45 biomarkers, 50 biomarkers, 55 biomarkers, 60 biomarkers, 65 biomarkers, 70 biomarkers, 75 biomarkers, 80 biomarkers, 85 biomarkers, 90 biomarkers, 95 biomarkers, 100 biomarkers, 110 biomarkers, 120 biomarkers, 130 biomarkers, 140 biomarkers, 150 biomarkers, 160 biomarkers, 170 biomarkers, 180 biomarkers, 190 biomarkers, 200 biomarkers, 210 biomarkers, 220 biomarkers, 230 biomarkers, 240 biomarkers, 250 biomarkers, 260 biomarkers, 270 biomarkers, 280 biomarkers, or 290 biomarkers, of Table 1. In some embodiments, the CAD proteomic signature comprises all the biomarkers of Table 1. In some embodiments, the CAD proteomic signature is analyzed based on the status of increased or decreased expression of the biomarkers therein according to Table 1. In some embodiments, the CAD proteomic signature is analyzed based on the level increased or decreased expression of the biomarkers therein according to Table 1.

In some embodiments, the CAD proteomic signature comprises Q969E1 (LEAP2), Q8NF37 (LPCAT1), Q01082 (SPTBN1), Q7Z333 (SETX), and P30481 (HLA-B).

In some embodiments, the CAD proteomic signature comprises Q969E1 (LEAP2), Q8NF37 (LPCAT1), Q01082 (SPTBN1), Q7Z333 (SETX), P30481 (HLA-B), Q5T8A7 (PPP1R26), Q9NX02 (NLRP2), P02144 (MB), Q9BQS2 (SYT15), and P62805 (HIST1H4A).

In some embodiments, the CAD proteomic signature comprises Q969E1 (LEAP2), Q8NF37 (LPCAT1), Q01082 (SPTBN1), Q7Z333 (SETX), P30481 (HLA-B), Q5T8A7 (PPP1R26), Q9NX02 (NLRP2), P02144 (MB), Q9BQS2 (SYT15), P62805 (HIST1H4A), P80370 (DLK1), P68366 (TUBA4A), P27797 (CALR), P05164 (MPO), and Q99439 (CNN2).

In some embodiments, the CAD proteomic signature comprises Q86YI8 (PHF13), Q9Y4D8 (HECTD4), Q9UIW2 (PLXNA1), Q6ZS81 (WDFY4), and Q9H329 (EPB41L4B).

In some embodiments, the CAD proteomic signature comprises Q86YI8 (PHF13), Q9Y4D8 (HECTD4), Q9UIW2 (PLXNA1), Q6ZS81 (WDFY4), Q9H329 (EPB41L4B), A2RUB1 (C17orf104), 015031 (PLXNB2), Q9NYF3 (FAM53C), 075146 (HIP1R), and P80362 (Ig kappa chain V-I region WAT).

In some embodiments, the CAD proteomic signature comprises Q86YI8 (PHF13), Q9Y4D8 (HECTD4), Q9UIW2 (PLXNA1), Q6ZS81 (WDFY4), Q9H329 (EPB41L4B), A2RUB1 (C₁₇orf104), 015031 (PLXNB2), Q9NYF3 (FAM53C), 075146 (HIP1R), P80362 (Ig kappa chain V-I region WAT), P01880 (IGHD), Q9COKO (BCL11B), AOAVI2 (FER1L5), Q86XJ1 (GAS2L3), and Q00688 (FKBP3).

In some embodiments, the CAD proteomic signature comprises Q969E1 (LEAP2), Q8NF37 (LPCAT1), Q01082 (SPTBN1), Q7Z333 (SETX), P30481 (HLA-B), Q86YI8 (PHF13), Q9Y4D8 (HECTD4), Q9UIW2 (PLXNA1), Q6ZS81 (WDFY4), and Q9H329 (EPB41L4B).

In some embodiments, the CAD proteomic signature comprises Q969E1 (LEAP2), Q8NF37 (LPCAT1), Q01082 (SPTBN1), Q7Z333 (SETX), P30481 (HLA-B), Q5T8A7 (PPP1R26), Q9NX02 (NLRP2), P02144 (MB), Q9BQS2 (SYT15), P62805 (HIST1H4A), Q86YI8 (PHF13), Q9Y4D8 (HECTD4), Q9UIW2 (PLXNA1), Q6ZS81 (WDFY4), Q9H329 (EPB41L4B), A2RUB1 (C17orf104), 015031 (PLXNB2), Q9NYF3 (FAM53C), 075146 (HIP1R), and P80362 (Ig kappa chain V-I region WAT).

In some embodiments, the CAD proteomic signature comprises Q969E1 (LEAP2), Q8NF37 (LPCAT1), Q01082 (SPTBN1), Q7Z333 (SETX), P30481 (HLA-B), Q5T8A7 (PPP1R26), Q9NX02 (NLRP2), P02144 (MB), Q9BQS2 (SYT15), P62805 (HIST1H4A), P80370 (DLK1), P68366 (TUBA4A), P27797 (CALR), P05164 (MPO), Q99439 (CNN2), Q969E1 (LEAP2), Q8NF37 (LPCAT1), Q01082 (SPTBN1), Q7Z333 (SETX), P30481 (HLA-B), Q5T8A7 (PPP1R26), Q9NX02 (NLRP2), P02144 (MB), Q9BQS2 (SYT15), P62805 (HIST1H4A), Q86YI8 (PHF13), Q9Y4D8 (HECTD4), Q9UIW2 (PLXNA1), Q6ZS81 (WDFY4), Q9H329 (EPB41L4B), A2RUB1 (C17orf104), 015031 (PLXNB2), Q9NYF3 (FAM53C), 075146 (HIP1R), and P80362 (Ig kappa chain V-I region WAT).

In some embodiments, the one or more biomarkers are indicative of safety, efficacy, diagnosis, prognosis, disease progression, response to a therapy, or any combination thereof.

Provided herein, in some aspects, is a method of subjecting an individual to a coronary artery disease (CAD) diagnosis determination, the method comprising: (a) obtaining mass spectrometry (MS) data from a sample, or a derivative thereof, obtained from the individual; and (b) analyzing the MS data according to a CAD proteomic signature, wherein the CAD proteomic signature comprises one or more biomarkers of Table 1; and (c) determining whether the individual has the CAD proteomic signature. In some embodiments, if the individual has the CAD proteomic signature the individual is diagnosed as has having CAD.

Provided herein, in some aspects, is a method of diagnosing an individual as having coronary artery disease (CAD), the method comprising: (a) obtaining mass spectrometry (MS) data from a sample, or a derivative thereof, obtained from the individual; and (b) analyzing the MS data according to a CAD proteomic signature, wherein the CAD proteomic signature comprises one or more biomarkers of Table 1; and (c) diagnosing the individual as having CAD based on the presence of the CAD proteomic signature.

Provided herein, in some aspects, is a method of treating an individual having coronary artery disease (CAD), the method comprising: (a) diagnosing an individual as having CAD according to the presence of a CAD proteomic signature in a sample, or a derivative thereof, obtained from the individual, wherein the CAD proteomic signature comprises one or more biomarkers of Table 1; and (b) administering to the individual a CAD treatment.

Provided herein, in some aspects, is a method for detecting a coronary artery disease (CAD) proteomic signature of an individual, (a) obtaining mass spectrometry (MS) data from a sample, or a derivative thereof, obtained from the individual; and (b) analyzing the MS data according to a CAD proteomic signature to detect the CAD proteomic signature, wherein the CAD proteomic signature comprises one or more biomarkers of Table 1. In some embodiments, the individual is suspected of having CAD.

In some embodiments, the presence of the CAD proteomic signature is determined by analyzing MS data according to the CAD proteomic signature.

In some embodiments, the methods further comprise obtaining the MS data from the sample, or the derivative thereof, obtained from the individual, such as by performing a mass spectrometry technique describe herein.

In some embodiments, the CAD treatment comprises a life style adjustment. In some embodiments, the life style adjustment is a diet, implementation of an exercise routine, cessation of smoking, and/or cessation of alcohol consumption.

In some embodiments, the CAD treatment comprises a pharmaceutical intervention. Pharmaceutical drugs and agents for treating CAD are known. It is within the level of a skilled person to choose the appropriate drug for treatment of the subject. In some embodiments, the pharmaceutical intervention comprises administration of a drug selected from the group consisting of a calcium channel blocker, histone deacetylase (HDAC) inhibitor (such as HDAC6), Ca2+/calmodulin (CaM)-dependent protein kinase II (CaMK II) inhibitor, guanylyl cyclase (sGC) activator, MMP inhibitor, statin, and anti-hypertesnive. In some embodiments, the pharmaceutical intervention comprises a drug is selected from the group consisting of amlodipine, tubastatin-a, forskolin, trichostatin A, KN-93, CFM-1571, ilomastat, CAY-10603, and rosuvastatin, or a pharmaceutical salt thereof. In some embodiments, the drug is selected from the group consisting of BRD-K52306726, BRD-K71361154, acetazolamide, rolipram, ruxolitinib, BRD-A59808129-001-01-7, BRD-K76876037, ZM336372, trehalose, SCHEMBL3092652, BMS-387032, BRD-K01425431, 4-hydroxy-retinoic acid, CHEMBL585951, CHEMBL1673039, HY-11007, primidone, BRD-K81417919, SPECTRUM_000826, tamoxifen, BRD-K00544996, CID 67066889, CX-5461, BRD-K63944563, SCHEMBL6851809, BRD-A86146706, FR-180204, CHEMBL552425, hexachlorophene, Aggc, SUGA1_008424, BRD-K96640811, anastrozole, wortmannin, vandetanib, AC1NWALF, OTSSP167, WZ3105, dihydroergotamine, BRD-K99839793, SR 33805 oxalate, AT-7519, sulfadoxine, SPECTRUM_001319, MLS003329219, trichostatin A, and rotenone, or a pharmaceutical salt thereof. In some embodiments, the drug is selected from the group consisting of 6-mercaptopurine, vincristine, bevacizumab, prednisone, thalidomide, zoledronic acid, paclitaxel, pemetrexed, topotecan, cabazitaxel, prednisolone, capecitabine, capecitabine, gemcitabine, capecitabine, docetaxel, oxaliplatin, cevipabulin, colchicine, probenecid, cyclophosphamide, daunorubicin, imatinib, 5-fluorouracil, epirubicin, trastuzumab, vinorelbine, rituximab, etoposide, etoposide, gemcitabine, mitoxantrone, mitoxantrone, topotecan, vinorelbine, davunetide, dexamethasone, gemcitabine, gemcitabine, gemcitabine, vinorelbine, hydrocortisone, irinotecan, pamidronic acid, vinorelbine, epothilone B, eribulin, gemcitabine, gemcitabine, gemcitabine, vinorelbine, irinotecan, temozolomide, irinotecan, cytarabine, L-asparaginase, prednisone, larotaxel, milataxel, topotecan, plinabulin, podophyllotoxin, vinorelbine, vinblastine, vinflunine, vinorelbine, vintafolide, AZD4831, GCS-1, GR-MD-2, BI 76563, CC-95251, eculizumab, IFX-1, IgG, ravulizumab, pegcetacoplan, CNGRC peptide-TNF alpha conjugate, stamulumab, BI 836845, MEDI-573, MORAb-4, lavendustin C, alirocumab, BMS-844421, evinacumab, PCSK9 inhibitor, CALAA-1, CX-229, emicizumab, moroctocog alfa, L19-IL2 monoclonal antibody-cytokine fusion protein, Ll9TNFalpha, ocriplasmin, carotuximab, AP01, ASP8232, hydralazine, hydrochlorothiazide, hydralazine, reserpine, isosorbide dinitrate, amrinone, anagrelide, cilostazol, dipyridamole, dyphylline, enoximone, medorinone, milrinone, nitroglycerin, pentoxifylline, theophylline, tolbutamide, alvespimycin, cisplatin, luminespib, retaspimycin, TAS-116, bapineuzumab, florbetaben F, florbetapir F18, collagenase Clostridium histolyticum, trilostane, activated recombinant human factor VII, apixaban, clopidogrel, rivaroxaban, enoxaparin, aspirin, rivaroxaban, rivaroxaban, ticlopidine, betrixaban, dalteparin, deligoparin, DPC 423, edoxaban, emicizumab, enoxaparin, F8, F9, fondaparinux, warfarin, heparin, idraparinux, nematode anticoagulant protein c2, rivaroxaban, RPR 12844, RPR 28566, tifacogin, AGN 2194, AR-H4718, clarithromycin, dexlansoprazole, diclofenac, esomeprazole magnesium, esomeprazole, naproxen, ilaprazole, lansoprazole, magnesium hydroxide, sodium bicarbonate, omeprazole, sodium bicarbonate, pantoprazole, rabeprazole, tenatoprazole, AZD425, baricitinib, methotrexate, brepocitinib, erlotinib, ruxolitinib, filgotinib, INCB52793, itacitinib, JAK1 inhibitor, jaktinib, methotrexate, tofacitinib, momelotinib, tofacitinib, upadacitinib, cedazuridine, cytidine deaminase inhibitor, and rosiptor, or any combination thereof.

In some embodiments, the method of treatment further comprises monitoring the CAD treatment. In some embodiments, the method comprises performing the CAD proteomic signature analysis following treatment and assessing changes indicative of an improvement in CAD, such as a return to a healthy state. In some embodiments, the method comprises monitoring one or more symptoms of CAD.

In some embodiments, the method further comprises obtaining the sample from the individual. In some embodiments, the sample, or the derivative thereof, is a blood sample or a derivative thereof. In some embodiments, the sample, or the derivative thereof, is a plasma sample. In some embodiments, the sample, or the derivative thereof, comprises a liquid fixative. In some embodiments, the sample is obtained and processed as described in other sections of the present application.

In some embodiments, obtaining MS data from the sample, or the derivative thereof, comprises performing a mass spectrometry analysis of the sample, or the derivative thereof, using a mass spectrometer. In some embodiments, the mass spectrometry analysis is performed according to the description provided herein.

In some embodiments, analyzing the MS data according to the CAD proteomic signature comprises subjecting the MS data to a method described herein. In some embodiments, analyzing the MS data according to the CAD proteomic signature comprises assessing the presence or absence or level of each of the one or more biomarkers of the CAD proteomic signature in the MS data. In some embodiments, agreement with a CAD proteomic signature is based on whether the one or more biomarkers of the CAD proteomic signature substantially agree (such as at least about 70%, including at least about any of 75%, 80%, 85%, 90%, or 95%, of the one or more biomarkers) with the increased expression or decreased expression classification of Table 1.

In some embodiments, the methods further comprise performing one or more of the following factor assessments of the individual: sex, age, body mass index (BMI), systolic blood pressure, diastolic blood pressure, total cholesterol, HDL, LDL, triglycerides, hyperlipidemia, hypertension, diabetes mellitus, insulin resistance, kidney disease, smoking status, level of physical activity, level of sleep, or quality of nutrition.

In some embodiments, the method further comprise performing a medical procedure on the individual to assess the presence of CAD, such as cardiac catheterization or coronary CT angiography.

TABLE 1
Identified biomarkers of a CAD signature.

| | | | | | | CAD vs. |
|---|---|---|---|---|---|---|
| | | | | | | Control |
| Accession | | | Type(s) | Cellular | CAD | (mean |
| No. | Protein Name and Description | Gene Name | Descriptor | Location | Signature | log2ratio) |
| Q969E1 | Liver-expressed antimicrobial peptide 2 OS = Homo sapiens | LEAP2 | Other | Extracellular | Increased | 2.1 |
| | PE = 1 SV = 1 - [LEAP2_HUMAN] | | | Space | expression | |
| Q8NF37 | Lysophosphatidylcholine acyltransferase 1 OS = Homo | LPCAT1 | Enzyme | Cytoplasm | Increased | 2.0 |
| | sapiens PE = 1 SV = 2 - [PCAT1_HUMAN] | | | | expression | |
| Q01082 | Spectrin beta chain, non-erythrocytic 1 OS = Homo sapiens | SPTBN1 | Other | Plasma | Increased | 1.7 |
| | PE = 1 SV = 2 - [SPTB2_HUMAN] | | | Membrane | expression | |
| Q7Z333 | Probable helicase senataxin OS = Homo sapiens PE = 1 | SETX | Enzyme | Nucleus | Increased | 1.5 |
| | SV = 4 - [SETX_HUMAN] | | | | expression | |
| P30481 | HLA class I histocompatibility antigen, B-44 alpha chain | HLA-B | Transmembrane | Plasma | Increased | 1.4 |
| | OS = Homo sapiens PE = 1 SV = 1 - [1B44_HUMAN] | | receptor | Membrane | expression | |
| Q5T8A7 | Protein phosphatase 1 regulatory subunit 26 OS = Homo | PPP1R26 | Other | Nucleus | Increased | 1.4 |
| | sapiens PE = 1 SV = 1 - [PPR26_HUMAN] | | | | expression | |
| Q9NX02 | NACHT, LRR and PYD domains-containing protein 2 | NLRP2 | Other | Nucleus | Increased | 1.3 |
| | OS = Homo sapiens PE = 1 SV = 1 - [NALP2_HUMAN] | | | | expression | |
| P02144 | Myoglobin OS = Homo sapiens PE = 1 SV = 2 - | MB | Transporter | Cytoplasm | Increased | 1.2 |
| | [MYG_HUMAN] | | | | expression | |
| Q9BQS2 | Synaptotagmin-15 OS = Homo sapiens PE = 2 SV = 3 - | SYT15 | Transporter | Cytoplasm | Increased | 1.2 |
| | [SYT15_HUMAN] | | | | expression | |
| P62805 | Histone H4 OS = Homo sapiens PE = 1 SV = 2 - | HIST1H4A | — | Nucleus | Increased | 1.2 |
| | [H4_HUMAN] | | | | expression | |
| P80370 | Protein delta homolog 1 OS = Homo sapiens PE = 1 SV = 3 - | DLK1 | Other | Extracellular | Increased | 1.1 |
| | [DLK1_HUMAN] | | | Space | expression | |
| P68366 | Tubulin alpha-4A chain OS = Homo sapiens PE = 1 SV = 1 - | TUBA4A | Other | Cytoplasm | Increased | 1.1 |
| | [TBA4A_HUMAN] | | | | expression | |
| P27797 | Calreticulin OS = Homo sapiens PE = 1 SV = 1 - | CALR | Transcription | Cytoplasm | Increased | 1.1 |
| | [CALR_HUMAN] | | regulator | | expression | |
| P05164 | Myeloperoxidase OS = Homo sapiens PE = 1 SV = 1 - | MPO | Enzyme | Cytoplasm | Increased | 1.1 |
| | [PERM_HUMAN] | | | | expression | |
| Q99439 | Calponin-2 OS = Homo sapiens PE = 1 SV = 4 - | CNN2 | Other | Cytoplasm | Increased | 1.0 |
| | [CNN2_HUMAN] | | | | expression | |
| P69905 | Hemoglobin subunit alpha OS = Homo sapiens PE = 1 SV = | HBA1 | — | Extracellular | Increased | 1.0 |
| | 2 - [HBA_HUMAN] | | | Space | expression | |
| P16885 | 1-phosphatidylinositol 4,5-bisphosphate phosphodiesterase | PLCG2 | Enzyme | Cytoplasm | Increased | 1.0 |
| | gamma-2 OS = Homo sapiens PE = 1 SV = 4 - | | | | expression | |
| | [PLCG2_HUMAN] | | | | | |
| P04179 | Superoxide dismutase [Mn], mitochondrial OS = Homo | SOD2 | Enzyme | Cytoplasm | Increased | 1.0 |
| | sapiens PE = 1 SV = 2 - [SODM_HUMAN] | | | | expression | |
| O75112 | LIM domain-binding protein 3 OS = Homo sapiens PE = 1 | LDB3 | Transporter | Cytoplasm | Increased | 1.0 |
| | SV = 2 - [LDB3_HUMAN] | | | | expression | |
| P17931 | Galectin-3 OS = Homo sapiens PE = 1 SV = 5 - | LGALS3 | Other | Extracellular | Increased | 0.9 |
| | [LEG3_HUMAN] | | | Space | expression | |
| P52758 | Ribonuclease UK114 OS = Homo sapiens PE = 1 SV = 1 - | HRSP12 | Enzyme | Cytoplasm | Increased | 0.9 |
| | [UK114_HUMAN] | | | | expression | |
| P05787 | Keratin, type II cytoskeletal 8 OS = Homo sapiens PE = 1 | KRT8 | Other | Cytoplasm | Increased | 0.9 |
| | SV = 7 - [K2C8_HUMAN] | | | | expression | |
| Q96AP7 | Endothelial cell-selective adhesion molecule OS = Homo | ESAM | Other | Plasma | Increased | 0.9 |
| | sapiens PE = 1 SV = 1 - [ESAM_HUMAN] | | | Membrane | expression | |
| Q8NA03 | Fibrous sheath-interacting protein 1 OS = Homo sapiens | FSIP1 | Other | Other | Increased | 0.9 |
| | PE = 2 SV = 1 - [FSIP1_HUMAN] | | | | expression | |
| P55056 | Apolipoprotein C-IV OS = Homo sapiens PE = 1 SV = 1 - | APOC4 | Transporter | Extracellular | Increased | 0.9 |
| | [APOC4_HUMAN] | | | Space | expression | |
| O14498 | Immunoglobulin superfamily containing leucine-rich | ISLR | Other | Extracellular | Increased | 0.9 |
| | repeat protein OS = Homo sapiens PE = 1 SV = 1 - | | | Space | expression | |
| | [ISLR_HUMAN] | | | | | |
| P09681 | Gastric inhibitory polypeptide OS = Homo sapiens PE = 1 | GIP | Other | Extracellular | Increased | 0.8 |
| | SV = 1 - [GIP_HUMAN] | | | Space | expression | |
| P03950 | Angiogenin OS = Homo sapiens PE = 1 SV = 1 - | ANG | Enzyme | Extracellular | Increased | 0.8 |
| | [ANGI_HUMAN] | | | Space | expression | |
| Q08ET2 | Sialic acid-binding Ig-like lectin 14 OS = Homo sapiens | SIGLEC14 | Other | Plasma | Increased | 0.8 |
| | PE = 1 SV = 1 - [SIG14_HUMAN] | | | Membrane | expression | |
| O15273 | Telethonin OS = Homo sapiens PE = 1 SV = 1 - | TCAP | Other | Cytoplasm | Increased | 0.8 |
| | [TELT_HUMAN] | | | | expression | |
| P78324 | Tyrosine-protein phosphatase non-receptor type substrate 1 | SIRPA | Phosphatase | Plasma | Increased | 0.8 |
| | OS = Homo sapiens PE = 1 SV = 2 - [SHPS1_HUMAN] | | | Membrane | expression | |
| P01031 | Complement C5 OS = Homo sapiens PE = 1 SV = 4 - | C5 | Cytokine | Extracellular | Increased | 0.8 |
| | [CO5_HUMAN] | | | Space | expression | |
| Q9BY66 | Lysine-specific demethylase 5D OS = Homo sapiens PE = 1 | KDM5D | Enzyme | Nucleus | Increased | 0.8 |
| | SV = 2 - [KDM5D_HUMAN] | | | | expression | |
| Q02325 | Plasminogen-like protein B OS = Homo sapiens PE = 1 | PLGLB1 | — | Extracellular | Increased | 0.8 |
| | SV = 1 - [PLGB_HUMAN] | | | Space | expression | |
| Q6ECI4 | Zinc finger protein 470 OS = Homo sapiens PE = 2 SV = 3 - | ZNF470 | Other | Nucleus | Increased | 0.8 |
| | [ZN470_HUMAN] | | | | expression | |
| P11509 | Cytochrome P450 2A6 OS = Homo sapiens PE = 1 SV = 3 - | CYP2A6 | Enzyme | Cytoplasm | Increased | 0.8 |
| | [CP2A6_HUMAN] | | | | expression | |
| O94804 | Serine/threonine-protein kinase 10 OS = Homo sapiens | STK10 | Kinase | Cytoplasm | Increased | 0.8 |
| | PE = 1 SV = 1 - [STK10_HUMAN] | | | | expression | |
| Q9NPC4 | Lactosylceramide 4-alpha-galactosyltransferase OS = Homo | A4GALT | Enzyme | Cytoplasm | Increased | 0.8 |
| | sapiens PE = 2 SV = 1 - [A4GAT_HUMAN] | | | | expression | |
| Q8WXG9 | G-protein coupled receptor 98 OS = Homo sapiens PE = 1 | GPR98 | G-protein | Plasma | Increased | 0.7 |
| | SV = 2 - [GPR98_HUMAN] | | coupled receptor | Membrane | expression | |
| Q9UNW1 | Multiple inositol polyphosphate phosphatase 1 OS = Homo | MINPP1 | Phosphatase | Cytoplasm | Increased | 0.7 |
| | sapiens PE = 1 SV = 1 - [MINP1_HUMAN] | | | | expression | |
| Q14997 | Proteasome activator complex subunit 4 OS = Homo | PSME4 | Other | Cytoplasm | Increased | 0.7 |
| | sapiens PE = 1 SV = 2 - [PSME4_HUMAN] | | | | expression | |
| Q9Y240 | C-type lectin domain family 11 member A OS = Homo | CLEC11A | Growth factor | Extracellular | Increased | 0.7 |
| | sapiens PE = 1 SV = 1 - [CLC11_HUMAN] | | | Space | expression | |
| Q9UJ72 | Annexin A10 OS = Homo sapiens PE = 1 SV = 3 - | ANXA10 | Other | Cytoplasm | Increased | 0.7 |
| | [ANX10_HUMAN] | | | | expression | |
| Q9BXS4 | Transmembrane protein 59 OS = Homo sapiens PE = 1 SV = | TMEM59 | Peptidase | Plasma | Increased | 0.7 |
| | 1 - [TMM59_HUMAN] | | | Membrane | expression | |
| P01024 | Complement C3 OS = Homo sapiens PE = 1 SV = 2 - | C3 | Peptidase | Extracellular | Increased | 0.7 |
| | [CO3_HUMAN] | | | Space | expression | |
| Q9H1Z8 | Augurin OS = Homo sapiens PE = 1 SV = 1 - | C2orf40 | Other | Extracellular | Increased | 0.7 |
| | [AUGN_HUMAN] | | | Space | expression | |
| Q99972 | Myocilin OS = Homo sapiens PE = 1 SV = 2 - | MYOC | Other | Cytoplasm | Increased | 0.6 |
| | [MYOC_HUMAN] | | | | expression | |
| P25774 | Cathepsin S OS = Homo sapiens PE = 1 SV = 3 - | CTSS | Peptidase | Cytoplasm | Increased | 0.6 |
| | [CATS_HUMAN] | | | | expression | |
| Q9H6X2 | Anthrax toxin receptor 1 OS = Homo sapiens PE = 1 SV = | ANTXR1 | Transmembrane | Plasma | Increased | 0.6 |
| | 2 - [ANTR1_HUMAN] | | receptor | Membrane | expression | |
| Q9UJX4 | Anaphase-promoting complex subunit 5 OS = Homo | ANAPC5 | Other | Nucleus | Increased | 0.6 |
| | sapiens PE = 1 SV = 2 - [APC5_HUMAN] | | | | expression | |
| P27169 | Serum paraoxonase/arylesterase 1 OS = Homo sapiens | PON1 | Phosphatase | Extracellular | Increased | 0.6 |
| | PE = 1 SV = 3 - [PON1_HUMAN] | | | Space | expression | |
| Q96KK5 | Histone H2A type 1-H OS = Homo sapiens PE = 1 SV = 3 - | HIST1H2AH | Other | Nucleus | Increased | 0.6 |
| | [H2A1H_HUMAN] | | | | expression | |
| P25786 | Proteasome subunit alpha type-1 OS = Homo sapiens PE = 1 | PSMA1 | Peptidase | Cytoplasm | Increased | 0.6 |
| | SV = 1 - [PSA1_HUMAN] | | | | expression | |
| Q6ZMJ2 | Scavenger receptor class A member 5 OS = Homo sapiens | SCARA5 | Transmembrane | Plasma | Increased | 0.6 |
| | PE = 2 SV = 1 - [SCAR5_HUMAN] | | receptor | Membrane | expression | |
| C9JN71 | Zinc finger protein 878 OS = Homo sapiens PE = 3 SV = 2 - | ZNF878 | Other | Other | Increased | 0.6 |
| | [ZN878_HUMAN] | | | | expression | |
| P15144 | Aminopeptidase N OS = Homo sapiens PE = 1 SV = 4 - | ANPEP | Peptidase | Plasma | Increased | 0.6 |
| | [AMPN_HUMAN] | | | Membrane | expression | |
| P68104 | Elongation factor 1-alpha 1 OS = Homo sapiens PE = 1 | EEF1A1 | Translation | Cytoplasm | Increased | 0.6 |
| | SV = 1 - [EF1A1_HUMAN] | | regulator | | expression | |
| O14976 | Cyclin-G-associated kinase OS = Homo sapiens PE = 1 | GAK | Kinase | Nucleus | Increased | 0.6 |
| | SV = 2 - [GAK_HUMAN] | | | | expression | |
| Q86UP2 | Kinectin OS = Homo sapiens PE = 1 SV = 1 - | KTN1 | Transmembrane | Plasma | Increased | 0.6 |
| | [KTN1_HUMAN] | | receptor | Membrane | expression | |
| Q9Y462 | Zinc finger protein 711 OS = Homo sapiens PE = 1 SV = 2 - | ZNF711 | Transcription | Nucleus | Increased | 0.6 |
| | [ZN711_HUMAN] | | regulator | | expression | |
| Q96CG8 | Collagen triple helix repeat-containing protein 1 | CTHRC1 | Other | Extracellular | Increased | 0.6 |
| | OS = Homo sapiens PE = 1 SV = 1 - [CTHR1_HUMAN] | | | Space | expression | |
| P01023 | Alpha-2-macroglobulin OS = Homo sapiens PE = 1 SV = 3 - | A2M | Transporter | Extracellular | Increased | 0.6 |
| | [A2MG_HUMAN] | | | Space | expression | |
| O14793 | Growth/differentiation factor 8 OS = Homo sapiens PE = 1 | MSTN | Growth factor | Extracellular | Increased | 0.6 |
| | SV = 1 - [GDF8_HUMAN] | | | Space | expression | |
| Q15628 | Tumor necrosis factor receptor type 1-associated DEATH | TRADD | Other | Cytoplasm | Increased | 0.6 |
| | domain protein OS = Homo sapiens PE = 1 SV = 2 - | | | | expression | |
| | [TRADD_HUMAN] | | | | | |
| Q9NZP8 | Complement C1r subcomponent-like protein OS = Homo | C1RL | Peptidase | Extracellular | Increased | 0.5 |
| | sapiens PE = 1 SV = 2 - [C1RL_HUMAN] | | | Space | expression | |
| Q9BQE5 | Apolipoprotein L2 OS = Homo sapiens PE = 1 SV = 1 - | APOL2 | Other | Cytoplasm | Increased | 0.5 |
| | [APOL2_HUMAN] | | | | expression | |
| P05019 | Insulin-like growth factor I OS = Homo sapiens PE = 1 | IGF1 | Growth factor | Extracellular | Increased | 0.5 |
| | SV = 1 - [IGF1_HUMAN] | | | Space | expression | |
| Q96HD1 | Cysteine-rich with EGF-like domain protein 1 OS = Homo | CRELD1 | Other | Other | Increased | 0.5 |
| | sapiens PE = 1 SV = 3 - [CREL1_HUMAN] | | | | expression | |
| Q15113 | Procollagen C-endopeptidase enhancer 1 OS = Homo | PCOLCE | Other | Extracellular | Increased | 0.5 |
| | sapiens PE = 1 SV = 2 - [PCOC1_HUMAN] | | | Space | expression | |
| Q9UBR2 | Cathepsin Z OS = Homo sapiens PE = 1 SV = 1 - | CTSZ | Peptidase | Cytoplasm | Increased | 0.5 |
| | [CATZ_HUMAN] | | | | expression | |
| Q92520 | Protein FAM3C OS = Homo sapiens PE = 1 SV = 1 - | FAM3C | Cytokine | Extracellular | Increased | 0.5 |
| | [FAM3C_HUMAN] | | | Space | expression | |
| Q06033 | Inter-alpha-trypsin inhibitor heavy chain H3 OS = Homo | ITIH3 | Other | Extracellular | Increased | 0.5 |
| | sapiens PE = 1 SV = 2 - [ITIH3_HUMAN] | | | Space | expression | |
| Q9NZT1 | Calmodulin-like protein 5 OS = Homo sapiens PE = 1 SV = | CALML5 | Other | Cytoplasm | Increased | 0.5 |
| | 2 - [CALL5_HUMAN] | | | | expression | |
| Q9HCU0 | Endosialin OS = Homo sapiens PE = 1 SV = 1 - | CD248 | Other | Plasma | Increased | 0.5 |
| | [CD248_HUMAN] | | | Membrane | expression | |
| P18428 | Lipopolysaccharide-binding protein OS = Homo sapiens | LBP | Transporter | Plasma | Increased | 0.5 |
| | PE = 1 SV = 3 - [LBP_HUMAN] | | | Membrane | expression | |
| A4D1P6 | WD repeat-containing protein 91 OS = Homo sapiens PE = 1 | WDR91 | Other | Cytoplasm | Increased | 0.5 |
| | SV = 2 - [WDR91_HUMAN] | | | | expression | |
| P13497 | Bone morphogenetic protein 1 OS = Homo sapiens PE = 1 | BMP1 | Peptidase | Extracellular | Increased | 0.5 |
| | SV = 2 - [BMP1_HUMAN] | | | Space | expression | |
| P36955 | Pigment epithelium-derived factor OS = Homo sapiens | SERPINF1 | Other | Extracellular | Increased | 0.5 |
| | PE = 1 SV = 4 - [PEDF_HUMAN] | | | Space | expression | |
| Q99973 | Telomerase protein component 1 OS = Homo sapiens PE = 1 | TEP1 | Enzyme | Nucleus | Increased | 0.5 |
| | SV = 2 - [TEP1_HUMAN] | | | | expression | |
| P31948 | Stress-induced-phosphoprotein 1 OS = Homo sapiens PE = 1 | STIP1 | Other | Cytoplasm | Increased | 0.5 |
| | SV = 1 - [STIP1_HUMAN] | | | | expression | |
| Q92743 | Serine protease HTRA1 OS = Homo sapiens PE = 1 SV = 1 - | HTRA1 | Peptidase | Extracellular | Increased | 0.5 |
| | [HTRA1_HUMAN] | | | Space | expression | |
| P35542 | Serum amyloid A-4 protein OS = Homo sapiens PE = 1 | SAA4 | Transporter | Extracellular | Increased | 0.5 |
| | SV = 2 - [SAA4_HUMAN] | | | Space | expression | |
| O15321 | Transmembrane 9 superfamily member 1 OS = Homo | TM9SF1 | Transporter | Plasma | Increased | 0.5 |
| | sapiens PE = 2 SV = 2 - [TM9S1_HUMAN] | | | Membrane | expression | |
| Q96S96 | Phosphatidylethanolamine-binding protein 4 OS = Homo | PEBP4 | Other | Cytoplasm | Increased | 0.5 |
| | sapiens PE = 1 SV = 3 - [PEBP4_HUMAN] | | | | expression | |
| P07306 | Asialoglycoprotein receptor 1 OS = Homo sapiens PE = 1 | ASGR1 | Transmembrane | Plasma | Increased | 0.5 |
| | SV = 2 - [ASGR1_HUMAN] | | receptor | Membrane | expression | |
| P45877 | Peptidyl-prolyl cis-trans isomerase C OS = Homo sapiens | PPIC | Enzyme | Cytoplasm | Increased | 0.5 |
| | PE = 1 SV = 1 - [PPIC_HUMAN] | | | | expression | |
| O60259 | Kallikrein-8 OS = Homo sapiens PE = 1 SV = 1 - | KLK8 | Peptidase | Extracellular | Increased | 0.5 |
| | [KLK8_HUMAN] | | | Space | expression | |
| P63104 | 14-3-3 protein zeta/delta OS = Homo sapiens PE = 1 SV = 1 - | YWHAZ | Enzyme | Cytoplasm | Increased | 0.5 |
| | [1433Z_HUMAN] | | | | expression | |
| Q9H0R5 | Guanylate-binding protein 3 OS = Homo sapiens PE = 1 | GBP3 | Enzyme | Cytoplasm | Increased | 0.5 |
| | SV = 3 - [GBP3_HUMAN] | | | | expression | |
| P28300 | Protein-lysine 6-oxidase OS = Homo sapiens PE = 1 SV = 2 - | LOX | Enzyme | Extracellular | Increased | 0.5 |
| | [LYOX_HUMAN] | | | Space | expression | |
| P04424 | Argininosuccinate lyase OS = Homo sapiens PE = 1 SV = 4 - | ASL | Enzyme | Cytoplasm | Increased | 0.5 |
| | [ARLY_HUMAN] | | | | expression | |
| Q9NWD8 | Transmembrane protein 248 OS = Homo sapiens PE = 2 | TMEM248 | Other | Other | Increased | 0.5 |
| | SV = 1 - [TM248_HUMAN] | | | | expression | |
| P61019 | Ras-related protein Rab-2A OS = Homo sapiens PE = 1 | RAB2A | Enzyme | Cytoplasm | Increased | 0.4 |
| | SV = 1 - [RAB2A_HUMAN] | | | | expression | |
| A6NH11 | Glycolipid transfer protein domain-containing protein 2 | GLTPD2 | Other | Other | Increased | 0.4 |
| | OS = Homo sapiens PE = 1 SV = 2 - [GLTD2_HUMAN] | | | | expression | |
| P33908 | Mannosyl-oligosaccharide 1,2-alpha-mannosidase IA | MAN1A1 | Enzyme | Cytoplasm | Increased | 0.4 |
| | OS = Homo sapiens PE = 1 SV = 3 - [MA1A1_HUMAN] | | | | expression | |
| Q7Z6K1 | THAP domain-containing protein 5 OS = Homo sapiens | THAP5 | Transcription | Nucleus | Increased | 0.4 |
| | PE = 1 SV = 2 - [THAP5_HUMAN] | | regulator | | expression | |
| P02545 | Prelamin-A/C OS = Homo sapiens PE = 1 SV = 1 - | LMNA | Other | Nucleus | Increased | 0.4 |
| | [LMNA_HUMAN] | | | | expression | |
| P80108 | Phosphatidylinositol-glycan-specific phospholipase D | GPLD1 | Enzyme | Cytoplasm | Increased | 0.4 |
| | OS = Homo sapiens PE = 1 SV = 3 - [PHLD_HUMAN] | | | | expression | |
| Q96NZ9 | Proline-rich acidic protein 1 OS = Homo sapiens PE = 1 | PRAP1 | Other | Nucleus | Increased | 0.4 |
| | SV = 2 - [PRAP1_HUMAN] | | | | expression | |
| Q9UGM3 | Deleted in malignant brain tumors 1 protein OS = Homo | DMBT1 | Transmembrane | Plasma | Increased | 0.4 |
| | sapiens PE = 1 SV = 2 - [DMBT1_HUMAN] | | receptor | Membrane | expression | |
| Q674X7 | Kazrin OS = Homo sapiens PE = 1 SV = 2 - | KAZN | Other | Plasma | Increased | 0.4 |
| | [KAZRN_HUMAN] | | | Membrane | expression | |
| Q9UDV7 | Zinc finger protein 282 OS = Homo sapiens PE = 2 SV = 3 - | ZNF282 | Transcription | Nucleus | Increased | 0.4 |
| | [ZN282_HUMAN] | | regulator | | expression | |
| Q8NBP7 | Proprotein convertase subtilisin/kexin type 9 OS = Homo | PCSK9 | Peptidase | Extracellular | Increased | 0.4 |
| | sapiens PE = 1 SV = 3 - [PCSK9_HUMAN] | | | Space | expression | |
| Q8N130 | Sodium-dependent phosphate transport protein 2C | SLC34A3 | Transporter | Plasma | Increased | 0.4 |
| | OS = Homo sapiens PE = 1 SV = 2 - [NPT2C_HUMAN] | | | Membrane | expression | |
| P02786 | Transferrin receptor protein 1 OS = Homo sapiens PE = 1 | TFRC | Transporter | Plasma | Increased | 0.4 |
| | SV = 2 - [TFR1_HUMAN] | | | Membrane | expression | |
| P0DJI8 | Serum amyloid A-1 protein OS = Homo sapiens PE = 1 | SAA1 | Transporter | Extracellular | Increased | 0.4 |
| | SV = 1 - [SAA1_HUMAN] | | | Space | expression | |
| Q9BQS7 | Hephaestin OS = Homo sapiens PE = 2 SV = 3 - | HEPH | Transporter | Plasma | Increased | 0.4 |
| | [HEPH_HUMAN] | | | Membrane | expression | |
| O00584 | Ribonuclease T2 OS = Homo sapiens PE = 1 SV = 2 - | RNASET2 | Enzyme | Cytoplasm | Increased | 0.4 |
| | [RNT2_HUMAN] | | | | expression | |
| P07195 | L-lactate dehydrogenase B chain OS = Homo sapiens PE = 1 | LDHB | Enzyme | Cytoplasm | Increased | 0.4 |
| | SV = 2 - [LDHB_HUMAN] | | | | expression | |
| P60900 | Proteasome subunit alpha type-6 OS = Homo sapiens PE = 1 | PSMA6 | Peptidase | Cytoplasm | Increased | 0.4 |
| | SV = 1 - [PSA6_HUMAN] | | | | expression | |
| P00740 | Coagulation factor IX OS = Homo sapiens PE = 1 SV = 2 - | F9 | Peptidase | Extracellular | Increased | 0.4 |
| | [FA9_HUMAN] | | | Space | expression | |
| Q9Y279 | V-set and immunoglobulin domain-containing protein 4 | VSIG4 | Other | Plasma | Increased | 0.4 |
| | OS = Homo sapiens PE = 1 SV = 1 - [VSIG4_HUMAN] | | | Membrane | expression | |
| P08493 | Matrix Gla protein OS = Homo sapiens PE = 1 SV = 2 - | MGP | Other | Extracellular | Increased | 0.4 |
| | [MGP_HUMAN] | | | Space | expression | |
| Q9ULK2 | Ataxin-7-like protein 1 OS = Homo sapiens PE = 2 SV = 3 - | ATXN7L1 | Other | Other | Increased | 0.4 |
| | [AT7L1_HUMAN] | | | | expression | |
| P07148 | Fatty acid-binding protein, liver OS = Homo sapiens PE = 1 | FABP1 | Transporter | Cytoplasm | Increased | 0.4 |
| | SV = 1 - [FABPL_HUMAN] | | | | expression | |
| Q9H4G4 | Golgi-associated plant pathogenesis-related protein 1 | GLIPR2 | Other | Cytoplasm | Increased | 0.4 |
| | OS = Homo sapiens PE = 1 SV = 3 - [GAPR1_HUMAN] | | | | expression | |
| Q8N1L4 | Putative inactive cytochrome P450 family member 4Z2 | CYP4Z2P | Other | Other | Increased | 0.4 |
| | OS = Homo sapiens PE = 5 SV = 2 - [CP4Z2_HUMAN] | | | | expression | |
| P02751 | Fibronectin OS = Homo sapiens PE = 1 SV = 4 - | FN1 | Enzyme | Extracellular | Increased | 0.4 |
| | [FINC_HUMAN] | | | Space | expression | |
| Q92785 | Zinc finger protein ubi-d4 OS = Homo sapiens PE = 1 SV = | DPF2 | Transcription | Nucleus | Increased | 0.4 |
| | 2 - [REQU_HUMAN] | | regulator | | expression | |
| Q9BQ39 | ATP-dependent RNA helicase DDX50 OS = Homo sapiens | DDX50 | Enzyme | Nucleus | Increased | 0.3 |
| | PE = 1 SV = 1 - [DDX50_HUMAN] | | | | expression | |
| P17813 | Endoglin OS = Homo sapiens PE = 1 SV = 2 - | ENG | Transmembrane | Plasma | Increased | 0.3 |
| | [EGLN_HUMAN] | | receptor | Membrane | expression | |
| Q7L8W6 | Diphthine--ammonia ligase OS = Homo sapiens PE = 1 | DPH6 | Enzyme | Cytoplasm | Increased | 0.3 |
| | SV = 3 - [DPH6_HUMAN] | | | | expression | |
| P04217 | Alpha-1B-glycoprotein OS = Homo sapiens PE = 1 SV = 4 - | A1BG | Other | Extracellular | Increased | 0.3 |
| | [A1BG_HUMAN] | | | Space | expression | |
| P05543 | Thyroxine-binding globulin OS = Homo sapiens PE = 1 | SERPINA7 | Transporter | Extracellular | Increased | 0.3 |
| | SV = 2 - [THBG_HUMAN] | | | Space | expression | |
| P61981 | 14-3-3 protein gamma OS = Homo sapiens PE = 1 SV = 2 - | YWHAG | Other | Cytoplasm | Increased | 0.3 |
| | [1433G_HUMAN] | | | | expression | |
| P28072 | Proteasome subunit beta type-6 OS = Homo sapiens PE = 1 | PSMB6 | Peptidase | Nucleus | Increased | 0.3 |
| | SV = 4 - [PSB6_HUMAN] | | | | expression | |
| Q93070 | Ecto-ADP-ribosyltransferase 4 OS = Homo sapiens PE = 2 | ART4 | Enzyme | Nucleus | Increased | 0.3 |
| | SV = 2 - [NAR4_HUMAN] | | | | expression | |
| Q12841 | Follistatin-related protein 1 OS = Homo sapiens PE = 1 | FSTL1 | Other | Extracellular | Increased | 0.3 |
| | SV = 1 - [FSTL1_HUMAN] | | | Space | expression | |
| P04196 | Histidine-rich glycoprotein OS = Homo sapiens PE = 1 SV = | HRG | Other | Extracellular | Increased | 0.3 |
| | 1 - [HRG_HUMAN] | | | Space | expression | |
| P02760 | Protein AMBP OS = Homo sapiens PE = 1 SV = 1 - | AMBP | Transporter | Extracellular | Increased | 0.3 |
| | [AMBP_HUMAN] | | | Space | expression | |
| Q7Z494 | Nephrocystin-3 OS = Homo sapiens PE = 1 SV = 1 - | NPHP3 | Other | Extracellular | Increased | 0.2 |
| | [NPHP3_HUMAN] | | | Space | expression | |
| P00746 | Complement factor D OS = Homo sapiens PE = 1 SV = 5 - | CFD | Peptidase | Extracellular | Increased | 0.2 |
| | [CFAD_HUMAN] | | | Space | expression | |
| P41271 | Neuroblastoma suppressor of tumorigenicity 1 OS = Homo | NBL1 | Other | Nucleus | Increased | 0.2 |
| | sapiens PE = 1 SV = 2 - [NBL1_HUMAN] | | | | expression | |
| P25445 | Tumor necrosis factor receptor superfamily member 6 | FAS | Transmembrane | Plasma | Increased | 0.2 |
| | OS = Homo sapiens PE = 1 SV = 1 - [TNR6_HUMAN] | | receptor | Membrane | expression | |
| P98160 | Basement membrane-specific heparan sulfate proteoglycan | HSPG2 | Enzyme | Extracellular | Increased | 0.2 |
| | core protein OS = Homo sapiens PE = 1 SV = 4 - | | | Space | expression | |
| | [PGBM_HUMAN] | | | | | |
| P35555 | Fibrillin-1 OS = Homo sapiens PE = 1 SV = 3 - | FBN1 | Other | Extracellular | Increased | 0.2 |
| | [FBN1_HUMAN] | | | Space | expression | |
| Q9NSI6 | Bromodomain and WD repeat-containing protein 1 | BRWD1 | Transcription | Nucleus | Increased | 0.2 |
| | OS = Homo sapiens PE = 1 SV = 4 - [BRWD1_HUMAN] | | regulator | | expression | |
| Q16853 | Membrane primary amine oxidase OS = Homo sapiens | AOC3 | Enzyme | Plasma | Increased | 0.2 |
| | PE = 1 SV = 3 - [AOC3_HUMAN] | | | Membrane | expression | |
| O75071 | EF-hand calcium-binding domain-containing protein 14 | EFCAB14 | Other | Other | Increased | 0.2 |
| | OS = Homo sapiens PE = 2 SV = 1 - [EFC14_HUMAN] | | | | expression | |
| Q8N2E2 | von Willebrand factor D and EGF domain-containing | VWDE | Other | Other | Decreased | −0.1 |
| | protein OS = Homo sapiens PE = 2 SV = 4 - | | | | expression | |
| | [VWDE_HUMAN] | | | | | |
| Q14432 | cGMP-inhibited 3′,5′-cyclic phosphodiesterase A | PDE3A | Enzyme | Cytoplasm | Decreased | −0.1 |
| | OS = Homo sapiens PE = 1 SV = 3 - [PDE3A_HUMAN] | | | | expression | |
| Q8N114 | Protein shisa-5 OS = Homo sapiens PE = 1 SV = 1 - | SHISA5 | Other | Nucleus | Decreased | −0.2 |
| | [SHSA5_HUMAN] | | | | expression | |
| P13473 | Lysosome-associated membrane glycoprotein 2 OS = Homo | LAMP2 | Enzyme | Plasma | Decreased | −0.3 |
| | sapiens PE = 1 SV = 2 - [LAMP2_HUMAN] | | | Membrane | expression | |
| P19256 | Lymphocyte function-associated antigen 3 OS = Homo | CD58 | Transmembrane | Plasma | Decreased | −0.3 |
| | sapiens PE = 1 SV = 1 - [LFA3_HUMAN] | | receptor | Membrane | expression | |
| Q8IV32 | Coiled-coil domain-containing protein 71 OS = Homo | CCDC71 | Other | Nucleus | Decreased | −0.3 |
| | sapiens PE = 2 SV = 3 - [CCD71_HUMAN] | | | | expression | |
| Q12766 | HMG domain-containing protein 3 OS = Homo sapiens | HMGXB3 | Transcription | Nucleus | Decreased | −0.3 |
| | PE = 2 SV = 2 - [HMGX3_HUMAN] | | regulator | | expression | |
| Q5T2S8 | Armadillo repeat-containing protein 4 OS = Homo sapiens | ARMC4 | Other | Extracellular | Decreased | −0.3 |
| | PE = 1 SV = 1 - [ARMC4_HUMAN] | | | Space | expression | |
| Q5T0F9 | Coiled-coil and C2 domain-containing protein 1B | CC2D1B | Transcription | Nucleus | Decreased | −0.3 |
| | OS = Homo sapiens PE = 1 SV = 1 - [C2D1B_HUMAN] | | regulator | | expression | |
| Q9H1E3 | Nuclear ubiquitous casein and cyclin-dependent kinase | NUCKS1 | Kinase | Nucleus | Decreased | −0.3 |
| | substrate 1 OS = Homo sapiens PE = 1 SV = 1 - | | | | expression | |
| | [NUCKS_HUMAN] | | | | | |
| Q5UCC4 | ER membrane protein complex subunit 10 OS = Homo | EMC10 | Other | Cytoplasm | Decreased | −0.3 |
| | sapiens PE = 1 SV = 1 - [EMC10_HUMAN] | | | | expression | |
| Q92896 | Golgi apparatus protein 1 OS = Homo sapiens PE = 1 SV = 2 - | GLG1 | Other | Cytoplasm | Decreased | −0.3 |
| | [GSLG1_HUMAN] | | | | expression | |
| Q9Y6Z7 | Collectin-10 OS = Homo sapiens PE = 2 SV = 2 - | COLEC10 | Other | Cytoplasm | Decreased | −0.3 |
| | [COL10_HUMAN] | | | | expression | |
| O60613 | 15 kDa selenoprotein OS = Homo sapiens PE = 1 SV = 3 - | SEP15 | Enzyme | Cytoplasm | Decreased | −0.3 |
| | [SEP15_HUMAN] | | | | expression | |
| Q96KN2 | Beta-Ala-His dipeptidase OS = Homo sapiens PE = 1 SV = 4 - | CNDP1 | Peptidase | Cytoplasm | Decreased | −0.3 |
| | [CNDP1_HUMAN] | | | | expression | |
| Q15021 | Condensin complex subunit 1 OS = Homo sapiens PE = 1 | NCAPD2 | Other | Nucleus | Decreased | −0.3 |
| | SV = 3 - [CND1_HUMAN] | | | | expression | |
| P31151 | Protein S100-A7 OS = Homo sapiens PE = 1 SV = 4 - | S100A7 | Other | Cytoplasm | Decreased | −0.4 |
| | [S10A7_HUMAN] | | | | expression | |
| Q8WXD2 | Secretogranin-3 OS = Homo sapiens PE = 1 SV = 3 - | SCG3 | Other | Extracellular | Decreased | −0.4 |
| | [SCG3_HUMAN] | | | Space | expression | |
| P01596 | Ig kappa chain V-I region CAR OS = Homo sapiens PE = 1 | Ig kappa | — | Extracellular | Decreased | −0.4 |
| | SV = 1 - [KV104_HUMAN] | chain V-I | | Space | expression | |
| | | region CAR | | | | |
| O43405 | Cochlin OS = Homo sapiens PE = 1 SV = 1 - | COCH | Other | Extracellular | Decreased | −0.4 |
| | [COCH_HUMAN] | | | Space | expression | |
| O43157 | Plexin-B1 OS = Homo sapiens PE = 1 SV = 3 - | PLXNB1 | Transmembrane | Plasma | Decreased | −0.4 |
| | [PLXB1_HUMAN] | | receptor | Membrane | expression | |
| P07900 | Heat shock protein HSP 90-alpha OS = Homo sapiens PE = 1 | HSP90AA1 | Enzyme | Cytoplasm | Decreased | −0.4 |
| | SV = 5 - [HS90A_HUMAN] | | | | expression | |
| Q13103 | Secreted phosphoprotein 24 OS = Homo sapiens PE = 1 | SPP2 | Other | Extracellular | Decreased | −0.4 |
| | SV = 1 - [SPP24_HUMAN] | | | Space | expression | |
| Q9Y5Y7 | Lymphatic vessel endothelial hyaluronic acid receptor 1 | LYVE1 | Transmembrane | Plasma | Decreased | −0.4 |
| | OS = Homo sapiens PE = 1 SV = 2 - [LYVE1_HUMAN] | | receptor | Membrane | expression | |
| Q15465 | Sonic hedgehog protein OS = Homo sapiens PE = 1 SV = 1 - | SHH | Peptidase | Extracellular | Decreased | −0.4 |
| | [SHH_HUMAN] | | | Space | expression | |
| P01699 | Ig lambda chain V-I region VOR OS = Homo sapiens PE = 1 | Ig lambda | Other | Other | Decreased | −0.4 |
| | SV = 1 - [LV101_HUMAN] | chain V-I | | | expression | |
| | | region VOR | | | | |
| P56202 | Cathepsin W OS = Homo sapiens PE = 1 SV = 2 - | CTSW | Peptidase | Cytoplasm | Decreased | −0.4 |
| | [CATW_HUMAN] | | | | expression | |
| P01871 | Ig mu chain C region OS = Homo sapiens PE = 1 SV = 3 - | IGHM | Transmembrane | Plasma | Decreased | −0.4 |
| | [IGHM_HUMAN] | | receptor | Membrane | expression | |
| Q8WWV6 | High affinity immunoglobulin alpha and immunoglobulin | FCAMR | Transmembrane | Plasma | Decreased | −0.4 |
| | mu Fc receptor OS = Homo sapiens PE = 1 SV = 1 - | | receptor | Membrane | expression | |
| | [FCAMR_HUMAN] | | | | | |
| P07988 | Pulmonary surfactant-associated protein B OS = Homo | SFTPB | Other | Extracellular | Decreased | −0.5 |
| | sapiens PE = 1 SV = 3 - [PSPB_HUMAN] | | | Space | expression | |
| Q9P126 | C-type lectin domain family 1 member B OS = Homo | CLEC1B | Transmembrane | Plasma | Decreased | −0.5 |
| | sapiens PE = 1 SV = 2 - [CLC1B_HUMAN] | | receptor | Membrane | expression | |
| P62714 | Serine/threonine-protein phosphatase 2A catalytic subunit | PPP2CB | Phosphatase | Cytoplasm | Decreased | −0.5 |
| | beta isoform OS = Homo sapiens PE = 1 SV = 1 - | | | | expression | |
| | [PP2AB_HUMAN] | | | | | |
| P18206 | Vinculin OS = Homo sapiens PE = 1 SV = 4 - | VCL | Enzyme | Plasma | Decreased | −0.5 |
| | [VINC_HUMAN] | | | Membrane | expression | |
| P05067 | Amyloid beta A4 protein OS = Homo sapiens PE = 1 SV = 3 - | APP | Other | Plasma | Decreased | −0.5 |
| | [A4_HUMAN] | | | Membrane | expression | |
| Q93100 | Phosphorylase b kinase regulatory subunit beta OS = Homo | PHKB | Kinase | Cytoplasm | Decreased | −0.5 |
| | sapiens PE = 1 SV = 3 - [KPBB_HUMAN] | | | | expression | |
| P01717 | Ig lambda chain V-IV region Hil OS = Homo sapiens PE = 1 | Ig lambda | Other | Extracellular | Decreased | −0.5 |
| | SV = 1 - [LV403_HUMAN] | chain V-IV | | Space | expression | |
| | | region Hil | | | | |
| P04433 | Ig kappa chain V-III region VG (Fragment) OS = Homo | Ig kappa | Other | Extracellular | Decreased | −0.5 |
| | sapiens PE = 1 SV = 1 - [KV309_HUMAN] | chain V-III | | Space | expression | |
| | | region VG | | | | |
| | | (Fragment) | | | | |
| Q15166 | Serum paraoxonase/lactonase 3 OS = Homo sapiens PE = 1 | PON3 | Enzyme | Extracellular | Decreased | −0.5 |
| | SV = 3 - [PON3_HUMAN] | | | Space | expression | |
| O75368 | SH3 domain-binding glutamic acid-rich-like protein | SH3BGRL | Other | Cytoplasm | Decreased | −0.5 |
| | OS = Homo sapiens PE = 1 SV = 1 - [SH3L1_HUMAN] | | | | expression | |
| P01622 | Ig kappa chain V-III region Ti OS = Homo sapiens PE = 1 | Ig kappa | | Extracellular | Decreased | −0.5 |
| | SV = 1 - [KV304_HUMAN] | chain V-III | | Space | expression | |
| | | region Ti | | | | |
| P08572 | Collagen alpha-2(IV) chain OS = Homo sapiens PE = 1 | COL4A2 | Other | Extracellular | Decreased | −0.5 |
| | SV = 4 - [CO4A2_HUMAN] | | | Space | expression | |
| P30041 | Peroxiredoxin-6 OS = Homo sapiens PE = 1 SV = 3 - | PRDX6 | Enzyme | Cytoplasm | Decreased | −0.6 |
| | [PRDX6_HUMAN] | | | | expression | |
| P26439 | 3 beta-hydroxysteroid dehydrogenase/Delta 5-->4- | HSD3B2 | Enzyme | Cytoplasm | Decreased | −0.6 |
| | isomerase type 2 OS = Homo sapiens PE = 1 SV = 2 - | | | | expression | |
| | [3BHS2_HUMAN] | | | | | |
| Q9C099 | Leucine-rich repeat and coiled-coil domain-containing | LRRCC1 | Transporter | Nucleus | Decreased | −0.6 |
| | protein 1 OS = Homo sapiens PE = 1 SV = 2 - | | | | expression | |
| | [LRCC1_HUMAN] | | | | | |
| P20810 | Calpastatin OS = Homo sapiens PE = 1 SV = 4 - | CAST | Peptidase | Cytoplasm | Decreased | −0.6 |
| | [ICAL_HUMAN] | | | | expression | |
| P10144 | Granzyme B OS = Homo sapiens PE = 1 SV = 2 - | GZMB | Peptidase | Cytoplasm | Decreased | −0.6 |
| | [GRAB_HUMAN] | | | | expression | |
| Q9BT88 | Synaptotagmin-11 OS = Homo sapiens PE = 1 SV = 2 - | SYT11 | Transporter | Cytoplasm | Decreased | −0.6 |
| | [SYT11_HUMAN] | | | | expression | |
| Q8N1E6 | F-box/LRR-repeat protein 14 OS = Homo sapiens PE = 1 | FBXL14 | Enzyme | Cytoplasm | Decreased | −0.6 |
| | SV = 1 - [FXL14_HUMAN] | | | | expression | |
| Q9BUQ8 | Probable ATP-dependent RNA helicase DDX23 | DDX23 | Enzyme | Nucleus | Decreased | −0.6 |
| | OS = Homo sapiens PE = 1 SV = 3 - [DDX23_HUMAN] | | | | expression | |
| P01824 | Ig heavy chain V-II region WAH OS = Homo sapiens PE = 1 | Ig heavy | Other | Other | Decreased | −0.6 |
| | SV = 1 - [HV206_HUMAN] | chain V-II | | | expression | |
| | | region WAH | | | | |
| P02042 | Hemoglobin subunit delta OS = Homo sapiens PE = 1 SV = | HBD | Transporter | Other | Decreased | −0.6 |
| | 2 - [HBD_HUMAN] | | | | expression | |
| Q15149 | Plectin OS = Homo sapiens PE = 1 SV = 3 - | PLEC | Other | Cytoplasm | Decreased | −0.6 |
| | [PLEC_HUMAN] | | | | expression | |
| P21266 | Glutathione S-transferase Mu 3 OS = Homo sapiens PE = 1 | GSTM3 | Enzyme | Cytoplasm | Decreased | −0.6 |
| | SV = 3 - [GSTM3_HUMAN] | | | | expression | |
| Q9UBB4 | Ataxin-10 OS = Homo sapiens PE = 1 SV = 1 - | ATXN10 | Other | Cytoplasm | Decreased | −0.6 |
| | [ATX10_HUMAN] | | | | expression | |
| P55000 | Secreted Ly-6/uPAR-related protein 1 OS = Homo sapiens | SLURP1 | Cytokine | Extracellular | Decreased | 0.6 |
| | PE = 1 SV = 2 - [SLUR1_HUMAN] | | | Space | expression | |
| P06311 | Ig kappa chain V-III region IARC/BL41 OS = Homo | Ig kappa | — | Extracellular | Decreased | −0.6 |
| | sapiens PE = 1 SV = 1 - [KV311_HUMAN] | chain V-III | | Space | expression | |
| | | region IARC/ | | | | |
| | | BL41 | | | | |
| P00742 | Coagulation factor X OS = Homo sapiens PE = 1 SV = 2 - | F10 | Peptidase | Extracellular | Decreased | −0.6 |
| | [FA10_HUMAN] | | | Space | expression | |
| P01609 | Ig kappa chain V-I region Scw OS = Homo sapiens PE = 1 | Ig kappa | — | — | Decreased | 0.6 |
| | SV = 1 - [KV117_HUMAN] | chain V-I | | | expression | |
| | | region Scw | | | | |
| P01602 | Ig kappa chain V-I region HK102 (Fragment) OS = Homo | IGKV1-5 | Other | Extracellular | Decreased | −0.6 |
| | sapiens PE = 4 SV = 1 - [KV110_HUMAN] | | | Space | expression | |
| P40145 | Adenylate cyclase type 8 OS = Homo sapiens PE = 1 SV = 1 - | ADCY8 | Enzyme | Plasma | Decreased | −0.6 |
| | [ADCY8_HUMAN] | | | Membrane | expression | |
| Q86TI2 | Dipeptidyl peptidase 9 OS = Homo sapiens PE = 1 SV = 3 - | DPP9 | Peptidase | Cytoplasm | Decreased | −0.6 |
| | [DPP9_HUMAN] | | | | expression | |
| Q6ZMR5 | Transmembrane protease serine 11A OS = Homo sapiens | TMPRSS11A | Peptidase | Other | Decreased | −0.6 |
| | PE = 1 SV = 1 - [TM11A_HUMAN] | | | | expression | |
| P20648 | Potassium-transporting ATPase alpha chain 1 OS = Homo | ATP4A | Transporter | Plasma | Decreased | −0.7 |
| | sapiens PE = 2 SV = 5 - [ATP4A_HUMAN] | | | Membrane | expression | |
| Q6ZRS2 | Helicase SRCAP OS = Homo sapiens PE = 1 SV = 3 - | SRCAP | Transcription | Cytoplasm | Decreased | −0.7 |
| | [SRCAP_HUMAN] | | regulator | | expression | |
| P29375 | Lysine-specific demethylase 5A OS = Homo sapiens PE = 1 | KDM5A | Transcription | Nucleus | Decreased | −0.7 |
| | SV = 3 - [KDM5A_HUMAN] | | regulator | | expression | |
| Q9HC56 | Protocadherin-9 OS = Homo sapiens PE = 1 SV = 2 - | PCDH9 | Other | Plasma | Decreased | −0.7 |
| | [PCDH9_HUMAN] | | | Membrane | expression | |
| P0CG05 | Ig lambda-2 chain C regions OS = Homo sapiens PE = 1 | IGLC2 | — | Extracellular | Decreased | −0.7 |
| | SV = 1 - [LAC2_HUMAN] | | | Space | expression | |
| P37840 | Alpha-synuclein OS = Homo sapiens PE = 1 SV = 1 - | SNCA | Enzyme | Cytoplasm | Decreased | −0.7 |
| | [SYUA_HUMAN] | | | | expression | |
| Q70EL4 | Ubiquitin carboxyl-terminal hydrolase 43 OS = Homo | USP43 | Peptidase | Nucleus | Decreased | −0.7 |
| | sapiens PE = 1 SV = 2 - [UBP43_HUMAN] | | | | expression | |
| Q7Z443 | Polycystic kidney disease protein 1-like 3 OS = Homo | PKD1L3 | Ion channel | Plasma | Decreased | −0.7 |
| | sapiens PE = 1 SV = 1 - [PK1L3_HUMAN] | | | Membrane | expression | |
| Q96M20 | Cyclic nucleotide-binding domain-containing protein 2 | CNBD2 | Other | Cytoplasm | Decreased | −0.7 |
| | OS = Homo sapiens PE = 2 SV = 2 - [CNBD2_HUMAN] | | | | expression | |
| P01702 | Ig lambda chain V-I region NIG-64 OS = Homo sapiens | Ig lambda | Other | Extracellular | Decreased | −0.7 |
| | PE = 1 SV = 1 - [LV104_HUMAN] | chain V-I | | Space | expression | |
| | | region NIG-64 | | | | |
| Q9P2X0 | Dolichol-phosphate mannosyltransferase subunit 3 | DPM3 | Enzyme | Cytoplasm | Decreased | −0.7 |
| | OS = Homo sapiens PE = 1 SV = 2 - [DPM3_HUMAN] | | | | expression | |
| A0M8Q6 | Ig lambda-7 chain C region OS = Homo sapiens PE = 1 | IGLC7 | Other | Extracellular | Decreased | −0.7 |
| | SV = 2 - [LAC7_HUMAN] | | | Space | expression | |
| P01611 | Ig kappa chain V-I region Wes OS = Homo sapiens PE = 1 | Ig kappa | Other | Extracellular | Decreased | −0.7 |
| | SV = 1 - [KV119_HUMAN] | chain V-I | | Space | expression | |
| | | region Wes | | | | |
| P17483 | Homeobox protein Hox-B4 OS = Homo sapiens PE = 1 | HOXB4 | Transcription | Nucleus | Decreased | −0.7 |
| | SV = 2 - [HXB4_HUMAN] | | regulator | | expression | |
| P32119 | Peroxiredoxin-2 OS = Homo sapiens PE = 1 SV = 5 - | PRDX2 | Enzyme | Cytoplasm | Decreased | −0.7 |
| | [PRDX2_HUMAN] | | | | expression | |
| Q8TCU4 | Alstrom syndrome protein 1 OS = Homo sapiens PE = 1 | ALMS1 | Other | Cytoplasm | Decreased | −0.7 |
| | SV = 3 - [ALMS1_HUMAN] | | | | expression | |
| P04070 | Vitamin K-dependent protein C OS = Homo sapiens PE = 1 | PROC | Peptidase | Extracellular | Decreased | −0.8 |
| | SV = 1 - [PROC_HUMAN] | | | Space | expression | |
| P16519 | Neuroendocrine convertase 2 OS = Homo sapiens PE = 2 | PCSK2 | Peptidase | Extracellular | Decreased | −0.8 |
| | SV = 2 - [NEC2_HUMAN] | | | Space | expression | |
| P29084 | Transcription initiation factor IIE subunit beta OS = Homo | GTF2E2 | Transcription | Nucleus | Decreased | −0.8 |
| | sapiens PE = 1 SV = 1 - [T2EB_HUMAN] | | regulator | | expression | |
| P56730 | Neurotrypsin OS = Homo sapiens PE = 2 SV = 2 - | PRSS12 | Peptidase | Extracellular | Decreased | −0.8 |
| | [NETR_HUMAN] | | | Space | expression | |
| P23458 | Tyrosine-protein kinase JAK1 OS = Homo sapiens PE = 1 | JAK1 | Kinase | Cytoplasm | Decreased | −0.8 |
| | SV = 2 - [JAK1_HUMAN] | | | | expression | |
| A6NNP5 | Coiled-coil domain-containing protein 169 OS = Homo | CCDC169 | Other | Other | Decreased | −0.8 |
| | sapiens PE = 2 SV = 4 - [CC169_HUMAN] | | | | expression | |
| Q08495 | Dematin OS = Homo sapiens PE = 1 SV = 3 - | DMTN | Other | Plasma | Decreased | −0.8 |
| | [DEMA_HUMAN] | | | Membrane | expression | |
| A6NKL6 | Transmembrane protein 200C OS = Homo sapiens PE = 2 | TMEM200C | Other | Other | Decreased | −0.8 |
| | SV = 2 - [T200C_HUMAN] | | | | expression | |
| Q9H583 | HEAT repeat-containing protein 1 OS = Homo sapiens | HEATR1 | Other | Nucleus | Decreased | −0.8 |
| | PE = 1 SV = 3 - [HEAT1_HUMAN] | | | | expression | |
| P20851 | C4b-binding protein beta chain OS = Homo sapiens PE = 1 | C4BPB | Other | Extracellular | Decreased | −0.8 |
| | SV = 1 - [C4BPB_HUMAN] | | | Space | expression | |
| P01715 | Ig lambda chain V-IV region Bau OS = Homo sapiens PE = 1 | Ig lambda | — | Extracellular | Decreased | −0.8 |
| | SV = 1 - [LV401_HUMAN] | chain V-IV | | Space | expression | |
| | | region Bau | | | | |
| Q9UBC9 | Small proline-rich protein 3 OS = Homo sapiens PE = 1 | SPRR3 | Other | Cytoplasm | Decreased | −0.9 |
| | SV = 2 - [SPRR3_HUMAN] | | | | expression | |
| Q9Y305 | Acyl-coenzyme A thioesterase 9, mitochondrial OS = Homo | ACOT9 | Enzyme | Cytoplasm | Decreased | −0.9 |
| | sapiens PE = 1 SV = 2 - [ACOT9_HUMAN] | | | | expression | |
| Q6P4Q7 | Metal transporter CNNM4 OS = Homo sapiens PE = 1 SV = | CNNM4 | Transporter | Plasma | Decreased | −0.9 |
| | 3 - [CNNM4_HUMAN] | | | Membrane | expression | |
| Q6P1J6 | Phospholipase B1, membrane-associated OS = Homo | PLB1 | Enzyme | Cytoplasm | Decreased | −0.9 |
| | sapiens PE = 1 SV = 3 - [PLB1_HUMAN] | | | | expression | |
| P06889 | Ig lambda chain V-IV region MOL OS = Homo sapiens | Ig lambda | Other | Other | Decreased | −0.9 |
| | PE = 1 SV = 1 - [LV405_HUMAN] | chain V-IV | | | expression | |
| | | region MOL | | | | |
| Q02218 | 2-oxoglutarate dehydrogenase, mitochondrial OS = Homo | OGDH | Enzyme | Cytoplasm | Decreased | −0.9 |
| | sapiens PE = 1 SV = 3 - [ODO1_HUMAN] | | | | expression | |
| Q68CZ6 | HAUS augmin-like complex subunit 3 OS = Homo sapiens | HAUS3 | Other | Cytoplasm | Decreased | −0.9 |
| | PE = 1 SV = 1 - [HAUS3_HUMAN] | | | | expression | |
| P12270 | Nucleoprotein TPR OS = Homo sapiens PE = 1 SV = 3 - | TPR | Other | Nucleus | Decreased | −0.9 |
| | [TPR_HUMAN] | | | | expression | |
| Q8TEP8 | Centrosomal protein of 192 kDa OS = Homo sapiens PE = 1 | CEP192 | Other | Cytoplasm | Decreased | −0.9 |
| | SV = 2 - [CE192_HUMAN] | | | | expression | |
| P04206 | Ig kappa chain V-III region GOL OS = Homo sapiens PE = 1 | Ig kappa | Other | Extracellular | Decreased | −0.9 |
| | SV = 1 - [KV307_HUMAN] | chain V-III | | Space | expression | |
| | | region GOL | | | | |
| P01710 | Ig lambda chain V-II region BO OS = Homo sapiens PE = 1 | Ig lambda | Other | Other | Decreased | −0.9 |
| | SV = 1 - [LV207_HUMAN] | chain V-II | | | expression | |
| | | region BO | | | | |
| O75636 | Ficolin-3 OS = Homo sapiens PE = 1 SV = 2 - | FCN3 | Other | Extracellular | Decreased | −0.9 |
| | [FCN3_HUMAN] | | | Space | expression | |
| P12872 | Promotilin OS = Homo sapiens PE = 1 SV = 1 - | MLN | Other | Extracellular | Decreased | −0.9 |
| | [MOTI_HUMAN] | | | Space | expression | |
| O00541 | Pescadillo homolog OS = Homo sapiens PE = 1 SV = 1 - | PES1 | Other | Nucleus | Decreased | −0.9 |
| | [PESC_HUMAN] | | | | expression | |
| Q9Y664 | Kaptin OS = Homo sapiens PE = 1 SV = 2 - | KPTN | Other | Plasma | Decreased | −0.9 |
| | [KPTN_HUMAN] | | | Membrane | expression | |
| P07360 | Complement component C8 gamma chain OS = Homo | C8G | Transporter | Extracellular | Decreased | −0.9 |
| | sapiens PE = 1 SV = 3 - [CO8G_HUMAN] | | | Space | expression | |
| P04209 | Ig lambda chain V-II region NIG-84 OS = Homo sapiens | Ig lambda | Other | Extracellular | Decreased | −0.9 |
| | PE = 1 SV = 1 - [LV211_HUMAN] | chain V-II | | Space | expression | |
| | | region NIG-84 | | | | |
| O00522 | Krev interaction trapped protein 1 OS = Homo sapiens | KRIT1 | Other | Plasma | Decreased | −0.9 |
| | PE = 1 SV = 2 - [KRIT1_HUMAN] | | | Membrane | expression | |
| Q9Y6R7 | IgGFc-binding protein OS = Homo sapiens PE = 1 SV = 3 - | FCGBP | Other | Extracellular | Decreased | −1.0 |
| | [FCGBP_HUMAN] | | | Space | expression | |
| O43866 | CD5 antigen-like OS = Homo sapiens PE = 1 SV = 1 - | CD5L | Transmembrane | Plasma | Decreased | −1.0 |
| | [CD5L_HUMAN] | | receptor | Membrane | expression | |
| Q9H2Y9 | Solute carrier organic anion transporter family member | SLCO5A1 | Transporter | Plasma | Decreased | −1.0 |
| | 5A1 OS = Homo sapiens PE = 2 SV = 2 - [SO5A1_HUMAN] | | | Membrane | expression | |
| A1Z1Q3 | O-acetyl-ADP-ribose deacetylase MACROD2 OS = Homo | MACROD2 | Enzyme | Nucleus | Decreased | −1.0 |
| | sapiens PE = 1 SV = 1 - [MACD2_HUMAN] | | | | expression | |
| P01714 | Ig lambda chain V-III region SH OS = Homo sapiens PE = 1 | Ig lambda | Other | Extracellular | Decreased | −1.0 |
| | SV = 1 - [LV301_HUMAN] | chain V-III | | Space | expression | |
| | | region SH | | | | |
| Q9BWM7 | Sideroflexin-3 OS = Homo sapiens PE = 1 SV = 2 - | SFXN3 | Transporter | Cytoplasm | Decreased | −1.1 |
| | [SFXN3_HUMAN] | | | | expression | |
| Q9H9H5 | MAP6 domain-containing protein 1 OS = Homo sapiens | MAP6D1 | Other | Cytoplasm | Decreased | −1.1 |
| | PE = 1 SV = 1 - [MA6D1_HUMAN] | | | | expression | |
| Q2M2H8 | Probable maltase-glucoamylase-like protein LOC93432 | Probable | Other | Other | Decreased | −1.1 |
| | OS = Homo sapiens PE = 2 SV = 3 - [MGAL_HUMAN] | maltase- | | | expression | |
| | | glucoamylase- | | | | |
| | | like protein | | | | |
| | | LOC93432 | | | | |
| P32320 | Cytidine deaminase OS = Homo sapiens PE = 1 SV = 2 - | CDA | Enzyme | Nucleus | Decreased | −1.1 |
| | [CDD_HUMAN] | | | | expression | |
| Q7L5N1 | COP9 signalosome complex subunit 6 OS = Homo sapiens | COPS6 | Other | Nucleus | Decreased | −1.1 |
| | PE = 1 SV = 1 - [CSN6_HUMAN] | | | | expression | |
| O95486 | Protein transport protein Sec24A OS = Homo sapiens PE = 1 | SEC24A | Transporter | Cytoplasm | Decreased | −1.2 |
| | SV = 2 - [SC24A_HUMAN] | | | | expression | |
| Q5VU43 | Myomegalin OS = Homo sapiens PE = 1 SV = 1 - | PDE4DIP | Enzyme | Cytoplasm | Decreased | −1.2 |
| | [MYOME_HUMAN] | | | | expression | |
| P01707 | Ig lambda chain V-II region TRO OS = Homo sapiens PE = 1 | Ig lambda | — | Extracellular | Decreased | −1.2 |
| | SV = 1 - [LV204_HUMAN] | chain V-II | | Space | expression | |
| | | region TRO | | | | |
| P80422 | Ig gamma lambda chain V-II region DOT OS = Homo | Ig gamma | — | Extracellular | Decreased | −1.2 |
| | sapiens PE = 1 SV = 1 - [LV212_HUMAN] | lambda | | Space | expression | |
| | | chain V-II | | | | |
| | | region DOT | | | | |
| O75344 | Inactive peptidyl-prolyl cis-trans isomerase FKBP6 | FKBP6 | Enzyme | Nucleus | Decreased | −1.2 |
| | OS = Homo sapiens PE = 1 SV = 1 - [FKBP6_HUMAN] | | | | expression | |
| Q7Z5P9 | Mucin-19 OS = Homo sapiens PE = 1 SV = 2 - | MUC19 | Other | Cytoplasm | Decreased | −1.2 |
| | [MUC19_HUMAN] | | | | expression | |
| Q9HCS7 | Pre-mRNA-splicing factor SYF1 OS = Homo sapiens PE = 1 | XAB2 | Other | Nucleus | Decreased | −1.3 |
| | SV = 2 - [SYF1_HUMAN] | | | | expression | |
| P17022 | Zinc finger protein 18 OS = Homo sapiens PE = 2 SV = 2 - | ZNF18 | Transcription | Nucleus | Decreased | −1.3 |
| | [ZNF18_HUMAN] | | regulator | | expression | |
| Q8N9V6 | Ankyrin repeat domain-containing protein 53 OS = Homo | ANKRD53 | Transcription | Cytoplasm | Decreased | −1.3 |
| | sapiens PE = 2 SV = 3 - [ANR53_HUMAN] | | regulator | | expression | |
| Q13136 | Liprin-alpha-1 OS = Homo sapiens PE = 1 SV = 1 - | PPFIA1 | Phosphatase | Plasma | Decreased | −1.3 |
| | [LIPA1_HUMAN] | | | Membrane | expression | |
| P01706 | Ig lambda chain V-II region BOH OS = Homo sapiens | Ig lambda | Other | Extracellular | Decreased | −1.3 |
| | PE = 1 SV = 1 - [LV203_HUMAN] | chain V-II | | Space | expression | |
| | | region BOH | | | | |
| Q9NV88 | Integrator complex subunit 9 OS = Homo sapiens PE = 1 | INTS9 | Other | Nucleus | Decreased | −1.4 |
| | SV = 2 - [INT9_HUMAN] | | | | expression | |
| Q92835 | Phosphatidylinositol 3,4,5-trisphosphate 5-phosphatase 1 | INPP5D | Phosphatase | Cytoplasm | Decreased | −1.4 |
| | OS = Homo sapiens PE = 1 SV = 2 - [SHIP1_HUMAN] | | | | expression | |
| Q9H3P7 | Golgi resident protein GCP60 OS = Homo sapiens PE = 1 | ACBD3 | Other | Cytoplasm | Decreased | −1.4 |
| | SV = 4 - [GCP60_HUMAN] | | | | expression | |
| Q96DT5 | Dynein heavy chain 11, axonemal OS = Homo sapiens | DNAH11 | Enzyme | Cytoplasm | Decreased | −1.4 |
| | PE = 1 SV = 3 - [DYH11_HUMAN] | | | | expression | |
| Q8IXY8 | Peptidyl-prolyl cis-trans isomerase-like 6 OS = Homo | PPIL6 | Enzyme | Other | Decreased | −1.4 |
| | sapiens PE = 2 SV = 1 - [PPIL6_HUMAN] | | | | expression | |
| A3KN83 | Protein strawberry notch homolog 1 OS = Homo sapiens | SBNO1 | Enzyme | Other | Decreased | −1.5 |
| | PE = 1 SV = 1 - [SBNO1_HUMAN] | | | | expression | |
| Q9Y6I3 | Epsin-1 OS = Homo sapiens PE = 1 SV = 2 - | EPN1 | Other | Plasma | Decreased | −1.5 |
| | [EPN1_HUMAN] | | | Membrane | expression | |
| P01591 | Immunoglobulin J chain OS = Homo sapiens PE = 1 SV = 4 - | IGJ | Other | Extracellular | Decreased | −1.6 |
| | [IGJ_HUMAN] | | | Space | expression | |
| Q6YHU6 | Thyroid adenoma-associated protein OS = Homo sapiens | THADA | Other | Cytoplasm | Decreased | −1.6 |
| | PE = 1 SV = 1 - [THADA_HUMAN] | | | | expression | |
| O14983 | Sarcoplasmic/endoplasmic reticulum calcium ATPase 1 | ATP2A1 | Transporter | Cytoplasm | Decreased | −1.6 |
| | OS = Homo sapiens PE = 1 SV = 1 - [AT2A1_HUMAN] | | | | expression | |
| Q00688 | Peptidyl-prolyl cis-trans isomerase FKBP3 OS = Homo | FKBP3 | Enzyme | Nucleus | Decreased | −1.7 |
| | sapiens PE = 1 SV = 1 - [FKBP3_HUMAN] | | | | expression | |
| Q86XJ1 | GAS2-like protein 3 OS = Homo sapiens PE = 1 SV = 1 - | GAS2L3 | Other | Other | Decreased | −1.7 |
| | [GA2L3_HUMAN] | | | | expression | |
| A0AVI2 | Fer-1-like protein 5 OS = Homo sapiens PE = 2 SV = 2 - | FER1L5 | Other | Other | Decreased | −1.7 |
| | [FR1L5_HUMAN] | | | | expression | |
| Q9C0K0 | B-cell lymphoma/leukemia 11B OS = Homo sapiens PE = 1 | BCL11B | Transcription | Nucleus | Decreased | −1.8 |
| | SV = 1 - [BC11B_HUMAN] | | regulator | | expression | |
| P01880 | Ig delta chain C region OS = Homo sapiens PE = 1 SV = 2 - | IGHD | Other | Extracellular | Decreased | −1.9 |
| | [IGHD_HUMAN] | | | Space | expression | |
| P80362 | Ig kappa chain V-I region WAT OS = Homo sapiens PE = 1 | Ig kappa | Other | Extracellular | Decreased | −1.9 |
| | SV = 1 - [KV125_HUMAN] | chain V-I | | Space | expression | |
| | | region WAT | | | | |
| O75146 | Huntingtin-interacting protein 1-related protein OS = Homo | HIP1R | Other | Cytoplasm | Decreased | −2.0 |
| | sapiens PE = 1 SV = 2 - [HIP1R_HUMAN] | | | | expression | |
| Q9NYF3 | Protein FAM53C OS = Homo sapiens PE = 1 SV = 1 - | FAM53C | Other | Other | Decreased | −2.2 |
| | [FA53C_HUMAN] | | | | expression | |
| O15031 | Plexin-B2 OS = Homo sapiens PE = 1 SV = 3 - | PLXNB2 | Transmembrane | Plasma | Decreased | −2.4 |
| | [PLXB2_HUMAN] | | receptor | Membrane | expression | |
| A2RUB1 | Uncharacterized protein C17orf104 OS = Homo sapiens | C17orf104 | Other | Cytoplasm | Decreased | −2.5 |
| | PE = 2 SV = 3 - [CQ104_HUMAN] | | | | expression | |
| Q9H329 | Band 4.1-like protein 4B OS = Homo sapiens PE = 2 SV = 2 - | EPB41L4B | Transporter | Cytoplasm | Decreased | −2.5 |
| | [E41LB_HUMAN] | | | | expression | |
| Q6ZS81 | WD repeat- and FYVE domain-containing protein 4 | WDFY4 | Other | Other | Decreased | −2.6 |
| | OS = Homo sapiens PE = 1 SV = 3 - [WDFY4_HUMAN] | | | | expression | |
| Q9UIW2 | Plexin-A1 OS = Homo sapiens PE = 1 SV = 3 - | PLXNA1 | Transmembrane | Plasma | Decreased | −2.8 |
| | [PLXA1_HUMAN] | | receptor | Membrane | expression | |
| Q9Y4D8 | Probable E3 ubiquitin-protein ligase HECTD4 OS = Homo | HECTD4 | Other | Nucleus | Decreased | −4.3 |
| | sapiens PE = 1 SV = 5 - [HECD4_HUMAN] | | | | expression | |
| Q86YI8 | PHD finger protein 13 OS = Homo sapiens PE = 1 SV = 2 - | PHF13 | Other | Nucleus | Decreased | −4.4 |
| | [PHF13_HUMAN] | | | | expression | |

### IV. Devices and Systems

Provided herein are devices and system useful for the methods described herein. In some embodiments, provided is a microfluidic device for separation of components, or products thereof, of a sample, e.g., a size-exclusion chromatography microfluidic device or a reversed-phase liquid chromatography microfluidic device. In some embodiments, provided herein a system that integrated steps of a method described herein. In some embodiments, the system comprises a microfluidic device for separation of components, or products thereof, of a sample, and other features useful for completing and/or integrating steps of a method described herein. In some embodiments, the system comprises features for automation, such as robotics.

### A. Microfluidic Devices for Separation of Components of a Sample

In some aspects, provided herein are microfluidic device configured to separate components of a sample. In some embodiments, the microfluidic device comprises a plurality of interconnected channels comprising a medium useful for separation (such as a porous medium or a reversed-phase medium). The microfluidic devices comprising a plurality of interconnected channels are useful for the efficient and efficacious separation of a diverse array of components of a sample, and thus enable concurrent proteomics, peptidomics, and metabolomics analyses of, e.g., complex biological samples.

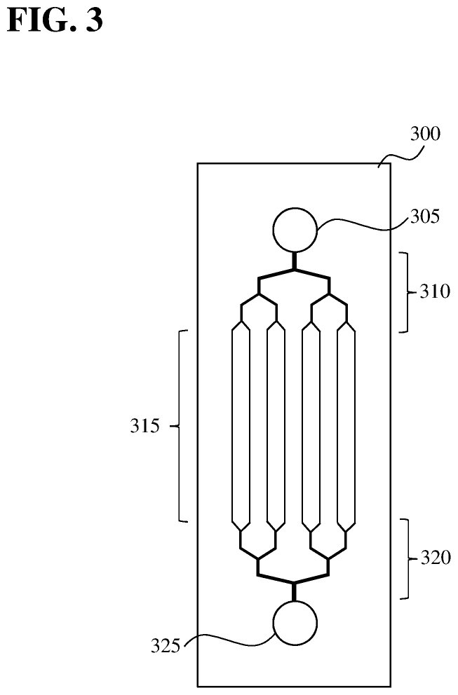

A schematic of an exemplary microfluidic device 300 is provided in FIG. 3. The microfluidic device 300 comprises an input port 305 in fluidic communication with an upstream network of connection channels 310 connecting the input port 305 with a plurality of interconnected channels 315. The microfluidic device 300 is configured to receive a fluid via the input port 305, and to direct portions of the fluid to each of the interconnected channels 315 via the upstream network of connection channels 310. The interconnected channels 315 are also in fluidic communication with a downstream network of connection channels 320, which terminate at an output port 325. The microfluidic device is configured to direct eluate from each of the plurality of interconnected channels to an output feature, such as a single output port 325, via the downstream network of connection channels 320. In some embodiments, the input port is configured to interface with a sample injector and/or mobile phase source (such as a pump). In some embodiments, the output port is configured to interface with a downstream tool or feature useful for the methods described herein. In some embodiments, the output port is configured to interface with a collection device, such as a fraction collector. In some embodiments, the output port is configured to interface with an electrospray ionization source.

In some embodiments, the microfluidic device comprises a plurality of interconnected channels. In some embodiments, the plurality of interconnected channels is configured as a plurality of interconnected parallel channels. In such embodiments, the term “parallel” indicates that a fluid input into the microfluidic device is split and the portions of the fluid travel through different channels, or different sections thereof, of the interconnected channels simultaneously, and is not intended to be construed as a limitation regarding the shape of the interconnected channels (e.g., that interconnected parallel channels can only be straight lines configured in a geometrically parallel fashion). In some embodiments, the plurality of interconnected channels comprises one or more channels comprising a substantially linear feature of a channel. In some embodiments, the plurality of interconnected channels comprises one or more channels comprising a non-linear feature of a channel, such as comprising a divergent, staggered, or waveform geometry. In some embodiments, the microfluidic devices described herein may comprise any number of interconnected channels, such that the interconnected channels are in fluid communication with an input port(s) of the microfluidic device, and the interconnected channels are configured to receive a portion of a fluid introduced to the microfluidic device via the input port(s). In some embodiments, the plurality of interconnected channels of a microfluidic device comprises 8 or more interconnected channels. In some embodiments, the plurality of interconnected channels of a microfluidic device has any of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 interconnected channels. In some embodiments, the plurality of interconnected channels of a microfluidic device comprises 32 interconnected channels. In some embodiments, the plurality of interconnected channels of a microfluidic device comprises 64 interconnected channels.

In some embodiments, each of the plurality of interconnected channels of a microfluidic device are in fluidic communication with an input port of the microfluidic device. In some embodiments, each of the plurality of interconnected channels of a microfluidic device are in fluidic communication with an input port of the microfluidic device via an upstream network of connection channels. In some embodiments, the upstream network of connection channels is configured to direct and split flow of a fluid introduced via an input port of a microfluidic device such that a portion of the fluid is delivered to each interconnected channel. In some embodiments, the upstream network of connection channels, or portions thereof, is connected to a proximal region of each of a plurality of interconnected channels. In some embodiments, the proximal region of an interconnected channel is the region of the interconnected channel first subjected to a fluid introduced via an input port of a microfluidic device. The upstream network of connection channels may be configured in numerous ways and provide one or more functions in addition to directing a fluid to an interconnected channel, e.g., a mixing function and/or a filtering function and/or a dilution function. In some embodiments, the upstream network of connection channels comprises a series of diverging channels configured to split fluid flow from the input port of a microfluidic device to each of the plurality of interconnected channels. In some embodiments, the series of diverging channels of an upstream network of connection channels are structuring using a 1 to 2 split (e.g., from the upstream to downstream direction based on intended fluid flow, one channel splits into two channels). For example, in some embodiments, for a microfluidic device having a single input port and 32 interconnected channels, an upstream network of connection channels comprises a single channel that splits to two channels, wherein each of the two channels splits to two channels (now four total channels), wherein each of the four channels splits to two channels (now eight total channels), wherein each of the eight channels splits to two channels (now 16 channels), wherein the each of the 16 channels splits to two channels (now 32 channels), and wherein each of the 32 channels is connected to one of the 32 interconnected channels. In some embodiments, the upstream network of connection channels comprises a 1 to 2 split, 1 to 3 split, a 1 to 4 split, a 1 to 5 split, a 1 to 6 split, a 1 to 7 split, a 1 to 8 split, a 1 to 9 split, a 1 to 10 split, a 1 to 11 split, a 1 to 12 split, or any combination thereof. In some embodiments, the channels of an upstream network of connection channels after a split (i.e., split channels) have a smaller cross-sectional dimension (such as height and/or width) as compared to the channel from which they originate.

In some embodiments, each of the plurality of interconnected channels of a microfluidic device is in fluidic communication with an output port of the microfluidic device. In some embodiments, each of the plurality of interconnected channels of a microfluidic device is in fluidic communication with an output port of the microfluidic device via a downstream network of connection channels. In some embodiments, the downstream network of connection channels is configured to direct and combine flow of a fluid from each of a plurality of interconnected channels to an output port of a microfluidic device (including, e.g., more than one output port of a microfluidic device). In some embodiments, the downstream network of connection channels, or portions thereof, is connected to a distal region of each of a plurality of interconnected channels. In some embodiments, the distal region of an interconnected channel is the region of the interconnected channel from which a fluid exits the interconnected channel to a downstream feature. The downstream network of connection channels may be configured in numerous ways and provide one or more functions in addition to directing a fluid to an output port, e.g., a mixing function and/or a filtering function and/or a dilution function. In some embodiments, the downstream network of connection channels comprises a series of converging channels configured to combine fluid flow from a plurality of interconnected channels of to microfluidic device to an output port. In some embodiments, the series of converging channels of a downstream network of connection channels are structuring using a 2 to 1 split (e.g., from the upstream to downstream direction based on intended fluid flow, two channel converge into one channel). For example, in some embodiments, for a microfluidic device having a single output port and 32 interconnected channels, a downstream network of connection channels comprises a 32 channels, each of the 32 channels of the downstream network of connection channels is connected to a channel of the 32 interconnected channels, wherein pairs of the 32 channels of the downstream network of connection channels converge to a single channel (now 16 channels), wherein pairs of the 16 channels of the downstream network of connection channels converge to a single channel (now 8 channels), wherein pairs of the 8 channels of the downstream network of connection channels converge to a single channel (now 4 channels), wherein pairs of the 4 channels of the downstream network of connection channels converge to a single channel (now 2 channels), wherein the two channels of the downstream network of connection channels converge to a single channel in fluid communication with the output port. In some embodiments, the downstream network of connection channels comprises a 2 to 1 convergence, a 3 to 1 convergence, a 4 to 1 convergence, a 5 to 1 convergence, a 6 to 1 convergence, a 7 to 1 convergence, a 8 to 1 convergence, a 9 to 1 convergence, a 10 to 1 convergence, a 11 to 1 convergence, a 12 to 1 convergence, or any combination thereof. In some embodiments, the channel of a downstream network of connection channels after a convergence (i.e., a converged channel) has a larger cross-sectional dimension (such as height and/or width) as compared to the channel from which they originate.

In some embodiments, the plurality of interconnected channels of a microfluidic device are only connected via an upstream network of connection channels and/or a downstream network of connection channels. For example, in some embodiments, one interconnected channel is not connected to another interconnected channel, except via an upstream network of connection channels and/or a downstream network of connection channels.

In some embodiments, each of the plurality of interconnected channels of a microfluidic device has a length of about 2 cm to about 50 cm, such as about 5 cm to about 20 cm. In some embodiments, the length of an interconnected channel is at least about 5 cm, such as at least about any of 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, 41 cm, 42 cm, 43 cm, 44 cm, 45 cm, 46 cm, 47 cm, 48 cm, 49 cm, or 50 cm. In some embodiments, the length of an interconnected channel is less than about 50 cm, such as less than about any of 49 cm, 48 cm, 47 cm, 46 cm, 45 cm, 44 cm, 43 cm, 42 cm, 41 cm, 40 cm, 39 cm, 38 cm, 37 cm, 36 cm, 35 cm, 34 cm, 33 cm, 32 cm, 31 cm, 30 cm, 29 cm, 28 cm, 27 cm, 26 cm, 25 cm, 24 cm, 23 cm, 22 cm, 21 cm, 20 cm, 19 cm, 18 cm, 17 cm, 16 cm, 15 cm, 14 cm, 13 cm, 12 cm, 11 cm, 10 cm, 9 cm, 8 cm, 7 cm, 6 cm, 5 cm, 4 cm, 3 cm, or 2 cm. In some embodiments, the length of an interconnected channel is about any 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, 30 cm, 31 cm, 32 cm, 33 cm, 34 cm, 35 cm, 36 cm, 37 cm, 38 cm, 39 cm, 40 cm, 41 cm, 42 cm, 43 cm, 44 cm, 45 cm, 46 cm, 47 cm, 48 cm, 49 cm, or 50 cm.

In some embodiments, the total length of a plurality of interconnected channels is about 20 cm to about 3,200 cm. In some embodiments, the total length of a plurality of interconnected channels is greater than about 20 cm, such as greater than about any of 50 cm, 75 cm, 100 cm, 150 cm, 200 cm, 250 cm, 300 cm, 350 cm, 400 cm, 450 cm, 500 cm, 600 cm, 700 cm, 800 cm, 900 cm, 1,000 cm, 1,250 cm, 1,500 cm, 1,750 cm, 2,000 cm, 2,250 cm, 2,500 cm, 2,750 cm, or 3,000 cm.

The channels of the interconnected channels, upstream network of connection channels, and downstream network of connection channels described herein may be formed having various cross-sectional shapes and sizes. In some embodiments, the cross-section shape and size of a channel described herein may change at different points of the channel. In some embodiments, the channel has a cross-sectional shape comprising a rectangle, a square, or a circle.

In some embodiments, the interconnected channel of a microfluidic device has a cross-sectional dimension of about 1 μm to about 15 μm, such as about 3 μm to about 10 μm. In some embodiments, the interconnected channel of a microfluidic device has a cross-sectional dimension of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a microfluidic device has a cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a microfluidic device has a largest cross-sectional dimension of about 1 μm to about 15 μm, such as about 3 μm to about 10 μm. In some embodiments, the interconnected channel of a microfluidic device has a largest cross-sectional dimension of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a microfluidic device has a largest cross-sectional dimension of about any of 1 μm, 2 μm, 3 μm, 4 μm, 5 μm, 6 μm, 7 μm, 8 μm, 9 μm, 10 μm, 11 inn, 12 μm, 14 μm, or 15 μm. In some embodiments, the interconnected channel of a microfluidic device has a smallest cross-sectional dimension of about 1 μm or more, such as about any of 2 μm or more, 3 μm or more, 4 μm or more, 5 μm or more, 6 μm or more, 7 μm or more, 8 μm or more, 9 μm or more, or 10 μm or more. In some embodiments, the interconnected channel of a microfluidic device has a smallest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a microfluidic device has a cross-sectional dimension perpendicular to a largest cross-sectional dimension of about 1 μm to about 15 μm, such as about 3 μm to about 10 μm. In some embodiments, the interconnected channel of a microfluidic device has a cross-sectional dimension perpendicular to a largest cross-sectional dimension of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a microfluidic device has a cross-sectional dimension perpendicular to a largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm. In some embodiments, the interconnected channel of a microfluidic device has a largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm, and a cross-sectional dimension perpendicular to the largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a microfluidic device has a width (such as measured across a cross-sectional dimension) of about 1 μm to about 15 μm, such as any of about 1 μm to about 6 μm, about 3 μm to about 10 μm, or about 6 μm to about 12 μm. In some embodiments, the interconnected channel of a microfluidic device has a width (such as measured across a cross-sectional dimension) of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a microfluidic device has a width (such as measured across a cross-sectional dimension) of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a microfluidic device has a height (such as measured across a cross-sectional dimension) of about 1 μm to about 15 μm, such as any of about 1 μm to about 6 μm, about 3 μm to about 10 μm, or about 6 μm to about 12 μm. In some embodiments, the interconnected channel of a microfluidic device has a height (such as measured across a cross-sectional dimension) of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a microfluidic device has a height (such as measured across a cross-sectional dimension) of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the plurality of interconnected channels of a microfluidic device are formed via a pillar array. In some embodiments, the pillar array is an amorphous pillar array. In some embodiments, the pillar array is a non-amorphous pillar array. In some embodiments, the pillar array forms an inner surface of each of the plurality of interconnected channels of a microfluidic device comprises.

In some embodiments, the microfluidic device comprises a quartz substrate. In some embodiments, the microfluidic device comprises a monolithic substrate forming the plurality of interconnected channels. In some embodiments, the microfluidic device comprises a quartz monolithic substrate. In some embodiments, the microfluidic device comprises a three-dimensional (3D) printed substrate.

In some embodiments, the interconnected channels of a microfluidic device are in an open tubular format. In some embodiments, the channels of the microfluidic device comprise an inner surface material. In some embodiments, the inner surface material is configured as a separation medium, such as a size-exclusion chromatography medium. In some embodiments, the inner surface material has a dimension, such as a thickness, based on the desired separation.

Provided herein, in some aspects, are methods of making the microfluidic devices described herein. Methods of making microfluidic devices are well known in the art. See, e.g., Gale et al., MDPI Inventions, 3, 2018, which is incorporated herein by reference in its entirety. In some embodiments, the method comprises a masking technique. In some embodiments, the method comprises an etching technique. In some embodiments, the method comprises a three-dimension (3D) printing technique.

i. Size-Exclusion Chromatography (SEC) Microfluidic Devices

In some embodiments, the microfluidic device configured for separating components of a sample is a size-exclusion chromatography (SEC) microfluidic device. In such embodiments, the SEC microfluidic device comprises a size-exclusion chromatography (SEC) medium positioned at least in a plurality of interconnected channels of the SEC chromatography device, such as conjugated to an inner surface of the channels. In some embodiments, the SEC medium is further positioned in an upstream network of connection channels. In some embodiments, the SEC medium is further positioned in a downstream network of connection channels.

In some embodiments, the SEC medium is an inner surface material of a plurality of interconnected channels of a SEC microfluidic device. In some embodiments, the inner surface comprises an average pore size of about 10 nm to about 500 nm. In some embodiments, the inner surface comprises an average pore size of at least about 10 nm, such as at least about any of 20 nm, 30 nm, 40 nm, 50 nm, 60 nm, 70 nm, 80 nm, 90 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, or 500 nm. In some embodiments, the inner surface comprises an average pore size of less than about 500 nm, such as less than about any of 475 nm, 450 nm, 425 nm, 400 nm, 375 nm, 350 nm, 325 nm, 300 nm, 275 nm, 250 nm, 225 nm, 200 nm, 175 nm, 150 nm, 125 nm, 100 nm, 90 nm, 80 nm, 70 nm, 60 nm, 50 nm, 40 nm, 30 nm, 20 nm, or 10 nm. In some embodiments, the inner surface comprises an average pore size of about any of 10 nm, 20 nm, 30 nm, 40 nm, 50 nm, 60 nm, 70 nm, 80 nm, 90 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, or 500 nm.

In some embodiments, the inner surface material is configured to leave an open space in each channel of a plurality of interconnected channels, such as found in an open tubular format. In some embodiments, the inner surface material has a thickness of about 0.5 μm to about 2 μm. In some embodiments, the inner surface material has a thickness of at least about 0.5 μm, such as at least about any of 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, or 2 μm. In some embodiments, the inner surface material has a thickness of less than about 2 μm, such as less than about any of 1.9 μm, 1.8 μm, 1.7 μm, 1.6 μm, 1.5 μm, 1.4 μm, 1.3 μm, 1.2 μm, 1.1 μm, 1 μm, 0.9 μm, 0.8 μm, 0.7 μm, 0.6 μm, or 0.5 μm. In some embodiments, the inner surface material has a thickness of about any of 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, or 2 μm.

In some embodiments, the inner surface material is made using a plasma etching technique and/or a three-dimensional (3D) printing technique.

In some embodiments, the SEC microfluidic device comprises a plurality of interconnected channels. In some embodiments, the SEC microfluidic devices described herein may comprise any number of interconnected channels, such that the interconnected channels are in fluid communication with an input port of the SEC microfluidic device, and the interconnected channels are configured to receive a portion of a fluid introduced to the SEC microfluidic device via the input port. In some embodiments, the plurality of interconnected channels of a SEC microfluidic device comprises 8 or more interconnected channels. In some embodiments, the plurality of interconnected channels of a SEC microfluidic device has any of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 interconnected channels. In some embodiments, the plurality of interconnected channels of a SEC microfluidic device comprises 32 interconnected channels. In some embodiments, the plurality of interconnected channels of a SEC microfluidic device comprises 64 interconnected channels.

In some embodiments, each of the plurality of interconnected channels of a SEC microfluidic device are in fluidic communication with an input port of the SEC microfluidic device. In some embodiments, each of the plurality of interconnected channels of a SEC microfluidic device are in fluidic communication with an input port of the SEC microfluidic device via an upstream network of connection channels. In some embodiments, the upstream network of connection channels is configured to direct and split flow of a fluid introduced via an input port of a SEC microfluidic device such that a portion of the fluid is delivered to each interconnected channel. In some embodiments, the upstream network of connection channels, or portions thereof, is connected to a proximal region of each of a plurality of interconnected channels. In some embodiments, the proximal region of an interconnected channel is the region of the interconnected channel first subjected to a fluid introduced via an input port of a SEC microfluidic device. The upstream network of connection channels may be configured in numerous ways and provide one or more functions in addition to directing a fluid to an interconnected channel, e.g., a mixing function and/or a filtering function and/or a dilution function. In some embodiments, the upstream network of connection channels comprises a series of diverging channels configured to split fluid flow from the input port of a SEC microfluidic device to each of the plurality of interconnected channels. In some embodiments, the series of diverging channels of an upstream network of connection channels are structuring using a 1 to 2 split (e.g., from the upstream to downstream direction based on intended fluid flow, one channel splits into two channels). For example, in some embodiments, for a SEC microfluidic device having a single input port and 32 interconnected channels, an upstream network of connection channels comprises a single channel that splits to two channels, wherein each of the two channels splits to two channels (now four total channels), wherein each of the four channels splits to two channels (now eight total channels), wherein each of the eight channels splits to two channels (now 16 channels), wherein the each of the 16 channels splits to two channels (now 32 channels), and wherein each of the 32 channels is connected to one of the 32 interconnected channels. In some embodiments, the upstream network of connection channels comprises a 1 to 2 split, 1 to 3 split, a 1 to 4 split, a 1 to 5 split, a 1 to 6 split, a 1 to 7 split, a 1 to 8 split, a 1 to 9 split, a 1 to 10 split, a 1 to 11 split, a 1 to 12 split, or any combination thereof. In some embodiments, the channels of an upstream network of connection channels after a split (i.e., split channels) have a smaller cross-sectional dimension (such as height and/or width) as compared to the channel from which they originate.

In some embodiments, each of the plurality of interconnected channels of a SEC microfluidic device is in fluidic communication with an output port of the SEC microfluidic device. In some embodiments, each of the plurality of interconnected channels of a SEC microfluidic device is in fluidic communication with an output port of the SEC microfluidic device via a downstream network of connection channels. In some embodiments, the downstream network of connection channels is configured to direct and combine flow of a fluid from each of a plurality of interconnected channels to an output port of a SEC microfluidic device (including, e.g., more than one output port of a microfluidic device). In some embodiments, the downstream network of connection channels, or portions thereof, is connected to a distal region of each of a plurality of interconnected channels. In some embodiments, the distal region of an interconnected channel is the region of the interconnected channel from which a fluid exits the interconnected channel to a downstream feature. The downstream network of connection channels may be configured in numerous ways and provide one or more functions in addition to directing a fluid to an output port, e.g., a mixing function and/or a filtering function and/or a dilution function. In some embodiments, the downstream network of connection channels comprises a series of converging channels configured to combine fluid flow from a plurality of interconnected channels of a SEC microfluidic device to an output port. In some embodiments, the series of converging channels of a downstream network of connection channels are structuring using a 2 to 1 split (e.g., from the upstream to downstream direction based on intended fluid flow, two channel converge into one channel). For example, in some embodiments, for a SEC microfluidic device having a single output port and 32 interconnected channels, a downstream network of connection channels comprises a 32 channels, each of the 32 channels of the downstream network of connection channels is connected to a channel of the 32 interconnected channels, wherein pairs of the 32 channels of the downstream network of connection channels converge to a single channel (now 16 channels), wherein pairs of the 16 channels of the downstream network of connection channels converge to a single channel (now 8 channels), wherein pairs of the 8 channels of the downstream network of connection channels converge to a single channel (now 4 channels), wherein pairs of the 4 channels of the downstream network of connection channels converge to a single channel (now 2 channels), wherein the two channels of the downstream network of connection channels converge to a single channel in fluid communication with the output port. In some embodiments, the downstream network of connection channels comprises a 2 to 1 convergence, a 3 to 1 convergence, a 4 to 1 convergence, a 5 to 1 convergence, a 6 to 1 convergence, a 7 to 1 convergence, a 8 to 1 convergence, a 9 to 1 convergence, a 10 to 1 convergence, a 11 to 1 convergence, a 12 to 1 convergence, or any combination thereof. In some embodiments, the channel of a downstream network of connection channels after a convergence (i.e., a converged channel) has a larger cross-sectional dimension (such as height and/or width) as compared to the channel from which they originate.

In some embodiments, the plurality of interconnected channels of a SEC microfluidic device are only connected via an upstream network of connection channels and/or a downstream network of connection channels. For example, in some embodiments, one interconnected channel is not connected to another interconnected channel, except via an upstream network of connection channels and/or a downstream network of connection channels.

In some embodiments, each of the plurality of interconnected channels of a SEC microfluidic device has a length of about 2 cm to about 30 cm, such as about 5 cm to about 20 cm. In some embodiments, the length of an interconnected channel is at least about 5 cm, such as at least about any of 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, or 30 cm. In some embodiments, the length of an interconnected channel is less than about 30 cm, such as less than about any of 29 cm, 28 cm, 27 cm, 26 cm, 25 cm, 24 cm, 23 cm, 22 cm, 21 cm, 20 cm, 19 cm, 18 cm, 17 cm, 16 cm, 15 cm, 14 cm, 13 cm, 12 cm, 11 cm, 10 cm, 9 cm, 8 cm, 7 cm, 6 cm, 5 cm, 4 cm, 3 cm, or 2 cm. In some embodiments, the length of an interconnected channel is about any 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, or 30 cm.

In some embodiments, the total length of a plurality of interconnected channels is about 20 cm to about 3,200 cm. In some embodiments, the total length of a plurality of interconnected channels is greater than about 20 cm, such as greater than about any of 50 cm, 75 cm, 100 cm, 150 cm, 200 cm, 250 cm, 300 cm, 350 cm, 400 cm, 450 cm, 500 cm, 600 cm, 700 cm, 800 cm, 900 cm, 1,000 cm, 1,250 cm, 1,500 cm, 1,750 cm, 2,000 cm, 2,250 cm, 2,500 cm, 2,750 cm, or 3,000 cm.

plurality of interconnected channels. The channels of the interconnected channels, upstream network of connection channels, and downstream network of connection channels described herein may be formed having various cross-sectional shapes and sizes. In some embodiments, the cross-section shape and size of a channel described herein may change at different points of the channel. In some embodiments, the channel has a cross-sectional shape comprising a rectangle, a square, or a circle.

In some embodiments, the interconnected channel of a SEC microfluidic device has a cross-sectional dimension of about 1 μm to about 15 μm, such as about 3 μm to about 10 μm. In some embodiments, the interconnected channel of a SEC microfluidic device has a cross-sectional dimension of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a SEC microfluidic device has a cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a SEC microfluidic device has a largest cross-sectional dimension of about 1 μm to about 15 μm, such as about 3 μm to about 10 μm. In some embodiments, the interconnected channel of a SEC microfluidic device has a largest cross-sectional dimension of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a SEC microfluidic device has a largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm. In some embodiments, the interconnected channel of a SEC microfluidic device has a smallest cross-sectional dimension of about 1 μm or more, such as about any of 2 μm or more, 3 μm or more, 4 μm or more, 5 μm or more, 6 μm or more, 7 μm or more, 8 μm or more, 9 μm or more, or 10 μm or more. In some embodiments, the interconnected channel of a SEC microfluidic device has a smallest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of an SEC microfluidic device has a cross-sectional dimension perpendicular to a largest cross-sectional dimension of about 1 μm to about 15 μm, such as about 3 μm to about 10 μm. In some embodiments, the interconnected channel of a SEC microfluidic device has a cross-sectional dimension perpendicular to a largest cross-sectional dimension of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a SEC microfluidic device has a cross-sectional dimension perpendicular to a largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm. In some embodiments, the interconnected channel of a SEC microfluidic device has a largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm, and a cross-sectional dimension perpendicular to the largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a SEC microfluidic device has a width (such as measured across a cross-sectional dimension) of about 1 μm to about 15 μm, such as any of about 1 μm to about 6 μm, about 3 μm to about 10 μm, or about 6 μm to about 12 μm. In some embodiments, the interconnected channel of a SEC microfluidic device has a width (such as measured across a cross-sectional dimension) of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a SEC microfluidic device has a width (such as measured across a cross-sectional dimension) of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a SEC microfluidic device has a height (such as measured across a cross-sectional dimension) of about 1 μm to about 15 μm, such as any of about 1 μm to about 6 μm, about 3 μm to about 10 μm, or about 6 μm to about 12 μm. In some embodiments, the interconnected channel of a SEC microfluidic device has a height (such as measured across a cross-sectional dimension) of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a SEC microfluidic device has a height (such as measured across a cross-sectional dimension) of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the plurality of interconnected channels of a SEC microfluidic device are formed via a pillar array. In some embodiments, the pillar array is an amorphous pillar array. In some embodiments, the pillar array is a non-amorphous pillar array. In some embodiments, the pillar array forms an inner surface of each of the plurality of interconnected channels of a SEC microfluidic device comprises.

In some embodiments, the SEC microfluidic device comprises a quartz substrate. In some embodiments, the SEC microfluidic device comprises a monolithic substrate forming the plurality of interconnected channels. In some embodiments, the SEC microfluidic device comprises a quartz monolithic substrate. In some embodiments, the SEC microfluidic device comprises a three-dimensional (3D) printed substrate.

In some embodiments, the interconnected channels of a SEC microfluidic device are in an open tubular format.

ii. Reversed-Phase Liquid Chromatography (RPLC) Microfluidic Device

In some embodiments, the microfluidic device configured for separating components of a sample is a reversed-phase chromatography (RPLC) microfluidic device. In such embodiments, the RPLC microfluidic device comprises a size-exclusion chromatography (RPLC) medium positioned at least in a plurality of interconnected channels of the RPLC chromatography device. In some embodiments, the RPLC medium is further positioned in an upstream network of connection channels. In some embodiments, the RPLC medium is further positioned in a downstream network of connection channels.

In some embodiments, the reversed-phased medium comprises an alkyl moiety, such as an alkyl moiety of any carbon chain length. In some embodiments, the reversed-phased medium comprises an alkyl moiety having a carbon chain length of between C₂ and C₂₀. In some embodiments, the reversed-phased medium comprises an alkyl moiety having a carbon chain length of any of: C₂, C₄, C₈, or C₁₈. In some embodiments, the reversed-phased medium comprises a RPLC moiety mixture comprising two or more of an alkyl moiety having a carbon chain length of between C₂ and C₂₀. In some embodiments, the reversed-phased medium comprises a RPLC moiety mixture comprising two or more of the following alkyl moieties: C₂, C₄, C₈, and C₁₈. In some embodiments, the reversed-phased medium comprises a RPLC moiety mixture comprising three or more of an alkyl moiety having a carbon chain length of between C₂ and C₂₀. In some embodiments, the reversed-phased medium comprises a RPLC moiety mixture comprising three or more of the following alkyl moieties: C₂, C₄, C₈, and C₁₈. In some embodiments, the RPLC moiety mixture comprises the following alkyl moieties: C₂, C₄, C₈, and C₁₈.

The alkyl moieties of a reversed-phase medium may be based on a desired separation. In some embodiments, the alkyl moieties of the RPLC moiety mixture are present in equimolar amounts.

In some embodiments, the alkyl moieties of a reversed-phase medium, such as a RPLC moiety mixture, are covalently coupled to surfaces of each of the plurality of interconnected channels of the RPLC microfluidic device. For example, in some embodiments, the inner surface of an interconnected plurality of parallel channels comprises silica (SiO₂).

In some embodiments, the RPLC microfluidic device comprises a plurality of interconnected channels. In some embodiments, the RPLC microfluidic devices described herein may comprise any number of interconnected channels, such that the interconnected channels are in fluid communication with an input port of the RPLC microfluidic device, and the interconnected channels are configured to receive a portion of a fluid introduced to the RPLC microfluidic device via the input port. In some embodiments, the plurality of interconnected channels of a RPLC microfluidic device comprises 8 or more interconnected channels. In some embodiments, the plurality of interconnected channels of a RPLC microfluidic device has any of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 interconnected channels. In some embodiments, the plurality of interconnected channels of a RPLC microfluidic device comprises 32 interconnected channels. In some embodiments, the plurality of interconnected channels of a RPLC microfluidic device comprises 64 interconnected channels.

In some embodiments, each of the plurality of interconnected channels of a RPLC microfluidic device are in fluidic communication with an input port of the RPLC microfluidic device. In some embodiments, each of the plurality of interconnected channels of a RPLC microfluidic device are in fluidic communication with an input port of the RPLC microfluidic device via an upstream network of connection channels. In some embodiments, the upstream network of connection channels is configured to direct and split flow of a fluid introduced via an input port of a RPLC microfluidic device such that a portion of the fluid is delivered to each interconnected channel. In some embodiments, the upstream network of connection channels, or portions thereof, is connected to a proximal region of each of a plurality of interconnected channels. In some embodiments, the proximal region of an interconnected parallel channel is the region of the interconnected channel first subjected to a fluid introduced via an input port of a RPLC microfluidic device. The upstream network of connection channels may be configured in numerous ways and provide one or more functions in addition to directing a fluid to an interconnected channel, e.g., a mixing function and/or a filtering function and/or a dilution function. In some embodiments, the upstream network of connection channels comprises a series of diverging channels configured to split fluid flow from the input port of a RPLC microfluidic device to each of the plurality of interconnected channels. In some embodiments, the series of diverging channels of an upstream network of connection channels are structuring using a 1 to 2 split (e.g., from the upstream to downstream direction based on intended fluid flow, one channel splits into two channels). For example, in some embodiments, for a RPLC microfluidic device having a single input port and 32 interconnected channels, an upstream network of connection channels comprises a single channel that splits to two channels, wherein each of the two channels splits to two channels (now four total channels), wherein each of the four channels splits to two channels (now eight total channels), wherein each of the eight channels splits to two channels (now 16 channels), wherein the each of the 16 channels splits to two channels (now 32 channels), and wherein each of the 32 channels is connected to one of the 32 interconnected channels. In some embodiments, the upstream network of connection channels comprises a 1 to 2 split, 1 to 3 split, a 1 to 4 split, a 1 to 5 split, a 1 to 6 split, a 1 to 7 split, a 1 to 8 split, a 1 to 9 split, a 1 to 10 split, a 1 to 11 split, a 1 to 12 split, or any combination thereof. In some embodiments, the channels of an upstream network of connection channels after a split (i.e., split channels) have a smaller cross-sectional dimension (such as height and/or width) as compared to the channel from which they originate.

In some embodiments, each of the plurality of interconnected channels of a RPLC microfluidic device is in fluidic communication with an output port of the RPLC microfluidic device. In some embodiments, each of the plurality of interconnected channels of a RPLC microfluidic device is in fluidic communication with an output port of the RPLC microfluidic device via a downstream network of connection channels. In some embodiments, the downstream network of connection channels is configured to direct and combine flow of a fluid from each of a plurality of interconnected channels to an output port of a RPLC microfluidic device (including, e.g., more than one output port of a microfluidic device). In some embodiments, the downstream network of connection channels, or portions thereof, is connected to a distal region of each of a plurality of interconnected channels. In some embodiments, the distal region of an interconnected channel is the region of the interconnected channel from which a fluid exits the interconnected channel to a downstream feature. The downstream network of connection channels may be configured in numerous ways and provide one or more functions in addition to directing a fluid to an output port, e.g., a mixing function and/or a filtering function and/or a dilution function. In some embodiments, the downstream network of connection channels comprises a series of converging channels configured to combine fluid flow from a plurality of interconnected channels of a RPLC microfluidic device to an output port. In some embodiments, the series of converging channels of a downstream network of connection channels are structuring using a 2 to 1 split (e.g., from the upstream to downstream direction based on intended fluid flow, two channel converge into one channel). For example, in some embodiments, for a RPLC microfluidic device having a single output port and 32 interconnected channels, a downstream network of connection channels comprises a 32 channels, each of the 32 channels of the downstream network of connection channels is connected to a channel of the 32 interconnected channels, wherein pairs of the 32 channels of the downstream network of connection channels converge to a single channel (now 16 channels), wherein pairs of the 16 channels of the downstream network of connection channels converge to a single channel (now 8 channels), wherein pairs of the 8 channels of the downstream network of connection channels converge to a single channel (now 4 channels), wherein pairs of the 4 channels of the downstream network of connection channels converge to a single channel (now 2 channels), wherein the two channels of the downstream network of connection channels converge to a single channel in fluid communication with the output port. In some embodiments, the downstream network of connection channels comprises a 2 to 1 convergence, a 3 to 1 convergence, a 4 to 1 convergence, a 5 to 1 convergence, a 6 to 1 convergence, a 7 to 1 convergence, a 8 to 1 convergence, a 9 to 1 convergence, a 10 to 1 convergence, a 11 to 1 convergence, a 12 to 1 convergence, or any combination thereof. In some embodiments, the channel of a downstream network of connection channels after a convergence (i.e., a converged channel) has a larger cross-sectional dimension (such as height and/or width) as compared to the channel from which they originate.

In some embodiments, the plurality of interconnected channels of a RPLC microfluidic device are only connected via an upstream network of connection channels and/or a downstream network of connection channels. For example, in some embodiments, one interconnected channel is not connected to another interconnected channel, except via an upstream network of connection channels and/or a downstream network of connection channels.

In some embodiments, each of the plurality of interconnected channels of a RPLC microfluidic device has a length of about 2 cm to about 30 cm, such as about 5 cm to about 20 cm. In some embodiments, the length of an interconnected channel is at least about 5 cm, such as at least about any of 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, or 30 cm. In some embodiments, the length of an interconnected channel is less than about 30 cm, such as less than about any of 29 cm, 28 cm, 27 cm, 26 cm, 25 cm, 24 cm, 23 cm, 22 cm, 21 cm, 20 cm, 19 cm, 18 cm, 17 cm, 16 cm, 15 cm, 14 cm, 13 cm, 12 cm, 11 cm, 10 cm, 9 cm, 8 cm, 7 cm, 6 cm, 5 cm, 4 cm, 3 cm, or 2 cm. In some embodiments, the length of an interconnected channel is about any 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, or 30 cm.

In some embodiments, the total length of a plurality of interconnected channels is about 20 cm to about 3,200 cm. In some embodiments, the total length of a plurality of interconnected channels is greater than about 20 cm, such as greater than about any of 50 cm, 75 cm, 100 cm, 150 cm, 200 cm, 250 cm, 300 cm, 350 cm, 400 cm, 450 cm, 500 cm, 600 cm, 700 cm, 800 cm, 900 cm, 1,000 cm, 1,250 cm, 1,500 cm, 1,750 cm, 2,000 cm, 2,250 cm, 2,500 cm, 2,750 cm, or 3,000 cm.

The channels of the interconnected channels, upstream network of connection channels, and downstream network of connection channels described herein may be formed having various cross-sectional shapes and sizes. In some embodiments, the cross-section shape and size of a channel described herein may change at different points of the channel. In some embodiments, the channel has a cross-sectional shape comprising a rectangle, a square, or a circle.

In some embodiments, the interconnected channel of a RPLC microfluidic device has a cross-sectional dimension of about 1 μm to about 15 μm, such as about 3 μm to about 10 μm. In some embodiments, the interconnected channel of a RPLC microfluidic device has a cross-sectional dimension of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a RPLC microfluidic device has a cross-sectional dimension of about any of 1 μm, 2 μm, 3 μm, 4 μm, 5 μm, 6 μm, 7 μm, 8 μm, 9 μm, 10 μm, 11 inn, 12 μm, 14 μm, or 15 μm.

In some embodiments, the interconnected channel of a RPLC microfluidic device has a largest cross-sectional dimension of about 1 μm to about 15 μm, such as about 3 μm to about 10 μm. In some embodiments, the interconnected channel of a RPLC microfluidic device has a largest cross-sectional dimension of about 15 μm or less, such as about any of 14 μm or less, 13 inn or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a RPLC microfluidic device has a largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm. In some embodiments, the interconnected channel of a RPLC microfluidic device has a smallest cross-sectional dimension of about 1 μm or more, such as about any of 2 μm or more, 3 μm or more, 4 μm or more, 5 μm or more, 6 μm or more, 7 μm or more, 8 μm or more, 9 μm or more, or 10 μm or more. In some embodiments, the interconnected channel of a RPLC microfluidic device has a smallest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a RPLC microfluidic device has a cross-sectional dimension perpendicular to a largest cross-sectional dimension of about 1 μm to about 15 μm, such as about 3 μm to about 10 μm. In some embodiments, the interconnected channel of a RPLC microfluidic device has a cross-sectional dimension perpendicular to a largest cross-sectional dimension of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a RPLC microfluidic device has a cross-sectional dimension perpendicular to a largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm. In some embodiments, the interconnected channel of a RPLC microfluidic device has a largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm, and a cross-sectional dimension perpendicular to the largest cross-sectional dimension of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a RPLC microfluidic device has a width (such as measured across a cross-sectional dimension) of about 1 μm to about 15 μm, such as any of about 1 μm to about 6 μm, about 3 μm to about 10 μm, or about 6 μm to about 12 μm. In some embodiments, the interconnected channel of a RPLC microfluidic device has a width (such as measured across a cross-sectional dimension) of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a RPLC microfluidic device has a width (such as measured across a cross-sectional dimension) of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the interconnected channel of a RPLC microfluidic device has a height (such as measured across a cross-sectional dimension) of about 1 μm to about 15 μm, such as any of about 1 μm to about 6 μm, about 3 μm to about 10 μm, or about 6 μm to about 12 μm. In some embodiments, the interconnected channel of a RPLC microfluidic device has a height (such as measured across a cross-sectional dimension) of about 15 μm or less, such as about any of 14 μm or less, 13 μm or less, 12 μm or less, 11 μm or less, 10 μm or less, 9 μm or less, 8 μm or less, 7 μm or less, 6 μm or less, 5 μm or less, 4 μm or less, 3 μm or less, 2 μm or less, or 1 μm or less. In some embodiments, the interconnected channel of a RPLC microfluidic device has a height (such as measured across a cross-sectional dimension) of about any of 1 μm, 2 μm, 3 μm, 4 μm, 5 μm, 6 μm, 7 μm, 8 μm, 9 μm, 10 μm, 11 μm, 12 μm, 14 μm, or 15 μm.

In some embodiments, the plurality of interconnected channels of a RPLC microfluidic device are formed via a pillar array. In some embodiments, the pillar array is an amorphous pillar array. In some embodiments, the pillar array is a non-amorphous pillar array. In some embodiments, the pillar array forms an inner surface of each of the plurality of interconnected channels of a RPLC microfluidic device comprises.

In some embodiments, the RPLC microfluidic device comprises a quartz substrate. In some embodiments, the RPLC microfluidic device comprises a monolithic substrate forming the plurality of interconnected channels. In some embodiments, the RPLC microfluidic device comprises a quartz monolithic substrate. In some embodiments, the RPLC microfluidic device comprises a three-dimensional (3D) printed substrate.

In some embodiments, the interconnected channels of a RPLC microfluidic device are in an open tubular format.

In some embodiments, the RPLC microfluidic device comprises an online divert feature. In some embodiments, the online divert feature is a valve and/or a channel, such as a channel subject to fluid flow therethrough. In some embodiments, the online divert feature is positioned between the plurality of interconnected channels of the RPLC microfluidic device and the electrospray ionization device. In some embodiments, the online divert feature is in fluid communication with a waste, e.g., such that a certain portion or portions of RPLC eluate may be diverted away from the mass spectrometer interface.

### V. Compositions Obtained from the Methods and/or Devices Described Herein

Provided herein, in certain aspects, is a collection of compositions obtained from any of the methods and/or devices described herein. In some embodiments, each composition of the collection of compositions is a RPLC microfluidic device eluate.

In some embodiments, as used herein, a composition refers to any mixture of two or more products, substances, liquids, and/or components, including proteins, peptides, nucleic acids, metabolites, other biomolecules, and derivatives thereof. In some embodiments, the composition may be a solution, a suspension, liquid, powder, a paste, aqueous, non-aqueous, or any combination thereof.

### VI. Kits, Components, and Compositions (Such as Consumables)

In some aspects, provided herein are kits, components, and compositions (such as consumables) of the methods, devices, and systems described herein. In some embodiments, the kit comprises a microfluidic liquid chromatography device, such as a SEC microfluidic device and/or a RPLC microfluidic device. In some embodiments, the kit comprises compositions and/or compositions useful for the methods, devices, and systems described herein, such as reagents, e.g., a liquid fixative. In some embodiments, the kit comprises instructions for use according to the disclosure herein.

### VII. Analysis Methods

In some embodiments, the mass spectrometry technique includes assessment of a signal associated with a component, or a sub-population thereof, e.g., peak detection. Many suitable techniques for assessing signals measured by a mass spectrometer are known in the art. In some embodiments, the mass spectrometry technique includes determining ionization intensity associated with a component, or a sub-population thereof. In some embodiments, the mass spectrometry technique includes determining peak height associated with a component, or a sub-population thereof. In some embodiments, the mass spectrometry technique includes determining peak area associated with a component, or a sub-population thereof. In some embodiments, the mass spectrometry technique includes determining peak volume associated with a component, or a sub-population thereof. In some embodiments, the mass spectrometry technique includes identifying peptide products by amino acid sequence. In some embodiments, the mass spectrometry technique includes manually interpreting and validating the peptide product amino acid sequence assignments. In some embodiments, the mass spectrometry technique includes identifying the first polypeptide by a protein identifier. In some embodiments, the mass spectrometry technique includes identifying one or more of the plurality of polypeptides by a protein identifier, which may be identified in a commercially available or in-house generated database (from recombinant proteins or other synthetic standards of peptides or metabolites) search or a library search.

In some embodiments, the identification of products of a polypeptide is achieved using spectral libraries. Use of spectral libraries can allow for the imputation of knowledge gained regarding a polypeptide system and results in increased speed of data analysis and decreased error.

Any one of the mass spectrometry techniques described can be applied to the methods described herein. In some embodiments, the one or more biomolecules and/or the component eluted from a RPLC microfluidic device are subjected to a mass spectrometer. In some embodiments, a mass spectrometry analysis is performed on the one or more biomolecules and/or the component of a test sample using the mass spectrometer. In some embodiments, the mass spectrometry analysis includes an analysis of the fraction subjected to the RPLC technique using the RPLC microfluidic device. In some embodiments, the mass spectrometry analysis includes obtaining one or more data sets comprising information obtained from the mass spectrometer for each fraction subjected to the RPLC technique using the RPLC microfluidic device. In some embodiments, the single data set includes information obtained from a mass spectrometer from a single fraction subjected to a RPLC technique, such as a RPLC technique described herein, using a RPLC microfluidic device. In some embodiments, each of the one or more data sets includes mass-to-charge (rn/z) and abundance information for ions of the one or more biomolecules and/or the component introduced to a mass spectrometer.

In some aspects, the methods provided herein can further include steps of analyzing one or more outputs of the mass spectrometry technique. In some embodiments, the methods provided further include analyzing at least one of the one or more data sets that include information obtained from the mass spectrometer.

In some embodiments, at least one of the one or more data sets is used to determine the identities of each of a plurality of the one or more biomolecules in the test sample. Reference herein to “identities” refers to the names of biomolecules in the test sample. For instance, in some embodiments at least one of the one or more data sets is to determine the protein names of any proteins from the test sample, or products thereof, introduced to the mass spectrometer. In some embodiments, the m/z information in at least one of the one or more data sets is used to determine the identities of each of a plurality of the one or more biomolecules in the test sample.

In some embodiments, at least one of the one or more data sets is used to determine the quantities of each of a plurality of the one or more biomolecules in the test sample. In some embodiments, the quantities of one or more identified biomolecules are determined. Reference herein to “identified biomolecules” refers to biomolecules of the test sample whose identities have been determined. In some embodiments, the abundance information in at least one of the one or more data sets is used to determine the quantities of each of a plurality of the one or more biomolecules in the test sample. It is within the level of the skilled artisan to determine appropriate techniques for identifying or quantifying biomolecules from a test sample or products thereof that are introduced to a mass spectrometer based on the outputs, e.g., m/z or abundance information, of subsequent mass spectrometry techniques.

In some embodiments, at least one data set is used to identify or quantify one or more biomolecules of the test sample. For instance, a single data set can include data associated with a single fraction (e.g., any of the fractions described in Section II-C), and the single data set can be used to identify or quantify biomolecules or products thereof present in that fraction and introduced to the mass spectrometer. In some embodiments, a plurality of data sets is used to identify or quantify one or more biomolecules of the test sample, for instance in order to identify or quantify biomolecules or products thereof present in a plurality of fractions introduced to the mass spectrometer. Any number of data sets associated with any number of fractions introduced to the mass spectrometer can be used to identify or quantify the associated biomolecules or products thereof.

In some embodiments, the methods provided herein further include identifying a signature that includes one or more identified biomolecules from the determined identities. Reference herein to a “signature” refers to a set of identified biomolecules. The signature can include all or a subset of the identified biomolecules in a test sample. In some embodiments, identifying the signature further includes selecting a subset of the one or more identified biomolecules originally in the signature. In some embodiments, the subset of the one or more identified biomolecules is selected based on the measured quantities of the one or more identified biomolecules. For instance, the subset of the one or more identified biomolecules can be selected to include high-abundance biomolecules.

In other embodiments, the methods provided herein further include identifying a signature by, based on at least one of the one or more data sets, measuring the quantities of each of a plurality of the one or more biomolecules in the test sample; selecting a subset of the plurality of the one or more biomolecules in the test sample based on the measured quantities; and determining the identities of each of the subset of the plurality of the one or more biomolecules in the test sample. That is, quantities of a plurality of biomolecules in the test sample can first be determined without identifying the plurality of the biomolecules, and a subset of the plurality of biomolecules can be selected based on the measured quantities. Then, the identities of the subset of the plurality of biomolecules can be determined.

In some embodiments, the subset of the one or more identified biomolecules is selected based on differential measured quantities of the one or more identified biomolecules compared to a reference sample. In some embodiments, the subset of the plurality of the one or more biomolecules in the test sample is selected based on differential measured quantities of the plurality of the one or more biomolecules in the test sample compared to a reference sample. In some embodiments, the subset of the one or more identified biomolecules (or the subset of the plurality of the one or more biomolecules) is selected based on differential measured quantities compared to a plurality of reference samples. In some embodiments, identified biomolecules and associated measured quantities are determined for a plurality of test samples, and the subset of the one or more identified biomolecules (or the subset of the plurality of the biomolecules or products thereof) to be included in the signature is selected based on differential measured quantities between the plurality of test samples and a plurality of reference samples. In some embodiments, the signature includes identified biomolecules with higher quantities, identified biomolecules with lower quantities, or both, relative to a reference sample or a plurality of reference samples.

In some embodiments, the test sample and the reference sample are chosen in order to identify a signature of identified biomolecules that are differentially expressed or that have differential quantities between subjects or groups of subjects having different health or disease states. In some embodiments, the reference sample is a sample from a healthy subject or a control subject. For instance, in some embodiments, the test sample is a sample from a diseased subject, and the reference sample is a sample from a healthy subject or a control subject. In some embodiments, the test sample is a sample from a subject having a pre-condition related to a disease, and the reference sample is a sample from a healthy subject or a control subject.

Reference herein to a “control subject” refers to a subject that is healthy or has a disease or pre-condition unrelated to that of the subject providing the test sample. In some embodiments, both the test sample and the reference sample are samples from diseased subjects, but the diseased subjects have diseases in different states. For instance, in some embodiments, the test sample is a sample from a subject with a disease in an active state, and the reference sample is a sample from a subject with the disease in an inactive state. In some embodiments, the inactive state is remission. Remission is either the reduction or disappearance of the signs and symptoms of the disease. The term can also be used to refer to the period during which this diminution occurs. A remission can be considered a partial remission or a complete remission. In some embodiments, both the test sample and the reference sample are samples from diseased subjects, but the diseased subjects have diseases in different stages. Patients can be classified as having certain disease stages based on etiology, pathophysiology, and severity, and patients having a disease at the same stage may require similar treatment and have similar expected outcomes. In some embodiments, the test sample is a sample from a subject with a disease at an advanced stage, and the reference sample is a sample from a subject with the disease at an early stage. Other exemplary disease stages include Stage 1 (e.g., a disease with no complications), Stage 2 (e.g., the disease with local complications), and Stage 3 (e.g., the disease is involved in multiple systems or has systemic complications).

Thus, provided herein in some embodiments is a signature that includes a plurality of the identified molecules, or a subset thereof, that have been identified using any of the methods provided herein. In some embodiments, provided herein is a signature that includes the subset of identified biomolecules identified using any of the methods provided herein.

In some embodiments, the provided methods further include subjecting all or a subset of the identified biomolecules of the signature to further analyses. In some embodiments, the provided methods further include providing all or a subset of the identified biomolecules of the signature as input to one or more processes each configured to analyze the type of data being provided. For instance, the identified biomolecules can include protein names, and the protein names can be provided as input to a process configured to analyze aspects of or relationships among the provided proteins or products thereof (e.g., to perform protein-protein network analysis).

In some embodiments, the provided methods further include providing all or a subset of the identified biomolecules of the signature as input to one or more processes each configured to perform gene enrichment analysis; one or more processes each configured to perform pathway analysis; and/or one or more processes each configured to perform network analysis. Also provided herein in some embodiments is a method of analyzing biomolecules of a sample, the method including providing the identified biomolecules of any of the signatures provided herein as input to one or more processes each configured to perform gene enrichment analysis; one or more processes each configured to perform pathway analysis; and/or one or more processes each configured to perform network analysis. Such processes can be used to identify patterns and relationships across pairs or groups within the identified biomolecules provided as input.

In some embodiments, the identified biomolecules of the signature are provided as input to one or more processes each configured to perform gene enrichment analysis; one or more processes each configured to perform pathway analysis; and one or more processes each configured to perform network analysis.

In some embodiments, identified biomolecules of one or more molecular types of the signature are provided as the input. In some embodiments, the one or more molecular types include proteins. In some embodiments, the one or more molecular types include RNAs, including coding and/or non-coding RNAs. In some embodiments, the one or more molecular types include peptides. In some embodiments, the one or more molecular types include metabolites. In some embodiments, the one or more molecular types include any combination of proteins, RNAs (coding and/or non-coding RNAs), peptides, and metabolites. In some embodiments, the one or more molecular types consist only of proteins.

In some embodiments, the identified biomolecules of the signature are provided as input to one or more processes each configured to perform gene enrichment analysis. Gene enrichment analysis (also known as gene set enrichment analysis or functional enrichment analysis) includes methods that can be used to identify groups of biomolecules (e.g., groups of genes or proteins) that are over-represented in a set of provided biomolecules. These methods can also be used to identify regulators of provided biomolecules, for instance transcription factors or kinases whose activity affects the expression or activity of any genes or proteins provided as input. These methods rely on statistical approaches to identify significantly enriched or depleted groups of biomolecules among the biomolecules provided as input. In some instances, the biomolecules are grouped based on their involvement in the same biological pathways. These methods also rely on gene ontologies (GOs) in order to group biomolecules. GOs are known in the art and include human-curated representations of the relationships among various biomolecules. GOs include those describing cellular components, molecular functions, or biological processes. Reference herein to a particular GO, for instance a cellular component GO, also refers to all sub-ontologies contained within the larger ontology (e.g., reference to the cellular component GO includes reference to sub-ontologies within the cellular component GO).

In some embodiments, the one or more processes configured to perform gene enrichment analysis include a process configured to identify one or more GOs associated with at least one of the identified biomolecules of the signature provided as input, or at least one of the products thereof (i.e., at least one of the products of an identified biomolecule provided as input). In some embodiments, the one or more processes configured to perform gene enrichment analysis include a process configured to identify one or more cellular component GOs associated with at least one of the identified biomolecules of the signature provided as input, or at least one of the products thereof; a process configured to identify one or more molecular pathway GOs associated with at least one of the identified biomolecules of the signature provided as input, or at least one of the products thereof; and/or a process configured to identify one or more biological process GOs associated with at least one of the identified biomolecules of the signature provided as input, or at least one of the products thereof. In some embodiments, the one or more processes configured to perform gene enrichment analysis include a process configured to identify one or more cellular component GOs associated with at least one of the identified biomolecules of the signature provided as input, or at least one of the products thereof; and a process configured to identify one or more molecular pathway GOs associated with at least one of the identified biomolecules of the signature provided as input, or at least one of the products thereof.

In any of the preceding embodiments, the one or more processes configured to perform gene enrichment analysis identify GOs that are enriched or highly represented in the identified biomolecules provided as input, or products thereof. In some embodiments, the identified GOs are associated with a plurality or majority of the identified biomolecules provided as input, or products thereof. In some embodiments, the number of identified biomolecules, or products thereof, associated with the identified GOs is higher than would be expected by chance (e.g., higher than the number that would be associated on average with a randomly chosen GO).

In some embodiments, the one or more processes configured to perform gene enrichment analysis include a process configured to identify one or more regulators of at least one of the identified biomolecules of the signature provided as input, or at least one of the products thereof. Regulators include any biomolecules capable of affecting the abundance or activity of any of the biomolecules in the test sample, including transcription factors, small molecules, small regulatory RNAs (e.g., microRNAs or siRNAs), kinases, and phosphatases. In some embodiments, the one or more processes configured to perform gene enrichment analysis include a process configured to identify one or more transcription factors regulating at least one of the identified biomolecules of the signature provided as input, or at least one of the products thereof. In some embodiments, the one or more processes configured to perform gene enrichment analysis include a process configured to identify one or more kinases regulating at least one of the identified biomolecules of the signature provided as input, or at least one of the products thereof. In some embodiments, the one or more processes configured to perform gene enrichment analysis include a process configured to identify one or more transcription factors regulating at least one of the identified biomolecules of the signature provided as input, or at least one of the products thereof; and a process configured to identify one or more kinases regulating at least one of the identified biomolecules of the signature provided as input, or at least one of the products thereof.

In any of the preceding embodiments, the one or more processes configured to perform gene enrichment analysis identify regulators (e.g., transcription factors or kinases) that regulate a plurality or majority of the identified biomolecules provided as input, or products thereof. In some embodiments, the number of identified biomolecules, or products thereof, regulated by the identified regulators is higher than would be expected by chance (e.g., higher than the number that would be regulated on average by a randomly chosen regulator).

Exemplary methods for performing gene enrichment analysis include the standard gene set enrichment analysis (GSEA) algorithm, the Simpler Enrichment Analysis (SEA) algorithm, and the Spectral Gene Set Enrichment (SGSE) algorithm. Exemplary tools for performing gene enrichment analysis include or are provided by the Nucleic Acid SeQuence Analysis Resource (NASQAR), PlantRegMap, Molecular Signatures Database (MSigDB), Broad Institute, WebGestalt (for instance using the Over-Representation Analysis (ORA), GSEA, or Network Topology-based Analysis (NSA) algorithms), Enrichr, GeneSCF, DAVID, Metascape, AmiGO2, Genomic region enrichment of annotations tool (GREAT), Functional Enrichment Analysis (FunRich), FuncAssociate, InterMine, ToppGene, Quantitative Set Analysis for Gene Expression (QuSAGE), Blast2GO, and g:Profiler). Exemplary tools for performing gene enrichment analysis also include those that can identify transcription factors or kinases regulating the proteins provided as input, including Transcription Factor Enrichment Analysis (TFEA) and Kinase Enrichment Analysis (KEA), respectively.

In some embodiments, the identified biomolecules of the signature are provided as input to one or more processes each configured to perform pathway analysis. Pathway analysis includes methods that can be used to identify, given a list of biomolecules as input, any biological pathways represented among or enriched in the provided biomolecules. Biological pathways include metabolic pathways and signaling pathways. These methods can rely on GOs as well as on human-curated pathway collections and interaction networks, for instance those from resources KEGG, WikiPathways, Reactome, Pathway Studio, and Ingenuity Pathway Analysis. These pathway collections and interaction networks can be compiled from published materials and can include information on genes, proteins, metabolic pathways, molecular interactions, and biochemical reactions associated with specific organisms. They can also map how these various biomolecules and pathways are organized in a cellular structure or larger reaction pathway. Pathway analysis also includes methods of pathway-based modeling. Types of pathway-based models and available tools for developing these models include partial differential equations/Boolean models (available tools include CellNetAnalyzer); network flow models (available tools include NetPhorest and NetworKlN); transcriptional regulatory network-based reconstruction methods (available tools include ARACNe); and probabilistic graph models (PGMs, available tools include PARADIGM).

In some embodiments, the one or more processes configured to perform pathway analysis include a process configured to identify one or more pathways, e.g., molecular, signaling, or metabolic pathways, associated with at least one of the identified biomolecules of the signature provided as input, or at least one of the products thereof. In some embodiments, the one or more processes configured to perform pathway analysis include a process configured to identify one or more molecular pathways associated with at least one of the identified biomolecules of the signature provided as input, or at least one of the products thereof; a process configured to identify one or more signaling pathways associated with at least one of the identified biomolecules of the signature provided as input, or at least one of the products thereof; and/or a process configured to identify one or more metabolic pathways associated with at least one of the identified biomolecules of the signature provided as input, or at least one of the products thereof. In some embodiments, the one or more processes configured to perform pathway analysis include a process configured to identify one or more signaling pathways associated with at least one of the identified biomolecules of the signature provided as input, or at least one of the products thereof.

In any of the preceding embodiments, the one or more processes configured to perform pathway analysis identify one or more pathways that are enriched or highly represented in the identified biomolecules provided as input, or in products thereof. In some embodiments, the one or more identified pathways (e.g., signaling pathways) each include a plurality or a majority of the identified biomolecules provided as input, or of products thereof. In some embodiments, the number of identified biomolecules or products thereof included in each of the one or more identified pathways is higher than would be expected by chance (e.g., higher than the number that would be included on average in a randomly chosen pathway).

Exemplary methods for performing pathway analysis include over-representation analysis (ORA); functional class scoring (FCS); pathway topology analysis (PTA), including Signaling Pathway Impact Analysis (SPIA), EnrichNet, Gene Graph Enrichment Analysis (GGEA), and TopoGSA; and network enrichment analysis (NEA). Exemplary tools for performing pathway analysis include those provided through STRING, Cytoscape, Ingenuity, Pathways Studio, Pathways Studio Viewer, PTA: PathwayGuide, MetaCore, Wiki Pathways, CellNetAnalyzer, NetPhorest/NetworKlN, ARACNe, and Paradigm.

In some embodiments, the identified biomolecules of the signature are provided as input to one or more processes each configured to perform network analysis. Network analysis includes methods that can be used to identify, given a list of biomolecules as input, the relationships among the biomolecules provided as input. Relationships include physical or functional interactions. These networks can be constructed based on, for instance, predicted co-expression, co-localization, genetic interaction, physical interaction, and predicted and shared protein domain data. Nodes or vertices can be used to represent the identified biomolecules provided as input, and edges each connecting two nodes (or a node to itself) can be used to represent a predicted or identified relationship between the connected nodes. Types of networks include transcriptional regulatory networks, virus-host networks, metabolic networks, protein-protein interaction networks, disease networks, and drug effect networks (e.g., a network of biomolecules whose expression or activity is affected by a particular drug). Networks can be identified in a provided list of biomolecules using interaction databases, which can be built automatically or via human curation. Human curated interaction databases include BioGRID and IntAct. Network analysis can be used to analyze the interconnectedness of (i.e., the relationships among) the provided identified biomolecules, including to detect clusters of nodes (i.e., identified biomolecules) that are similar or part of a tightly connected group, for instance a group of nodes with a high number of edges connecting one another. Network analysis can also be used to identify hubs of a network. Hubs of a network are nodes having a high or higher than average number of edges connecting them to other nodes in the network. In biological networks, these hubs can be central regulators of their associated pathways. Thus, in some aspects, the identification of drugs targeting these hubs may broadly affect pathways or processes that have been affected by disease.

In some embodiments, the one or more processes configured to perform network analysis include a process configured to identify one or more networks each associated with at least one of the identified biomolecules of the signature provided as input, or at least one of the products thereof. In some embodiments, the one or more processes configured to perform network analysis include a process configured to identify one or more hubs of one or more networks each associated with at least one of the identified biomolecules of the signature provided as input, or at least one of the products thereof.

In some embodiments, the one or more processes configured to perform network analysis include a process configured to identify one or more molecular pathways each associated with at least one of the identified biomolecules of the signature provided as input, or at least one of the products thereof; and/or a process configured to identify one or more signaling pathways each associated with at least one of the identified biomolecules of the signature provided as input, or at least one of the products thereof. In some embodiments, the one or more processes configured to perform network analysis include a process configured to identify one or more molecular pathways each associated with at least one of the identified biomolecules of the signature provided as input, or at least one of the products thereof; and a process configured to identify one or more signaling pathways each associated with at least one of the identified biomolecules of the signature provided as input, or at least one of the products thereof.

In some embodiments, the one or more processes configured to perform network analysis include a process configured to identify one or more protein-protein interaction networks each associated with at least one of the identified biomolecules of the signature provided as input, or at least one of the products thereof. In some embodiments, the process is further configured to identify one or more hubs associated with the one or more identified protein-protein interaction networks.

In some embodiments, the one or more processes configured to perform network analysis include a process configured to identify one or more drugs each targeting at least one of the identified biomolecules of the signature provided as input, or at least one of the products thereof. In some embodiments, the one or more processes configured to perform network analysis include two processes configured to identify one or more drugs each targeting at least one of the identified biomolecules of the signature provided as input, or at least one of the products thereof. In some embodiments, the process or each of the two processes is configured to identify one or more drugs each targeting at least one hub of a network comprising a plurality of the identified biomolecules of the signature provided as input. In some embodiments, the process or each of the two processes is configured (1) to identify one or more networks associated with at least one of the identified biomolecules of the signature provided as input, (2) to identify one or more hubs of the one or more identified networks, and (3) to identify one or more drugs each targeting at least one of the identified hubs. In any of the preceding embodiments, the network or one or more networks are protein-protein interaction networks.

In any of the preceding embodiments, the process is configured to identify one or more networks or hubs thereof each associated with a plurality of the identified biomolecules of the signature provided as input, or a plurality of products thereof. In some embodiments, the number of identified biomolecules or products thereof associated with the identified one or more networks is higher than would be expected by chance.

Exemplary network clustering algorithms include or are available through the Girvin-Newman method, Markov Cluster Algorithm, HotNet algorithm, HyperModules Cytoscape App, and Reactome FI Network and ReactomeFlViz. Exemplary tools for performing network analysis include GeneMANIA (which can be used, for instance, to identify protein-protein interaction networks), HotNet, HyperModules, and Reactome Cytoscape FI App, as well as L1000 fireworks display (L1000 FWD) and the iLINCS chemical perturbation (piNET) algorithm, both of which can be used to identify drugs that target genes or proteins provided as input.

In some embodiments, the identified biomolecules of the signature are provided as input to one or more processes each configured to perform gene enrichment analysis (e.g., any of the processes described above that are configured to perform gene enrichment analysis); one or more processes each configured to perform pathway analysis (e.g., any of the processes described above that are configured to perform pathway analysis); and one or more processes each configured to perform network analysis (e.g., any of the processes described above that are configured to perform network analysis). In some embodiments, the one or more processes configured to perform gene enrichment analysis include a process configured to identify one or more cellular component gene ontologies each associated with at least one of the identified components provided as input, or at least one of the products thereof. In some embodiments, the one or more processes configured to perform gene enrichment analysis include a process configured to identify one or more molecular pathway gene ontologies each associated with at least one of the identified components provided as input, or at least one of the products thereof. In some embodiments, the one or more processes configured to perform gene enrichment analysis include a process configured to identify one or more transcription factors regulating at least one of the identified components provided as input, or at least one of the products thereof. In some embodiments, the one or more processes configured to perform gene enrichment analysis include a process configured to identify one or more kinases regulating at least one of the identified components provided as input, or at least one of the products thereof. In some embodiments, the one or more processes configured to perform pathway analysis include a process configured to identify one or more signaling pathways each associated with at least one of the identified components provided as input, or at least one of the products thereof. In some embodiments, the one or more processes configured to perform network analysis include a process configured to identify one or more networks each associated with at least one of the identified components provided as input, or at least one of the products thereof. In some embodiments, the one or more networks are protein-protein interaction networks. In some embodiments, the one or more processes configured to perform network analysis include one or more processes configured to identify one or more drugs each targeting at least one of the identified components provided as input, or at least one of the products thereof. In some embodiments, the one or more processes configured to identify one or more drugs each targeting at least one of the identified components provided as input, or at least one of the products thereof, is configured to identify one or more drugs each targeting a hub of a protein-protein interaction network that includes a plurality of the identified components provided as input, or a plurality of products thereof.

Also provided herein in some embodiments is a method of analyzing a signature of identified biomolecules, said method including providing a plurality of identified biomolecules to each of a plurality of processes each configured to perform gene enrichment analysis, pathway analysis, or network analysis, wherein: the providing is performed in any order; the plurality of identified biomolecules includes a protein set, a transcriptomic set, a peptide set, and/or a metabolite set; and the plurality of processes include: a process configured to perform gene enrichment analysis to identify one or more gene ontologies each associated with at least one of the plurality of identified biomolecules provided as input, or at least one of the products thereof; a process configured to perform pathway analysis to identify one or more signaling pathways each associated with at least one of the plurality of identified biomolecules provided as input, or at least one of the products thereof; a process configured to perform gene enrichment analysis to identify one or more transcription factors regulating at least one of the plurality of identified biomolecules provided as input, or at least one of the products thereof; a process configured to perform gene enrich analysis to identify one or more kinases regulating a gene product of at least one of the plurality of identified biomolecules provided as input, or at least one of the products thereof; a process configured to perform network analysis to identify one or more networks each associated with at least one of the plurality of identified biomolecules provided as input, or at least one of the products thereof; and each of two processes configured to perform network analysis to identify one or more drugs each targeting at least one of the plurality of identified biomolecules provided as input, or at least one of the products thereof.

In some embodiments, the plurality of identified biomolecules includes a protein set. In some embodiments, the plurality of identified biomolecules includes only proteins. In some embodiments, the one or more networks is a protein-protein interaction network. In some embodiments, each of the two processes configured to perform network analysis to identify one or more drugs each targeting at least one of the plurality of identified biomolecules provided as input, or at least one of the products thereof, is configured to identify one or more drugs each targeting a hub of a protein-protein interaction network that includes a plurality of the identified biomolecules provided as input, or a plurality of products thereof.

Also provided herein in some embodiments is a method of analyzing a protein signature, the method including providing a plurality of proteins to each of a plurality of processes each configured to perform gene enrichment analysis, pathway analysis, or network analysis, wherein the providing is performed in any order, and the plurality of processes include: a process configured to perform gene enrichment analysis to identify one or more gene ontologies each associated with at least one of the plurality of proteins provided as input, or at least one of the products thereof; a process configured to perform pathway analysis to identify one or more signaling pathways each associated with at least one of the plurality of proteins provided as input, or at least one of the products thereof; a process configured to perform gene enrichment analysis to identify one or more transcription factors regulating at least one of the plurality of proteins provided as input, or at least one of the products thereof; a process configured to perform gene enrich analysis to identify one or more kinases regulating a gene product of at least one of the plurality of proteins provided as input, or at least one of the products thereof; a process configured to perform network analysis to identify one or more networks each associated with at least one of the plurality of proteins provided as input, or at least one of the products thereof; and each of two processes configured to perform network analysis to identify one or more drugs each targeting at least one of the plurality of proteins provided as input, or at least one of the products thereof.

In some embodiments, the one or more networks is a protein-protein interaction network. In some embodiments, each of the two processes configured to perform network analysis to identify one or more drugs each targeting at least one of the plurality of identified biomolecules provided as input, or at least one of the products thereof, is configured to identify one or more drugs each targeting a hub of a protein-protein interaction network that includes a plurality of the identified biomolecules provided as input, or a plurality of products thereof.

### VIII. Exemplary Embodiments

Provided herein are embodiments of the subject matter of the application.

Embodiment 1. A method for processing a test sample for a mass spectrometry analysis, the method comprising: (a) subjecting the test sample to a size-exclusion chromatography (SEC) technique using a SEC microfluidic device, wherein the test sample comprises one or more biomolecules and a chaotropic agent, and wherein the SEC microfluidic device comprises a plurality of interconnected channels; (b) collecting a plurality of fractions eluted from the SEC microfluidic device; (c) subjecting one or more of the plurality of fractions from the SEC microfluidic device to a proteolytic technique; and (d) individually subjecting one or more fractions from one or both of steps (b) and (c) to a reversed-phase liquid chromatography (RPLC) technique using a RPLC microfluidic device under conditions to prepare a component of each of the one or more fractions for introduction to a mass spectrometer, wherein the RPLC microfluidic device comprises a plurality of interconnected channels comprising a reversed-phase medium, and wherein the RPLC microfluidic device is coupled to an electrospray ionization source.

Embodiment 2. The method of embodiment 1, wherein the test sample a biological sample.

Embodiment 3. The method of embodiment 1 or 2, wherein the test sample is from an individual.

Embodiment 4. The method of any one of embodiments 1-3, wherein the test sample has a concentration of the chaotropic agent of about 5 M to about 8 M.

Embodiment 5. The method of any one of embodiments 1-4, wherein the chaotropic agent comprises guanidine or a salt thereof, guanidinium or a salt thereof, potassium or a salt thereof, lithium or a salt thereof, magnesium or a salt thereof, or sodium or a salt thereof.

Embodiment 6. The method of any one of embodiments 1-3, wherein the chaotropic agent is guanidine hydrochloride or guanidinium chloride.

Embodiment 7. The method of any one of embodiments 1-6, wherein the chaotropic agent in the test sample is from a liquid fixative.

Embodiment 8. The method of any one of embodiments 1-7, wherein the test sample has a concentration of a viscosity modifying agent of about 5% to about 40%.

Embodiment 9. The method of embodiment 8, wherein the viscosity modifying agent is glycerol.

Embodiment 10. The method of embodiment 8 or 9, wherein the test sample comprises at least about 6 M guanidine and about 10% to about 30% glycerol.

Embodiment 11. The method of any one of embodiments 1-10, wherein the test sample subjected to the SEC technique using the SEC microfluidic device has a volume of about 1 μL to about 200 μL.

Embodiment 12. The method of any one of embodiments 1-11, wherein the range of the concentration of the mobile phase chaotropic agent of the SEC technique is within about +/−40% of the pre-determined concentration of the chaotropic agent of the test sample.

Embodiment 13. The method of any one of embodiments 1-12, wherein the SEC technique comprises use of a SEC mobile phase having a concentration of a mobile phase chaotropic agent within a range of the chaotropic agent in the test sample.

Embodiment 14. The method of any one of embodiments 1-13, wherein the mobile phase chaotropic agent of the SEC technique is the same as the chaotropic agent of the test sample.

Embodiment 15. The method of any one of embodiments 1-13, wherein the mobile phase chaotropic agent of the SEC technique is different than the chaotropic agent of the test sample.

Embodiment 16. The method of any one of embodiments 1-15, wherein the SEC mobile phase comprises a mobile phase chaotropic agent at a concentration of about 4 M to about 8 M.

Embodiment 17. The method of any one of embodiments 1-16, wherein the mobile phase chaotropic agent of the SEC technique comprises guanidine or a salt thereof, guanidinium or a salt thereof, lithium or a salt thereof, magnesium or a salt thereof, or sodium or a salt thereof.

Embodiment 18. The method of any one of embodiments 1-17, wherein the mobile phase chaotropic agent of the SEC technique is selected from the group consisting of guanidine hydrochloride, guanidinium chloride, guanidinium thiocynante, lithium perchlorate, lithium acetate, magnesium chloride, potassium acetate, and sodium iodide.

Embodiment 19. The method of any one of embodiments 1-18, wherein the SEC mobile phase comprises a mobile phase viscosity modifying agent.

Embodiment 20. The method of embodiment 20, wherein the mobile phase viscosity modifying agent of the SEC technique has a concentration of about 5% to about 40%.

Embodiment 21. The method of embodiment 19 or 20, wherein the viscosity modifying agent is glycerol.

Embodiment 22. The method of any one of embodiments 19-21, wherein the mobile phase viscosity modifying agent of the SEC technique is the same as the viscosity modifying agent of the liquid fixative.

Embodiment 23. The method of any one of embodiments 19-21, wherein the mobile phase viscosity modifying agent of the SEC technique is different than the viscosity modifying agent of the liquid fixative.

Embodiment 24. The method of any one of embodiments 19-21, wherein the test sample comprises at least about 6 M guanidine and about 10% to about 30% glycerol.

Embodiment 25. The method of any one of embodiments 1-24, wherein the SEC technique is an isocratic SEC technique.

Embodiment 26. The method of any one of embodiments 1-25, wherein the SEC technique comprises use of a mobile phase flow rate of about 1 μL/minute to about 5 μL/minute.

Embodiment 27. The method of any one of embodiments 1-26, wherein the SEC technique is performed at an elevated temperature.

Embodiment 28. The method of any one of embodiments 1-27, wherein the SEC technique is performed at a temperature of about 45° C. to about 60° C.

Embodiment 29. The method of embodiment 27 or 28, wherein the SEC technique is performed at a substantially consistent temperature.

Embodiment 30. The method of any one of embodiments 1-29, wherein the SEC microfluidic device comprises a SEC medium.

Embodiment 31. The method of embodiment 30, wherein the SEC medium is a material having an average pore size of about 10 nm to about 500 nm.

Embodiment 32. The method of embodiment 30 or 31, wherein the SEC medium is an inner surface of each of the plurality of interconnected channels.

Embodiment 33. The method of any one of embodiments 1-32, wherein the inner surface material of the plurality of interconnected channels of the SEC microfluidic device has a thickness of about 0.5 μm to about 2 μm.

Embodiment 34. The method of any one of embodiments 1-33, wherein the plurality of interconnected channels of the SEC microfluidic device are configured in an open tubular format.

Embodiment 35. The method of any one of embodiments 1-34, wherein the plurality of interconnected channels of the SEC microfluidic device comprises 8 or more interconnected channels.

Embodiment 36. The method of embodiment 35, wherein the plurality of interconnected channels of the SEC microfluidic device comprises 32 interconnected channels.

Embodiment 37. The method of embodiment 35, wherein the plurality of interconnected channels of the SEC microfluidic device comprises 64 interconnected channels.

Embodiment 38. The method of any one of embodiments 1-37, wherein each of the plurality of interconnected channels of the SEC microfluidic device are in fluidic communication with an input port of the SEC microfluidic device via an upstream network of connection channels.

Embodiment 39. The method of embodiment 38, wherein the upstream network of connection channels, or portions thereof, is connected to a proximal region of each of the plurality of interconnected channels.

Embodiment 40. The method of embodiment 38 or 39, wherein the upstream network of connection channels comprises a series of diverging channels configured to split fluid flow from the input port of the SEC microfluidic device to each of the plurality of interconnected channels.

Embodiment 41. The method of any one of embodiments 1-40, wherein each of the plurality of interconnected channels of the SEC microfluidic device is in fluidic communication with an output port of the SEC microfluidic device via a downstream network of connection channels.

Embodiment 42. The method of embodiment 41, wherein the downstream network of connection channels, or portions thereof, is connected to a distal region of each of the plurality of interconnected channels.

Embodiment 43. The method of embodiment 41 or 42, wherein the downstream network of connection channels comprises a series of converging channels configured to combine fluid flow from the plurality of interconnected channels of the SEC microfluidic device to the output port.

Embodiment 44. The method of any one of embodiments 41-43, wherein the plurality of interconnected channels of the SEC microfluidic device are only connected via the upstream network of connection channels or the downstream network of connection channels.

Embodiment 45. The method of any one of embodiments 1-44, wherein each of the plurality of interconnected channels of the SEC microfluidic device has a length of about 2 cm to about 50 cm.

Embodiment 46. The method of any one of embodiments 1-45, wherein each of the plurality of interconnected channels of the SEC microfluidic device has a width of about 1 μm to about 15 μm.

Embodiment 47. The method of any one of embodiments 1-46, wherein each of the plurality of interconnected channels of the SEC microfluidic device has a depth of about 1 μm to about 15 μm.

Embodiment 48. The method of any one of embodiments 1-47, wherein the plurality of interconnected channels of the SEC microfluidic device are formed via a pillar array.

Embodiment 49. The method of embodiment 48, wherein the pillar array is an amorphous pillar array.

Embodiment 50. The method of embodiment 48, wherein the pillar array is a non-amorphous pillar array.

Embodiment 51. The method of any one of embodiments 32-50, wherein the pillar array forms an inner surface of each of the plurality of interconnected channels of the SEC microfluidic device.

Embodiment 52. The method of any one of embodiments 1-51, wherein the SEC microfluidic device comprises a quartz substrate.

Embodiment 53. The method of any one of embodiments 1-42, wherein the SEC microfluidic device comprises a monolithic substrate forming the plurality of interconnected channels.

Embodiment 54. The method of any one of embodiments 1-53, wherein the SEC microfluidic device comprises a quartz monolithic substrate.

Embodiment 55. The method of any one of embodiments 1-44, wherein the SEC microfluidic device comprises a three-dimensional (3D) printed substrate.

Embodiment 56. The method of any one of embodiments 1-55, wherein collecting the plurality of fractions eluted from the SEC microfluidic device is performed using a fraction collector.

Embodiment 57. The method of any one of embodiments 1-56, wherein each of the plurality of fractions is collected from the SEC microfluidic device based on time.

Embodiment 58. The method of embodiment 57, wherein each of the plurality of fractions is collected from the SEC microfluidic device for a period of about 30 seconds to about 5 minutes.

Embodiment 59. The method of embodiment 57 or 58, wherein each of the plurality of fractions is collected from the SEC microfluidic device for a uniform amount of time.

Embodiment 60. The method of embodiment 47 or 58, wherein a fraction of the plurality of fractions is collected from the SEC microfluidic device for a different amount of time than another fraction of the plurality of fractions.

Embodiment 61. The method of any one of embodiments 1-56, wherein each of the plurality of fractions is collected from the SEC microfluidic device based on volume of eluate from the SEC microfluidic device.

Embodiment 62. The method of embodiment 61, wherein each of the plurality of fractions collected from the SEC microfluidic device has a volume of about 1 μL to about 20 μL.

Embodiment 63. The method of embodiment 61 or 62, wherein each of the plurality of fractions collected from the SEC microfluidic device has a uniform volume.

Embodiment 64. The method of embodiment 62 or 63, wherein a fraction of the plurality of fractions collected from the SEC microfluidic device has different volume than another fraction of the plurality of fractions.

Embodiment 65. The method of any one of embodiments 1-64, wherein the plurality of fraction is about 5 to about 50 fractions.

Embodiment 66. The method of embodiment 65, wherein the plurality of fraction is about 12 to about 24 fractions.

Embodiment 67. The method of any one of embodiments 1-66, wherein the proteolytic technique comprises an enzyme-based digestion technique.

Embodiment 68. The method of embodiment 67, wherein the enzyme-based digestion technique comprise the use of an enzyme selected from the group consisting of trypsin, chymotrypsin, pepsin, LysC, LysN, AspN, GluC and ArgC, or a combination thereof.

Embodiment 69. The method of embodiment 67 or 68, wherein the enzyme-based digestion technique comprises a step of diluting the fraction eluted from the SEC microfluidic device.

Embodiment 70. The method of embodiment 69, wherein the diluting comprises admixing the fraction eluted from the SEC microfluidic device with water to reach a concentration of the chaotropic agent.

Embodiment 71. The method of embodiment 70, wherein the final concentration of the concentration of the chaotropic agent for the enzymatic digestion is about 0.5 M.

Embodiment 72. The method of any one of embodiments 67-71, wherein the enzyme-based digestion technique does not comprise a buffer exchange step.

Embodiment 73. The method of any one of embodiments 67-72, wherein the enzyme-based digestion technique does not comprise an alkylation step.

Embodiment 74. The method of any one of embodiments 67-72, wherein the enzyme-based digestion technique does not comprise a reduction step.

Embodiment 75. The method of any one of embodiments 1-66, wherein the proteolytic technique comprises a non-enzyme-based approach.

Embodiment 76. The method of any one of embodiments 1-75, wherein the method further comprises subjecting one or more of the plurality of fractions from the SEC microfluidic device and/or one or more of the plurality of fractions subjected to the proteolytic technique to a quantitative labeling technique, wherein the quantitative labeling technique is performed prior to the reversed-phase liquid chromatography (RPLC) technique using the RPLC microfluidic device.

Embodiment 77. The method of embodiment 76, wherein the quantitative labeling technique comprises use of an isobaric mass tag.

Embodiment 78. The method of embodiment 76 or 77, wherein the quantitative labeling technique comprises use of a Tandem Mass Tag (TMT).

Embodiment 79. The method of any one of embodiments 76-78, wherein the quantitative labeling technique comprises a desalting step.

Embodiment 80. The method of any one of embodiments 1-79, wherein the method further comprises admixing an internal standard with one or more of the plurality of fractions from the SEC microfluidic device and/or one or more of the plurality of fractions subjected to the proteolytic technique, wherein the admixing of the internal standard is performed prior to the reversed-phase liquid chromatography (RPLC) technique using the RPLC microfluidic device.

Embodiment 81. The method of embodiment 79, wherein the internal standard is an isotopically-labeled peptide.

Embodiment 82. The method of any one of embodiments 1-81, wherein the one or more fractions subjected to the RPLC technique comprises one or more fractions, or portions thereof, obtained from: (i) zero or more fractions obtained from the SEC microfluidic device; and (ii) one or more of the plurality of fractions subjected to the proteolytic technique.

Embodiment 83. The method of any one of embodiments 1-82, wherein each of the one or more fractions subjected to the RPLC technique comprises the respective fraction of origin admixed with an aqueous solution.

Embodiment 84. The method of any one of embodiments 1-83, wherein the fraction subjected to the RPLC technique has a volume of about 1 μL to about 50 μL.

Embodiment 85. The method of any one of embodiments 1-84, wherein the RPLC technique comprise use of a RPLC mobile phase.

Embodiment 86. The method of embodiment 85, wherein the RPLC technique comprises a mobile phase flow rate of the RPLC mobile phase of about 0.05 μL/minute to about 2 μL/minute.

Embodiment 87. The method of any one of embodiments 1-86, wherein the RPLC technique is a gradient RPLC technique.

Embodiment 88. The method of any one of embodiments 1-87, wherein the RPLC technique is performed at an elevate temperature.

Embodiment 89. The method of any one of embodiments 1-37, wherein the RPLC technique is performed at a temperature of about 30° C. to about 100° C.

Embodiment 90. The method of embodiment 88 or 89, wherein the RPLC technique is performed at a substantially consistent temperature.

Embodiment 91. The method of any one of embodiments 1-90, wherein the reversed-phased medium comprises a RPLC moiety mixture comprising two or more of the following alkyl moieties: C₂, C₄, C₈, and C₁₈.

Embodiment 92. The method of embodiment 91, wherein the RPLC moiety mixture comprises three or more of the following alkyl moieties: C₂, C₄, C₈, and C₁₈.

Embodiment 93. The method of embodiment 91, wherein the RPLC moiety mixture comprises the following alkyl moieties: C₂, C₄, C₈, and C₁₈.

Embodiment 94. The method of any one of embodiments 91-93, wherein the alkyl moieties of the RPLC moiety mixture are present in equimolar amounts.

Embodiment 95. The method of any one of embodiments 91-94, wherein the alkyl moieties of the RPLC moiety mixture are covalently coupled to surfaces of each of the plurality of interconnected channels of the RPLC microfluidic device.

Embodiment 96. The method of embodiment 95, wherein surfaces of each of the plurality of interconnected channels comprise silica (SiO₂).

Embodiment 97. The method of any one of embodiments 1-96, wherein the plurality of interconnected channels of the RPLC microfluidic device comprises 8 or more interconnected channels.

Embodiment 98. The method of embodiment 97, wherein the plurality of interconnected channels of the RPLC microfluidic device comprises 32 interconnected channels.

Embodiment 99. The method of embodiment 97, wherein the plurality of interconnected channels of the RPLC microfluidic device comprises 64 interconnected channels.

Embodiment 100. The method of any one of embodiments 1-85, wherein each of the plurality of interconnected channels of the RPLC microfluidic device are in fluidic communication with an input port of the RPLC microfluidic device via an upstream network of connection channels.

Embodiment 101. The method of embodiment 100, wherein the upstream network of connection channels, or portions thereof, is connected to a proximal region of each of the plurality of interconnected channels.

Embodiment 102. The method of embodiment 100 or 101, wherein the upstream network of connection channels comprises a series of diverging channels configured to split fluid flow from the input port of the RPLC microfluidic device to each of the plurality of interconnected channels.

Embodiment 103. The method of any one of embodiments 1-102, wherein each of the plurality of interconnected channels of the RPLC microfluidic device is in fluidic communication with an output port of the RPLC microfluidic device via a downstream network of connection channels.

Embodiment 104. The method of embodiment 103, wherein the downstream network of connection channels, or portions thereof, is connected to a distal region of each of the plurality of interconnected channels.

Embodiment 105. The method of embodiment 103 and 104, wherein the downstream network of connection channels comprises a series of converging channels configured to combine fluid flow from the plurality of interconnected channels of the RPLC microfluidic device to the output port.

Embodiment 106. The method of any one of embodiments 103-105, wherein the plurality of interconnected channels of the RPLC microfluidic device are only connected via the upstream network of connection channels or the downstream network of connection channels.

Embodiment 107. The method of any one of embodiments 1-106, wherein each of the plurality of interconnected channels of the RPLC microfluidic device has a length of about 2 cm to about 50 cm.

Embodiment 108. The method of any one of embodiments 1-107, wherein each of the plurality of interconnected channels of the RPLC microfluidic device has a width of about 1 μm to about 15 μm.

Embodiment 109. The method of any one of embodiments 1-108, wherein each of the plurality of interconnected channels of the RPLC microfluidic device has a depth of about 1 μm to about 15 μm.

Embodiment 110. The method of any one of embodiments 1-109, wherein the plurality of interconnected channels of the RPLC microfluidic device are formed via a pillar array.

Embodiment 111. The method of embodiment 110, wherein the pillar array is an amorphous pillar array.

Embodiment 112. The method of embodiment 110, wherein the pillar array is a non-amorphous pillar array.

Embodiment 113. The method of any one of embodiments 110-112, wherein the pillar array forms an inner surface of each of the plurality of interconnected channels of the RPLC microfluidic device comprises.

Embodiment 114. The method of any one of embodiments 1-113, wherein the RPLC microfluidic device comprises an online divert feature.

Embodiment 115. The method of embodiment 114, wherein the online divert feature is a valve and/or a channel.

Embodiment 116. The method of embodiment 114 or 115, wherein the online divert feature is positioned between the plurality of interconnected channels of the RPLC microfluidic device and the electrospray ionization device.

Embodiment 117. The method of any one of embodiments 1-116, wherein the RPLC microfluidic device comprises a quartz substrate.

Embodiment 118. The method of any one of embodiments 1-117, wherein the RPLC microfluidic device comprises a monolithic substrate forming the plurality of interconnected channels.

Embodiment 119. The method of any one of embodiments 1-118, wherein the RPLC microfluidic device comprises a quartz monolithic substrate.

Embodiment 120. The method of any one of embodiments 1-119, wherein the RPLC microfluidic device comprises a three-dimensional (3D) printed substrate.

Embodiment 121. The method of any one of embodiments 1-120, wherein the RPLC microfluidic device is configured in an open tubular format.

Embodiment 122. The method of any one of embodiments 1-121, wherein the RPLC microfluidic device is configured for online desalting.

Embodiment 123. The method of any one of embodiments 1-122, wherein the electrospray ionization source is a nano-electrospray ionization source.

Embodiment 124. The method of any one of embodiments 1-1231, wherein the electrospray ionization source is a heated electrospray ionization source.

Embodiment 125. The method of any one of embodiments 1-124, wherein the sample is selected from the group consisting of a blood sample, cerebrospinal fluid (CSF) sample, ascitic fluid sample, seminal fluid sample, and nipple aspirate fluid sample.

Embodiment 126. The method of any one of embodiments 1-125, wherein the sample has a volume of about 10 μL to about 200 μL.

Embodiment 127. The method of any one of embodiments 1-126, wherein the sample is a blood sample.

Embodiment 128. The method of any one of embodiments 1-107, when the sample from the individual is a blood sample, the method further comprises preparing a plasma sample.

Embodiment 129. The method of embodiment 128, wherein preparing the plasms sample comprises subjecting the blood sample to a plasma generation technique.

Embodiment 130. The method of embodiment 129, wherein the plasma generation technique comprises subjecting the sample to a polysulphone medium.

Embodiment 131. The method of embodiment 130, wherein the polysulphone medium is an asymmetric polysulphone material.

Embodiment 132. The method of any one of embodiments 129-131, wherein the plasma generation technique is a capillary action filtration technique.

Embodiment 133. The method of any one of embodiments 129-132, wherein the volume of the blood sample subjected to the plasma generation technique is about 10 μL to about 200 μL.

Embodiment 134. The method of any one of embodiments 129-133, further comprising admixing the generated plasma sample with the liquid fixative to generate the test sample.

Embodiment 135. The method of embodiment 134, wherein the test sample is not further depleted prior to subjecting the test sample to the SEC technique.

Embodiment 136. The method of any one of embodiments 129-135, wherein the plasma generation technique is performed at an ambient temperature.

Embodiment 137. The method of any one of embodiments 129-136, wherein the sample has not been subjected to a depletion step prior to the plasma generation technique.

Embodiment 138. The method of any one of embodiments 1-137, further comprising subjecting the components, or products thereof, eluted from the RPLC microfluidic device to the mass spectrometer.

Embodiment 139. The method of embodiment 138, further comprising performing a mass spectrometry analysis of the components, or products thereof, of the sample using the mass spectrometer.

Embodiment 140. The method of embodiment 139, wherein the mass spectrometry analysis comprises an analysis of each fraction subjected to the RPLC technique using the RPLC microfluidic device.

Embodiment 141. The method of embodiment 139 or 140, wherein the mass spectrometry analysis comprises obtaining one or more data sets comprising information obtained from the mass spectrometer for each fraction subjected to the RPLC technique using the RPLC microfluidic device.

Embodiment 142. The method of embodiment 141, wherein a single data set comprises information obtained from the mass spectrometer from a single fraction subjected to the RPLC technique using the RPLC microfluidic device.

Embodiment 143. The method of embodiment 141 or 142, wherein each of the one or more data set comprises mass-to-charge (m/z) and abundance information for ions of the components, or products thereof, introduced to the mass spectrometer.

Embodiment 144. A collection of compositions obtained from any one of the methods of embodiments 1-143, wherein each composition of the collection of compositions is a RPLC microfluidic device eluate.

Embodiment 145. A method of analyzing a collection of compositions using mass spectrometry, the method comprising: (a) subjecting each composition of the collection of compositions to a mass spectrometer; and (b) performing a mass spectrometry analysis of each composition of the collection of compositions, wherein the collection of compositions is obtained from a processing technique comprising fractionation of a test sample using a SEC technique comprising use of a SEC microfluidic device followed by application of each fraction, or a product thereof, to a RPLC technique comprising use of a RPLC microfluidic device.

Embodiment 146. The method of embodiment 145, wherein the SEC fraction is further processed via a proteolysis technique.

Embodiment 147. The method of any of embodiments 141-143, further comprising, based on at least one of the one or more data sets, determining the identities of each of a plurality of the one or more biomolecules in the test sample.

Embodiment 148. The method of embodiment any of embodiments 141-143 and 147, further comprising, based on at least one of the one or more data sets, measuring the quantities of each of a plurality of the one or more biomolecules in the test sample.

Embodiment 149. The method of embodiment 147 or 148, further comprising identifying a signature comprising one or more identified biomolecules from the determined identities.

Embodiment 150. The method of embodiment 149, wherein the identifying further comprises selecting a subset of the one or more identified biomolecules based on the measured quantities of the one or more identified biomolecules.

Embodiment 151. The method of any of embodiments 148-150, wherein the subset of the one or more identified biomolecules is selected based on differential measured quantities of the one or more identified biomolecules compared to a reference sample.

Embodiment 152. The method of any of embodiments 141-143, further comprising identifying a signature comprising one or more identified biomolecules, the identifying comprising: based on at least one of the one or more data sets, measuring the quantities of each of a plurality of the one or more biomolecules in the test sample; selecting a subset of the plurality of the one or more biomolecules in the sample based on the measured quantities; and determining the identities of each of the subset of the plurality of the one or more biomolecules in the test sample.

Embodiment 153. The method of embodiment 152, wherein the subset of the plurality of the one or more biomolecules in the test sample is selected based on differential measured quantities of the plurality of the one or more biomolecules in the test sample compared to a reference sample.

Embodiment 154. The method of embodiment 151 or 153, wherein the test sample is a sample from a diseased subject and the reference sample is a sample from a healthy subject or a control subject.

Embodiment 155. The method of embodiment 151 or 153, wherein the test sample is a sample from a subject having a pre-condition related to a disease and the reference sample is a sample from a healthy subject or a control subject.

Embodiment 156. The method of embodiment 151 or 153, wherein the test sample is a sample from a subject with a disease in an active state and the reference sample is a sample from a subject with the disease in an inactive state, optionally wherein the inactive state is remission.

Embodiment 157. The method of embodiment 151 or 153, wherein the test sample is a sample from a subject with a disease at an advanced stage and the reference sample is a sample from a subject with the disease at an early stage.

Embodiment 158. A signature comprising a plurality of the identified biomolecules or a subset thereof identified by the method of any of embodiments 149-157.

Embodiment 159. A signature comprising the subset of identified biomolecules identified by the method of any of embodiments 150-158.

Embodiment 160. The method of any of embodiments 147-157, further comprising providing all or a subset of the identified biomolecules of the signature as input to one or more processes configured to perform gene enrichment analysis, one or more processes configured to perform pathway analysis, and/or one or more processes configured to perform network analysis.

Embodiment 161. A method of analyzing biomolecules of a sample, the method comprising providing the identified biomolecules of the signature of embodiment 158 or 159 as input to one or more processes configured to perform gene enrichment analysis, one or more processes configured to perform pathway analysis, and/or one or more processes configured to perform network analysis.

Embodiment 162. The method of embodiment 160 or 161, wherein identified biomolecules of one or more molecular types of the signature are provided as the input.

Embodiment 163. The method of embodiment 162, wherein the one or more molecular types comprise proteins.

Embodiment 164. The method of embodiment 163, wherein the one or more molecular types consist only of proteins.

Embodiment 165. The method of any of embodiments 160-164, wherein the one or more processes configured to perform gene enrichment analysis comprise a process configured to identify one or more gene ontologies each associated with at least one of the identified biomolecules of the signature provided as input, or at least one of the products thereof.

Embodiment 166. The method of any of embodiments 160-165, wherein the one or more processes configured to perform gene enrichment analysis comprise: a process configured to identify one or more cellular component gene ontologies each associated with at least one of the identified biomolecules of the signature provided as input, or at least one of the products thereof; a process configured to identify one or more molecular pathway gene ontologies each associated with at least one of the identified biomolecules of the signature provided as input, or at least one of the products thereof; and/or a process configured to identify one or more biological process gene ontologies each associated with at least one of the identified biomolecules of the signature provided as input, or at least one of the products thereof.

Embodiment 167. The method of any of embodiments 160-166, wherein the one or more processes configured to perform gene enrichment analysis comprise a process configured to identify one or more regulators of at least one of the identified biomolecules of the signature provided as input, or at least one of the products thereof.

Embodiment 168. The method of any of embodiments 160-167, wherein the one or more processes configured to perform gene enrichment analysis comprise: a process configured to identify one or more transcription factors regulating at least one of the identified biomolecules of the signature provided as input, or at least one of the products thereof; and/or a process configured to identify one or more kinases regulating at least one of the identified biomolecules of the signature provided as input, or at least one of the products thereof.

Embodiment 169. The method of any of embodiments 160-168, wherein the one or more processes configured to perform pathway analysis comprise a process configured to identify one or more pathways each associated with at least one of the identified biomolecules of the signature provided as input, or at least one of the products thereof.

Embodiment 170. The method of any of embodiments 160-169, wherein the one or more processes configured to perform pathway analysis comprise: a process configured to identify one or more molecular pathways each associated with at least one of the identified biomolecules of the signature provided as input, or at least one of the products thereof; a process configured to identify one or more signaling pathways each associated with at least one of the identified biomolecules of the signature provided as input, or at least one of the products thereof; and/or a process configured to identify one or more metabolic pathways each associated with at least one of the identified biomolecules of the signature provided as input, or at least one of the products thereof.

Embodiment 171. The method of any of embodiments 160-170, wherein the one or more processes configured to perform network analysis comprise a process configured to identify one or more networks each associated with at least one of the identified biomolecules of the signature provided as input, or at least one of the products thereof.

Embodiment 172. The method of any of embodiments 160-171, wherein the one or more processes configured to perform network analysis comprise: a process configured to identify one or more molecular pathways each associated with at least one of the identified biomolecules of the signature provided as input, or at least one of the products thereof; and/or a process configured to identify one or more signaling pathways each associated with at least one of the identified biomolecules of the signature provided as input, or at least one of the products thereof.

Embodiment 173. The method of any of embodiments 160-172, wherein the one or more processes configured to perform network analysis comprise a process configured to identify one or more hubs of one or more networks each associated with at least one of the identified biomolecules of the signature provided as input, or at least one of the products thereof.

Embodiment 174. The method of any of embodiments 160-173, wherein the one or more processes configured to perform network analysis comprise a process configured to identify one or more drugs each targeting at least one of the identified biomolecules of the signature provided as input, or at least one of the products thereof, optionally wherein the process is configured to identify one or more drugs each targeting at least one hub of a network comprising a plurality of the identified biomolecules of the signature provided as input.

Embodiment 175. The method of any of embodiments 160-174, wherein the one or more processes configured to perform network analysis comprises two processes configured to identify one or more drugs each targeting at least one of the identified biomolecules of the signature provided as input, or at least one of the products thereof, optionally wherein the two processes are configured to identify one or more drugs each targeting at least one hub of a network comprising a plurality of the identified biomolecules of the signature provided as input.

Embodiment 176. A method of analyzing a signature of identified biomolecules, comprising providing a plurality of identified biomolecules to each of a plurality of processes each configured to perform gene enrichment analysis, pathway analysis, or network analysis, wherein: the providing is performed in any order; the plurality of identified biomolecules comprises a protein set, a transcriptomic set, a peptide set, and/or a metabolite set; and the plurality of processes comprise: a process configured to perform gene enrichment analysis to identify one or more gene ontologies each associated with at least one of the plurality of identified biomolecules provided as input, or at least one of the products thereof; a process configured to perform pathway analysis to identify one or more signaling pathways each associated with at least one of the plurality of identified biomolecules provided as input, or at least one of the products thereof; a process configured to perform gene enrichment analysis to identify one or more transcription factors regulating at least one of the plurality of identified biomolecules provided as input, or at least one of the products thereof; a process configured to perform gene enrich analysis to identify one or more kinases regulating a gene product of at least one of the plurality of identified biomolecules provided as input, or at least one of the products thereof; a process configured to perform network analysis to identify one or more networks each associated with at least one of the plurality of identified biomolecules provided as input, or at least one of the products thereof; and each of two processes configured to perform network analysis to identify one or more drugs each targeting at least one of the plurality of identified biomolecules provided as input, or at least one of the products thereof.

Embodiment 177. A method of analyzing a protein signature, comprising providing a plurality of proteins to each of a plurality of processes each configured to perform gene enrichment analysis, pathway analysis, or network analysis, wherein the providing is performed in any order, and the plurality of processes comprise: a process configured to perform gene enrichment analysis to identify one or more gene ontologies each associated with at least one of the plurality of proteins provided as input, or at least one of the products thereof; a process configured to perform pathway analysis to identify one or more signaling pathways each associated with at least one of the plurality of proteins provided as input, or at least one of the products thereof; a process configured to perform gene enrichment analysis to identify one or more transcription factors regulating at least one of the plurality of proteins provided as input, or at least one of the products thereof; a process configured to perform gene enrich analysis to identify one or more kinases regulating a gene product of at least one of the plurality of proteins provided as input, or at least one of the products thereof; a process configured to perform network analysis to identify one or more networks each associated with at least one of the plurality of proteins provided as input, or at least one of the products thereof; and each of two processes configured to perform network analysis to identify one or more drugs each targeting at least one of the plurality of proteins provided as input, or at least one of the products thereof.

Embodiment 178. A size-exclusion chromatography (SEC) microfluidic device comprising: an input port; an upstream network of connection channels; and a plurality of interconnected channels, wherein each channel of the plurality of interconnected channels is in an open tubular format, wherein each channel of the plurality of interconnected channels comprises an inner surface comprising a SEC medium, and wherein each channel of the plurality of interconnected channels is in fluidic communication with the input port via the upstream network of connection channels.

Embodiment 179. The SEC microfluidic device of embodiment 178, wherein the inner surface comprising the SEC medium has a thickness of about 0.5 μm to about 2 μm.

Embodiment 180. The SEC microfluidic device of embodiment 178 or 179, wherein the SEC medium is a material having an average pore size of about 10 nm to about 500 nm.

Embodiment 181. The SEC microfluidic device of any one of embodiments 178-180, wherein the plurality of interconnected channels of the SEC microfluidic device comprises between 8 and 100 interconnected channels.

Embodiment 182. The SEC microfluidic device of any one of embodiments 178-181, wherein the plurality of interconnected channels of the SEC microfluidic device comprises 8 or more interconnected channels.

Embodiment 183. The SEC microfluidic device of any one of embodiments 178-182, wherein the plurality of interconnected channels of the SEC microfluidic device comprises 32 interconnected channels.

Embodiment 184. The SEC microfluidic device of any one of embodiments 178-182, wherein the plurality of interconnected channels of the SEC microfluidic device comprises 64 interconnected channels.

Embodiment 185. The SEC microfluidic device of any one of embodiments 178-184, wherein the upstream network of connection channels, or portions thereof, is connected to a proximal region of each of the plurality of interconnected channels.

Embodiment 186. The SEC microfluidic device of any one of embodiments 178-185, wherein the upstream network of connection channels comprises a series of diverging channels configured to split fluid flow from the input port of the SEC microfluidic device to each of the plurality of interconnected channels.

Embodiment 187. The SEC microfluidic device of any one of embodiments 178-186, wherein each of the plurality of interconnected channels of the SEC microfluidic device is in fluidic communication with an output port of the SEC microfluidic device via a downstream network of connection channels.

Embodiment 188. The SEC microfluidic device of embodiment 187, wherein the downstream network of connection channels comprises a series of converging channels configured to combine fluid flow from the plurality of interconnected channels of the SEC microfluidic device to the output port.

Embodiment 189. The SEC microfluidic device of any one of embodiments 178-188, wherein each of the plurality of interconnected channels of the SEC microfluidic device has a length of about 2 cm to about 30 cm.

Embodiment 190. The SEC microfluidic device of any one of embodiments 178-189, wherein each of the plurality of interconnected channels of the SEC microfluidic device has a width of about 1 μm to about 15 μm.

Embodiment 191. The SEC microfluidic device of any one of embodiments 178-190, wherein each of the plurality of interconnected channels of the SEC microfluidic device has a depth of about 1 μm to about 15 μm.

Embodiment 192. The SEC microfluidic device of any one of embodiments 178-191, wherein the plurality of interconnected channels of the SEC microfluidic device are formed via a pillar array.

Embodiment 193. The SEC microfluidic device of embodiment 192, wherein the pillar array is an amorphous pillar array.

Embodiment 194. The SEC microfluidic device of embodiment 192, wherein the pillar array is a non-amorphous pillar array.

Embodiment 195. The SEC microfluidic device of any one of embodiments 192-194, wherein the pillar array forms an inner surface of each of the plurality of interconnected channels of the SEC microfluidic device.

Embodiment 196. The SEC microfluidic device of any one of embodiments 178-195, wherein the SEC microfluidic device comprises a quartz substrate.

Embodiment 197. The SEC microfluidic device of any one of embodiments 178-196, wherein the SEC microfluidic device comprises a monolithic substrate forming the plurality of interconnected channels.

Embodiment 198. The SEC microfluidic device of any one of embodiments 178-197, wherein the SEC microfluidic device comprises a quartz monolithic substrate.

Embodiment 199. The SEC microfluidic device of any one of embodiments 178-198, wherein the SEC microfluidic device comprises a three-dimensional (3D) printed substrate.

Embodiment 200. A reversed-phase liquid chromatography (RPLC) microfluidic device comprising: an input port; an upstream network of connection channels; and a plurality of interconnected channels, wherein each channel of the plurality of interconnected channels is in an open tubular format, wherein each channel of the plurality of interconnected channels comprises an inner surface comprising a RPLC medium, and wherein each channel the plurality of interconnected channels is in fluidic communication with the input port via the upstream network of connection channels.

Embodiment 201. The RPLC microfluidic device of embodiment 200, wherein the RPLC medium comprises an alkyl moiety having about 2 to about 20 carbons.

Embodiment 202. The RPLC microfluidic device of embodiment 200 or 201, wherein the RPLC medium comprises one or more of C₂, C₄, C₈, and C₁₈.

Embodiment 203. The RPLC microfluidic device of any one of embodiments 200-202, wherein RPLC medium comprises a RPLC moiety mixture comprising two or more of the following alkyl moieties: C₂, C₄, C₈, and C₁₈.

Embodiment 204. The RPLC microfluidic device of embodiment 203, wherein the RPLC moiety mixture comprises three or more of the following alkyl moieties: C₂, C₄, C₈, and C₁₈

Embodiment 205. The RPLC microfluidic device of embodiment 203 or 204, wherein the RPLC moiety mixture comprises the following alkyl moieties: C₂, C₄, C₈, and C₁₈.

Embodiment 206. The RPLC microfluidic device of any one of embodiments 203-205, wherein the alkyl moieties of the RPLC moiety mixture are present in equimolar amounts.

Embodiment 207. The RPLC microfluidic device of any one of embodiments 200-206, wherein the RPLC medium is conjugated to the inner surface of each channel of the interconnected plurality of parallel channels via silica (SiO₂).

Embodiment 208. The RPLC microfluidic device of any one of embodiments 200-207, wherein the plurality of interconnected channels of the RPLC microfluidic device comprises between 8 and 100 interconnected channels.

Embodiment 209. The RPLC microfluidic device of any one of embodiments 200-208, wherein the plurality of interconnected channels of the RPLC microfluidic device comprises 8 or more interconnected channels.

Embodiment 210. The RPLC microfluidic device of any one of embodiments 200-209, wherein the plurality of interconnected channels of the RPLC microfluidic device comprises 32 interconnected channels.

Embodiment 211. The RPLC microfluidic device of any one of embodiments 200-209, wherein the plurality of interconnected channels of the RPLC microfluidic device comprises 64 interconnected channels.

Embodiment 212. The RPLC microfluidic device of any one of embodiments 200-211, wherein the upstream network of connection channels, or portions thereof, is connected to a proximal region of each of the plurality of interconnected channels.

Embodiment 213. The RPLC microfluidic device of any one of embodiments 200-212, wherein the upstream network of connection channels comprises a series of diverging channels configured to split fluid flow from the input port of the RPLC microfluidic device to each of the plurality of interconnected channels.

Embodiment 214. The RPLC microfluidic device of any one of embodiments 200-213, wherein each of the plurality of interconnected channels of the RPLC microfluidic device is in fluidic communication with an output port of the RPLC microfluidic device via a downstream network of connection channels.

Embodiment 215. The RPLC microfluidic device of embodiment 214, wherein the downstream network of connection channels comprises a series of converging channels configured to combine fluid flow from the plurality of interconnected channels of the RPLC microfluidic device to the output port.

Embodiment 216. The RPLC microfluidic device of any one of embodiments 200-215, wherein each of the plurality of interconnected channels of the RPLC microfluidic device has a length of about 2 cm to about 30 cm.

Embodiment 217. The RPLC microfluidic device of any one of embodiments 200-216, wherein each of the plurality of interconnected channels of the RPLC microfluidic device has a width of about 1 μm to about 15 μm.

Embodiment 218. The RPLC microfluidic device of any one of embodiments 200-217, wherein each of the plurality of interconnected channels of the RPLC microfluidic device has a depth of about 1 μm to about 15 μm.

Embodiment 219. The RPLC microfluidic device of any one of embodiments 200-218, wherein the plurality of interconnected channels of the RPLC microfluidic device are formed via a pillar array.

Embodiment 220. The RPLC microfluidic device of embodiment 219, wherein the pillar array is an amorphous pillar array.

Embodiment 221. The RPLC microfluidic device of embodiment 219, wherein the pillar array is a non-amorphous pillar array.

Embodiment 222. The RPLC microfluidic device of any one of embodiments 219-221, wherein the pillar array forms an inner surface of each of the plurality of interconnected channels of the RPLC microfluidic device.

Embodiment 223. The RPLC microfluidic device of any one of embodiments 219-221, wherein the RPLC microfluidic device comprises a quartz substrate.

Embodiment 224. The RPLC microfluidic device of any one of embodiments 219-223, wherein the RPLC microfluidic device comprises a monolithic substrate forming the plurality of interconnected channels.

Embodiment 225. The RPLC microfluidic device of any one of embodiments 219-224, wherein the RPLC microfluidic device comprises a quartz monolithic substrate.

Embodiment 226. The RPLC microfluidic device of any one of embodiments 219-225, wherein the RPLC microfluidic device comprises a three-dimensional (3D) printed substrate.

Embodiment 227. A method for processing a test sample, the method comprising: (a) subjecting the test sample to a size-exclusion chromatography (SEC) technique using a SEC microfluidic device, wherein the test sample comprises one or more biomolecules and a chaotropic agent, and wherein the SEC microfluidic device comprises a plurality of interconnected channels; (b) collecting one or more fractions eluted from the SEC microfluidic device; (c) subjecting one or more of the fractions collected from the SEC microfluidic device to a proteolytic technique; and (d) subjecting one or more of fractions to a reversed-phase liquid chromatography (RPLC) technique to prepare a fraction for introduction to a mass spectrometer, wherein the one or more RPLC-fractions comprises (i) zero or more fractions obtained from the SEC microfluidic device; and (ii) zero or more fractions subjected to the proteolytic technique.

Embodiment 228. A method of analyzing a composition, the method comprising: (a) subjecting the composition to a mass spectrometer; and (b) performing a mass spectrometry analysis of the composition, wherein the composition is obtained from a processing technique comprising fractionation of a sample using a SEC technique comprising use of a SEC microfluidic device followed by application of one or more fractions from the SEC microfluidic technique, or a product thereof, to a RPLC technique.

Embodiment 229. A method of analyzing a signature of identified components, comprising performing gene enrichment analysis, pathway analysis, and network analysis in any order, wherein: the signature of identified components comprises a protein set, a transcriptomic set, a peptide set, and/or a metabolite set; and the performing comprises: a process configured to perform gene enrichment analysis; a process configured to perform pathway analysis; a process configured to perform gene enrichment analysis; and a process configured to perform network analysis to identify drug targets.

Embodiment 230. A method of subjecting an individual to a coronary artery disease (CAD) diagnosis determination, the method comprising: (a) obtaining mass spectrometry (MS) data from a sample, or a derivative thereof, obtained from the individual; and (b) analyzing the MS data according to a CAD proteomic signature, wherein the CAD proteomic signature comprises one or more biomarkers of Table 1; and (c) determining whether the individual has the CAD proteomic signature.

Embodiment 231. The method of embodiment 230, wherein if the individual has the CAD proteomic signature, the individual is diagnosed has having CAD.

Embodiment 232. A method of diagnosing an individual as having coronary artery disease (CAD), the method comprising: (a) obtaining mass spectrometry (MS) data from a sample, or a derivative thereof, obtained from the individual; and (b) analyzing the MS data according to a CAD proteomic signature, wherein the CAD proteomic signature comprises one or more biomarkers of Table 1; and (c) diagnosing the individual as having CAD based on the presence of the CAD proteomic signature.

Embodiment 233. A method of treating an individual having coronary artery disease (CAD), the method comprising: (a) diagnosing an individual as having CAD according to the presence of a CAD proteomic signature in a sample, or a derivative thereof, obtained from the individual, wherein the CAD proteomic signature comprises one or more biomarkers of Table 1; and (b) administering to the individual a CAD treatment.

Embodiment 234. The method of embodiment 233, wherein the presence of the CAD proteomic signature is determined by analyzing MS data according to the CAD proteomic signature.

Embodiment 235. The method of embodiment 234, further comprising obtaining the MS data from the sample, or the derivative thereof, obtained from the individual.

Embodiment 236. The method of any one of embodiments 233-235, wherein the CAD treatment comprises a life style adjustment.

Embodiment 237. The method of any one of embodiments 233-236, wherein the CAD treatment comprises a pharmaceutical intervention.

Embodiment 238. The method of embodiment 237, wherein the pharmaceutical intervention comprises administration of a drug selected from the group consisting of a calcium channel blocker, histone deacetylase (HDAC) inhibitor (such as HDAC6), Ca2+/calmodulin (CaM)-dependent protein kinase II (CaMK II) inhibitor, guanylyl cyclase (sGC) activator, MMP inhibitor, statin, and anti-hypertesnive.

Embodiment 239. The method of embodiment 237 or 238, wherein the pharmaceutical intervention comprises a drug is selected from the group consisting of amlodipine, tubastatin-a, forskolin, trichostatin A, KN-93, CFM-1571, ilomastat, CAY-10603, and rosuvastatin, or a pharmaceutical salt thereof.

Embodiment 240. The method of embodiment 237 or 238, wherein the drug is selected from the group consisting of BRD-K52306726, BRD-K71361154, acetazolamide, rolipram, ruxolitinib, BRD-A59808129-001-01-7, BRD-K76876037, ZM336372, trehalose, SCHEMBL3092652, BMS-387032, BRD-K01425431, 4-hydroxy-retinoic acid, CHEMBL585951, CHEMBL1673039, HY-11007, primidone, BRD-K81417919, SPECTRUM_000826, tamoxifen, BRD-K00544996, CID 67066889, CX-5461, BRD-K63944563, SCHEMBL6851809, BRD-A86146706, FR-180204, CHEMBL552425, hexachlorophene, Aggc, SUGA1_008424, BRD-K96640811, anastrozole, wortmannin, vandetanib, AC1NWALF, OTSSP167, WZ3105, dihydroergotamine, BRD-K99839793, SR 33805 oxalate, AT-7519, sulfadoxine, SPECTRUM_001319, MLS003329219, trichostatin A, and rotenone, or a pharmaceutical salt thereof.

Embodiment 241. A method for detecting a coronary artery disease (CAD) proteomic signature of an individual, (a) obtaining mass spectrometry (MS) data from a sample, or a derivative thereof, obtained from the individual; and (b) analyzing the MS data according to a CAD proteomic signature to detect the CAD proteomic signature, wherein the CAD proteomic signature comprises one or more biomarkers of Table 1.

Embodiment 242. The method of embodiment 241, wherein the individual is suspected of having CAD.

Embodiment 243. The method of any one of embodiments 230-242, wherein the CAD proteomic signature comprises increased expression of the one or more biomarkers according to Table 1 as compared to a reference.

Embodiment 244. The method of any one of embodiments 230-243, wherein the CAD proteomic signature comprises decreased expression of the one or more biomarkers according to Table 1 as compared to a reference.

Embodiment 245. The method of any one of embodiments 230-244, wherein the CAD proteomic signature comprises one or more biomarkers associated with a calcium signaling pathway, histone regulation, HIF-1 signaling pathway, cAMP signaling pathway, beta-adrenergic signaling pathway, PI3K-Akt signaling pathway, complement and/or coagulation cascade, sphingolipid signaling pathway, natural killer cell mediated cytotoxicity, adipocytoknie signaling pathway, DNA damage, calcium energy, metaboloimcs, cellular adhesion, inflammation, hypoxia, and histone methylation.

Embodiment 246. The method of any one of embodiments 230-245, wherein the one or more biomarkers comprise a subset thereof comprising one or more biomarkers associated with a transcription factor.

Embodiment 247. The method of any one of embodiments 230-246, wherein the one or more biomarkers comprise a subset thereof comprising one or more biomarkers associated with a kinase.

Embodiment 248. The method of any one of embodiments 230-247, wherein the one or more biomarkers comprise at least 10 biomarkers of Table 1.

Embodiment 249. The method of any one of embodiments 230-248, wherein the one or more biomarkers comprise at least 25 biomarkers of Table 1.

Embodiment 250. The method of any one of embodiments 230-249, wherein the one or more biomarkers comprise at least 50 biomarkers of Table 1.

Embodiment 251. The method of any one of embodiments 230-250, wherein the one or more biomarkers comprise all biomarkers of Table 1.

Embodiment 252. The method of any one of embodiments 230-251, further comprising obtaining the sample from the individual.

Embodiment 253. The method of any one of embodiments 230-252, wherein the sample, or the derivative thereof, is a blood sample or a derivative thereof.

Embodiment 254. The method of embodiment 253, wherein the sample, or the derivative thereof, is a plasma sample.

Embodiment 255. The method of embodiment 254, wherein the sample, or the derivative thereof, comprises a liquid fixative.

Embodiment 256. The method of any one of embodiments 230-255, wherein the obtaining MS data from the sample, or the derivative thereof, comprises performing a mass spectrometry analysis of the sample, or the derivative thereof, using a mass spectrometer.

Embodiment 257. The method of embodiment 256, wherein the mass spectrometry analysis is performed according to the method of embodiments 140-143.

Embodiment 258. The method of any one of embodiments 230-257, wherein the analyzing the MS data according to the CAD proteomic signature comprises subjecting the MS data to a method of any one of embodiments 161-177.

Embodiment 259. The method of any one of embodiments 230-258, wherein the analyzing the MS data according to the CAD proteomic signature comprises assessing the presence or absence or level of each of the one or more biomarkers of the CAD proteomic signature in the MS data.

Embodiment 260. The method of any one of embodiments 230-259, further comprising performing one or more of the following factor assessments of the individual: sex, age, body mass index (BMI), systolic blood pressure, diastolic blood pressure, total cholesterol, HDL, LDL, triglycerides, hyperlipidemia, hypertension, diabetes mellitus, insulin resistance, kidney disease, smoking status, level of physical activity, level of sleep, or quality of nutrition.

Embodiment 261. The method of any one of embodiments 230-260, further comprising performing a medical procedure on the individual to assess the presence of CAD.

### IX. EXAMPLES

The following examples are included for illustrative purposes only and are not intended to limit the scope of the invention

### Example 1: Plasma Proteomics Discovery Method

This example demonstrates a comprehensive, quantitative plasma proteomics method for the unbiased discovery, and follow-up targeted analysis, of disease specific protein biosignatures from a prick-test procured blood specimen. This example demonstrates a method integrating multiple innovative technologies that work in unison together to achieve an unpresented level of analysis accuracy, precision, sensitivity, and specificity.

The volume equivalent of freshly procured non-depleted human plasma contained in one drop of blood (about 10-15 μL) was immediately mixed with a liquid fixative at room temperature (RT) to solubilize and preserve its protein and other biological analytes, including primary and secondary metabolites, native peptides, microRNAs, circular and long non-coding RNAs, and mitochondrial RNAs. The plasma extraction from a single blood drop was achieved with capillary action filtration through a commercially available asymmetric Polysulphone™ material, and directly mixed with 40 μL of a liquid fixative of 7 M guanidine HCl in 90% water/10% glycerol. This solution functions as a liquid fixative due to its strong chaotropic activity and thus eliminates protease activity, achieves maximum preservation of chemical integrity of metabolites, eliminates protein-protein binding, imparts a maximum hydrodynamic radius to its constituent analytes, and enhances liquid viscosity, for efficient size exclusion chromatographic (SEC) separation. Additionally, the liquid fixative effectively neutralizes all human pathogens (e.g., viruses, bacteria, etc.) with chemical or toxicological hazards. This configuration is amenable to point-of-care devices for the procurement and chemical fixation of plasma and its protein and metabolite content.

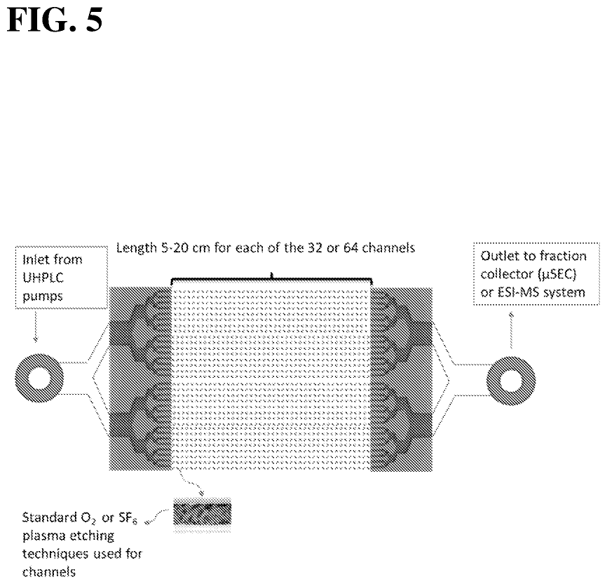

Approximately 5 μL of the preserved plasma specimen was then subjected to direct multi-segmented fractionation with microfluidic ultra-high performance SEC (μUHSEC). This fractionation was achieved with an open tubular device, (Bioinspired Arterial architecture (BioArtery™) (FIG. 5). The open tubular geometry of the BioArtery™ μUHSEC device used herein was composed of quartz having 32 interconnected channels of a length of 10 cm, a width of 5 μm, and a depth of 5 μm. The inner surface of each of these channels was comprised of an amorphous subnetwork with an average pore size of 50-80 nm, resulting from using standard O₂ plasma etching procedures. The dimensions allowed the accommodation of various chromatographic capacities, analyte separation efficiencies, and analyte peak densities, as required to achieve the necessary sensitivity, specificity, and reproducibility of the overall discovery and targeted proteomics methods. Furthermore, the micro-fluidic dimensions of the 12 BioArtery™ μUHSEC device increased analytical sensitivity at low specimen starting volumes. The 12 BioArtery™ μUHSEC device allowed the partitioning and chemical preservation of a wide spectrum of biological analytes including intact hydrophilic and hydrophobic proteins, native peptides, and metabolites, and is amenable to downstream discovery analysis with high-resolution mass spectrometry detection.

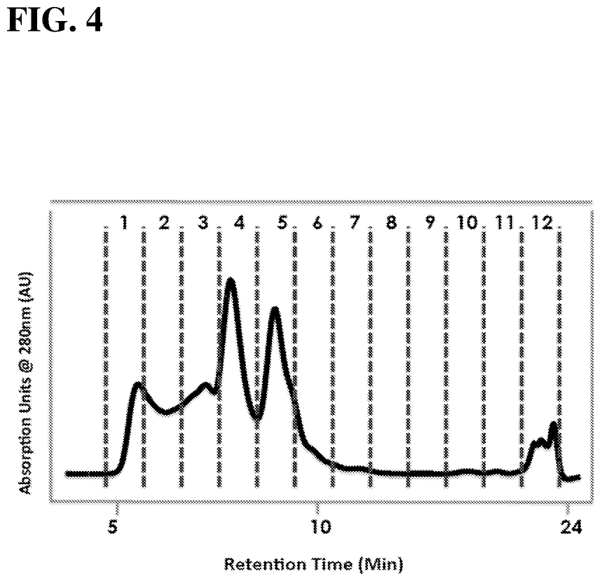

The SEC mobile phase comprised the same components of the liquid fixative, thus eliminating the need for pre-analytical steps, such as clean-up steps. As such, the method demonstrated herein minimizes pre-analytical variables, and thus reduces the measurement standard deviation. The protein content for each segment was determined with UV absorbance at 280 nm, or fluorescence excitation at 290 nm and emission at 320-400 nm. A representative μUHSEC trace is depicted in FIG. 4.

The enhanced performance of the 12 BioArtery™ μUHSEC device was benchmarked against the commercially available packed OEC column TSKgel Super SW3000 1 mm×30 cm×4 μm particle. The same segments with defined total protein amounts therein, and identical downstream analytical procedures described below, were used for this analysis. Furthermore, the analysis also included commercially available systems suitability standards containing proteins of defined molecular weights, peptides, and metabolite mixtures at defined concentration levels. The minimum increase in sensitivity and subsequent proteome coverage was 20-30-fold using the 12 BioArtery™ μUHSEC device compared to the commercially available packed μSEC column TSKgel Super SW3000. The enhanced performance was subsequently utilized to monitor the 12 BioArtery™ μUHSEC performance with the system suitability standards to ensure method sensitivity and reproducibility.

Aliquots from each of the 12 BioArtery™ μUHSEC fractions were diluted 1:10 in purified water, and subjected to solution phase trypsin proteolysis (Promega). The aliquots were stoichiometrically corrected to 1:30 protein content and incubated with trypsin for 8 hours at 37° C. Namely, the protein amounts per segment ranged from 0.1 mg to 10 ng, and the trypsin amount was adjusted to be 30-fold less that protein in each aliquot. No reduction and alkylation steps were necessary due to the liquid fixative properties used for the solution phase proteolysis. The remainder of each of the fractions was preserved for follow-up targeted protein analyses purposes (See, Example 2). For the discovery relative quantitative analysis, each segment was then labeled with stoichiometrically normalized isobaric stable isotope tagging reagent at a 1:3 reagent—protein ratio. The BioArtery™ μUHSEC fractions are also amenable to label-free relative quantitative proteomics using standard data-independent acquisition (DDA) or data-independent acquisition (DIA) approaches.

After proteolysis, each of the 12 BioArtery™ μUHSEC fractions were subjected to a BioArtery™ RPLC device. The BioArtery™ RPLC device was a quartz lab chip having 32 interconnected channels. Each channel had a length of 10 cm, a width of 5 μm, and a depth of 5 μm. The inner channel surfaces were chemically modified with equimolar concentrations of C₂-C₄-C₈-C₁₈ alkyl groups. The C₂₋₄₋₈₋₁₈ surface chemistry affords the ability to separate a wide range of hydrophobic, amphipathic, and hydrophobic peptides, thus facilitating downstream electrospray ionization and mass spectrometry analysis. Using the BioArtery™ RPLC device each sample was on-line desalted, diverted away from the mass spectrometer with the on-line divert valve, and separated. The BioArtery™ RPLC device was coupled with an electrospray ionization source for sample introduction to the mass spectrometer. Electrospray ionization was performed with a heated electrospray source and a nitrogen nebulizer.

The performance of the BioArtery™ RPLC device was benchmarked against the commercially available 2 m-long monolithic C₁₈ capillary column (100 μm ID; GL Sciences). A 60-70% increase in the number of tryptic peptides was typically observed using the BioArtery™ RPLC device. This benchmarking exercise demonstrated the advanced performance of the proposed the BioArtery™ RPLC device against commercially available open tubular columns.

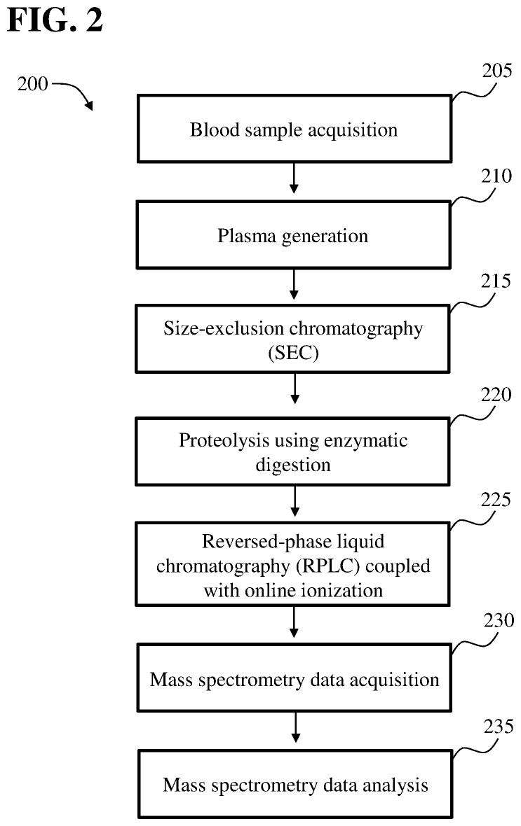

The ultra-high resolution mass spectrometry parameters were based on those reported in Garay-Baquero et al., 2020, JCI Insight 5, as described below. Briefly, higher energy collisional dissociation (HCD) and collision-induced dissociation (CID) fragmentation was performed for each labeled and desalted sample, corresponding to each of the SEC fractions. For the peptides and other larger molecules, the MS observation window was set between 380 and 1500 m/z. The top 10+2 and +3 multiply charged ions were further characterized by tandem MS (MS/MS). For small molecules (metabolites), the MS observation window was set between 80 and 600 m/z and only singly+1, and doubly charged ions, were monitored. Full MS scans were acquired at 120,000 full width at half maximum (FWHM), and MS/MS scans were acquired at a resolution of 30,000 FWHM. To enhance mass accuracy, the lock mass option was enabled for the 445.120025 rn/z ion (DMSO). An exemplary workflow of the discovery platform is shown in FIG. 2.

Spectral processing and false discovery rate (FDR)-corrected statistical analysis for the identification of differentially expressed proteins were performed. Unprocessed raw files were submitted to Proteome Discoverer 1.4 for target decoy search using Sequest. The UniProtKB Homo sapiens database containing 20,159 entries was utilized. The search allowed for up to two missed cleavages, a precursor mass tolerance of 10 ppm, a minimum peptide length of six and a maximum of two variable (one equal) modifications of: oxidation (M), deamidation (N, Q), or phosphorylation (S, T, Y). Methylthio (C) and TMT (K, peptide N-terminus) were set as fixed modifications. FDR corrected p-value at the peptide level was set at <0.05. Percent co-isolation excluding peptides from quantitation was set at 50. Reporter ion abundances from unique peptides only were taken into consideration for the quantitation of the respective protein.

Statistical analyses were based on Welch's two-sample t test for unequal variances to assess significant differences between groups followed by FDR correction for multiple-correction testing (p≤0.01). This Welch two-sample t test was appropriate since there was a balance of samples in groups, and each group was well above the suggested level of 15 per group, allowing control of the type I error rate even in non-normal distributions. Batch effect correction was performed using the ComBat method.

The results of the analysis demonstrated a broad proteome coverage that included the capture of a diverse set of proteins (e.g., secreted, endogenous cleavage products, secreted—soluble proteins, exosome or lipid microvesicle enriched proteins, etc.) spanning a large linear dynamic range (e.g., 12-orders of magnitude or more) from small volumes of non-depleted plasma or serum (e.g., less than 150 μL) in a high-throughput fashion. The method constituted a unitary, vertically integrated pipeline, given the high-degree of complimentary principles of operation between devices. Furthermore, the pipeline is highly amenable to automation and can be scaled-up to increase analysis capacity with minimum human intervention.

### Example 2: The PROMINIA Computational Biology Platform

A computational biology platform named “PROMINIA” (PROtein MINing Intelligent Algorithm) was developed. PROMINIA identifies disease specific signaling pathways and molecular networks derived from differentially expressed proteins that have been captured by the discovery proteomics method, such as described in Example 1. For instance, the discovery proteomics platform can be applied to identify a proteomic signature from diseased patients compared to suitable controls, and the proteomic signature can be further analyzed using the provided PROMINIA platform. The PROMINIA platform can be applied to a proteomic signature of any human disease in order to identify a molecular portrait of the disease. In some examples, the PROMINIA platform matches the molecular portrait of the disease with drug-specific molecular profiles, resulting in the identification of therapeutics for a given disease (such as an FDA-approved or known therapeutic, or a novel therapeutic for a given disease). Thus, in some aspects, the output of the PROMINIA platform includes drug hits that could have therapeutic potential for the patient whose biological sample (e.g., blood plasma) was analyzed.

To use the PROMINIA platform, a proteomic signature can be provided as input, and the PROMINIA platform includes a number of different steps for analyzing the proteomic signature. These analysis steps can include steps of identifying (i) cellular components, molecular pathways, and signaling pathways highly represented in the proteomic signature; (ii) transcription factors and kinases that regulate the proteins of the proteomic signature; (iii) protein-protein interaction networks describing the functional relationships among proteins of the proteomic signature, as well as sub-networks and hubs thereof; and (iv) known and novel drugs targeting proteins of the proteomic signature, including those targeting hubs of the protein-protein interaction networks of the proteomic signature.

The following describes the use of the PROMINIA platform as it was performed on a proteomic signature identified for an exemplary disease. The proteomic signature was identified using the discovery proteomics platform described in Example 1.

### A. Analysis of an Exemplary Proteomic Signature Using PROMINIA

Using the discovery proteomics platform described in Example 1, a proteomic signature was identified for an exemplary disease. Plasma samples were collected and processed as described in Example 1 from eight subjects having the exemplary disease as well as eight sex- and age-matched healthy control subjects. Sample proteins were identified using the discovery proteomics platform, and a proteomic signature of differentially expressed proteins was identified when comparing protein amounts between diseased and healthy subjects. Protein amounts were determined by quantifying the area of detected peaks in the mass spectrometry data (e.g., mass spectrum plots) generated using the samples. The proteomic signature included proteins up-regulated in the exemplary disease as well as proteins down-regulated in the exemplary disease.

After identification, the proteomic signature was analyzed using the PROMINIA platform. First, the proteomic signature was inserted into the ToppGene Suite (Chen J et al., Nucleic Acids Res, 37:W305-11, 2009) in order to identify cellular components associated with the proteomic signature. This analysis revealed cellular components that were highly enriched in the proteomic signature and that were highly relevant with the source (i.e., blood plasma) of the samples. The ToppGene Suite was also used to identify molecular pathways related to the proteomic signature.

Next, the proteomic signature was analyzed using the SPLA R Package (Tarca A L et al., Bioinformatics, 25:75-82, 2009) to identify the blood plasma protein-enriched and statistically significant (p<0.05) signaling pathways.

The proteomic signature was further analyzed with Transcription Factor Enrichment Analysis (TFEA, https://github.comiwzthu/enrichTF) and Kinase Enrichment Analysis (KEA, Lachmann A & Ma'ayan A. Bioinformatics, 25: 684-6, 2009) algorithms to identify the transcription factors and kinases, respectively, that are regulators of the proteomic signature.

The protein signature was then inserted into the GeneMANIA algorithm (Warde-Farley D et al., Nucleic Acids Res, 38:W214-220, 2010) to identify the protein networks, subnetworks, and hub proteins of the key subnetworks. The hubs can be evaluated for their functional importance in disease cellular and animal models (for instance, for novel disease gene identification). This analysis revealed a tightly connected protein network with hundreds of protein-protein interactions, indicating a high degree of functional interaction among proteins of the proteomic signature.

Ultimately, the proteomic signature was inserted into the L1000 FWD (Wang Z et al., Bioinformatics, 34: 2150-52, 2018) algorithm and the ILINCs (https://www.biorxiv.org/content/10.1101/826271v1) chemical perturbation algorithm to identify FDA-approved drugs that target the hubs of protein networks represented in the proteomic signature as well as novel drugs that target the hubs. This analysis revealed drugs that could be used to target the proteomic signature. These identified drugs included not only those already used in the treatment of the exemplary disease, but also those that have not been previously used for treatment of the exemplary disease. These drugs could be used as therapeutics for the patients for which the discovery proteomic analysis was performed. The therapeutic potential of the new drugs can be selected for further evaluation in disease cellular and animal models.

Taken together, these results demonstrate that the proteomic signature included disease-specific proteins and that the discovery proteomics platform identified and quantified these proteins in blood plasma samples of only about 10-15 pt. The PROMINIA platform identified not only known pathways and regulators involved in the pathogenesis of the exemplary disease, but also novel pathways and regulators that could be targeted for therapy. Similarly, as exemplified for an exemplary disease, the PROMINIA platform identified novel drugs never before used in the treatment of the disease that could be used as future therapeutics. Thus, these results demonstrate the predictive power of the PROMINIA platform as well as the predictive power of the discovery proteomics platform. The more complete identification of components from a sample achieved using the methods and/or devices described herein, such as shown in Example 1, further enables the identification of disease specific signaling pathways and molecular networks using PROMINIA.

### Example 3: Analysis of Coronary Artery Disease (CAD) Signatures Using PROMINIA

The following example describes the use of the PROMINIA platform as it was performed on a proteomic signatures of human Coronary Artery Disease (CAD) to identify a CAD proteomic signature.

Using the discovery proteomics platform described in Example 1, a proteomic signature was identified for CAD. Plasma samples were collected and processed as described in Example 1 from eight subjects having CAD as well as three sex- and age-matched healthy control subjects. The characteristics of the CAD study participants are shown in Table 2.

TABLE 2
Characteristics of CAD study participants.

| | Group 1 | Group 2 | | |
|---|---|---|---|---|
| | (CAD, 3- | (CAD, 1- | | p-value |
| | vessel | vessel | Group 3 | (group |
| | disease) | disease) | (control) | 1 + 2 vs. |
| Parameter | n = 4 | n = 4 | n = 3 | group 3) |
| Sex | Male | Male | Male | N/A |
| Age (year) | 51 ± 5 | 50 ± 6 | 50 ± 5 | 0.9 |
| BMI (kg/m²) | 26.3 ± 2.5 | 25.8 ± 2.1 | 25.4 ± 3.0 | 0.7 |
| Systolic blood | 130 ± 10 | 120 ± 20 | 120 ± 10 | 0.5 |
| pressure | | | | |
| (mmHg) | | | | |
| Diastolic blood | 90 ± 10 | 80 ± 20 | 80 ± 10 | 0.5 |
| pressure | | | | |
| (mmHg) | | | | |
| Total cholesterol | 190 ± 30 | 190 ± 20 | 180 ± 20 | 0.4 |
| (mg/dL) | | | | |
| HDL (mg/dL) | 35 ± 10 | 39 ± 12 | 40 ± 10 | 0.5 |
| LDL (mg/dL) | 130 ± 10 | 125 ± 15 | 120 ± 20 | 0.8 |
| Triglycerides | 190 ± 20 | 200 ± 20 | 190 ± 10 | 0.6 |
| (mg/dL) | | | | |

Sample proteins were identified using the discovery proteomics platform, and a proteomic signature of differentially expressed proteins was identified when comparing protein amounts between diseased and healthy subjects. Protein amounts were determined by quantifying the area of detected peaks in the mass spectrometry data (e.g., mass spectrum plots) generated using the samples. The proteomics study resulted in the quantification of 1,407 unique protein groups (p<0.05). A signature of 292 differentially expressed proteins was identified in proteomic blood plasma analysis from samples derived from healthy controls and patients with CAD. The proteomic signature included 139 proteins up-regulated as well as 153 proteins down-regulated in CAD patients relative to healthy controls.

### A. ToppGene Software Cellular Component and Pathway Computational Analysis Related to 292-Protein CAD Signature

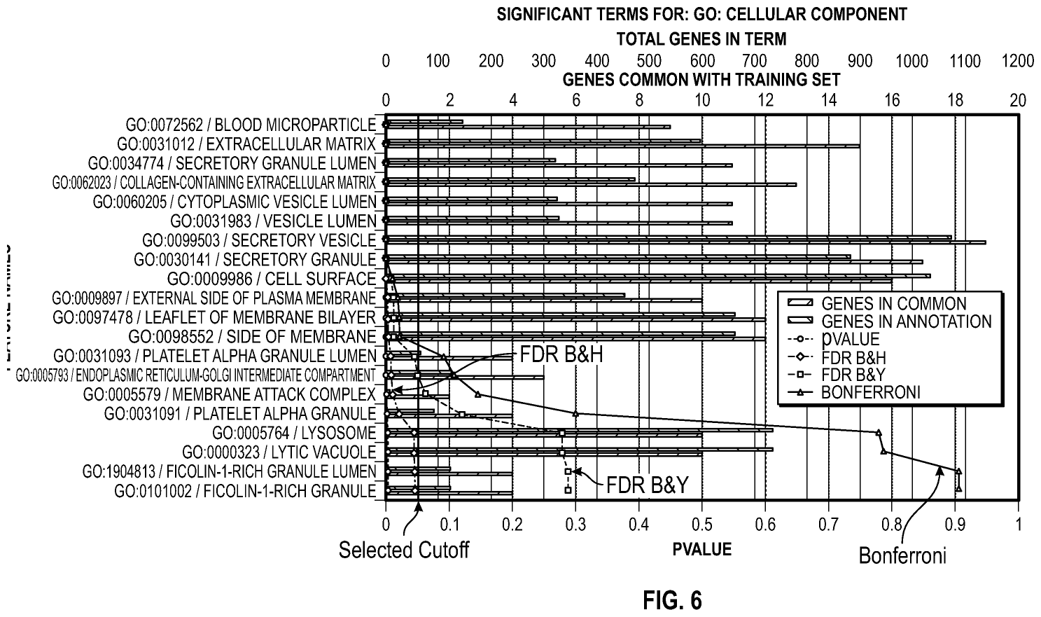

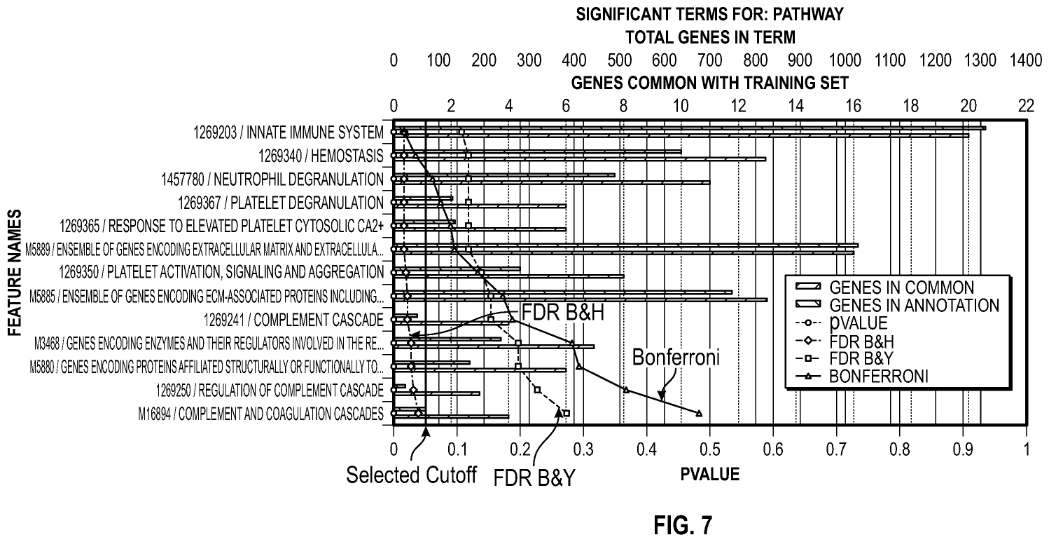

After identification, the 292 CAD-plasma protein proteomic signature derived from the analysis of blood plasma sample from CAD patients and healthy individuals was analyzed using the PROMINIA platform. First, the 292-protein CAD signature was inserted into the ToppGene Suite (Chen J et al., Nucleic Acids Res, 37:W305-11, 2009) in order to identify cellular components associated with the CAD signature. This analysis revealed cellular components that were highly enriched in the proteomic signature and that were highly relevant with the source (i.e., blood plasma) of the samples. The most enriched pathways related to blood microparticles, extracellular matrix, secretory vesicles and vesicle lumen cellular compartments, all highly relevant with the actual source of the tested samples (FIG. 6). The ToppGene Suite was also used to identify molecular pathways related to the 292-protein CAD signature. Immune system related (neutrophils, platelets, complement) pathways, extracellular matrix, and calcium-related pathways were highly enriched in the 292-protein CAD signature (FIG. 7).

These data show that the specific 292-protein CAD signature not only has “biomarker” capabilities but it is a protein signature that relates with the pathobiology of CAD disease.

### B. SPIA Algorithm-Identified Signaling Pathways Related to the 292-Protein CAD Signature

Next, the 292-protein CAD signature was analyzed using the signaling pathway impact analysis (SPIA) R Package (Tarca A L et al., Bioinformatics, 25:75-82, 2009) to identify the blood plasma protein-enriched and statistically significant (p<0.05) signaling pathways that correlate with CAD pathogenesis and pathobiology. As shown in Table 3, the analysis identified signaling pathways that are highly related with the pathogenesis molecular mechanisms related to CAD.

TABLE 3
SRIA-enriched pathways for differentially
expressed proteins in CAD (p < 0.05).
Parameter

Calcium signaling pathway
HIF-1 signaling pathway
cAMP signaling pathway
β-Adrenergic signaling
P13K-Akt signaling pathway
Complement and coagulation cascades
Sphingolipid signaling pathway
Natural killer cell mediated cytoxicity
Adipocytokine signaling pathway

These pathways may be separated into two main groups; The first group includes cardiovascular-related pathways, such as the calcium, cAMP, β-adrenergic and sphingolipid signaling pathways. The second group includes immune-related pathways, such as the complement, HIF1, natural killer immune cell, and adipocytokine signaling pathways.

Taken together, these data reveal the power of the plasma blood proteomic technology and also the value of the specific 292-protein CAD signature to identify proteins highly specific to CAD pathogenesis and not just random or surrogate biomarkers.

### C. Transcription Factor Enrichment Analysis (TFEA) Algorithm and Kinase Enrichment Analysis Related to the 292-Protein CAD Signature

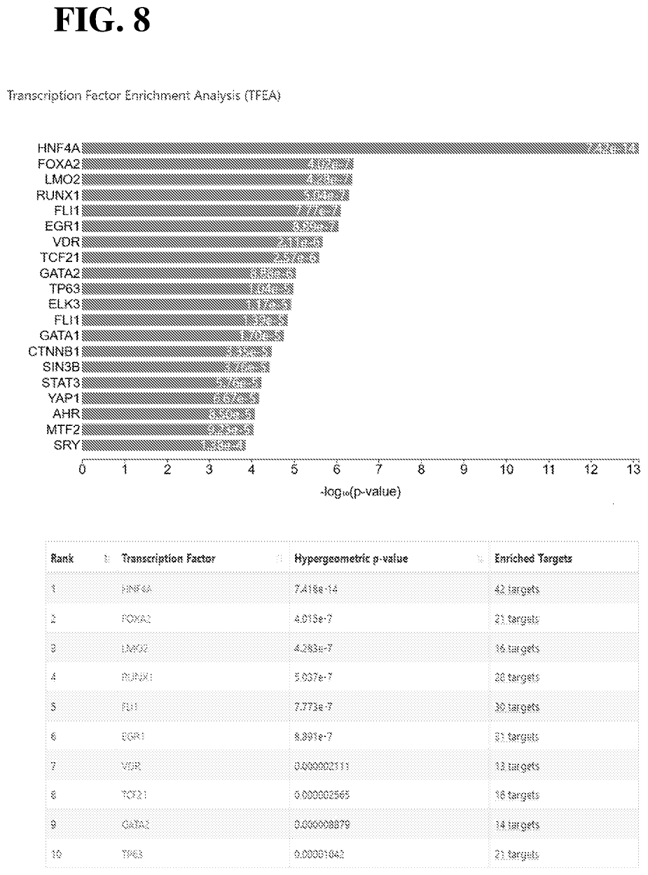

The 292-protein CAD signature was further analyzed with Transcription Factor Enrichment Analysis (TFEA, https://github.comiwzthu/enrichTF) algorithm to identify the transcription factors and kinases, respectively, that are regulators of the 292-protein CAD signature. The analysis revealed 20 transcription factors that are enriched in the 292-protein CAD network (FIG. 8). The top three transcription factors identified to regulate the CAD DEP network were HNF4A, FOXA2, and LMO2. Both HNF4A and FOXA2 are transcription factors that are primarily expressed in the liver and generally in the gastrointestinal tract.

Collectively, the identification of transcription factors that are highly related to CAD pathogenesis and being key regulators of the 292-protein signature, suggest the correlation of the identified protein signature with CAD pathogenesis.

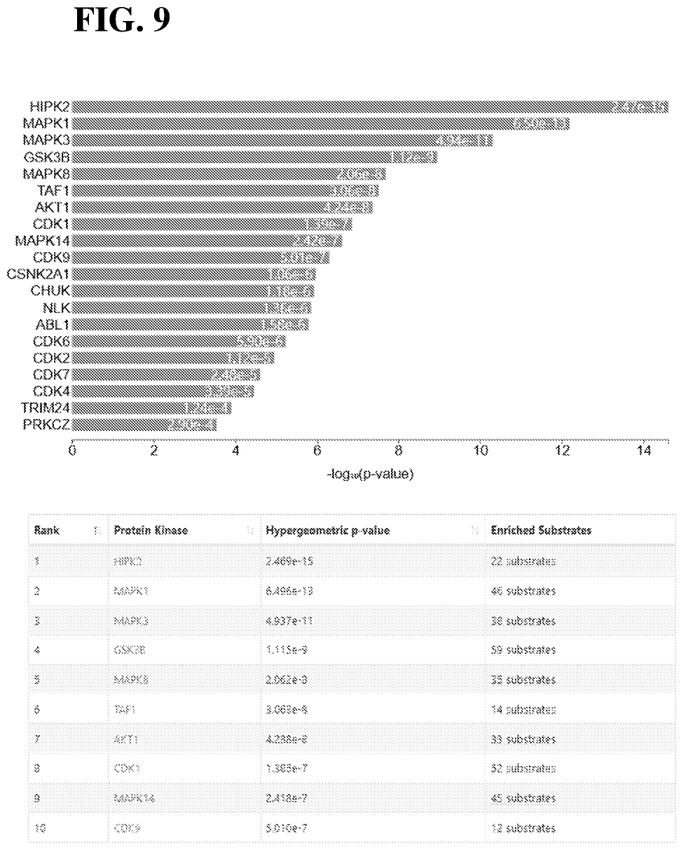

Next, the 292-protein CAD signature was inserted into the and Kinase Enrichment Analysis (KEA, Lachmann A & Ma′ayan A. Bioinformatics, 25: 684-6, 2009) to link the CAD signature with potential kinase regulators. Different kinase-substrate databases were used in order to compute the kinase enrichment probability based on the distribution of kinase-substrate proportions found to be associated with the input list of the 292 CAD proteins. Twenty proteins were statistical significantly enriched in the 292-protein CAD signature (FIG. 9). The top two kinases predicted to regulated the 292-protein CAD network were HIPK2 and MAPK1.

The transcription factor and kinase enrichment analyses revealed that the blood plasma proteomic analysis, in addition to its ability to identify a protein signature that has predictive ability to identify CAD, also contributes to the identification of novel genes that could relate with CAD pathobiology.

D. Organization of the 292-Protein CAD Signature into a Protein Protein Interaction Network and Identification of the Main Subnetworks and Key Hubs in its Subnetwork

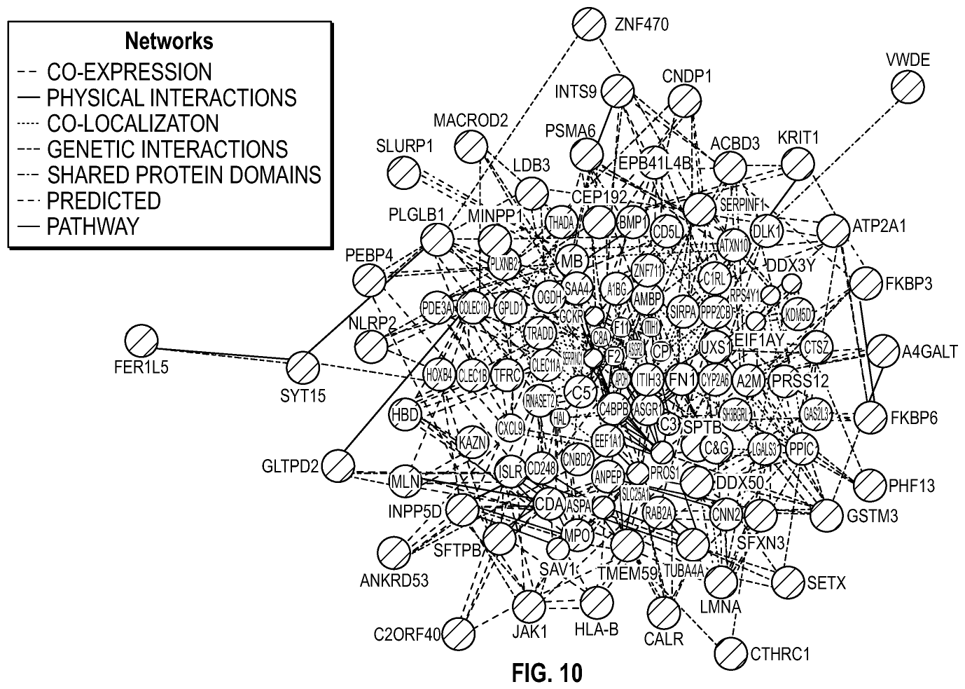

The 292-protein CAD signature was then inserted into the GeneMANIA algorithm (Warde-Farley D et al., Nucleic Acids Res, 38:W214-220, 2010) to identify the protein networks. The predicting networks of functional relationships among query and predicted proteins were identified based on predicted co-expression, co-localization, genetic interaction, physical interaction, predicted and shared protein domain data. As shown in FIG. 10, the analysis revealed a tight protein network and hundreds of protein-protein interactions, suggesting the functional significance and interaction between the 292 CAD proteins.

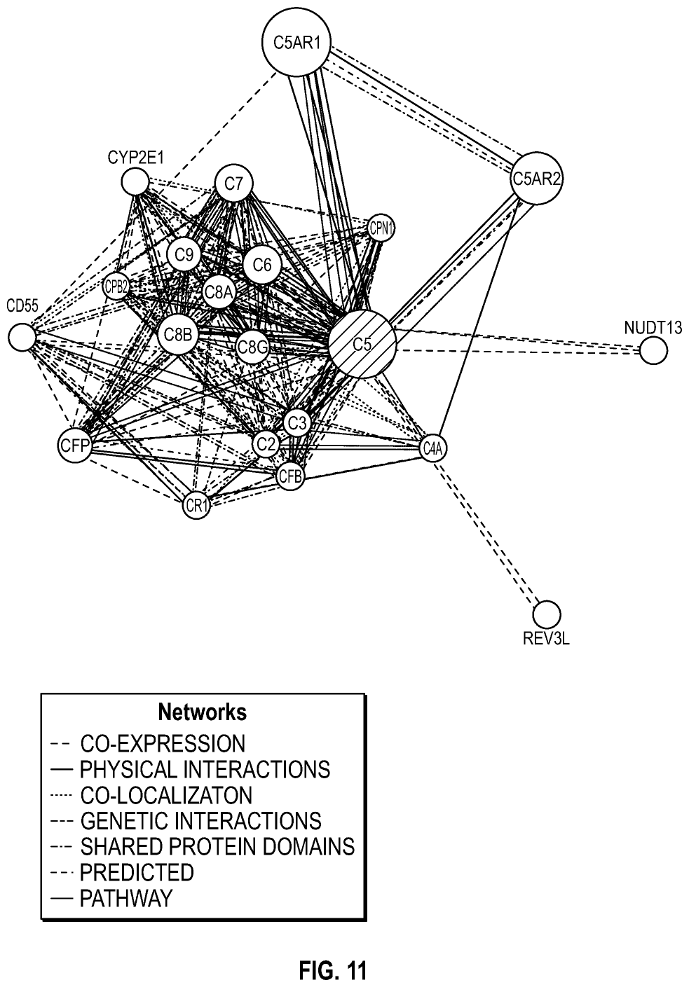

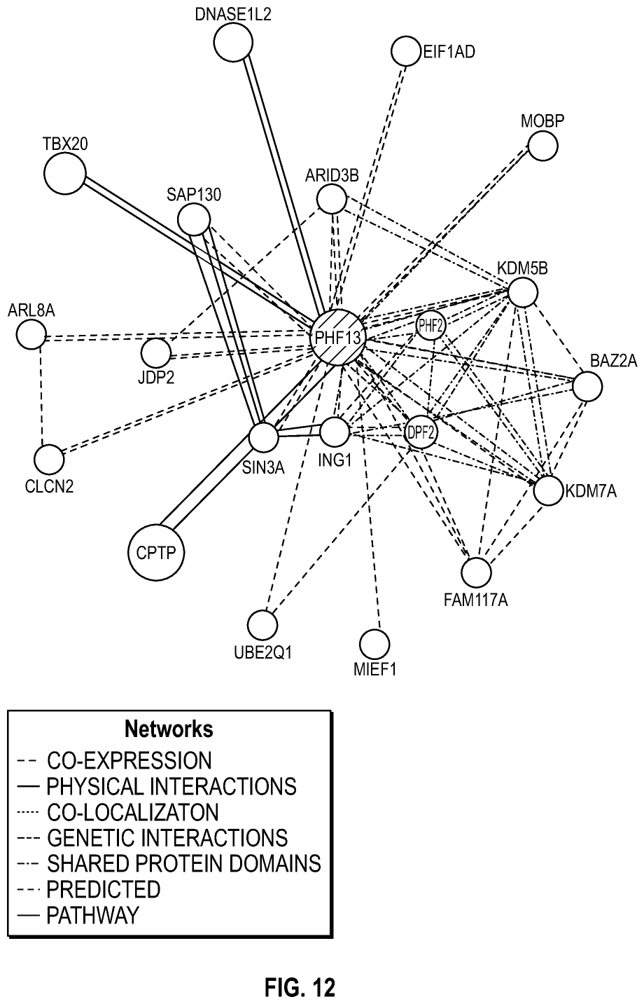

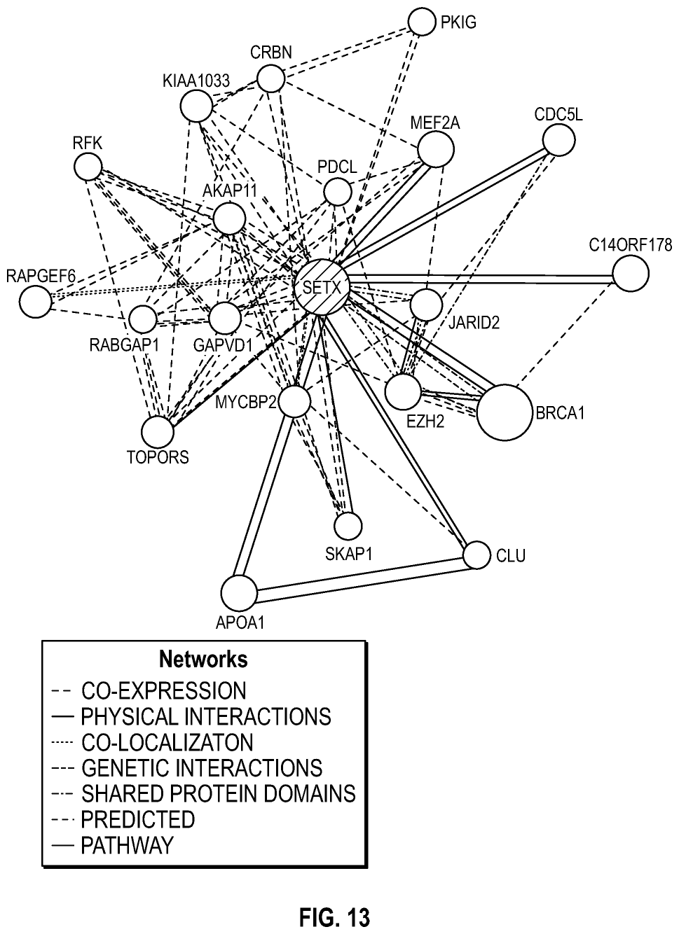

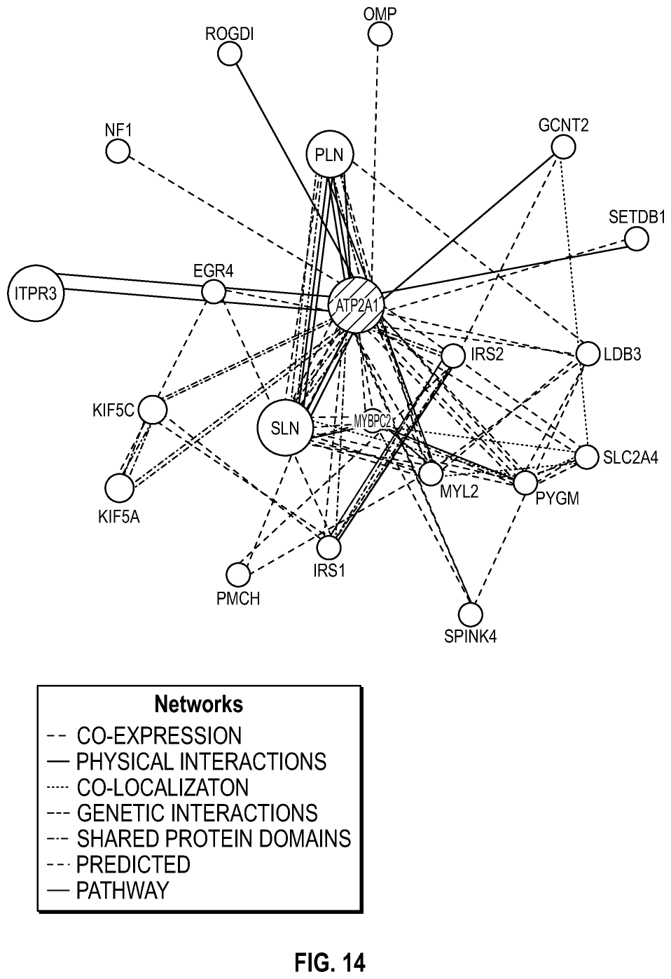

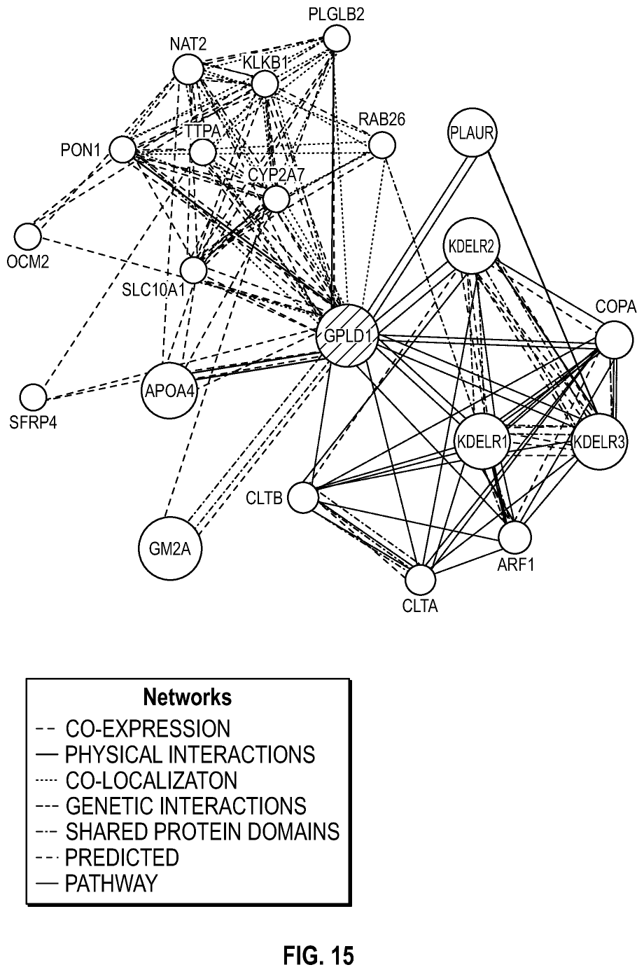

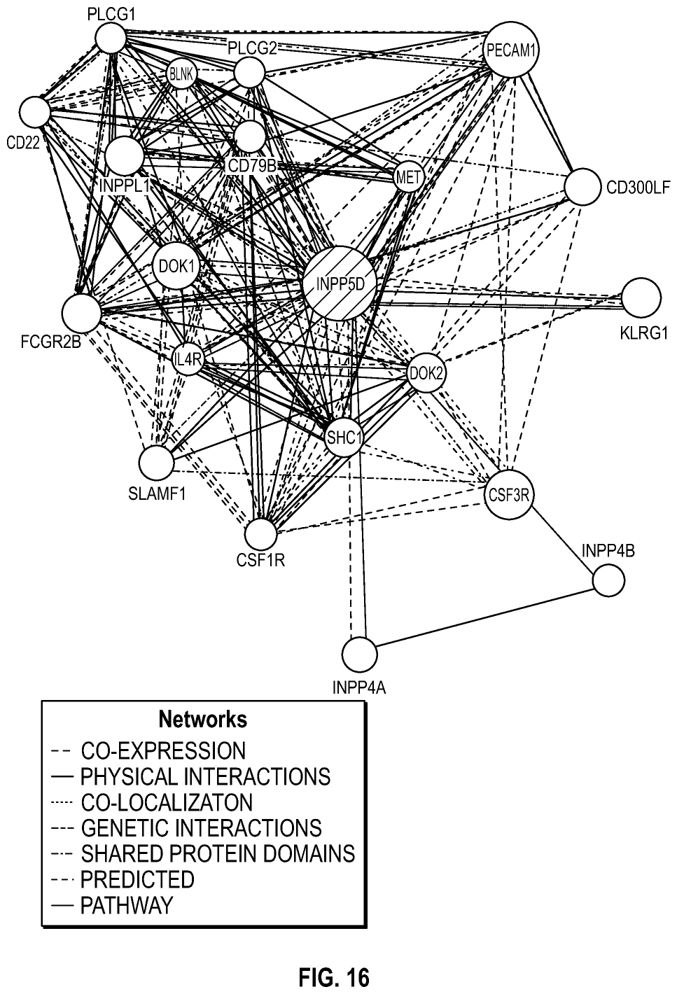

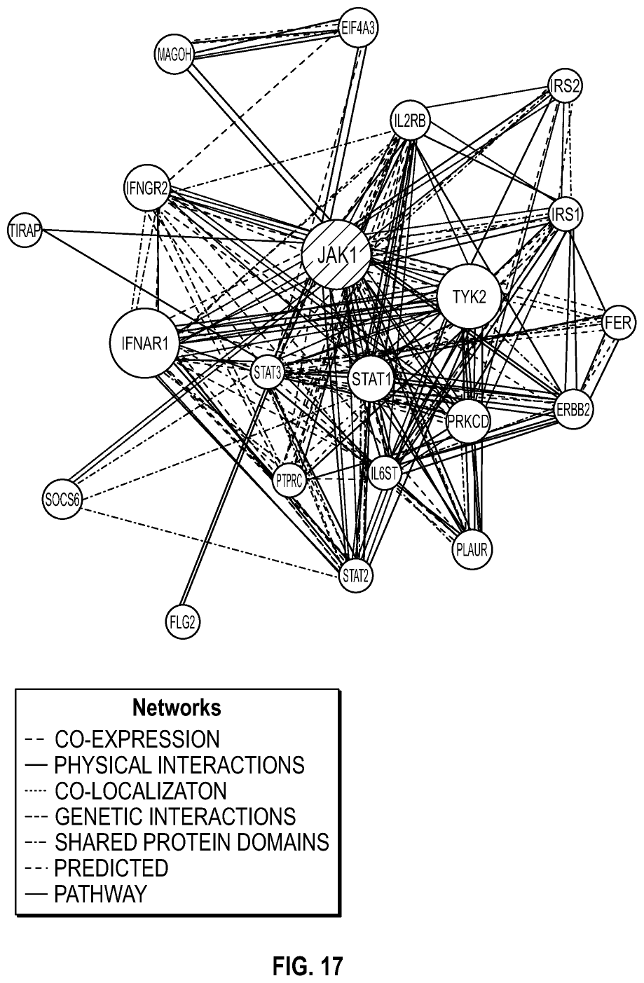

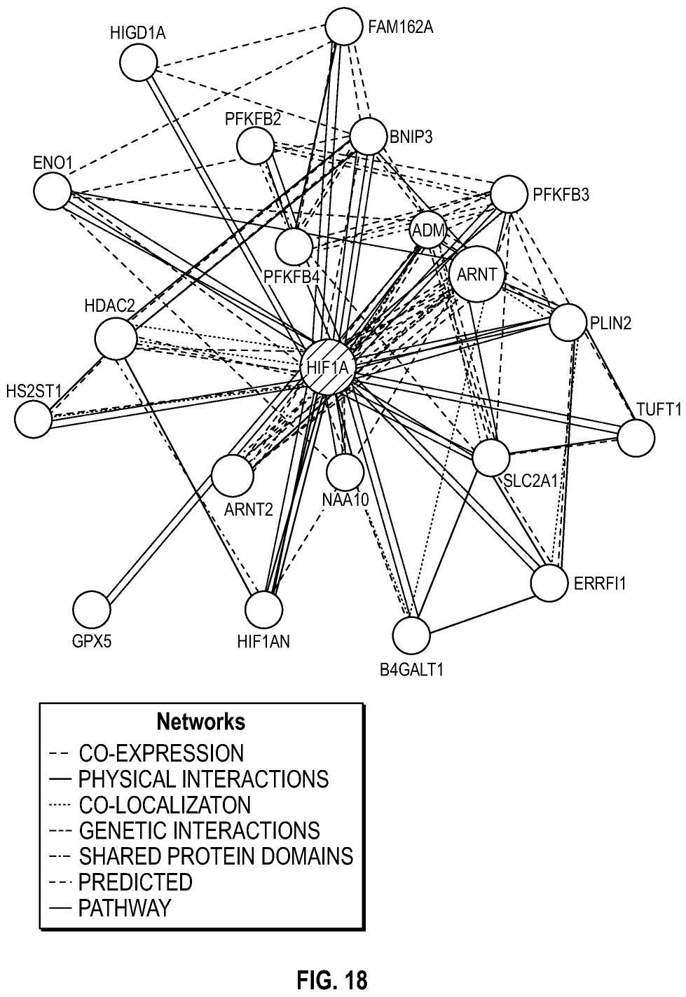

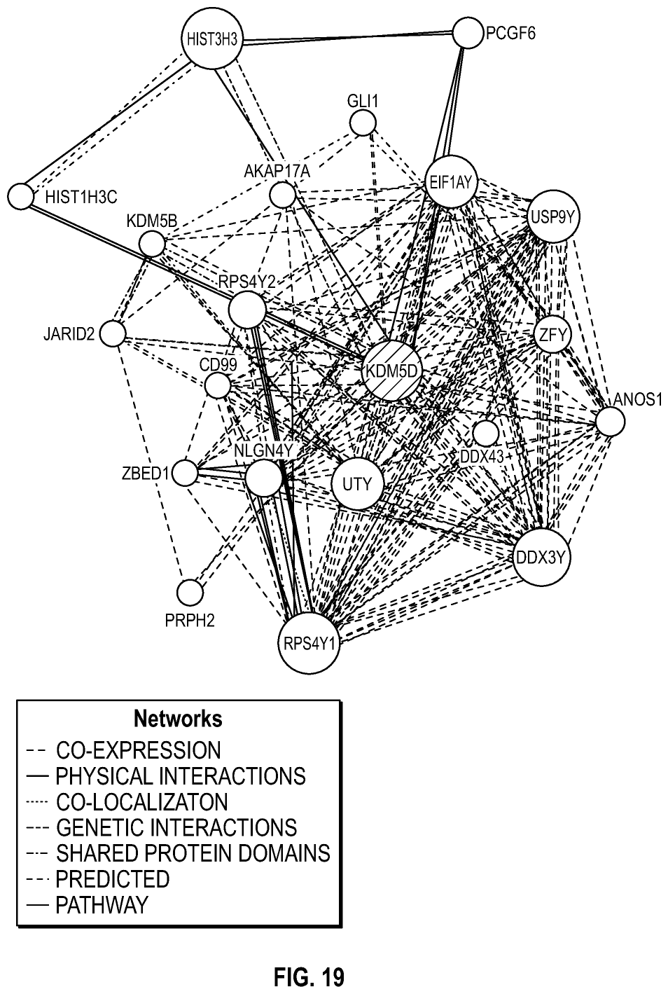

A protein subnetwork analysis was performed and also to identify the hub protein of the key subnetworks. The hubs were evaluated for their functional importance in disease cellular and animal models (for instance, for novel disease gene identification). The analysis identified the following nine subnetworks: a) complement subnetwork (hub protein: C5) (FIG. 11); b) histone regulation subnetwork (hub protein: PHF13) (FIG. 12); c) DNA damage subnetwork (hub protein: SETX) (FIG. 13); d) calcium energy subnetwork (hub protein: ATP2A1) (FIG. 14); e) metabolomics subnetwork (hub protein: GPLD1) (FIG. 15); f) cellular adhesion subnetwork (hub protein: INPP5D) (FIG. 16); g) inflammation subnetwork (hub protein: JAK1) (FIG. 17); h) hypoxia subnetwork (hub protein: HIF1A) (FIG. 18) and i) histone methylation subnetwork (hub protein: KDM5D) (FIG. 19).

Immune-related, metabolism-related, hypoxia-related, and histone-related subnetworks are highly enriched in the 292-protein CAD signature. Although the role of inflammation, hypoxia, and metabolism are well known and described to be involved in CAD pathogenesis, the data demonstrate for the first-time that histone regulatory genes, such PHF13, JARID2, and ARID3B, may be involved in CAD pathobiology.

Taken together, this analysis revealed a tightly connected protein network with hundreds of protein-protein interactions, indicating a high degree of functional interaction among proteins of the proteomic signature. These findings suggest that the PROMINIA platform could also reveal novel genes involved in the pathogenesis of the disease and the patient where the blood plasma came from.

E. Drug-CAD Protein Network Analysis Reveals Known and Novel Drugs that could have Therapeutic Potential in CAD Through Targeting the 292-Protein CAD Network

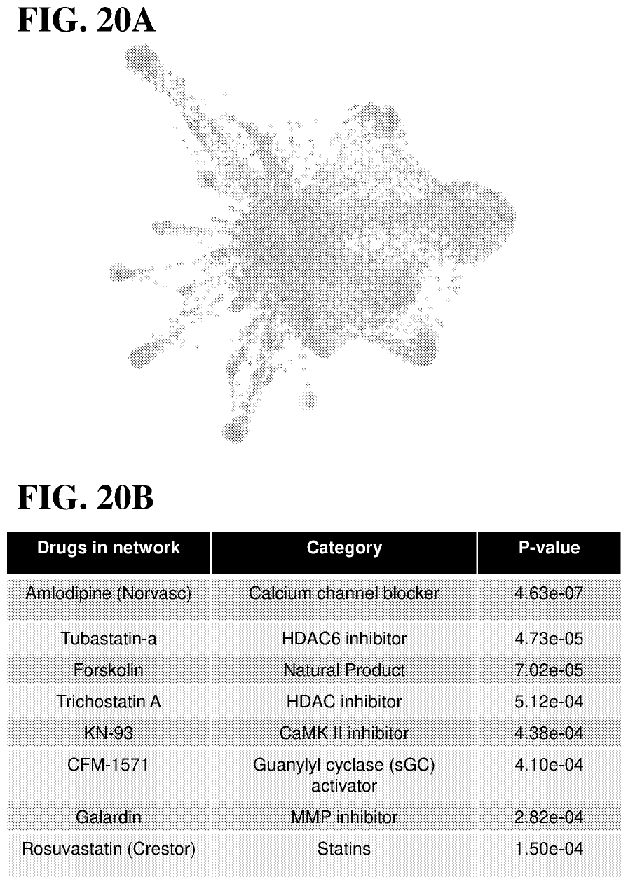

Ultimately, the proteomic signature was inserted into the L1000 FWD (Wang Z et al., Bioinformatics, 34: 2150-52, 2018) algorithm to identify FDA-approved drugs that target the hubs of protein networks represented in the 292-protein CAD signature. This analysis revealed eight drugs (p<0.001) that could be used to target the 292-protein CAD network (FIG. 20A), including Norvasc® (calcium channel blocker), tubastatin A (HDAC6 inhibitor), forskolin (natural product), trichostatin A (HDAC inhibitor), KN-93 (CaMK II inhibitor), CFM-1571 (guanylyl cyclase activator), Galardin® (metalloproteinase inhibitor) and Crestor® (rosuvastatin) (FIG. 20B). These identified drugs included not only those already used in the treatment of CAD (e.g., Norvasc® and Crestor®) but also those that have not been previously used for treatment of CAD (e.g., tubastatin A). These results demonstrate the identification of drugs for use as therapeutics for the patients for which the discovery proteomic analysis was performed.

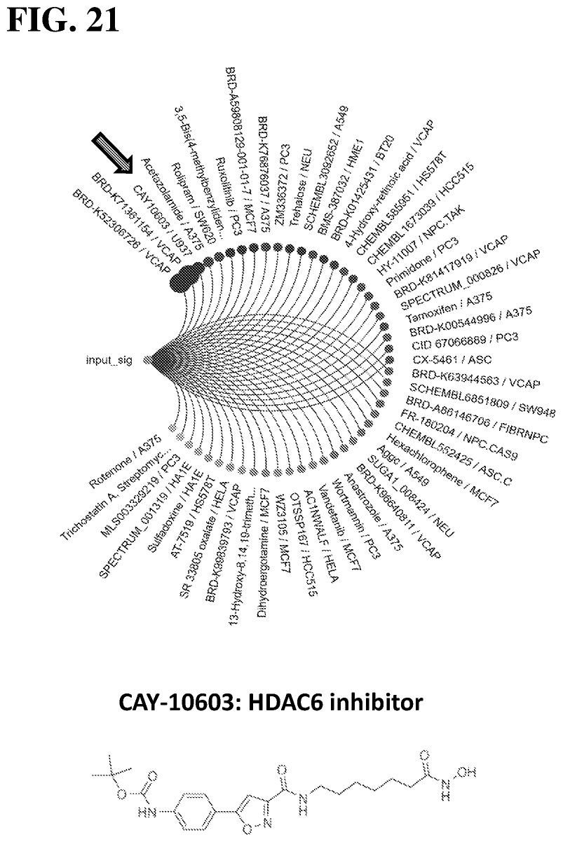

The ILINCs (https://www.biorxiv.org/content/10.1101/826271v1) chemical perturbation algorithm was used to identify novel drugs that target the hubs that could have therapeutic potential for CAD patients. This analysis identified CAY-10603 (FIG. 21), which is also an HDAC6 inhibitor, as one of the top drugs targeting the 292-protein CAD signature, suggesting that this category of epigenetic drug could have therapeutic potential for CAD patients.

Taken together, these results demonstrate that the 292-protein CAD signature included CAD-specific proteins and that the discovery proteomics platform identified and quantified these proteins in blood plasma samples of only about 10-15 pt. The PROMINIA platform identified not only known pathways and regulators involved in the pathogenesis of CAD, but also novel pathways and regulators that could be targeted for CAD therapy. Similarly, the PROMINIA platform identified novel drugs never before used in the treatment of CAD that could be used as a therapeutic. Thus, these results demonstrate the predictive power of the PROMINIA platform as well as the predictive power of the discovery proteomics platform. The more complete identification of components from a sample achieved using the methods and/or devices described herein, such as shown in Example 1, further enables the identification of disease specific signaling pathways and molecular networks using PROMINIA. Such analysis was repeated and confirmed the 292-protein CAD signature demonstrating that this work flow provide reproducible results. The 292-protein CAD signature was not independently verified by other techniques, such as ELISA or Luminox due to the incompatibility and lack of feasibility of measuring all of the identified biomarkers.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed is:

1. A method for processing a test sample for a mass spectrometry analysis, the method comprising:
(a) subjecting the test sample to a size-exclusion chromatography (SEC) technique using a SEC microfluidic device,
wherein the test sample comprises one or more biomolecules and a chaotropic agent, and
wherein the SEC microfluidic device comprises a plurality of interconnected channels;
(b) collecting a plurality of fractions eluted from the SEC microfluidic device;
(c) subjecting one or more of the plurality of fractions from the SEC microfluidic device to a proteolytic technique; and
(d) individually subjecting one or more fractions from one or both of steps (b) and (c) to a reversed-phase liquid chromatography (RPLC) technique using a RPLC microfluidic device under conditions to prepare a component of each of the one or more fractions for introduction to a mass spectrometer,
wherein the RPLC microfluidic device comprises a plurality of interconnected channels comprising a reversed-phase medium, and
wherein the RPLC microfluidic device is coupled to an electrospray ionization source.

2. The method of claim 1, wherein the test sample a biological sample.

3. The method of claim 1 or 2, wherein the test sample is from an individual.

4. The method of any one of claims 1-3, wherein the test sample has a concentration of the chaotropic agent of about 5 M to about 8 M.

5. The method of any one of claims 1-4, wherein the chaotropic agent comprises guanidine or a salt thereof, guanidinium or a salt thereof, potassium or a salt thereof, lithium or a salt thereof, magnesium or a salt thereof, or sodium or a salt thereof.

6. The method of any one of claims 1-3, wherein the chaotropic agent is guanidine hydrochloride or guanidinium chloride.

7. The method of any one of claims 1-6, wherein the chaotropic agent in the test sample is from a liquid fixative.

8. The method of any one of claims 1-7, wherein the test sample has a concentration of a viscosity modifying agent of about 5% to about 40%.

9. The method of claim 8, wherein the viscosity modifying agent is glycerol.

10. The method of claim 8 or 9, wherein the test sample comprises at least about 6 M guanidine and about 10% to about 30% glycerol.

11. The method of any one of claims 1-10, wherein the test sample subjected to the SEC technique using the SEC microfluidic device has a volume of about 1 μL to about 200 μL.

12. The method of any one of claims 1-11, wherein the range of the concentration of the mobile phase chaotropic agent of the SEC technique is within about +/−40% of the pre-determined concentration of the chaotropic agent of the test sample.

13. The method of any one of claims 1-12, wherein the SEC technique comprises use of a SEC mobile phase having a concentration of a mobile phase chaotropic agent within a range of the chaotropic agent in the test sample.

14. The method of any one of claims 1-13, wherein the mobile phase chaotropic agent of the SEC technique is the same as the chaotropic agent of the test sample.

15. The method of any one of claims 1-13, wherein the mobile phase chaotropic agent of the SEC technique is different than the chaotropic agent of the test sample.

16. The method of any one of claims 1-15, wherein the SEC mobile phase comprises a mobile phase chaotropic agent at a concentration of about 4 M to about 8 M.

17. The method of any one of claims 1-16, wherein the mobile phase chaotropic agent of the SEC technique comprises guanidine or a salt thereof, guanidinium or a salt thereof, lithium or a salt thereof, magnesium or a salt thereof, or sodium or a salt thereof.

18. The method of any one of claims 1-17, wherein the mobile phase chaotropic agent of the SEC technique is selected from the group consisting of guanidine hydrochloride, guanidinium chloride, guanidinium thiocynante, lithium perchlorate, lithium acetate, magnesium chloride, potassium acetate, and sodium iodide.

19. The method of any one of claims 1-18, wherein the SEC mobile phase comprises a mobile phase viscosity modifying agent.

20. The method of claim 19, wherein the mobile phase viscosity modifying agent of the SEC technique has a concentration of about 5% to about 40%.

21. The method of claim 19 or 20, wherein the viscosity modifying agent is glycerol.

22. The method of any one of claims 19-21, wherein the mobile phase viscosity modifying agent of the SEC technique is the same as the viscosity modifying agent of the liquid fixative.

23. The method of any one of claims 19-21, wherein the mobile phase viscosity modifying agent of the SEC technique is different than the viscosity modifying agent of the liquid fixative.

24. The method of any one of claims 19-21, wherein the test sample comprises at least about 6 M guanidine and about 10% to about 30% glycerol.

25. The method of any one of claims 1-24, wherein the SEC technique is an isocratic SEC technique.

26. The method of any one of claims 1-25, wherein the SEC technique comprises use of a mobile phase flow rate of about 1 μL/minute to about 5 μL/minute.

27. The method of any one of claims 1-26, wherein the SEC technique is performed at an elevated temperature.

28. The method of any one of claims 1-27, wherein the SEC technique is performed at a temperature of about 45° C. to about 60° C.

29. The method of claim 27 or 28, wherein the SEC technique is performed at a substantially consistent temperature.

30. The method of any one of claims 1-29, wherein the SEC microfluidic device comprises a SEC medium.

31. The method of claim 30, wherein the SEC medium is a material having an average pore size of about 10 nm to about 500 nm.

32. The method of claim 30 or 31, wherein the SEC medium is an inner surface of each of the plurality of interconnected channels.

33. The method of any one of claims 1-32, wherein the inner surface material of the plurality of interconnected channels of the SEC microfluidic device has a thickness of about 0.5 μm to about 2 μm.

34. The method of any one of claims 1-33, wherein the plurality of interconnected channels of the SEC microfluidic device are configured in an open tubular format.

35. The method of any one of claims 1-34, wherein the plurality of interconnected channels of the SEC microfluidic device comprises 8 or more interconnected channels.

36. The method of claim 35, wherein the plurality of interconnected channels of the SEC microfluidic device comprises 32 interconnected channels.

37. The method of claim 35, wherein the plurality of interconnected channels of the SEC microfluidic device comprises 64 interconnected channels.

38. The method of any one of claims 1-37, wherein each of the plurality of interconnected channels of the SEC microfluidic device are in fluidic communication with an input port of the SEC microfluidic device via an upstream network of connection channels.

39. The method of claim 38, wherein the upstream network of connection channels, or portions thereof, is connected to a proximal region of each of the plurality of interconnected channels.

40. The method of claim 38 or 39, wherein the upstream network of connection channels comprises a series of diverging channels configured to split fluid flow from the input port of the SEC microfluidic device to each of the plurality of interconnected channels.

41. The method of any one of claims 1-40, wherein each of the plurality of interconnected channels of the SEC microfluidic device is in fluidic communication with an output port of the SEC microfluidic device via a downstream network of connection channels.

42. The method of claim 41, wherein the downstream network of connection channels, or portions thereof, is connected to a distal region of each of the plurality of interconnected channels.

43. The method of claim 41 or 42, wherein the downstream network of connection channels comprises a series of converging channels configured to combine fluid flow from the plurality of interconnected channels of the SEC microfluidic device to the output port.

44. The method of any one of claims 41-43, wherein the plurality of interconnected channels of the SEC microfluidic device are only connected via the upstream network of connection channels or the downstream network of connection channels.

45. The method of any one of claims 1-44, wherein each of the plurality of interconnected channels of the SEC microfluidic device has a length of about 2 cm to about 50 cm.

46. The method of any one of claims 1-45, wherein each of the plurality of interconnected channels of the SEC microfluidic device has a width of about 1 μm to about 15 μm.

47. The method of any one of claims 1-46, wherein each of the plurality of interconnected channels of the SEC microfluidic device has a depth of about 1 μm to about 15 μm.

48. The method of any one of claims 1-47, wherein the plurality of interconnected channels of the SEC microfluidic device are formed via a pillar array.

49. The method of claim 48, wherein the pillar array is an amorphous pillar array.

50. The method of claim 48, wherein the pillar array is a non-amorphous pillar array.

51. The method of any one of claims 32-50, wherein the pillar array forms an inner surface of each of the plurality of interconnected channels of the SEC microfluidic device.

52. The method of any one of claims 1-51, wherein the SEC microfluidic device comprises a quartz substrate.

53. The method of any one of claims 1-42, wherein the SEC microfluidic device comprises a monolithic substrate forming the plurality of interconnected channels.

54. The method of any one of claims 1-53, wherein the SEC microfluidic device comprises a quartz monolithic substrate.

55. The method of any one of claims 1-44, wherein the SEC microfluidic device comprises a three-dimensional (3D) printed substrate.

56. The method of any one of claims 1-55, wherein collecting the plurality of fractions eluted from the SEC microfluidic device is performed using a fraction collector.

57. The method of any one of claims 1-56, wherein each of the plurality of fractions is collected from the SEC microfluidic device based on time.

58. The method of claim 57, wherein each of the plurality of fractions is collected from the SEC microfluidic device for a period of about 30 seconds to about 5 minutes.

59. The method of claim 57 or 58, wherein each of the plurality of fractions is collected from the SEC microfluidic device for a uniform amount of time.

60. The method of claim 47 or 58, wherein a fraction of the plurality of fractions is collected from the SEC microfluidic device for a different amount of time than another fraction of the plurality of fractions.

61. The method of any one of claims 1-56, wherein each of the plurality of fractions is collected from the SEC microfluidic device based on volume of eluate from the SEC microfluidic device.

62. The method of claim 61, wherein each of the plurality of fractions collected from the SEC microfluidic device has a volume of about 1 μL to about 20 μL.

63. The method of claim 61 or 62, wherein each of the plurality of fractions collected from the SEC microfluidic device has a uniform volume.

64. The method of claim 62 or 63, wherein a fraction of the plurality of fractions collected from the SEC microfluidic device has different volume than another fraction of the plurality of fractions.

65. The method of any one of claims 1-64, wherein the plurality of fraction is about 5 to about 50 fractions.

66. The method of claim 65, wherein the plurality of fraction is about 12 to about 24 fractions.

67. The method of any one of claims 1-66, wherein the proteolytic technique comprises an enzyme-based digestion technique.

68. The method of claim 67, wherein the enzyme-based digestion technique comprise the use of an enzyme selected from the group consisting of trypsin, chymotrypsin, pepsin, LysC, LysN, AspN, GluC and ArgC, or a combination thereof.

69. The method of claim 67 or 68, wherein the enzyme-based digestion technique comprises a step of diluting the fraction eluted from the SEC microfluidic device.

70. The method of claim 69, wherein the diluting comprises admixing the fraction eluted from the SEC microfluidic device with water to reach a concentration of the chaotropic agent.

71. The method of claim 70, wherein the final concentration of the concentration of the chaotropic agent for the enzymatic digestion is about 0.5 M.

72. The method of any one of claims 67-71, wherein the enzyme-based digestion technique does not comprise a buffer exchange step.

73. The method of any one of claims 67-72, wherein the enzyme-based digestion technique does not comprise an alkylation step.

74. The method of any one of claims 67-72, wherein the enzyme-based digestion technique does not comprise a reduction step.

75. The method of any one of claims 1-66, wherein the proteolytic technique comprises a non-enzyme-based approach.

76. The method of any one of claims 1-75, wherein the method further comprises subjecting one or more of the plurality of fractions from the SEC microfluidic device and/or one or more of the plurality of fractions subjected to the proteolytic technique to a quantitative labeling technique, wherein the quantitative labeling technique is performed prior to the reversed-phase liquid chromatography (RPLC) technique using the RPLC microfluidic device.

77. The method of claim 76, wherein the quantitative labeling technique comprises use of an isobaric mass tag.

78. The method of claim 76 or 77, wherein the quantitative labeling technique comprises use of a Tandem Mass Tag (TMT).

79. The method of any one of claims 76-78, wherein the quantitative labeling technique comprises a desalting step.

80. The method of any one of claims 1-79, wherein the method further comprises admixing an internal standard with one or more of the plurality of fractions from the SEC microfluidic device and/or one or more of the plurality of fractions subjected to the proteolytic technique, wherein the admixing of the internal standard is performed prior to the reversed-phase liquid chromatography (RPLC) technique using the RPLC microfluidic device.

81. The method of claim 79, wherein the internal standard is an isotopically-labeled peptide.

82. The method of any one of claims 1-81, wherein the one or more fractions subjected to the RPLC technique comprises one or more fractions, or portions thereof, obtained from: (i) zero or more fractions obtained from the SEC microfluidic device; and (ii) one or more of the plurality of fractions subjected to the proteolytic technique.

83. The method of any one of claims 1-82, wherein each of the one or more fractions subjected to the RPLC technique comprises the respective fraction of origin admixed with an aqueous solution.

84. The method of any one of claims 1-83, wherein the fraction subjected to the RPLC technique has a volume of about 1 μL to about 50 μL.

85. The method of any one of claims 1-84, wherein the RPLC technique comprise use of a RPLC mobile phase.

86. The method of claim 85, wherein the RPLC technique comprises a mobile phase flow rate of the RPLC mobile phase of about 0.05 μL/minute to about 2 μL/minute.

87. The method of any one of claims 1-86, wherein the RPLC technique is a gradient RPLC technique.

88. The method of any one of claims 1-87, wherein the RPLC technique is performed at an elevate temperature.

89. The method of any one of claims 1-37, wherein the RPLC technique is performed at a temperature of about 30° C. to about 100° C.

90. The method of claim 88 or 89, wherein the RPLC technique is performed at a substantially consistent temperature.

91. The method of any one of claims 1-90, wherein the reversed-phased medium comprises a RPLC moiety mixture comprising two or more of the following alkyl moieties: C₂, C₄, C₈, and C₁₈.

92. The method of claim 91, wherein the RPLC moiety mixture comprises three or more of the following alkyl moieties: C₂, C₄, C₈, and C₁₈.

93. The method of claim 91, wherein the RPLC moiety mixture comprises the following alkyl moieties: C₂, C₄, C₈, and C₁₈.

94. The method of any one of claims 91-93, wherein the alkyl moieties of the RPLC moiety mixture are present in equimolar amounts.

95. The method of any one of claims 91-94, wherein the alkyl moieties of the RPLC moiety mixture are covalently coupled to surfaces of each of the plurality of interconnected channels of the RPLC microfluidic device.

96. The method of claim 95, wherein surfaces of each of the plurality of interconnected channels comprise silica (SiO₂).

97. The method of any one of claims 1-96, wherein the plurality of interconnected channels of the RPLC microfluidic device comprises 8 or more interconnected channels.

98. The method of claim 97, wherein the plurality of interconnected channels of the RPLC microfluidic device comprises 32 interconnected channels.

99. The method of claim 97, wherein the plurality of interconnected channels of the RPLC microfluidic device comprises 64 interconnected channels.

100. The method of any one of claims 1-85, wherein each of the plurality of interconnected channels of the RPLC microfluidic device are in fluidic communication with an input port of the RPLC microfluidic device via an upstream network of connection channels.

101. The method of claim 100, wherein the upstream network of connection channels, or portions thereof, is connected to a proximal region of each of the plurality of interconnected channels.

102. The method of claim 100 or 101, wherein the upstream network of connection channels comprises a series of diverging channels configured to split fluid flow from the input port of the RPLC microfluidic device to each of the plurality of interconnected channels.

103. The method of any one of claims 1-102, wherein each of the plurality of interconnected channels of the RPLC microfluidic device is in fluidic communication with an output port of the RPLC microfluidic device via a downstream network of connection channels.

104. The method of claim 103, wherein the downstream network of connection channels, or portions thereof, is connected to a distal region of each of the plurality of interconnected channels.

105. The method of claims 103 and 104, wherein the downstream network of connection channels comprises a series of converging channels configured to combine fluid flow from the plurality of interconnected channels of the RPLC microfluidic device to the output port.

106. The method of any one of claims 103-105, wherein the plurality of interconnected channels of the RPLC microfluidic device are only connected via the upstream network of connection channels or the downstream network of connection channels.

107. The method of any one of claims 1-106, wherein each of the plurality of interconnected channels of the RPLC microfluidic device has a length of about 2 cm to about 50 cm.

108. The method of any one of claims 1-107, wherein each of the plurality of interconnected channels of the RPLC microfluidic device has a width of about 1 μm to about 15 μm.

109. The method of any one of claims 1-108, wherein each of the plurality of interconnected channels of the RPLC microfluidic device has a depth of about 1 μm to about 15 μm.

110. The method of any one of claims 1-109, wherein the plurality of interconnected channels of the RPLC microfluidic device are formed via a pillar array.

111. The method of claim 110, wherein the pillar array is an amorphous pillar array.

112. The method of claim 110, wherein the pillar array is a non-amorphous pillar array.

113. The method of any one of claims 110-112, wherein the pillar array forms an inner surface of each of the plurality of interconnected channels of the RPLC microfluidic device comprises.

114. The method of any one of claims 1-113, wherein the RPLC microfluidic device comprises an online divert feature.

115. The method of claim 114, wherein the online divert feature is a valve and/or a channel.

116. The method of claim 114 or 115, wherein the online divert feature is positioned between the plurality of interconnected channels of the RPLC microfluidic device and the electrospray ionization device.

117. The method of any one of claims 1-116, wherein the RPLC microfluidic device comprises a quartz substrate.

118. The method of any one of claims 1-117, wherein the RPLC microfluidic device comprises a monolithic substrate forming the plurality of interconnected channels.

119. The method of any one of claims 1-118, wherein the RPLC microfluidic device comprises a quartz monolithic substrate.

120. The method of any one of claims 1-119, wherein the RPLC microfluidic device comprises a three-dimensional (3D) printed substrate.

121. The method of any one of claims 1-120, wherein the RPLC microfluidic device is configured in an open tubular format.

122. The method of any one of claims 1-121, wherein the RPLC microfluidic device is configured for online desalting.

123. The method of any one of claims 1-122, wherein the electrospray ionization source is a nano-electrospray ionization source.

124. The method of any one of claims 1-123, wherein the electrospray ionization source is a heated electrospray ionization source.

125. The method of any one of claims 1-124, wherein the sample is selected from the group consisting of a blood sample, cerebrospinal fluid (CSF) sample, ascitic fluid sample, seminal fluid sample, and nipple aspirate fluid sample.

126. The method of any one of claims 1-125, wherein the sample has a volume of about 10 μL to about 200 μL.

127. The method of any one of claims 1-126, wherein the sample is a blood sample.

128. The method of any one of claims 1-127, when the sample from the individual is a blood sample, the method further comprises preparing a plasma sample.

129. The method of claim 128, wherein preparing the plasms sample comprises subjecting the blood sample to a plasma generation technique.

130. The method of claim 129, wherein the plasma generation technique comprises subjecting the sample to a polysulphone medium.

131. The method of claim 130, wherein the polysulphone medium is an asymmetric polysulphone material.

132. The method of any one of claims 129-131, wherein the plasma generation technique is a capillary action filtration technique.

133. The method of any one of claims 129-132, wherein the volume of the blood sample subjected to the plasma generation technique is about 10 μL to about 200 μL.

134. The method of any one of claims 129-133, further comprising admixing the generated plasma sample with the liquid fixative to generate the test sample.

135. The method of claim 134, wherein the test sample is not further depleted prior to subjecting the test sample to the SEC technique.

136. The method of any one of claims 129-135, wherein the plasma generation technique is performed at an ambient temperature.

137. The method of any one of claims 129-136, wherein the sample has not been subjected to a depletion step prior to the plasma generation technique.

138. The method of any one of claims 1-137, further comprising subjecting the components, or products thereof, eluted from the RPLC microfluidic device to the mass spectrometer.

139. The method of claim 138, further comprising performing a mass spectrometry analysis of the components, or products thereof, of the sample using the mass spectrometer.

140. The method of claim 139, wherein the mass spectrometry analysis comprises an analysis of each fraction subjected to the RPLC technique using the RPLC microfluidic device.

141. The method of claim 139 or 140, wherein the mass spectrometry analysis comprises obtaining one or more data sets comprising information obtained from the mass spectrometer for each fraction subjected to the RPLC technique using the RPLC microfluidic device.

142. The method of claim 141, wherein a single data set comprises information obtained from the mass spectrometer from a single fraction subjected to the RPLC technique using the RPLC microfluidic device.

143. The method of claim 141 or 142, wherein each of the one or more data set comprises mass-to-charge (m/z) and abundance information for ions of the components, or products thereof, introduced to the mass spectrometer.

144. A collection of compositions obtained from any one of the methods of claims 1-143, wherein each composition of the collection of compositions is a RPLC microfluidic device eluate.

145. A method of analyzing a collection of compositions using mass spectrometry, the method comprising:
(a) subjecting each composition of the collection of compositions to a mass spectrometer; and
(b) performing a mass spectrometry analysis of each composition of the collection of compositions,
wherein the collection of compositions is obtained from a processing technique comprising fractionation of a test sample using a SEC technique comprising use of a SEC microfluidic device followed by application of each fraction, or a product thereof, to a RPLC technique comprising use of a RPLC microfluidic device.

146. The method of claim 145, wherein the SEC fraction is further processed via a proteolysis technique.

147. The method of any of claims 141-143, further comprising, based on at least one of the one or more data sets, determining the identities of each of a plurality of the one or more biomolecules in the test sample.

148. The method of claim any of claims 141-143 and 147, further comprising, based on at least one of the one or more data sets, measuring the quantities of each of a plurality of the one or more biomolecules in the test sample.

149. The method of claim 147 or 148, further comprising identifying a signature comprising one or more identified biomolecules from the determined identities.

150. The method of claim 149, wherein the identifying further comprises selecting a subset of the one or more identified biomolecules based on the measured quantities of the one or more identified biomolecules.

151. The method of any of claims 148-150, wherein the subset of the one or more identified biomolecules is selected based on differential measured quantities of the one or more identified biomolecules compared to a reference sample.

152. The method of any of claims 141-143, further comprising identifying a signature comprising one or more identified biomolecules, the identifying comprising:
based on at least one of the one or more data sets, measuring the quantities of each of a plurality of the one or more biomolecules in the test sample;
selecting a subset of the plurality of the one or more biomolecules in the sample based on the measured quantities; and
determining the identities of each of the subset of the plurality of the one or more biomolecules in the test sample.

153. The method of claim 152, wherein the subset of the plurality of the one or more biomolecules in the test sample is selected based on differential measured quantities of the plurality of the one or more biomolecules in the test sample compared to a reference sample.

154. The method of claim 151 or 153, wherein the test sample is a sample from a diseased subject and the reference sample is a sample from a healthy subject or a control subject.

155. The method of claim 151 or 153, wherein the test sample is a sample from a subject having a pre-condition related to a disease and the reference sample is a sample from a healthy subject or a control subject.

156. The method of claim 151 or 153, wherein the test sample is a sample from a subject with a disease in an active state and the reference sample is a sample from a subject with the disease in an inactive state, optionally wherein the inactive state is remission.

157. The method of claim 151 or 153, wherein the test sample is a sample from a subject with a disease at an advanced stage and the reference sample is a sample from a subject with the disease at an early stage.

158. A signature comprising a plurality of the identified biomolecules or a subset thereof identified by the method of any of claims 149-157.

159. A signature comprising the subset of identified biomolecules identified by the method of any of claims 150-158.

160. The method of any of claims 147-157, further comprising providing all or a subset of the identified biomolecules of the signature as input to one or more processes configured to perform gene enrichment analysis, one or more processes configured to perform pathway analysis, and/or one or more processes configured to perform network analysis.

161. A method of analyzing biomolecules of a sample, the method comprising providing the identified biomolecules of the signature of claim 158 or 159 as input to one or more processes configured to perform gene enrichment analysis, one or more processes configured to perform pathway analysis, and/or one or more processes configured to perform network analysis.

162. The method of claim 160 or 161, wherein identified biomolecules of one or more molecular types of the signature are provided as the input.

163. The method of claim 162, wherein the one or more molecular types comprise proteins.

164. The method of claim 163, wherein the one or more molecular types consist only of proteins.

165. The method of any of claims 160-164, wherein the one or more processes configured to perform gene enrichment analysis comprise a process configured to identify one or more gene ontologies each associated with at least one of the identified biomolecules of the signature provided as input, or at least one of the products thereof.

166. The method of any of claims 160-165, wherein the one or more processes configured to perform gene enrichment analysis comprise:
a process configured to identify one or more cellular component gene ontologies each associated with at least one of the identified biomolecules of the signature provided as input, or at least one of the products thereof;
a process configured to identify one or more molecular pathway gene ontologies each associated with at least one of the identified biomolecules of the signature provided as input, or at least one of the products thereof; and/or
a process configured to identify one or more biological process gene ontologies each associated with at least one of the identified biomolecules of the signature provided as input, or at least one of the products thereof.

167. The method of any of claims 160-166, wherein the one or more processes configured to perform gene enrichment analysis comprise a process configured to identify one or more regulators of at least one of the identified biomolecules of the signature provided as input, or at least one of the products thereof.

168. The method of any of claims 160-167, wherein the one or more processes configured to perform gene enrichment analysis comprise:
a process configured to identify one or more transcription factors regulating at least one of the identified biomolecules of the signature provided as input, or at least one of the products thereof; and/or
a process configured to identify one or more kinases regulating at least one of the identified biomolecules of the signature provided as input, or at least one of the products thereof.

169. The method of any of claims 160-168, wherein the one or more processes configured to perform pathway analysis comprise a process configured to identify one or more pathways each associated with at least one of the identified biomolecules of the signature provided as input, or at least one of the products thereof.

170. The method of any of claims 160-169, wherein the one or more processes configured to perform pathway analysis comprise:
a process configured to identify one or more molecular pathways each associated with at least one of the identified biomolecules of the signature provided as input, or at least one of the products thereof;
a process configured to identify one or more signaling pathways each associated with at least one of the identified biomolecules of the signature provided as input, or at least one of the products thereof; and/or
a process configured to identify one or more metabolic pathways each associated with at least one of the identified biomolecules of the signature provided as input, or at least one of the products thereof.

171. The method of any of claims 160-170, wherein the one or more processes configured to perform network analysis comprise a process configured to identify one or more networks each associated with at least one of the identified biomolecules of the signature provided as input, or at least one of the products thereof.

172. The method of any of claims 160-171, wherein the one or more processes configured to perform network analysis comprise:
a process configured to identify one or more molecular pathways each associated with at least one of the identified biomolecules of the signature provided as input, or at least one of the products thereof; and/or
a process configured to identify one or more signaling pathways each associated with at least one of the identified biomolecules of the signature provided as input, or at least one of the products thereof.

173. The method of any of claims 160-172, wherein the one or more processes configured to perform network analysis comprise a process configured to identify one or more hubs of one or more networks each associated with at least one of the identified biomolecules of the signature provided as input, or at least one of the products thereof.

174. The method of any of claims 160-173, wherein the one or more processes configured to perform network analysis comprise a process configured to identify one or more drugs each targeting at least one of the identified biomolecules of the signature provided as input, or at least one of the products thereof, optionally wherein the process is configured to identify one or more drugs each targeting at least one hub of a network comprising a plurality of the identified biomolecules of the signature provided as input.

175. The method of any of claims 160-174, wherein the one or more processes configured to perform network analysis comprises two processes configured to identify one or more drugs each targeting at least one of the identified biomolecules of the signature provided as input, or at least one of the products thereof, optionally wherein the two processes are configured to identify one or more drugs each targeting at least one hub of a network comprising a plurality of the identified biomolecules of the signature provided as input.

176. A method of analyzing a signature of identified biomolecules, comprising providing a plurality of identified biomolecules to each of a plurality of processes each configured to perform gene enrichment analysis, pathway analysis, or network analysis, wherein:
the providing is performed in any order;
the plurality of identified biomolecules comprises a protein set, a transcriptomic set, a peptide set, and/or a metabolite set; and
the plurality of processes comprise:
a process configured to perform gene enrichment analysis to identify one or more gene ontologies each associated with at least one of the plurality of identified biomolecules provided as input, or at least one of the products thereof;
a process configured to perform pathway analysis to identify one or more signaling pathways each associated with at least one of the plurality of identified biomolecules provided as input, or at least one of the products thereof;
a process configured to perform gene enrichment analysis to identify one or more transcription factors regulating at least one of the plurality of identified biomolecules provided as input, or at least one of the products thereof;
a process configured to perform gene enrich analysis to identify one or more kinases regulating a gene product of at least one of the plurality of identified biomolecules provided as input, or at least one of the products thereof;
a process configured to perform network analysis to identify one or more networks each associated with at least one of the plurality of identified biomolecules provided as input, or at least one of the products thereof; and
each of two processes configured to perform network analysis to identify one or more drugs each targeting at least one of the plurality of identified biomolecules provided as input, or at least one of the products thereof.

177. A method of analyzing a protein signature, comprising providing a plurality of proteins to each of a plurality of processes each configured to perform gene enrichment analysis, pathway analysis, or network analysis, wherein the providing is performed in any order, and the plurality of processes comprise:
a process configured to perform gene enrichment analysis to identify one or more gene ontologies each associated with at least one of the plurality of proteins provided as input, or at least one of the products thereof;
a process configured to perform pathway analysis to identify one or more signaling pathways each associated with at least one of the plurality of proteins provided as input, or at least one of the products thereof;
a process configured to perform gene enrichment analysis to identify one or more transcription factors regulating at least one of the plurality of proteins provided as input, or at least one of the products thereof;
a process configured to perform gene enrich analysis to identify one or more kinases regulating a gene product of at least one of the plurality of proteins provided as input, or at least one of the products thereof;
a process configured to perform network analysis to identify one or more networks each associated with at least one of the plurality of proteins provided as input, or at least one of the products thereof; and
each of two processes configured to perform network analysis to identify one or more drugs each targeting at least one of the plurality of proteins provided as input, or at least one of the products thereof.

178. A size-exclusion chromatography (SEC) microfluidic device comprising:
an input port;
an upstream network of connection channels; and
a plurality of interconnected channels,
wherein each channel of the plurality of interconnected channels is in an open tubular format,
wherein each channel of the plurality of interconnected channels comprises an inner surface comprising a SEC medium, and
wherein each channel of the plurality of interconnected channels is in fluidic communication with the input port via the upstream network of connection channels.

179. The SEC microfluidic device of claim 178, wherein the inner surface comprising the SEC medium has a thickness of about 0.5 μm to about 2 μm.

180. The SEC microfluidic device of claim 178 or 179, wherein the SEC medium is a material having an average pore size of about 10 nm to about 500 nm.

181. The SEC microfluidic device of any one of claims 178-180, wherein the plurality of interconnected channels of the SEC microfluidic device comprises between 8 and 100 interconnected channels.

182. The SEC microfluidic device of any one of claims 178-181, wherein the plurality of interconnected channels of the SEC microfluidic device comprises 8 or more interconnected channels.

183. The SEC microfluidic device of any one of claims 178-182, wherein the plurality of interconnected channels of the SEC microfluidic device comprises 32 interconnected channels.

184. The SEC microfluidic device of any one of claims 178-182, wherein the plurality of interconnected channels of the SEC microfluidic device comprises 64 interconnected channels.

185. The SEC microfluidic device of any one of claims 178-184, wherein the upstream network of connection channels, or portions thereof, is connected to a proximal region of each of the plurality of interconnected channels.

186. The SEC microfluidic device of any one of claims 178-185, wherein the upstream network of connection channels comprises a series of diverging channels configured to split fluid flow from the input port of the SEC microfluidic device to each of the plurality of interconnected channels.

187. The SEC microfluidic device of any one of claims 178-186, wherein each of the plurality of interconnected channels of the SEC microfluidic device is in fluidic communication with an output port of the SEC microfluidic device via a downstream network of connection channels.

188. The SEC microfluidic device of claim 187, wherein the downstream network of connection channels comprises a series of converging channels configured to combine fluid flow from the plurality of interconnected channels of the SEC microfluidic device to the output port.

189. The SEC microfluidic device of any one of claims 178-188, wherein each of the plurality of interconnected channels of the SEC microfluidic device has a length of about 2 cm to about 30 cm.

190. The SEC microfluidic device of any one of claims 178-189, wherein each of the plurality of interconnected channels of the SEC microfluidic device has a width of about 1 μm to about 15 μm.

191. The SEC microfluidic device of any one of claims 178-190, wherein each of the plurality of interconnected channels of the SEC microfluidic device has a depth of about 1 μm to about 15 μm.

192. The SEC microfluidic device of any one of claims 178-191, wherein the plurality of interconnected channels of the SEC microfluidic device are formed via a pillar array.

193. The SEC microfluidic device of claim 192, wherein the pillar array is an amorphous pillar array.

194. The SEC microfluidic device of claim 192, wherein the pillar array is a non-amorphous pillar array.

195. The SEC microfluidic device of any one of claims 192-194, wherein the pillar array forms an inner surface of each of the plurality of interconnected channels of the SEC microfluidic device.

196. The SEC microfluidic device of any one of claims 178-195, wherein the SEC microfluidic device comprises a quartz substrate.

197. The SEC microfluidic device of any one of claims 178-196, wherein the SEC microfluidic device comprises a monolithic substrate forming the plurality of interconnected channels.

198. The SEC microfluidic device of any one of claims 178-197, wherein the SEC microfluidic device comprises a quartz monolithic substrate.

199. The SEC microfluidic device of any one of claims 178-198, wherein the SEC microfluidic device comprises a three-dimensional (3D) printed substrate.

200. A reversed-phase liquid chromatography (RPLC) microfluidic device comprising:
an input port;
an upstream network of connection channels; and
a plurality of interconnected channels,
wherein each channel of the plurality of interconnected channels is in an open tubular format,
wherein each channel of the plurality of interconnected channels comprises an inner surface comprising a RPLC medium, and
wherein each channel the plurality of interconnected channels is in fluidic communication with the input port via the upstream network of connection channels.

201. The RPLC microfluidic device of claim 200, wherein the RPLC medium comprises an alkyl moiety having about 2 to about 20 carbons.

202. The RPLC microfluidic device of claim 200 or 201, wherein the RPLC medium comprises one or more of C₂, C₄, C₈, and C₁₈.

203. The RPLC microfluidic device of any one of claims 200-202, wherein RPLC medium comprises a RPLC moiety mixture comprising two or more of the following alkyl moieties: C₂, C₄, C₈, and C₁₈.

204. The RPLC microfluidic device of claim 203, wherein the RPLC moiety mixture comprises three or more of the following alkyl moieties: C₂, C₄, C₈, and C₁₈.

205. The RPLC microfluidic device of claim 203 or 204, wherein the RPLC moiety mixture comprises the following alkyl moieties: C₂, C₄, C₈, and C₁₈.

206. The RPLC microfluidic device of any one of claims 203-205, wherein the alkyl moieties of the RPLC moiety mixture are present in equimolar amounts.

207. The RPLC microfluidic device of any one of claims 200-206, wherein the RPLC medium is conjugated to the inner surface of each channel of the interconnected plurality of parallel channels via silica (SiO₂).

208. The RPLC microfluidic device of any one of claims 200-207, wherein the plurality of interconnected channels of the RPLC microfluidic device comprises between 8 and 100 interconnected channels.

209. The RPLC microfluidic device of any one of claims 200-208, wherein the plurality of interconnected channels of the RPLC microfluidic device comprises 8 or more interconnected channels.

210. The RPLC microfluidic device of any one of claims 200-209, wherein the plurality of interconnected channels of the RPLC microfluidic device comprises 32 interconnected channels.

211. The RPLC microfluidic device of any one of claims 200-209, wherein the plurality of interconnected channels of the RPLC microfluidic device comprises 64 interconnected channels.

212. The RPLC microfluidic device of any one of claims 200-211, wherein the upstream network of connection channels, or portions thereof, is connected to a proximal region of each of the plurality of interconnected channels.

213. The RPLC microfluidic device of any one of claims 200-212, wherein the upstream network of connection channels comprises a series of diverging channels configured to split fluid flow from the input port of the RPLC microfluidic device to each of the plurality of interconnected channels.

214. The RPLC microfluidic device of any one of claims 200-213, wherein each of the plurality of interconnected channels of the RPLC microfluidic device is in fluidic communication with an output port of the RPLC microfluidic device via a downstream network of connection channels.

215. The RPLC microfluidic device of claim 214, wherein the downstream network of connection channels comprises a series of converging channels configured to combine fluid flow from the plurality of interconnected channels of the RPLC microfluidic device to the output port.

216. The RPLC microfluidic device of any one of claims 200-215, wherein each of the plurality of interconnected channels of the RPLC microfluidic device has a length of about 2 cm to about 30 cm.

217. The RPLC microfluidic device of any one of claims 200-216, wherein each of the plurality of interconnected channels of the RPLC microfluidic device has a width of about 1 μm to about 15 μm.

218. The RPLC microfluidic device of any one of claims 200-217, wherein each of the plurality of interconnected channels of the RPLC microfluidic device has a depth of about 1 μm to about 15 μm.

219. The RPLC microfluidic device of any one of claims 200-218, wherein the plurality of interconnected channels of the RPLC microfluidic device are formed via a pillar array.

220. The RPLC microfluidic device of claim 219, wherein the pillar array is an amorphous pillar array.

221. The RPLC microfluidic device of claim 219, wherein the pillar array is a non-amorphous pillar array.

222. The RPLC microfluidic device of any one of claims 219-221, wherein the pillar array forms an inner surface of each of the plurality of interconnected channels of the RPLC microfluidic device.

223. The RPLC microfluidic device of any one of claims 219-221, wherein the RPLC microfluidic device comprises a quartz substrate.

224. The RPLC microfluidic device of any one of claims 219-223, wherein the RPLC microfluidic device comprises a monolithic substrate forming the plurality of interconnected channels.

225. The RPLC microfluidic device of any one of claims 219-224, wherein the RPLC microfluidic device comprises a quartz monolithic substrate.

226. The RPLC microfluidic device of any one of claims 219-225, wherein the RPLC microfluidic device comprises a three-dimensional (3D) printed substrate.

227. A method for processing a test sample, the method comprising:
(a) subjecting the test sample to a size-exclusion chromatography (SEC) technique using a SEC microfluidic device,
wherein the test sample comprises one or more biomolecules and a chaotropic agent, and
wherein the SEC microfluidic device comprises a plurality of interconnected channels;
(b) collecting one or more fractions eluted from the SEC microfluidic device;
(c) subjecting one or more of the fractions collected from the SEC microfluidic device to a proteolytic technique; and
(d) subjecting one or more of fractions to a reversed-phase liquid chromatography (RPLC) technique to prepare a fraction for introduction to a mass spectrometer,
wherein the one or more RPLC-fractions comprises (i) zero or more fractions obtained from the SEC microfluidic device; and (ii) zero or more fractions subjected to the proteolytic technique.

228. A method of analyzing a composition, the method comprising:
(a) subjecting the composition to a mass spectrometer; and
(b) performing a mass spectrometry analysis of the composition,
wherein the composition is obtained from a processing technique comprising fractionation of a sample using a SEC technique comprising use of a SEC microfluidic device followed by application of one or more fractions from the SEC microfluidic technique, or a product thereof, to a RPLC technique.

229. A method of analyzing a signature of identified components, comprising performing gene enrichment analysis, pathway analysis, and network analysis in any order, wherein:
the signature of identified components comprises a protein set, a transcriptomic set, a peptide set, and/or a metabolite set; and
the performing comprises:
a process configured to perform gene enrichment analysis;
a process configured to perform pathway analysis;
a process configured to perform gene enrichment analysis; and
a process configured to perform network analysis to identify drug targets.

230. A method of subjecting an individual to a coronary artery disease (CAD) diagnosis determination, the method comprising:
(a) obtaining mass spectrometry (MS) data from a sample, or a derivative thereof, obtained from the individual; and
(b) analyzing the MS data according to a CAD proteomic signature,
wherein the CAD proteomic signature comprises one or more biomarkers of Table 1; and
(c) determining whether the individual has the CAD proteomic signature.

231. The method of claim 230, wherein if the individual has the CAD proteomic signature, the individual is diagnosed as has having CAD.

232. A method of diagnosing an individual as having coronary artery disease (CAD), the method comprising:
(a) obtaining mass spectrometry (MS) data from a sample, or a derivative thereof, obtained from the individual; and
(b) analyzing the MS data according to a CAD proteomic signature,
wherein the CAD proteomic signature comprises one or more biomarkers of Table 1; and
(c) diagnosing the individual as having CAD based on the presence of the CAD proteomic signature.

233. A method of treating an individual having coronary artery disease (CAD), the method comprising:
(a) diagnosing an individual as having CAD according to the presence of a CAD proteomic signature in a sample, or a derivative thereof, obtained from the individual,
wherein the CAD proteomic signature comprises one or more biomarkers of Table 1; and
(b) administering to the individual a CAD treatment.

234. The method of claim 233, wherein the presence of the CAD proteomic signature is determined by analyzing MS data according to the CAD proteomic signature.

235. The method of claim 234, further comprising obtaining the MS data from the sample, or the derivative thereof, obtained from the individual.

236. The method of any one of claims 233-235, wherein the CAD treatment comprises a life style adjustment.

237. The method of any one of claims 233-236, wherein the CAD treatment comprises a pharmaceutical intervention.

238. The method of claim 237, wherein the pharmaceutical intervention comprises administration of a drug selected from the group consisting of a calcium channel blocker, histone deacetylase (HDAC) inhibitor (such as HDAC6), Ca2+/calmodulin (CaM)-dependent protein kinase II (CaMK II) inhibitor, guanylyl cyclase (sGC) activator, MMP inhibitor, statin, and anti-hypertesnive.

239. The method of claim 237 or 238, wherein the pharmaceutical intervention comprises a drug is selected from the group consisting of amlodipine, tubastatin-a, forskolin, trichostatin A, KN-93, CFM-1571, ilomastat, CAY-10603, and rosuvastatin, or a pharmaceutical salt thereof.

240. The method of claim 237 or 238, wherein the drug is selected from the group consisting of BRD-K52306726, BRD-K71361154, acetazolamide, rolipram, ruxolitinib, BRD-A59808129-001-01-7, BRD-K76876037, ZM336372, trehalose, SCHEMBL3092652, BMS-387032, BRD-K01425431, 4-hydroxy-retinoic acid, CHEMBL585951, CHEMBL1673039, HY-11007, primidone, BRD-K81417919, SPECTRUM_000826, tamoxifen, BRD-K00544996, CID 67066889, CX-5461, BRD-K63944563, SCHEMBL6851809, BRD-A86146706, FR-180204, CHEMBL552425, hexachlorophene, Aggc, SUGA1_008424, BRD-K96640811, anastrozole, wortmannin, vandetanib, AC1NWALF, OTSSP167, WZ3105, dihydroergotamine, BRD-K99839793, SR 33805 oxalate, AT-7519, sulfadoxine, SPECTRUM_001319, MLS003329219, trichostatin A, and rotenone, or a pharmaceutical salt thereof.

241. A method for detecting a coronary artery disease (CAD) proteomic signature of an individual,
(a) obtaining mass spectrometry (MS) data from a sample, or a derivative thereof, obtained from the individual; and
(b) analyzing the MS data according to a CAD proteomic signature to detect the CAD proteomic signature,
wherein the CAD proteomic signature comprises one or more biomarkers of Table 1.

242. The method of claim 241, wherein the individual is suspected of having CAD.

243. The method of any one of claims 230-242, wherein the CAD proteomic signature comprises increased expression of the one or more biomarkers according to Table 1 as compared to a reference.

244. The method of any one of claims 230-243, wherein the CAD proteomic signature comprises decreased expression of the one or more biomarkers according to Table 1 as compared to a reference.

245. The method of any one of claims 230-244, wherein the CAD proteomic signature comprises one or more biomarkers associated with a calcium signaling pathway, histone regulation, HIF-1 signaling pathway, cAMP signaling pathway, beta-adrenergic signaling pathway, PI3K-Akt signaling pathway, complement and/or coagulation cascade, sphingolipid signaling pathway, natural killer cell mediated cytotoxicity, adipocytoknie signaling pathway, DNA damage, calcium energy, metaboloimcs, cellular adhesion, inflammation, hypoxia, and histone methylation.

246. The method of any one of claims 230-245, wherein the one or more biomarkers comprise a subset thereof comprising one or more biomarkers associated with a transcription factor.

247. The method of any one of claims 230-246, wherein the one or more biomarkers comprise a subset thereof comprising one or more biomarkers associated with a kinase.

248. The method of any one of claims 230-247, wherein the one or more biomarkers comprise at least 10 biomarkers of Table 1.

249. The method of any one of claims 230-248, wherein the one or more biomarkers comprise at least 25 biomarkers of Table 1.

250. The method of any one of claims 230-249, wherein the one or more biomarkers comprise at least 50 biomarkers of Table 1.

251. The method of any one of claims 230-250, wherein the one or more biomarkers comprise all biomarkers of Table 1.

252. The method of any one of claims 230-251, further comprising obtaining the sample from the individual.

253. The method of any one of claims 230-252, wherein the sample, or the derivative thereof, is a blood sample or a derivative thereof.

254. The method of claim 253, wherein the sample, or the derivative thereof, is a plasma sample.

255. The method of claim 254, wherein the sample, or the derivative thereof, comprises a liquid fixative.

256. The method of any one of claims 230-255, wherein the obtaining MS data from the sample, or the derivative thereof, comprises performing a mass spectrometry analysis of the sample, or the derivative thereof, using a mass spectrometer.

257. The method of claim 256, wherein the mass spectrometry analysis is performed according to the method of claims 140-143.

258. The method of any one of claims 230-257, wherein the analyzing the MS data according to the CAD proteomic signature comprises subjecting the MS data to a method of any one of claims 161-177.

259. The method of any one of claims 230-258, wherein the analyzing the MS data according to the CAD proteomic signature comprises assessing the presence or absence or level of each of the one or more biomarkers of the CAD proteomic signature in the MS data.

260. The method of any one of claims 230-259, further comprising performing one or more of the following factor assessments of the individual: sex, age, body mass index (BMI), systolic blood pressure, diastolic blood pressure, total cholesterol, HDL, LDL, triglycerides, hyperlipidemia, hypertension, diabetes mellitus, insulin resistance, kidney disease, smoking status, level of physical activity, level of sleep, or quality of nutrition.

261. The method of any one of claims 230-260, further comprising performing a medical procedure on the individual to assess the presence of CAD.
